(19) United States
(12) Patent Application Publication
(10) Pub. No.: US 2023/0355571 A1
(43) Pub. Date: Nov. 9, 2023

(54) METHOD FOR TREATING VIRAL AND BACTERIAL INFECTION THROUGH INHALATION THERAPY

(71) Applicants: George Edward Hoag, Storrs, CT (US); John Salerno, New York, NY (US)

(72) Inventors: George Edward Hoag, Storrs, CT (US); John Salerno, New York, NY (US)

(21) Appl. No.: 17/920,478

(22) PCT Filed: Apr. 21, 2021
(86) PCT No.: PCT/US2021/028451

§ 371 Date: Oct. 21, 2022

Related U.S. Application Data
(60) Provisional application No. 63/014,089, filed on Apr. 22, 2020.

(51) Int. Cl. A61K 31/352; A61K 31/015; A61K 31/047; A61K 31/198; A61K 47/26; A61K 9/00; A61P 31/14; A61K 47/02; A61P 11/06

(52) U.S. Cl. CPC A61K 31/352; A61K 31/015; A61K 31/047; A61K 31/198; A61K 47/26; A61K 9/0078; A61P 31/14; A61K 47/02; A61P 11/06

(57) ABSTRACT

Liquid pharmaceutical liquid compositions that are orally administered and methods for their use by administration to the lungs for multifunctional treatment of lung and respiratory diseases.

### CROSS-REFERENCE TO RELATED APPLICATIONS

This international application claims the benefit of the filing date of U.S. Provisional Application No. 63/014,089, filed Apr. 22, 2020, which is incorporated by reference in its entirety herein.

### TECHNICAL FIELD

Liquid pharmaceutical liquid compositions that are orally administered and methods for their use by administration to the lungs for multifunctional treatment of lung and respiratory diseases.

### BACKGROUND OF THE INVENTION

### Respiratory Tract Infection

Acute respiratory tract illnesses are illnesses of humans and a cause of disability and days lost from school or work. Lower respiratory tract infections are the leading cause of infectious disease deaths worldwide and are the fifth leading cause of death overall. Lower respiratory tract infections (LRTIs), which generally are considered to include acute bronchitis, bronchiolitis, influenza, and pneumonia.

Many viruses have characteristic seasonal patterns. Influenza virus and respiratory syncytial virus (RSV) infections peak in winter, but other respiratory viruses such as human metapneumovirus (hMPV), parainfluenza viruses (Para), and coronaviruses (CoronaV) are also prevalent in the fall and winter. Respiratory viruses include but are not limited to adenovirus (Adeno) and rhinovirus cause illness year-round. Respiratory viruses include; adenovirus, influenza A (H1N1, H1N2 and H3N2), influenza B (FluB), influenza C (FluC), parainfluenza virus (HPIV1, HPIV2, HPIV3, HPIV4), respiratory syncytial virus (RSV), human coronavirus (HCoV-229E, HCoV-NL63, HCoV-HKUJ, HCoVOC4), human metapneumovirus (hMPV), and the severe acute respiratory syndrome-associated CoVs, SARS-CoV-1 and in 2019 SARS-CoV-2.

Respiratory tract bacterial infections include the following: Bordetella pertussis, Chlamydophila pneumoniae, Mycoplasma pneumoniae, Streptococcus pneumoniae, Klebsiella pneumoniae, Staphylococcus aureus (MSSA and MRSA), Pseudomonas aeruginosa, Escherichia coli, Haemophilus influenza, Legionella pneumophila, and Acinetobacter and Enterobacter species. Two important bacterial lower respiratory tract infections include acute exacerbations of chronic obstructive pulmonary disease (AECOPD) and community-acquired pneumonia (CAP).

Lower respiratory tract infections are a persistent public health problem, causing more than two million deaths per year worldwide, with a rate of 36 deaths per 100,000 population (GBD 2016 Causes of Death Collaborators, 2017). The morbidity and mortality of respiratory infections could be even worse in developing countries, including China.

A substantial proportion of COPD exacerbations are associated with acute respiratory viral infection. Viral exacerbations result in longer recovery periods for individuals with COPD. The prevention or early treatment of viral infection in patients with COPD may attenuate the severity and frequency of COPD exacerbations and should lead to a decrease in health burden and thus an improvement in health-related quality of life. Additionally, viral infections may cause chronic infections in patients with COPD, and this may be related to disease severity.

Allergies and infections of the upper respiratory tract include the nose or nostrils, nasal cavity, mouth, throat (pharynx), and voice box (larynx). Upper respiratory tract infections commonly include nasal obstruction, sore throat, tonsillitis, pharyngitis, laryngitis, sinusitis, otitis media, and the common cold. While most infections are viral in nature others are bacterial. In 2015, there were an estimated 17.2 billion cases upper respiratory tract infections.

Viral infections result in the sequential activation of various immune cells to eliminate the virus from the host. While activation of immune responses is essential for inactivating invading viruses, they can also cause substantial collateral damage to host cells and the health of the host (Graham et al., 2005). Immunopathological responses can be impacted by past immune responses to unrelated infections, referred to as heterologous immunity (Selin et al., 1998). Heterologous immunity involves the T-cell memory pool such that T cells specific to past exposures to unrelated viruses may also contribute to the host's primary response to a second new virus. Heterologous immunity is influenced by the cytokine producing capacity of memory cells and these memory cells can be skewed in one cytokine direction or another may have the capacity to influence Th1 versus Th2 immune responses during infections (Welsh and Selin, 2002, Welsh et al, 2010).

It is well accepted that cytokines play an important role in innate and adaptive immune responses during viral infections. Immune cells are populations of white blood cells, such as circulating dendritic cells (DCs), neutrophils, natural killer (NK) cells, monocytes, eosinophils, and basophils, along with tissue-resident mast cells and macrophages (Iwasaki, et al., 2010). When virus is detected, a fast and coordinated innate immune response provides the first line of defense against the attack. For the immune system to properly function, synthesis and release of cytokines must be highly regulated and both sequentially and temporally coordinated (Hu et al., 2009). Proinflammatory cytokines also serve to recruit and activate T lymphocytes and other cells to mount a high coordinated response to a wide range of viral, fungal, bacterial, and parasitic pathogens (Iwasaki, et al., 2010). Thus, cascades of cytokines released by innate immune cells initially mount inflammatory or allergic responses then subsequently these responses should subside in a timely fashion. Cytokines and chemokines released by the innate immune cells includes tumor necrosis factor alpha (TNF-α), Interferon gamma (IFN-γ), interleukins (IL); IL-1β, IL-4, IL-6, IL-10, IL-12, IL-18, Chemokine (C—C motif) ligands 4 (CCL4, also known as macrophage inflammatory protein (MIP-1β) CCL4, CCL5 (also knowns as regulated on activation, normal T cell expressed and secreted, RANTES) and transforming growth factor-beta (TGF-β) (Lacy et al., 2011).

In severe viral respiratory infections, the innate immune response proceeds in a feed-forward state resulting in dysregulated and excessive immune responses including severe inflammation associated with the onset of a cytokine storm with more serious pathological changes observed, such as diffuse alveolar damage, hyaline membrane formation, fibrin exudates, and fibrotic healing (Shinya et al., 2012). The acute inflammatory response is also marked by the activation of pro-inflammatory cytokines or chemokines. These are associated with severe capillary damage, immunopathologic injury, and persistent organ dysfunction that can cause severe tissue and organ damage and death. A cytokine/chemokine-driven feed-forward inflammatory circuit may be responsible for the escalation of cytokine storm. In severe cases of respiratory diseases, inflammatory cytokines/chemokines produced in the lungs can spill over into general circulation and result in systemic cytokine storms, which are responsible for multi-organ dysfunction (Tisoncik et al., 2012).

Dysregulated and excessive immune responses can cause many diseases, severe tissue and organ damage and death. Proinflammatory responses are well known to play a central role in the pathogenesis of the acute phase of human coronavirus diseases, particularly SARS-CoV-1 (Ye et al., 2020) At the early stage of SARS-CoV-1 infection, in vitro cell experiments reveal a delayed release of cytokines and chemokines in respiratory epithelial cells, dendritic cells (DCs), and macrophages. In later phases cells secrete low levels of the antiviral factors interferons (IFNs) and high levels of proinflammatory cytokines (IL-1β, IL-6, and tumor necrosis factor (TNF)) and chemokines CCL-2, CCL-3, and CCL-5) (Law et al., 2005, Cheung et al. 2005, Lau et al., 2013). While high levels of cytokine and chemokine production are characteristic of the inflammatory phase of SARS-CoV and Middle East Respiratory Syndrome (MERS) and Influenza A, each disease has its own unique distributions of these pro-inflammatory factors.

Individuals infected with SARS viruses, such as MERS-CoV (Kim et al., 2016, Min et al., 2016, Ng et al., 2014), SARS-CoV (Cheung et al., 2005, Law et al., 2005, Channappanavar et al., 2017, Wong et al., 2006), and SARS-CoV-2 (Huang et al., 2020, Moore et al., 2020, Chen et al., 2020, Yang et al., 2020) have cytokine and chemokine levels that are elevated and also significantly higher in patients with severe cases compared to mild to moderate cases.

It has been demonstrated that the acute onset of proinflammatory immune responses, severe lung injury and ARDS caused by different pathogens critically depends on activation of the oxidative stress machinery that couples to innate immunity (Chow et al., 2003. Imai et al., 2008). ARDS was the prevalent cause of death is the 2003 SARS-CoV pandemic (Lew et al., 2003), the Spanish Influenza pandemic of 1918 (Tumpey et al., 2005) and the Avian Influenza A H5N1 virus (Beigel et al., 2006) and now SARS-CoV-2 (Weirsinga et al., 2020). Severe infections in humans the result of these viral respiratory diseases are accompanied by a combination of an aggressive pro-inflammatory response and an insufficient control of an anti-inflammatory response, resulting in both the cytokine and free-radical storms.

Recently, Siddiqui et al. (2020) reported that in the progression of COVID-19 disease there are two distinct and overlapping pathological subsets. The first phase is triggered by the virus itself and the second, the host response to the virus. They report whether in a native state, an immunoquiescent state, or in an immunosuppressed state, COVID-19 tends to present and follow these two phases at different levels of severity. They recommend a structured approach to clinical phenotyping be undertaken to distinguish the phase where the viral pathogenicity is dominant versus when the host inflammatory response overtakes the disease pathology. They recommend treatment in the first stage during viral replication be primarily targeted towards symptomatic relief and targeting patients at this early stage with a suitable anti-viral therapy that may reduce duration of symptoms, minimize contagiousness and prevent progression of severity into the second inflammatory phase.

In the second phase of COVID-19, pulmonary disease is established with viral multiplication and localized inflammation in the lung being common with viral pneumonia, cough, fever and possibly hypoxia being exhibited (Siddiqui et al., 2020). At this stage, blood tests frequently reveal lymphopenia and transaminitis with elevated systemic inflammatory markers. Hospital admission is commonly needed to monitor and manage patients at this stage of the disease with suitable anti-viral, as well as anti-inflammatory treatment.

Patients in the third more advanced stage of COVID-19 exhibit extra-pulmonary systemic hyperinflammation syndrome characterized with increased markers of systemic inflammation with a decrease in helper, suppressor and regulatory T cell counts (T-cell exhaustion) (Qin et al., 2020) along with increases in inflammatory cytokines and biomarkers reported by several groups. Shock, vasoplegia, respiratory failure, cardiopulmonary collapse, systemic organ involvement and myocarditis are commonly reported in the most serious cases.

To date the prognosis and recovery from the third stage of COVID-19 is poor, as evidenced by the current levels of mortality associated with this disease. Additional therapies including targeted cytokine antagonists, antiplatelet drugs that do not negatively impact other organ functioning, such as the liver, are critically needed to improve patient outcomes at this stage.

### Smoking

According to the CDC, more than 16 million Americans are living with a disease caused by cigarette smoking. Smoking causes cancer, heart disease, stroke, lung diseases, diabetes, and chronic obstructive pulmonary disease (COPD), which includes emphysema and chronic bronchitis. Smoking and second hand smoke is associated with some types of asthma and exacerbates its symptoms. Smoking also increases the risk for tuberculosis, certain eye diseases, and problems of the immune system, including rheumatoid arthritis. The World Health Organization (2018) reported that worldwide, an estimated 1.1 billion smoke cigarettes, tobacco use causes nearly 7 million deaths per year, and current trends show that tobacco use will cause more than 8 million deaths annually by 2030.

The U.S. Center for Disease Control (2018) stated that 15.5% of all adults, approximately 37.8 million people in the United States smoke cigarettes. Cigarette smoking is responsible for more than 480,000 deaths per year in the United States, including more than 41,000 deaths resulting from second hand smoke exposure; this is about one in five deaths annually, or 1,300 deaths every day. On average, smokers die 10 years earlier than nonsmokers.

Tobacco smoke is a complex mixture of gaseous compounds and particulates. Current literature shows 4800 identified gaseous and particulate bound compounds in cigarette smoke (Sahu, et al. 2013).

Airborne particulate matter (PM), and especially fine particles, has been associated with various adverse health effects. Environmental tobacco smoke (ETS) has also been identified as an important source of anthropogenic pollution in indoor environments, for example though second hand smoke. Cigarette smoke consist of gaseous pollutants; such as carbon monoxide (CO), sulfur dioxide (SO₂), nitric oxide (NO), nitrogen dioxide (NO₂), methane (CH₄), non-methane hydrocarbons (NMHC), carbonyls and volatile organic compounds (VOCs); and particulate matter (PM). The particulate concentration in tobacco smoke is generally very high at 10¹² particles per cigarette and has very small particle sizes, varying from 0.01 nm to 1.00 μm, with a count median size in the 186 to 198 nm range (Sahu, et al. 2013). Despite the small diameter of the smoke particles, smoke deposition efficiencies of 60 to 80% in the lung have been reported. The concentration of nicotine in cigarettes is variable depending upon the brand. A comprehensive study was conducted in 1998 in which the nicotine content was reported in 92 brands of cigarettes from the U.S., Canada and the United Kingdom (Kozlowski, et al. 1998). The total nicotine content of tobacco and percent nicotine (by weight of tobacco) averaged 10.2 mg (standard error of the mean (SEM) of 0.25 and range: 7.2 mg to 13.4 mg) and 1.5% (SEM of 0.03 and range 1.2% to 2%) in the United States, 13.5 mg (SEM of 0.49 and range: 8.0 mg to 18.3 mg) and 1.8% (SEM of 0.06 and range: 1.0% to 2.4%) in Canada, 12.5 mg (SEM of 0.33, range: 9 mg to 17.5 mg) and 1.7% (SEM 0.04, range: 1.3% to 2.4%) in the United Kingdom. However, the nicotine intake per cigarette averages 1.04 mg (+/−0.36), indicating the absorption and actual dose of nicotine from smoking a cigarette is much lower than the amount in the tobacco of a cigarette (Benowitz et al. 1984).

### Air Pollution

More than 80% of people living in urban areas that monitor air pollution are exposed to air quality levels exceeding World Health Organization (WHO) limits. As urban air quality declines, risks of stroke, heart disease, lung cancer, and chronic and acute respiratory diseases, including COPD and asthma, increase for the people who live in them. There are globally 4.2 million deaths each year directly attributed to air pollution and 91% of the world's population live in areas that exceed WHO air pollution criteria. In 2016, the WHO reported the annual median PM2.5 concentrations (μg/m³) in various regions of the world. Large portions of Asia, Africa and India have PM2.5 concentrations greater than 26 μg/m³. The WHO Air Quality Guideline (AQG) for PM2.5 air pollutant concentrations is 10 μg/m³. PM2.5 refers to atmospheric particulate matter (PM) that have a diameter of less than 2.5 μg (micrometers), which is about 3% the diameter of a human hair. Owing to their minute size, particles smaller than 2.5 μg are able to bypass the nose and throat and penetrate deep into the lungs and some may even enter the circulatory system. Studies report a close link between exposure to fine particles and premature death from heart and lung disease. Fine particles are also known to trigger or worsen chronic disease such as asthma, COPD, heart attack, bronchitis and other respiratory problems.

### Chronic Obstructive Pulmonary Disease (COPD)

COPD is currently the fourth leading cause of death in the world and is projected to be the third leading cause of death by 2030. Most typically, the prevalence of COPD is directly related to tobacco smoking, although in many countries outdoor, occupational, and indoor air pollution (e.g., resulting from the burning of wood and other biomass fuels) are also major COPD risk factors. More than one-quarter of all people that have COPD do not smoke cigarettes and it is thought that air pollution is a primary cause in these cases.

Patients with chronic obstructive pulmonary disease experience exertional breathlessness caused by bronchoconstriction, mucous secretion, and edema of the airway wall and loss of attachments to the terminal airways. The World Organization (WHO) predicts that chronic obstructive pulmonary disease will become the third leading cause of disease-related death globally by 2030.

COPD is a common, preventable, and treatable disease that is characterized by airflow limitations and chronic respiratory symptoms the results of alveolar and airway abnormalities, typically caused by exposure to noxious gases or particulate matter. Chronic airflow limitations caused by COPD are caused by a combination of small airways disease (e.g., chronic bronchiolitis) and parenchymal destruction (emphysema). Chronic inflammation results in structural changes in the lungs, including narrowing of the small airways and destruction of the lung parenchyma, leading to a decrease in alveolar attachments to the small airways and lessening of lung elastic recoil. These changes diminish the ability of the airways to remain open during expiration. Narrowing of the small airways also contributes to airflow limitation and mucociliary dysfunction. Airflow limitation is usually measured by spirometry as this is the most widely available and reproducible test of lung function (Global Initiative for Chronic Obstructive Lung Disease, 2019).

Mitochondrial dysfunction and enhanced oxidative stress are capable of triggering an essential cellular degradation process, known as autophagy. The role of autophagy in pulmonary disorders can be either deleterious or protective, depending on the stimuli. In cigarette-smoke-induced COPD, autophagy is critical in mediating apoptosis and cilia shortening in airway epithelia. Autophagy, in turn, accelerates lung aging and emphysema and contributes to COPD pathogenesis by promoting epithelial cell death. Autophagy increases in pulmonary cells, leading to inflammation and emphysematous destruction in experimental COPD. Autophagy is critical in mediating inflammation and mucus hyper-production in epithelia via NF-κB and Activator protein 1 (AP-1) transcription factor.

Spirometry is the most frequently performed pulmonary function test and plays an important role in diagnosing the presence and type of lung abnormality and classifying its severity. Spirometry is used for assessment and surveillance examinations for individuals with COPD, asthma and other diseases associated with breathing impairment. It is additionally used for evaluation of occupational lung diseases in determining whether to institute preventive or therapeutic measures, and in granting benefits to individuals with lung impairment. Forced Expiratory Volume in 1 second (FEV1) and Forced Vital Capacity (FVC) spirometry data are compared to reference data and can be expressed as percent predicted values, based on age, gender, height and race (American Thoracic Society 1995). Spirometry is also used as a measure to assess an individual's response to treatment. FEV1/FVC ratio, percent reversibility of FEV1 and percent normal FEV1 are commonly used assessment parameters to evaluate the severity of airway obstructive diseases, diagnosis and treatment effectiveness.

Several mechanisms may explain how cigarette smoke can cause airway inflammation and subsequent disease. Barnes (2004) identified one mechanism identified in the role that cigarette smoke can play in the imbalance of proinflammatory cytokines, for example, Interferon-1β (IL-1β), IL-6, IL-8, interferon-γ, tumor necrosis factor-α (TNF-α) and anti-inflammatory cytokines (for example the IL-1 receptor antagonist, IL-4, IL-10, IL-11, and IL-13). A second mechanism is oxidative stress due to imbalance between oxidants and anti-oxidant defense mechanisms in airways and lungs. Oxidants are released from alveolar macrophages as well as neutrophils of COPD patients. Activated inflammatory cells, attracted into the alveolar space by chemokines and cytokines, release myeloperoxidase and large amounts of hypochlorous acid (HOCl) in the 0.1-1.0 mM range, in the vicinity of airway and alveolar epithelial cells.

Cigarette smoke itself is also a rich source of oxidants, as each puff of cigarette smoke contains approximately 10¹⁵ oxidant radical molecules and 10¹⁷ Electron Spin Resonance (ESR)-detectable radicals per gram of tar (Cantin, 2010). Antioxidants are natural molecules in biological system that scavenge oxidants, including free radicals, and protect from effects or free radicals and other reactive oxygen species. Antioxidants can be synthesized endogenously in the body, or exogenously by food intake or by supplementation. In one embodiment of this present invention, antioxidants comprise part of a multifunctional composition that is inhaled by a patient to minimize reactive oxygen species present in the respiratory tract associated with COPD, asthma and other respiratory tract diseases.

Exposure to wood smoke was studied by Leonard et al. (2000) who reported that wood smoke is able to induce carbon centered as well as reactive hydroxyl (OH) radicals and can in turn cause cellular damage. They also reported that wood smoke can cause lipid peroxidation, DNA damage, Nuclear Factor kappa-Light-Chain-Enhancer of Activated B Cells (NF-κB) activation and TNF-α induction. These authors proposed that the OH radical plays an important role in these immune system responses and that iron present in wood smoke and H₂O₂ generated in the respiratory tract during phagocytosis of wood smoke particles creates OH free radicals and other reactive oxygen species (ROS) in the lungs. These authors suggest that wood smoke is capable of causing acute lung injury and may have the potential to act as a fibrogenic agent.

### Asthma

Asthma is a chronic inflammatory lung disease that results in airflow limitations, hyperreactivity and airway remodeling. There are approximately 235 million people worldwide who have asthma and globally, there were approximately 383,000 asthma-related deaths in 2015. (World Health Organization, 2018). Symptoms of asthma can be varied, with wheezing, shortness of breath, and coughing that occurs more frequently during the night and early morning. Asthma symptoms are frequently episodic and can be caused by various triggers, such as respiratory irritants; including cigarette smoke, second hand smoke, air pollution, specific allergens and exercise. Asthma often starts in early childhood and is characterized by intermittent wheezing and shortness of breath. While there are some similar clinical features of asthma and COPD, there are marked differences in the pattern of inflammation in the respiratory tract, with different inflammatory cells, mediators, consequences, and responses to therapy.

Asthma can be broadly classified as eosinophilic or non-eosinophilic on the basis of airway or peripheral blood cellular profiles, with approximately half of individuals falling into each category (Carr et al, (2018). Cytokines play a critical role in orchestrating, perpetuating and amplifying the inflammatory response in asthma. It has been reported that patients with severe asthma have airway inflammation that is similar to those with COPD (Barnes (2001, 2008). Eosinophilic asthma is thought to be a T helper cell 2 (Th2)-cell driven inflammatory disease, characterized by eosinophilic inflammation, Th2-cell associated cytokine production and airway hyper-responsiveness (Lloyd et al. (2010). In patients with eosinophilic asthma, Th2 associated cytokine secretion of IL-4, IL-5, IL-9, IL-13, IL-25, IL-33, thymic stromal lymphopoeitin (TSLP) and Granulocyte-Macrophage Colony Stimulating Factor (GM-CSF) are thought to drive the disease pathology. Patients with neutrophilic (non-eosinophilic) asthma have low- or non-Th2 associated cytokine production of IL-8, IL-17, IL-22, IL-23, interferon-gamma (IFNγ), tumor necrosis factor-α (TNFα), chemokine receptor 2 (CXCR2), IL-10 and IL-6 that drive the disease pathology (Carr et al. 2018).

### Heavy Metals and Smokers

According to the U.S. Department of Health and Human Services (2006), cigarette smoke inhaled by a smoker contains more than 4,000 chemicals and second hand smoke (SHS) is qualitatively similar. Heavy metals in tobacco smoke are of public health concern because of their potential toxicity and carcinogenicity. Richter et al. (2009) reporting on results of The National Health and Nutrition Examination Survey (NHANES) 1999-2004, concluded that individuals who smoked cigarettes had higher cadmium, lead, antimony, and barium levels than nonsmokers. Highest lead levels were in the youngest subjects. Lead levels among adults with high second-hand smoke exposure equaled those of smokers. Older smokers had cadmium levels signaling the potential for cadmium-related toxicity. Cadmium is a known Group 1 carcinogen. The findings of Richter et al. (2009) revealed second hand smoke-exposed children, a population particularly vulnerable to the toxic effects of lead at low levels of exposure, have higher levels of urine lead than children without SHS exposure. Urine lead levels respond rapidly to changes in body lead burdens and increased with increasing lead exposure.

Cadmium has been attributed a causative role in pulmonary emphysema among smokers. Cadmium concentration in lung tissues of smokers with Global Initiative for Chronic Obstructive Lung Disease (GOLD) Stage IV COPD (58±10.8 pack-years) was reported by Hassan, et al. (2014) to be directly proportional to the total tobacco consumption (“tobacco load”) among patients. Sunblad et al. (2016) published evidence for a link between local cadmium concentrations and alterations in innate immunity in the lungs. They reported that cadmium concentrations were markedly increased in cell-free Bronchial Lavage Fluid (BLF) of smokers compared to that of nonsmokers, irrespective of chronic obstructive pulmonary disease. In these smokers, the measured cadmium concentrations displayed positive correlations with macrophage TNF-α mRNA in BAL, neutrophil and Cytoxic T-Cell (CD8+) cell concentrations in blood, and finally with the inflammatory cytokines IL-6, IL-8, and matrix metallopeptidase 9 (MMP-9) protein in sputum. They also concluded that extracellular cadmium is enhanced in the bronchoalveolar space of long-term smokers and displays pro-inflammatory features. Local accumulation of cadmium in the lungs appears to be a critical component of predisposition to lung diseases among long-term smokers. This is particularly important considering that the biological half-life of cadmium in the human body is >25 years, a substantial period of time, suggesting the possibility of significant retention of cadmium in the lungs of long-term smokers.

### SUMMARY OF THE INVENTION

In an embodiment of the invention, a pharmaceutical composition includes at least one plant extract Transient Receptor Potential Cation Channel, Subfamily A, member 1 (TRPA1) antagonist, at least one thiol amino acid containing compound, at least one vitamin, at least one chelating agent, and at least one antioxidant. The plant extract TRPA1 antagonist can be 1,8-cineole, borneol, camphor, 2-methylisoborneol, fenchyl alcohol, cardamonin, or combinations. The thiol amino acid containing compound can be a naturally-occurring compound. The thiol amino acid containing compound can be glutathione, N-acetyl cysteine, carbocysteine, taurine, methionine, or combinations. The vitamin can be a cobalamin, methylcobalamin, hydroxycobalamin, adenosylcobalamin, cyanocobalamin, cholecalciferol, thiamin, dexpanthenol, biotin, nicotinic acid, nicotinamide, nicotinamide riboside, ascorbic acid, a provitamin, or combinations. The chelating agent can be glutathione, N-acetyl cysteine, citric acid, ascorbic acid, ethylenediaminetetraacetic acid (EDTA), or combinations. The antioxidant can be a naturally-occurring compound. The antioxidant can be berberine, catechin, curcumin, epicatechin, epigallocatechin, epigallocatechin-3-gallate, β-carotene, quercetin, kaempferol, luteolin, ellagic acid, resveratrol, silymarin, nicotinamide adenine dinucleotide, thymoquinone, 1,8-cineole, glutathione, N-acetyl cysteine, a cobalamin, methylcobalamin, hydroxycobalamin, adenosylcobalamin, cyanocobalamin, β-caryophyllene, xylitol, or combinations.

The pharmaceutical composition can include from about 0.05% to about 10% epigallocatechin-3-gallate and from about 0.1% to about 10% resveratrol. The pharmaceutical composition can include from about 0.05% to about 10% xylitol.

The pharmaceutical composition can further include a carrier. The carrier can be a liquid carrier. The carrier can include a liquid such as water, saline, deaired water, deaired saline, water purged with a pharmaceutically inert gas, saline purged with a pharmaceutically inert gas, or combinations. The carrier can include water or saline and a polysorbate, such as polysorbate 20 and Polysorbate 80.

The pharmaceutical composition can include a lubricating, emulsifying, and/or viscosity-increasing compound. The lubricating, emulsifying, and/or viscosity-increasing compound can be a carbomer, a polymer, acacia, alginic acid, carboxymethyl cellulose, ethylcellulose, hydroxyethyl cellulose, hydroxypropyl cellulose, methylcellulose, poloxamers, polyvinyl alcohol, lecithin, sodium alginate, tragacanth, guar gum, sodium hyaluronate, hyaluronic acid, xanthan gum, glycerin, vegetable glycerin, polyethylene glycol, polyethylene glycol(400), a polysorbate, polyoxyethylene(20)sorbitan monolaurate (polysorbate 20), polyoxyethylene(20)sorbitan monooleate (polysorbate 80), polyoxyethylene(20)sorbitan monopalmitate (polysorbate 40), polyoxyethylene(20)sorbitan monostearate (polysorbate 60), sorbitan trioctadecanoate, polyglyceryl-3 stearate, polyglyceryl-3 palmitate, polyglyceryl-2 laurate, polyglyceryl-5 laurate, polyglyceryl-5 oleate, polyglyceryl-5 dioleate, polyglyceryl-10 diisostearate, or combinations.

The pharmaceutical composition can include a pH-adjusting compound. The pH-adjusting compound can be sodium hydroxide, sodium bicarbonate, sodium carbonate, sodium citrate, benzoic acid, ascorbic acid, citric acid, or combinations.

The pharmaceutical composition can include a preservative. The preservative can be ethylenediaminetetraacetic acid (EDTA), benzalkonium chloride, benzoic acid, sorbic acid, or combinations.

The carrier can include from about 0% to about 95% vegetable glycerin and from about 5% to about 98% percent water. The carrier can further include from about 0.001% to about 1.00% sodium bicarbonate. The carrier can further include from about 0.001 to about 0.06% ethylene diamine tetraacetic acid (EDTA).

The pharmaceutical composition can further include an amino acid. The amino acid can be a proteinogenic amino acid. The amino acid can be an essential amino acid. The amino acid can be alanine, leucine, isoleucine, lysine, valine, methionine, L-theanine, phenylalanine, or combinations.

The pharmaceutical composition can include from about 0.05% to about 10% dexpanthenol, from about 0.05% to about 10% L-theanine, and from about 0.05% to about 10% taurine.

The pharmaceutical composition can further include a Cannabinoid Receptor Type 2 (CB2) agonist. The CB2 agonist can be a naturally-occurring CB2 agonist. For example, the CB2 agonist can be β-caryophyllene, cannabidiol, or cannabinol. The pharmaceutical composition can include from about 0.1% to about 1% β-caryophyllene.

The pharmaceutical composition can further include a cannabinoid compound, for example, cannabidiol. The pharmaceutical composition can include from about 0.005% to about 5% of a cannabinoid compound.

The pharmaceutical composition can further include nicotine. The pharmaceutical composition can include from about 0.01% to about 2.5% nicotine.

The pH of the pharmaceutical composition can be from about 6 to about 8, for example, about 7.2.

The ionic strength of the pharmaceutical composition can be equivalent to that of normal lung epithelial lining fluid.

The pharmaceutical composition can further include a liposome. The liposome can include the plant extract TRPA1 antagonist, thiol amino acid containing compound, vitamin, and/or antioxidant. The liposome can include the plant extract TRPA1 antagonist, thiol amino acid containing compound, vitamin, antioxidant, amino acid, and/or CB2 agonist.

The pharmaceutical composition can further include a micro- or nano-emulsion. The micro- or nano-emulsion can include the plant extract TRPA1 antagonist, thiol amino acid containing compound, vitamin, and/or antioxidant. The micro- or nano-emulsion can include the plant extract TRPA1 antagonist, thiol amino acid containing compound, vitamin, antioxidant, amino acid, and/or CB2 agonist.

In an embodiment, the pharmaceutical composition includes from about 0.1% to about 10% 1,8-cineole, from about 0.1% to about 10% N-acetyl cysteine, from about 0.1% to about 20% glutathione, from about 0.01% to about 1% ascorbic acid, from about 0.001% to about 1.0% methylcobalamin, and a carrier.

In an embodiment, the pharmaceutical composition includes about 0.8% 1,8-cineole, about 0.8% β-caryophyllene, about 1.35% N-acetyl cysteine, about 1.35% glutathione, about 0.01% ascorbic acid, about 0.003% methylcobalamin, about 0.8% Polysorbate 20, and sterile saline water including 0.9% sodium chloride (NaCl), and the pH is adjusted to about 7.2 with added sodium bicarbonate. In another embodiment, the pharmaceutical composition includes about 0.8% 1,8-cineole, about 0.8% β-caryophyllene, about 1.11% N-acetyl cysteine, about 1.11% glutathione, about 0.007% methylcobalamin, about 0.8% Polysorbate 20, and sterile saline water including 0.9% sodium chloride (NaCl), and the pH is adjusted to about 7.2 with added sodium bicarbonate. In an embodiment, the pharmaceutical composition further includes at least one of the following: about 0.05% EDTA, about 1% dexpanthenol, about 0.7% L-theanine, about 0.5% taurine, about 0.05% epigallocatechin-3-gallate, about 0.5% resveratrol, and about 3% cannabidiol. In yet another embodiment, the pharmaceutical composition further includes about 5% xylitol.

In an embodiment, the pharmaceutical composition includes about 1.7% 1,8-cineole, about 1.7% β-caryophyllene, about 1.2% N-acetyl cysteine, about 1.5% glutathione, about 0.01% ascorbic acid, about 0.003% methylcobalamin, about 1.7% Polysorbate 20, about 91% vegetable glycerin, and sterile deionized water, and the pH is adjusted to about 7.2 with added sodium bicarbonate. In an embodiment, the pharmaceutical composition further includes at least one of the following: about 0.05% EDTA, about 1% dexpanthenol, about 0.7% L-theanine, about 0.5% taurine, about 0.05% epigallocatechin-3-gallate, about 0.5% resveratrol, and about 3% cannabidiol. In an embodiment, the pharmaceutical composition further includes about 1.8% nicotine.

In an embodiment, the pharmaceutical composition of claim 1 includes from about 10 to about 30 g/L glutathione, from about 7 to about 25 g/L N-acetyl cysteine, from about 10 to about 30 g/L 1,8-cineole, and from about 0.02 to about 0.06 g/L of a cobalamin or methylcobalamin, and the pharmaceutical composition is a liquid. In an embodiment, the pharmaceutical composition further includes from about 6 to about 20 g/L Polysorbate 20, and from about 0 to about 1150 g/L glycerine, and the balance is water or saline. In an embodiment, the pharmaceutical composition further comprises from about 6 to about 20 g/L Polysorbate 20, and from about 500 to about 1150 g/L glycerine, and the balance is water or saline.

In an embodiment, the pharmaceutical composition includes about 20 g/L glutathione, about 15 g/L N-acetyl cysteine, about 20 g/L 1,8-cineole, about 0.04 g/L of a cobalamin or methylcobalamin, and about 1100 g/L vegetable glycerine, and the pharmaceutical composition is a liquid. In an embodiment, the pharmaceutical composition further includes about 12 g/L Polysorbate 20, and the balance is deionized water.

In an embodiment, the pharmaceutical composition comprises glutathione, N-acetyl cysteine, and a cobalamin or methylcobalamin. In an embodiment, the pharmaceutical composition further includes 1,8-cineole and/or β-caryophyllene.

In an embodiment, the pharmaceutical composition includes from about 0.5 to about 2% glutathione, from about 0.5 to about 2% N-acetyl cysteine, from about 0.4 to about 1.2% 1,8-cineole, from about 0.0002 to about 0.01% of a cobalamin or methylcobalamin, and from about 0.1 to about 1.2% β-caryophyllene. In an embodiment, the pharmaceutical composition further includes from about 0.1% to about 1.5% Polysorbate 20, and from about 0 to about 90% glycerine, and the balance is water or saline.

In an embodiment, the pharmaceutical composition includes about 1.1% glutathione, about 1.1% N-acetyl cysteine, about 0.8% 1,8-cineole, about 0.003% of a cobalamin or methylcobalamin, and about 0.8% β-caryophyllene. In an embodiment, the pharmaceutical composition further includes about 0.3% Polysorbate 20, and the balance is a sterile saline solution. In an embodiment, the sterile saline solution is an about 0.9% saline solution.

In an embodiment, the pharmaceutical composition includes from about 0.3 to about 1% glutathione, from about 0.3 to about 1% N-acetyl cysteine, and from about 0.001 to about 0.01% of a cobalamin or methylcobalamin. In an embodiment, the pharmaceutical composition further includes from about 0 to about 0.5% Polysorbate 20, and from about 0 to about 90% glycerin, and the balance is water or saline.

In another embodiment, the pharmaceutical composition includes from about 0.4 to about 2.5% 1,8-cineole, from about 0.1 to about 1.2% β-caryophyllene, from about 0.5 to about 10% xylitol, from about 0.1 to about 1.5% Polysorbate 20 alone or in combination with Polysorbate 80, the balance is water or saline and the pH is adjusted from about 3.0 to 7.0 with added sodium bicarbonate or citric acid. The pharmaceutical composition can be in an aerosolized or nebulized form. The pharmaceutical composition can also be delivered intranasally with a device including a pump or pressurized nasal spray.

In an embodiment, the pharmaceutical composition includes 1.0% 1,8-cineole, 0.5% β-caryophyllene, 5% xylitol, 1.0% Polysorbate 20, 91.5% purified sterile water, 0.82% sodium chloride and the pH is adjusted to 3.25 with about 0.49 g citric acid. The pharmaceutical composition can be in an aerosolized or nebulized form. The pharmaceutical composition can be delivered intranasally with a device including a pump or pressurized nasal spray.

In another embodiment, the pharmaceutical composition includes from about 0.5 to about 10% N-acetyl cysteine, from about 0.4 to about 2.5% 1,8-cineole, from about 0.1 to about 1.2% β-caryophyllene, from about 0.5 to about 10% xylitol, from about 0.0002 to about 0.01% of a cobalamin or methylcobalamin, from about 0.1% to about 1.5% Polysorbate 20 alone or in combination with Polysorbate 80, the balance is water or saline and the pH is adjusted from about 3.0 to 7.0 with added sodium bicarbonate or citric acid. The pharmaceutical composition can be in an aerosolized or nebulized form. The pharmaceutical composition can be delivered intranasally with a device including a pump or pressurized nasal spray.

In an embodiment, the pharmaceutical composition includes 2.5% N-acetyl cysteine, 1.0% 1,8-cineole, 0.5% β-caryophyllene, 5% xylitol, 0.0007 methylcobalamin, 1.0% Polysorbate 20, 89.18% purified sterile water, 0.82% sodium chloride and the pH is adjusted to 3.25 with about 0.49 g citric acid. The pharmaceutical composition can be in an aerosolized or nebulized form. The pharmaceutical composition can be delivered intranasally with a device including a pump or pressurized nasal spray.

In an embodiment, the pharmaceutical composition includes about 0.7% glutathione, about 0.7% N-acetyl cysteine, and about 0.003% of a cobalamin or methylcobalamin. In an embodiment, the balance is a sterile saline solution, such as an about 0.9% saline solution.

The pharmaceutical composition can be in an aerosolized or nebulized form.

A method for treating a respiratory disease includes administering to a patient's lungs the pharmaceutical composition of the invention in an aerosolized or nebulized form. The respiratory disease can be airway inflammation, chronic cough, asthma, chronic obstructive pulmonary disease (COPD), allergic rhinitis, or cystic fibrosis. The patient can be an active or former cigarette smoker; the patient can be currently or have been exposed to second-hand smoke; the patient can be currently or have been exposed to wood or forest fire smoke; and/or the patient can be currently or have been exposed to gaseous or particulate natural or man-made air pollutants. The pharmaceutical composition can be in liquid form, which can be aerosolized using a nebulizer, an ultrasonic vaporization device, a thermal vaping device, or a device that creates an aerosol or gas phase from a liquid. The pharmaceutical composition in a liquid phase and a pharmaceutically inert gas can be sealed in a gas tight container.

A cigarette smoking cessation and respiratory system treatment method according to the invention includes in a first step administering to a patient's lungs a first mixture of the pharmaceutical composition and nicotine, which is at a first concentration in the first mixture, in an aerosolized or nebulized form over a first period of time, and in a final step administering to the patient's lungs the pharmaceutical composition of the invention (without nicotine) in an aerosolized or nebulized form over a final period of time. The aerosolized or nebulized pharmaceutical composition and/or the nicotine can be administered to the patient's lungs by the patient inhaling the pharmaceutical composition and/or the nicotine in a series of puffs using a nebulizer, an ultrasonic vaporization device, a thermal vaping device, or a device that creates an aerosol, nebulized, or gas phase from the pharmaceutical composition and/or the nicotine. In the first step, the patient can inhale the first mixture in a number of puffs per day and ingest an amount of nicotine per day that approximates that in the patient's recent active cigarette smoking behavior. In the first step, the patient can inhale the first mixture in from about 50 to about 400 puffs, such as about 150 puffs, per day. In the first step, the patient can ingest from about 5 to about 40 mg, such as about 20 mg, of nicotine per day. In the first step, the patient can inhale from about 0.5 mL to about 2 mL, such as about 1 mL, of the first mixture per day. In the first step, the first concentration of nicotine can be from about 0.5% to about 4%, such as about 1.4%, of the first mixture. In the first step, the first period of time can be from about 2 weeks to about 4 months, such as from about 40 to about 60 days. In the final step, the patient can inhale from about 0.5 mL to about 2 mL, such as about 1 mL, of the pharmaceutical composition per day.

The method can further include at least one intermediate step of administering to the patient's lungs another mixture according to the invention of the pharmaceutical composition and nicotine, the nicotine being at another concentration in the other mixture that is less than the first concentration, in an aerosolized or nebulized form over another period of time. For example, the method can include a second step of administering to the patient's lungs a second mixture according to the invention of the pharmaceutical composition of the invention and nicotine, the nicotine being at a second concentration in the second mixture that is less than the first concentration, in an aerosolized or nebulized form over a second period of time. In the second step, the patient can inhale the second mixture in from about 40 to about 320 puffs, such as 125 puffs, per day. In the second step, the patient can ingest from about 4 to about 30 mg of nicotine, such as about 14 mg of nicotine, per day. In the second step, the patient can inhale from about 0.5 mL to about 2 mL, such as about 1 mL, of the second mixture per day. In the second step, the second concentration of nicotine can be from about 0.3% to about 3%, such as about 1%, of the second mixture. In the second step, the second period of time can be from about 2 weeks to about 2 months, such as from about 14 to about 30 days.

The method can further include a third step of administering to the patient's lungs a third mixture according to the invention of the pharmaceutical composition and nicotine, the nicotine being at a third concentration in the third mixture that is less than the second concentration, in an aerosolized or nebulized form over a third period of time. In the third step, the patient can inhale the third mixture in from about 25 to about 200 puffs, such as about 75 puffs, per day. In the third step, the patient can ingest from about 2 to about 15 mg of nicotine, such as about 5 mg, of nicotine per day. In the third step, the patient can inhale from about 0.5 mL to about 2 mL, such as about 1 mL, of the third mixture per day. In the third step, the third concentration of nicotine can be from about 0.1% to about 1%, such as about 0.4%, of the third mixture. In the third step, the third period of time is from about 2 weeks to about 2 months, such as from about 14 to about 30 days.

In an embodiment of the cigarette smoking cessation and respiratory system treatment method according to the invention, the pharmaceutical composition includes from about 0.5% to about 5% (e.g., about 1.4%) glutathione, from about 0.3% to about 3% (e.g., about 1%) N-acetyl cysteine, from about 0.3% to about 3% (e.g., about 0.8%) 1,8-cineole, from about 0.0002% to about 0.002% (e.g., about 0.0007%) methylcobalamin, and from about 0.1% to about 1.2% (e.g., about 0.4%) β-caryophyllene. The pharmaceutical composition can further include from about 0% to about 2% (e.g., about 0.7%) Polysorbate 20 and from about 0% to about 90% (e.g., about 80%) glycerine, and the balance can be water or saline.

In an embodiment of the cigarette smoking cessation and respiratory system treatment method according to the invention, the pharmaceutical composition includes about 1.4% glutathione, about 1% N-acetyl cysteine, about 0.8% 1,8-cineole, about 0.0007% methylcobalamin, and about 0.4% β-caryophyllene. The pharmaceutical composition can further include about 0.7% Polysorbate 20 and about 80% glycerine, and the balance can be water or saline.

For example, a nebulizer can creates the aerosol, nebulized, or gas phase from the pharmaceutical composition and/or the nicotine.

### BRIEF DESCRIPTION OF THE DRAWINGS

The foregoing summary, as well as the following detailed description of preferred embodiments of the present application, may be better understood when read in conjunction with the appended drawings. It should be understood, however, that the application is not limited to the precise embodiments shown in the drawings.

FIG. 1 provides a graph presenting the results of FEV1 spirometry testing over time on five patients in a pre-clinical trial. It can be seen that there was a linear rate of FEV1 improvement overtime with a substantial improvement in spirometry results.

FIG. 2 provides a graph illustrating the comparison between the FEV1 patient treatment results percent normal FEV1 before treatment (light gray bars) and after treatment (black bars).

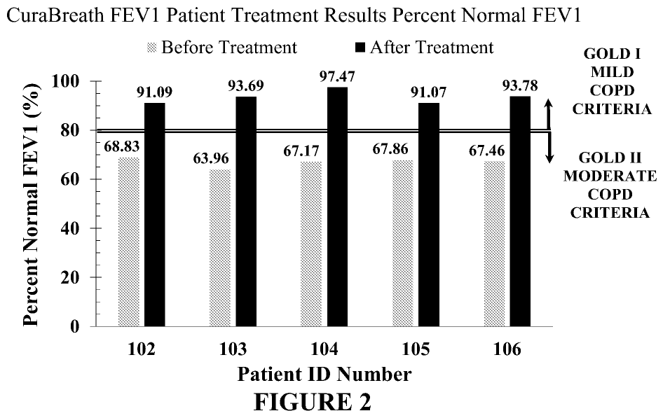

FIG. 3 provides a graph illustrating the comparison between the FEV1 patient treatment results before treatment (light gray solid bars) and after treatment (black solid bars), as well as the normal FEV1 (striped bars) calculated based on age, sex, height, and race.

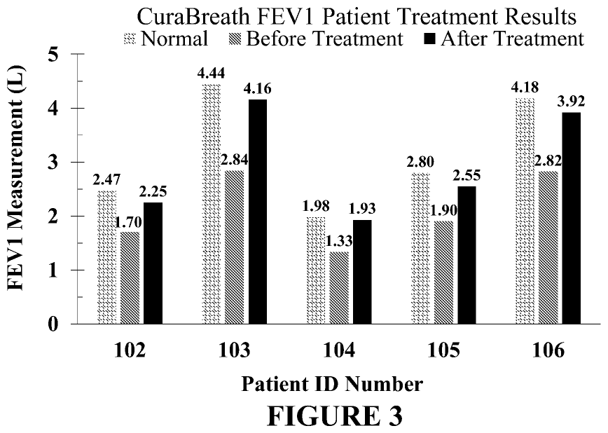

FIG. 4 provides a graph presenting the results of percent FEV1 reversibility for each of the five patients.

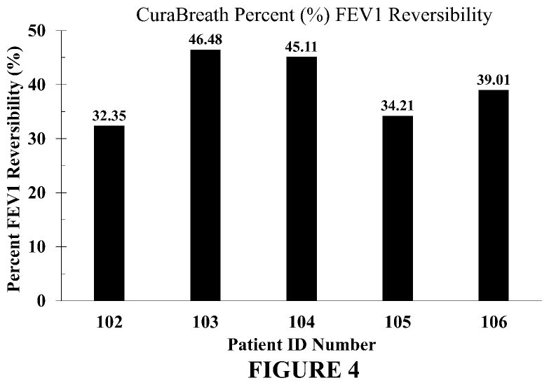

FIG. 5 provides a graph presenting the mean results of FEV1 before treatment (light gray bar) and after treatment (black bar). T Test analysis indicates that the results are significant at the P=0.0001 level.

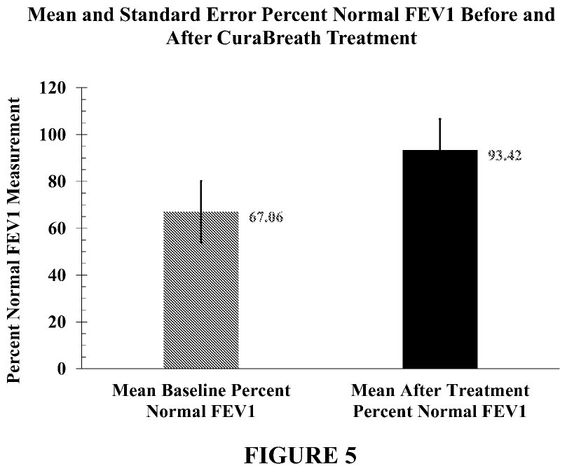

FIG. 6 provides a list of complete blood count test results conducted before and after nebulization of the pharmaceutical composition disclosed in Table A2 by nine patients.

FIG. 7 provides a list of comprehensive metabolic panel test results conducted before and after nebulization of the pharmaceutical composition disclosed in Table A2 by nine patients.

FIG. 8 provides a list of automated differential test results of individual white blood cells (lymphocytes) conducted before and after nebulization of the pharmaceutical composition disclosed in Table A2 by nine patients.

FIG. 9 provides a list of lymphocyte subset test results of individual white blood cells (lymphocytes) conducted before and after nebulization of the pharmaceutical composition disclosed in Table A2 by nine patients.

### DETAILED DESCRIPTION OF THE INVENTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent parts can be employed and other methods developed without parting from the spirit and scope of the invention. Each and every reference cited herein is hereby incorporated by reference in its entirety as if it had been individually incorporated. U.S. Provisional Application No. 62/749,446, filed Oct. 23, 2018, and International Application No. PCT/US2019/057722, filed Oct. 23, 2019, are each hereby incorporated by reference in their entireties.

This present invention relates to methods of use and compositions of liquids that are transferred to gas and aerosol phases for inhalation drug treatment of lung and respiratory tract diseases. More particularly this invention relates to methods of use and composition of liquids that orally administered to the lungs through vaporization and aerosol generating devices providing a multifunctional treatment for lung and respiratory diseases comprising plant-based TRPA1 antagonists, natural thiol amino acid containing compounds, CB₂ agonists, amino acids, naturally occurring antioxidants, vitamins and bioflavonoid compounds, and heavy metal complexing compounds. This present invention also relates to multifunctional liquid compositions including cannabinoid compounds, plant-based TRPA1 antagonists, natural thiol amino acid containing compounds, CB₂ agonists, amino acids, naturally occurring antioxidants, vitamins, and bioflavonoid compounds and heavy metal complexing compounds. This invention relates to compositions and methods of use of liquids to reduce lung damage in patients who are exposed to cigarette smoke from actively smoking cigarettes or second hand cigarette smoke, forest fire smoke, and other types of smoke inhalation, including those who may have been active cigarette smokers or exposed to cigarette smoke in the past.

This present invention relates to methods of use and compositions of pharmaceutical liquid compositions that are transferred to gas and aerosol phases for inhalation drug treatment of lung and respiratory tract diseases. More particularly this invention relates to methods of use and compositions of liquids that are orally administered to the lungs through vaporization and aerosol generating devices providing multifunctional treatment for lung and respiratory diseases comprising plant-based Transient Receptor Potential Cation Channel, Subfamily A, member 1 (TRPA1) antagonists, natural thiol amino acid containing compounds, one or more vitamins, naturally occurring antioxidants, heavy metal complexing compounds and carriers. This invention also includes pharmaceutical liquid compositions and methods of use including amino acids, natural Cannabinoid Receptor Type 2 (CB₂) receptor agonists, cannabinoid compounds and nicotine. Even more specifically, this invention relates to methods of use and compositions of liquids to reduce lung damage in patients who are exposed to air pollution, cigarette smoke from actively smoking cigarettes, second hand cigarette smoke, and wood smoke. In addition, this invention also relates to methods of use and compositions of liquids for smoking cessation (helping smokers to quit smoking) and respiratory system treatment.

COPD includes chronic bronchitis and emphysema. Environmental exposure, primarily from cigarette smoking, causes high oxidative stress and is the main factor of chronic obstructive pulmonary disease development. Cigarette smoke also contributes to the imbalance of oxidant/antioxidant due to exogenous reactive oxygen species associated with cigarette smoke. Reactive oxygen species endogenously released during the inflammatory process and mitochondrial dysfunction contribute to the progression of COPD. Reactive oxygen species (ROS) and reactive nitrogen species (RNS) can oxidize different biomolecules such as DNA, proteins, and lipids leading to epithelial cell injury and death.

Structural changes to essential components of the lung are caused by oxidative stress, contributing to irreversible damage of both parenchyma and airway walls. In addition, oxidative stress may result in alterations in the local immune response. However, cells can be protected against oxidative stress by enzymatic and non-enzymatic antioxidant systems. Attenuation of oxidative stress results in reduced pulmonary damage and a decrease in local infections, contributing to attenuation of the progression of COPD. Attenuation of oxidative stress in the lungs by inhalation of naturally occurring antioxidants is one embodiment of this present invention.

Pharmacological therapy for COPD is used to reduce symptoms, reduce the frequency and severity of exacerbations, and improve exercise tolerance and health status. To date, there is no conclusive clinical trial evidence that any existing medications for COPD modify the long-term decline in lung function. Drug treatment in patients with COPD is typically focused on bronchodilation by inhaled anticholinergics and β2-agonists. Anti-inflammatory therapy is another treatment regime in COPD patients and includes inhaled corticosteroids, oral glucocorticoids, PDE4 inhibitors, antibiotics, mucoregulators and antioxidants. Bronchodilators are medications that increase FEV1 and/or change other spirometric measurements. They act by altering airway smooth muscle tone and improvement in expiratory flow and reflect widening of the airways rather than changes in lung elastic recoil. It is not uncommon for COPD patient treatments to include combination treatments, such as inhaled corticosteroids with long acting bronchodilator therapy. To improve lung function, patient reported outcomes and to prevent exacerbations, triple inhaled therapy has also been developed using long-acting antimuscarinic antagonists (LAMAs), long acting β2-agonists (LABAs) and inhaled corticosteroids in a single inhaler. The use of anticholinergics, short-acting β2-agonists, inhaled corticosteroids, LAMAs, and LABAs all have significant reported side effects. Increasing FEV1 responses of patients through bronchodilation is one embodiment of this present invention.

Neither inhaled corticosteroids, nor high dosages of oral corticosteroids affect the number of inflammatory cells or concentrations of cytokines and proteases in induced sputum from COPD patients. The inhaled corticosteroid, dexamethasone does not inhibit basal or stimulated release of IL-8 by alveolar macrophages in COPD patients compared to healthy smokers. Corticosteroids inhibit apoptosis and thus stimulate survival of neutrophils. Corticosteroids are known to reduce serum IL-8 levels, which may result in a reduction in the influx of neutrophils. Treatment with inhaled corticosteroids reduces the concentration of exhaled NO and H₂O₂ in exhaled air.

One embodiment in this present invention is an alternative treatment of COPD patients using corticosteroids and bronchiodilators with a multifunctional inhaled aerosolized pharmaceutical liquid composition comprising natural antioxidants, natural anti-inflammatory compounds and vitamins. In another embodiment of this present invention are combinations of inhaled aerosolized pharmaceutical liquid composition comprising natural antioxidants, natural anti-inflammatory compounds, and vitamins with existing prescription corticosteroids and bronchodilators.

Similar to COPD, there is strong evidence that both endogenous and exogenous reactive oxygen species and reactive nitrogen species play a major role in the airway inflammation and affect asthma severity. Cigarette smoke, inhalation of airborne pollutants (ozone, nitrogen dioxide, sulfur dioxide) and particulate matter in the air can trigger symptoms of asthma. A clear relationship between traffic density and asthma exacerbations has been also been demonstrated. Cigarette smoke is related to asthma exacerbations, especially in young children, and there is a dose-dependent relationship between exposure to cigarette smoke and rates of asthma.

The goals of asthma treatment are to reduce symptoms and limit exacerbations. Currently, it is recommended that all patients with asthma have short-acting beta-2 agonists (SABA) inhalers (such as albuterol, levalbuterol, terbutaline, metaproterenol and pirbuterol) for rescue therapy. For patients with moderate-to-severe persistent asthma, long-acting beta-2 agonists (LABA) for example, salmeterol and formoterol or leukotriene inhibitors are often added to inhaled corticosteroid treatments. Commonly used corticosteroids include; beclomethasone, triamcinolone, flunisolide, ciclesonide, budesonide, fluticasone and mometasone. Antimuscarinic drugs are also used for alleviating bronchoconstriction and dyspnea in asthma patients. There are both short- and long-acting anti-muscarinic drugs available. Select use of biologic agents can be considered for those patients with more severe, difficult-to-control forms of asthma. Omalizumab was the first approved biologic for eosinophilic asthma and works by binding to immunoglobulin E (IgE) and downregulating activation of airway inflammation. Omalizumab is FDA approved for treatment of moderate to severe allergic asthma, in patients older than 6 years and improves asthma symptoms, reduces exacerbations and eosinophil counts. Newer biologic agents targeting IL-5 pathways are also available, including; mepolizumab, reslizumab and benralizumab. IL-5 is a major cytokine responsible for the growth, differentiation, and survival of eosinophils, which play a significant role in airway inflammation in asthma patients. It is evident that a major strategy in the control of eosinophilic asthma is to antagonize production of interleukin cytokines, particularly IL-5. Unfortunately, existing synthetic biologics on the market come with very severe side effects and at very high costs, frequently in the tens of thousands of dollars per year for treatment.

One embodiment in this present invention is an alternative treatment of individuals with asthma currently using corticosteroids, short- and long-acting beta-2 agonists and antimuscarinic drugs with a multifunctional inhaled aerosolized pharmaceutical liquid composition comprising natural antioxidants, natural anti-inflammatory compounds and vitamins.

One embodiment in this present invention is an inhaled aerosolized pharmaceutical liquid composition and method treatment to reduce the concentration of heavy metals in the lungs of current and former cigarette smokers, individuals exposed to second hand cigarette smoke and individuals exposed to air pollutants using metal chelates in the liquid compositions.

### Inhalation Therapy

Inhalation refers to a process by which a gas or substance enters the lungs. Inhalation can occur through a gas or substance, e.g., a substance, such as a pharmaceutical composition according to the invention, in an aerosol form, passing through the mouth or nose (or a stoma (hole) into the trachea in the case of an individual who has had a tracheotomy), the respiratory tract, and into the lungs. Thus, unless otherwise indicated, the terms “inhalation”, “administration”, and other similar terms include administering a substance to the lungs by inhalation through the mouth (i.e., orally) and by inhalation through the nose (i.e., nasally) (as well as by inhalation through a stoma (hole) into the trachea in the case of an individual who has had a tracheotomy).

The particle size of inhaled cigarette smoke is typically between 0.1 and 1.0 microns (μm). The particle sizes of inhaled cigarette smoke varied between 186 nm and 198 nm in an experimental device developed by Sahu et al. (2013) at a puff volume of 35 mL/puff. When the puff volume was increased to 85 mL/puff the particle size increased to about 300 nm. Cigarette smokers typically retain approximately 30-66% of the particulate phase contained in cigarette smoke and the amount of particulate absorption by the smoker's respiratory tract is related to size and solubility of the substance. Sahu et al. (2013) calculated that 61.3% of inhaled cigarette smoke particles are deposited in the human respiratory tract. In contrast, E-cigarette aerosol is best described as a mist, which is an aerosol formed by condensation or atomization composed of spherical liquid droplets in the sub-micrometer to 200 μm size range. Alderman et al (2014) reported particle size measurements for e-cigarettes to be in the 260-320 nm count median diameter range.

Many types of medical conditions can be treated by inhalation of various natural and synthetic liquid substances. These chemical substances can be administered to a patient using different type of inhalation drug delivery systems applicators including: nebulizers, in which a liquid medicine is turned into a mist that is subsequently inhaled to the lungs; Metered Dose Inhalers (MDIs) which comprise a pressurized inhaler that delivers medication by using a propellant spray (e.g., a mixture of drug and a propellant); Soft Mist Inhalers (SMI) which is a multi-dose, propellant-free, hand-held, aerosol generating liquid inhaler that uses a compressed spring, instead of a compressed gas, to generated an aerosol; ultrasonic vaping devices and thermal aerosolization devices, including vaping devices, that are trigged to atomize a stored liquid in a reservoir by heating with a heating element or coil to generate an aerosolized mixture (i.e., vapor) that is inhaled by users. Nebulizers are commercially available to vaporize solutions or stable suspensions of a liquid into an aerosol mist either by means of a compressed gas, through a venturi orifice or by means of ultrasonic action.

The liquid compositions presented in this application for the instant invention can be vaporized or aerosolized by any of the above, or any other orally or nasally administered liquid-based inhalation drug delivery systems. A person ordinarily skilled in the art would recognize that the liquids set forth in this present invention can be used to treat respiratory and lung diseases and can also be administered by any type of device that creates a vapor or aerosolized liquid that can be orally administered to a patient.

Particle size plays an important role in lung deposition, along with particle velocity and settling time. As particle size increases above 3 μm, aerosol deposition shifts from the periphery of the lung to the conducting airways. Oropharyngeal deposition increases as particle size increases above 6 μm. Exhaled loss is high with very small particles of 1 μm or less. Consequently, particle sizes of 1-5 μm effectively reach the lung periphery, whereas 5-10 μm particles deposit mostly in the conducting airways, and 10-100 μm particles deposit mostly in the nose and mouth (America Association for Respiratory Care, 2017). The preferred particle size of the aerosolized liquids in this present invention is about 1 μm to about 5 μm.

In an embodiment of this present invention, liquid compositions and methods of use of the aerosolizable liquid compositions include a nicotine salt as part of a nicotine replacement therapy cigarette smoking cessation system, while providing simultaneous treatment of the lung and respiratory tract diseases and impact from a person's history of cigarette smoking. In one embodiment of this present invention, an aerosolizable liquid composition comprises a nicotine salt, a plant-based TRPA1 antagonists, natural thiol amino acid containing compounds, CB₂ agonists, amino acids, naturally occurring antioxidants, vitamins, and flavonoid compounds and heavy metal complexing compounds.

In another embodiment of this present invention a liquid composition and methods of use wherein the liquid is either vaporized, aerosolized, or both, and breathed in by a patient to reduce inflammation in the individual's respiratory tract associated with COPD, asthma, cystic fibrosis and other respiratory diseases associated with diminished lung capacity. In yet another embodiment of this present invention is a multifunctional composition that reduces the concentration and effects of reactive oxygen species in the lungs resulting from one or more diseases, including exposure to cigarette smoke, other types of smoke, and air pollutants.

Yet another embodiment of this present invention are aerosolizable liquid compositions and methods of use to reduce reactive oxygen species in the lungs, including lung epithelial lining fluid, epithelial cells, neutrophils, eosinophils, macrophages, lymphocytes, monocytes and tissues in the lungs of patients with diseases that result in an imbalance of oxidant/antioxidant concentrations from endogenous causation of reactive oxygen species. Yet another embodiment of this present invention are aerosolizable liquid compositions and methods of use to reduce inflammatory cytokines in the lungs, including lung epithelial lining fluid, epithelial cells, neutrophils, eosinophils, macrophages, lymphocytes, monocytes and tissues in the lungs of patients the result of cigarette smoking, asthma, COPD and other respiratory diseases present in the epithelial lining fluid that covers the mucosa of the alveoli, the small airways, and the large airways. In an embodiment of this present invention, inflammatory cytokines that are inhibited are Interferon-1β (IL-1β), IL-6, IL-8, IL-12, interferon-γ, tumor necrosis factor-α (TNF-α). In another embodiment of this present invention are liquid compositions that activate anti-inflammatory cytokines including, IL-1 receptor antagonist (IL-1r), IL-4, IL-10, IL-11, and IL-13).

A pharmaceutical composition according to the invention can further comprise, or can be administered together with, one or more additional therapeutic agents. In some embodiments, the additional one or more therapeutic agents may be present in a pharmaceutical composition in addition to plant extract components of the pharmaceutical composition. The one or more additional therapeutic agents may be a prescription drug or a non-prescription (i.e., over-the-counter) drug. For example, any additional therapeutic agent also may be used in the treatment of a lung or respiratory tract disorder, such as asthma, COPD, emphysema, and chronic bronchitis. For example, the one or more additional therapeutic agents can include a short acting beta2-adrenoceptor agonist (SABA) (e.g., salbutamol, albuterol, terbutaline, metaproterenol, pirbuterol), an anticholinergic (e.g., ipratropium, tiotropium, aclidinium, umeclidinium bromide), an adrenergic agonist (e.g., epinephrine), a corticosteroid (e.g., beclomethasone, triamcinolone, flunisolide, ciclesonide, budesonide, fluticasone propionate, mometasone), a long acting beta2-adrenoceptor agonist (LABA) (e.g., salmeterol, formoterol, indacaterol), a leukotriene receptor antagonist (e.g., montelukast, zafirlukast), a 5-LOX inhibitor (e.g., zileuton), an antimuscarinic, a bronchodialator, xylitol, and/or combinations of two or more of these.

A pharmaceutical composition according to the invention can further comprise, or can be administered together with, one or more additional antiviral agents. In some embodiments, the additional one or more antiviral agents may be present in a pharmaceutical composition in addition to plant extract components of the pharmaceutical composition. The one or more additional antiviral agents may be a prescription drug or a non-prescription (i.e., over-the-counter) drug. For example, any additional antiviral agent also may be used in the treatment of a lung or respiratory tract disorder, such as asthma, COPD, emphysema, and chronic bronchitis. For example, the one or more additional antiviral agents can include amantadine, rimantadine, zanamivir, oseltamivir, ribavirin, acyclovir, ganciclovir, laninamivir, zanamivir, peramivir, ganciclovir, cidofovir, chloroquine, hydroxychloroquine, ivermectin, lopinavar, remdesivir, and foscarnet, and/or combinations of two or more of these.

A pharmaceutical composition according to the invention can further comprise, or can be administered together with, one or more additional antibacterial agents. In some embodiments, the additional one or more antibacterial agents may be present in a pharmaceutical composition in addition to plant extract components of the pharmaceutical composition. The one or more additional antibacterial agents may be a prescription drug or a non-prescription (i.e., over-the-counter) drug. For example, any additional antibacterial agent also may be used in the treatment of a lung or respiratory tract disorder, such as asthma, COPD, emphysema, and chronic bronchitis. For example, the one or more antibacterial agents can include tobramycin, gentamicin, amikacin, imipenem-cilastatin, ceftazidime, fluoroquinolones, colistin, ciprofloxacin, aztreonam, polymyxins, colistimethate, pentamidine, and/or combinations of two or more of these.

This disclosure also relates to the use of one or more water soluble natural thiol amino acid containing compounds including; glutathione, N-acetyl cysteine and carbocysteine in a liquid that is aerosolized, vaporized or both, for inhalation to reduce, neutralize and/or inhibit the formation of reactive oxygen species, reactive nitrogen species and other types of free radical species that can otherwise cause damage to the upper and/or lower respiratory tracts of a person. This disclosure further relates to the use the of the water soluble natural sulfonic amino acid, taurine that can react with endogenously produced hypochlorous acid in the lungs to form a much less toxic taurine chloramine (Tau-Cl). Taurine acts in our compositions to neutralize reactive oxidant species and to neutralize inflammatory cytokines by the formation of Tau-Cl. Optional additives to the liquid compositions in this present invention include preservatives if the composition is not prepared sterile, additional antioxidants, flavoring agents, volatile oils, buffering agents and surfactants.

In the present invention, an “inflammatory disease” or “inflammation” is a broad indication that refers to any disease that designates inflammation of the respiratory tract as a main cause or inflammation caused by disease. Specifically, an inflammatory disease includes may include general or localized inflammatory diseases (for example: allergies; immune-complex disease; hay fever; and respiratory diseases (for example, asthma; epiglottitis; bronchitis; emphysema; rhinitis; cystic fibrosis; interstitial pneumonitis; chronic obstructive pulmonary disease, acute respiratory distress syndrome; coniosis; alveolitis; bronchiolitis; pharyngitis; pleurisy; or sinusitis); but not limited to those. In this present invention inflammatory respiratory diseases may also be caused by exogenous environmental and occupational exposures to particulate and non-particulate air pollutants, that are collectively either indoor or outdoor air pollutants, including in an enclosed or semi-enclosed space, such as an automobile, bus, train, boat or any other transportation or space-related related vehicle.

In this present invention a “vapor” is defined as diffused matter (such as smoke or fog) suspended floating in the air and impairing its transparency and also a substance in the gaseous state as distinguished from the liquid or solid state. A vapor therefore can be a compound in a gas phase, for example, the volatilization of a volatile liquid being transferred from a liquid phase to a gaseous phase, as well as being suspended liquid particles. In this present invention an “aerosol” is defined as is a suspension of fine solid particles or liquid droplets, in air or another gas.

One embodiment of this present disclosure are compositions and methods of use to antagonize, inactivate or block TRPA1 activation in the lungs from exogenous chemicals that would otherwise cause TRPA1 activation, for example, from cigarette smoke, by inhalation of aerosolized natural plant compound TRPA1 antagonists that are inhaled using electronic vaping devices, ultrasonic vaporization devices or other thermal aerosolization or vaporization devices, nebulizers or other types of devices that are used to transfer a liquid to aerosol and/or gas phases then inhaled by an person. Another embodiment of this disclosure is to limit damage of lung tissues from reactive oxygen species, for example, from cigarette and other exogenous sources of smoke and exogenous air pollutants by natural thiol amino acid containing compounds, CB₂ agonists, amino acids, naturally occurring antioxidants, phytochemicals and flavonoid compounds, vitamins and heavy metal complexing compounds that are inhaled using electronic vaping devices, ultrasonic vaporization devices or other thermal aerosolization or vaporization devices, nebulizers or other types of devices that are used to transfer a liquid to aerosol and/or gas phases then inhaled by an person. Yet another complementary feature of this present invention comprises plant-based TRPA1 antagonists, natural thiol amino acid containing compounds, CB₂ agonists, amino acids, naturally occurring antioxidants, vitamins and bioflavonoid compounds and heavy metal complexing compounds to a liquid that is inhaled using electronic vaping devices, ultrasonic vaporization devices or other thermal aerosolization or vaporization devices, nebulizers or other types of devices that are used to transfer a liquid to aerosol and/or gas phases then inhaled by an person that have one or more antioxidant, anti-inflammatory, antiallergenic, antiviral, or anti-carcinogenic properties.

This disclosure relates in part to a method of reducing damage to the lungs from current and past cigarette smoking and other exogenous or endogenous chemicals or particulate matter.

Yet another feature of this disclosure is a method to inhibit or neutralize the release of calcitonin gene related peptide (CGRP) in lung tissues through inactivation of TRPA1. CGRP is a member of the calcitonin family of peptides, existing in two forms: α-CGRP and β-CGRP. CGRP is released when TRPA1 is activated in the lungs through the activation of TRPA1 by cigarette smoke. Cigarette smoke initially causes an increase in the extracellular level of reactive oxygen species, which in turn activates lung epithelial TRPA1. Activation of TRPA1 then transduces this stimulation induced by cigarette smoke into the transcriptional regulation of lung inflammation via an influx of Ca²⁺. In another embodiment of this present invention is a liquid composition, when vaporized, aerosolized or both, and breathed into the respiratory tract results in an increase in concentrations of compounds in the lungs that are natural TRPA1 antagonists, natural TRPM8 agonists, natural thiol amino acid containing compounds, CB₂ agonists, amino acids, antioxidants, bioflavinoid compounds, vitamins, and metal chelates. In yet another embodiment of this present invention is a liquid composition containing mostly naturally occurring compounds, when vaporized aerosolized or both, and breathed into the respiratory tract results in an increase in concentrations of compounds in the lungs that are TRPA1 antagonists, TRPM8 agonists, natural thiol amino acid containing compounds, CB₂ agonists, amino acids, antioxidants, bioflavinoid compounds, vitamins, and natural metal chelates. The effects of breathing in vaporized, aerosolized or both, naturally occurring chemicals comprised in the liquids set forth in this present invention is to decrease one or more but not limited to tissue damage, inflammation, excess mucous accumulation, cough and cancer caused by reactive oxygen species the result of an imbalance in oxidant/antioxidant chemistry in the lungs. A reduction of inflammation in the lungs by breathing in gaseous and aerosolized phases of liquids set forth in this present invention include modulation of the immune system response, an increase bacteriostatic and fungistatic conditions in the lungs, and inhibition of production of tumor necrosis factor-a (TNF-α), interleukin-1β (IL-1β), interleukin-4 (IL-4), interleukin-5 (IL-5),leukotriene B4 (LTB4), thromboxane B2 (TXB2) and prostaglandin E2 (PGE2).

This disclosure yet further relates in part to cannabinoid compounds (both phytocannabinoid and synthetic cannabinoids), including but not limited to: 9-Tetrahydrocannabinol (delta-9-THC), 9-THC Propyl Analogue (THC-V), Cannabidiol (CBD), Cannabidiol Propyl Analogue (CBD-V), Cannabinol (CBN), Cannabichromene (CBC), Cannabichromene Propyl Analogue (CBC-V), Cannabigerol (CBG), cannabinoid terpenoids, and cannabinoid flavonoids; cannabinol (CBN) that are combined with TRPA1 antagonists, TRPM8 agonists, natural thiol amino acid containing compounds, CB₂ agonists, amino acids, antioxidants, vitamins, bioflavinoid compounds and natural metal chelates. Because of its lack of psychoactive properties, cannabidiol is a preferred phytocannabinoid in this disclosure.

Surprisingly, it has been found in this present invention that natural compounds can be combined to control gating to inhibit TRPA1 activation and therefore, can reduce inflammation and the effects of inflammation in the lungs the result of TRPA1 activation caused by exogenous and endogenous chemicals, including cigarette smoke. Yet in further compositions of this present disclosure 1,8-cineole and/or borneol are TRPA1 antagonists. Yet further compositions of this present disclosure include 1,8-cineole and/or borneol with natural thiol amino acid containing compounds. Yet further compositions of this present invention include CB₂ agonists. The preferred CB₂ agonists in this present invention is β-caryophyllene. Preferred compositions in this present invention include; 1,8-cineole as a TRPA1 antagonist and TRPM8 agonist; n-acetyl cysteine and glutathione that are naturally occurring thiol amino acid containing compounds that are also antioxidants; and an emulsifying compound and water. In yet another preferred composition, vitamin C (ascorbic acid) and vitamin B12 (methylcobalamin) are added to 1,8-cineole, N-acetyl cysteine and glutathione to increase the multifunctional properties of the aerosolized or vaporized liquids set forth in this present invention. Yet further compositions of this present disclosure include 1,8-cineole and/or borneol with water soluble antioxidants, bioflavinoid compounds, heavy metal chelators, emulsifying compounds and water.

This disclosure relates to the use of the bioflavinoid compound thymoquinone in a liquid that is used to become vaporized for inhalation to impart antioxidant, anti-inflammatory, antiallergenic, antiviral and anti-carcinogenic properties to the lungs of individuals exposed to cigarette smoke. Additionally, this disclosure relates to the use of the bioflavinoid compound thymoquinone in a liquid that is used to become aerosolized or vaporized for inhalation to decrease inflammation mediators, including IL-8, neutrophil elastase, TNF-α and malondialdehyde in the upper and lower respiratory tracts.

This disclosure relates to the use of the bioflavinoid compound berberine in a liquid that is used to become aerosolized or vaporized for inhalation to impart antioxidant, anti-inflammatory, antiallergenic, antiviral and anti-carcinogenic properties to the lungs of individuals exposed to cigarette smoke. Additionally, this disclosure relates to the use of the bioflavinoid compound berberine in a liquid that is used to become aerosolized vaporized for inhalation to decrease inflammation mediators, including IL-8, neutrophil elastase, TNF-α and malondialdehyde in the upper and lower respiratory tracts.

Yet another feature of this disclosure relates to the use of the bioflavinoid compound curcumin in a liquid that is used to become vaporized for inhalation to neutralize and/or inhibit the formation of reactive oxygen species and other types of free radical species that can otherwise cause damage to the upper and/or lower respiratory tract. Curcumin is known to have antioxidant and anti-inflammatory properties. The anti-inflammatory effect of curcumin is most likely mediated through its ability to inhibit cyclooxygenase-2 (COX-2), lipoxygenase (LOX), and inducible nitric oxide synthase (iNOS). Because inflammation is closely linked to tumor promotion, curcumin with its potent anti-inflammatory property will exert chemopreventive effects on carcinogenesis.

Another feature of this disclosure relates to the use of additional natural compounds that exhibit anti-inflammatory properties in respiratory therapies, including; andrographolide, astragalin, cardamonin, kaempferol, luteolin, naringin, oroxylin A, quercetin, geniposide, genistein, ellagic acid, Escin, Glycyrrhizin, Hydroxysafflor yellow A, baicalein, baicalin, cepharanthine, columbianadin, esculin, imperatorin, imperatorin, isoorientin, isovitexin, moracin M, orientin, phillyrin, platycodin D, resveratrol, schisantherin A, silymarin, tectorigenin, triptolide, paeonol, zingerone, paeonol, protocatechuic acid, limonene, linalool, phillyrin, asperuloside, prime-O-glucosylcimifugin, cannabidiol, flavone, tricetin, luteolin, apigenin-7-glucoside, baicalei, baicalin, afzelin, hyperoside, quercitrin, morin, quercetin, fisetin, tectorigenin, eriodictyol, naringin, hesperidin, sakuranetin, taraxastero, vitexin, mogroside V, triptolide, minnelide, esculentoside, columbianadin, esculin, and imperatorin. Further, this disclosure relates to compositions and methods to reduction inflammation of the respiratory tract including extracts and essential oils from the following plants; Acanthopanax senticosus, Aconitum tanguticum, Alisma orientale Juzepzuk, Angelica decursiva, Antrodia camphorate, Alstonia scholaris, Artemisia annua, Azadirachta indica, Callicarpa japonica Thunb., Canarium lyi C. D. Dai & Yakovlev, Chrysanthemum indicum, Coscinium fenestratum Cnidium monnieri, Eleusine indica, Eucalyptus cinerea, Eucalyptus globulus, Euterpe oleracea Mart., Galla chinensis., Ginkgo biloba., Gleditsia sinensis, Glycyrrhiza uralensis, Houttuynia cordata, Juglans regia L. kernel, Lonicera japonica flos, Lysimachia clethroides Duby, Melaleuca linariifolia, Mikania glomerata Spreng, Mikania laevigata Schultz, Mikania laevigata Schultz, Nigella sativa, Paeonia sulfruticosa, Phellodendri cortex, Punica granatum, Rabdosia japonica var. glaucocalyx, Rosmarinus officinalis, Schisandra chinensis Baillon, Stemona tuberosa, Taraxacum officinale, Taraxacum mongolicum hand.-Mazz, Thymus satureioides, Uncaria tomentosa and Viola yedoensis.

Aerosolizable pharmaceutical liquid compositions of this present invention can also be comprised of carriers that enable the liquids and resulting aerosolized compounds to be most effectively delivered into the lungs, generally but not limited to nebulizers, ultrasonic vaporization devices and thermal electronic vaporization systems, such as e-cigarettes and other types of vaping devices. The carrier composition may include such compounds, but not limited to sterile water, pH buffers, acids, bases, surfactants, emulsifiers, glycols, vegetable glycerin and inorganic salts to make the composition isotonic with lung epithelial lining fluid.

Yet another feature of this invention is a lubricating viscosity modifier added to the liquid that is used to become aerosolized or vaporized for inhalation. The lubricating viscosity modifier can be selected from one or more of the group including a carbomer, polymers, acacia, alginic acid, carboxymethyl cellulose, ethylcellulose, hydroxyethyl cellulose, hydroxypropyl cellulose, methylcellulose, poloxamers, polyvinyl alcohol, sodium alginate, tragacanth, guar gum, sodium hyaluronate, hyaluronic acid, xanthan gum, glycerin, vegetable glycerin, polyethylene glycol, and polyethylene glycol (400).

Yet another feature of this invention is a stable suspension creating ingredient that can be added to one or more of the ingredients individually or to the bulk liquid added to the liquid that is used to become aerosolized or vaporized for inhalation. The stable suspension creating ingredient can be selected from one or more of the group of an emulsifiers or liposomes. Liposomes can entrap both hydrophobic and hydrophilic compounds and can be used in this present invention to target, localize or specifically absorb or adsorb the chemicals into or onto specific tissues, fluids or cell types in the lungs. A liposome has an aqueous solution core surrounded by a hydrophobic membrane, in the form of a lipid bilayer. Solutes dissolved in the liposome core cannot readily pass through the bilayer. Hydrophobic chemicals associate with the bilayer. A liposome can be hence loaded with hydrophobic and/or hydrophilic molecules. While the majority of the compounds comprising this present invention are hydrophilic, some are more hydrophobic, such as 1,8-cineole, β-caryophyllene, resveratrol, thymoquinone, epigallocatechin gallate and other catechin compounds, curcumin and borneol. Compositions including any of these compounds or other hydrophobic compounds at concentrations greater than their solubility in the aqueous bulk solutions may require them to be emulsified in the bulk solution in oil-in-water (O/W) micro- and nano-emulsions or to have individual hydrophobic compounds incorporated in liposome structures. A person ordinarily skilled in the art would readily understand that a variety of methods could be used to create stable homogeneous suspensions with the mixtures of hydrophilic and hydrophobic compounds set forth in this present invention.

Yet another feature of this disclosure is the use of a pH buffer to adjust the pH of the liquid to that of healthy epithelial lung fluid of approximately 7.2. Another feature of this present invention is the addition of salts to result in liquid compositions that are isotonic with epithelial lung fluids.

A feature of this instant invention presents liquid formulations and methods of use to treat various respiratory diseases associated with exposure to cigarette smoke and other types of smoke and excessive imbalances of oxidants and antioxidants in the lungs, creating reactive oxygen species that subsequently result in inflammation, DNA damage and a cascade of cytokine, neuropeptide and nociceptor activation. Cigarette smoke can generate 10¹⁵ reactive oxygen species radicals per puff and the compositions and methods of use of the presented liquids that are aerosolized in this present invention are intended to decrease damage in the respiratory system of active cigarette smokers, former cigarette smokers and those exposed to second hand smoke. It is understood by individuals ordinarily skilled in the art that both the short- and long-term health of individuals who are active cigarette smokers have the greatest potential to improve by the cessation of smoking. However, the addictive nature of nicotine, in part, makes it difficult for active cigarette smokers to stop smoking. This invention discloses compositions and methods of use of nicotine-containing liquids that can be aerosolized in a ultrasonic vaporization device, a thermal vaporization system, such as vaping devices and e-cigarettes, that also provides a multifunctional treatment for lung and respiratory diseases comprising plant-based TRPA1 antagonists, CB₂ agonists, natural thiol amino acid containing compounds, naturally occurring antioxidants, amino acids and flavonoid compounds and heavy metal complexing compounds. Methods of use of this coupled nicotine-respiratory system drug treatment include both the complete cessation of cigarette smoking or substitution with the nicotine-containing respiratory system drug treatment compositions disclosed in this present invention. If a cigarette smoker is not able to complete quit smoking cigarettes, a portion of their daily nicotine consumption can be substitute by using the nicotine-containing compositions disclosed in this patent. Both complete cessation of cigarette smoking, as well substituting a portion of an individual's daily nicotine consumption from cigarettes by inhalation of the nicotine-containing aerosolizable pharmaceutical liquid compositions disclosed in this present invention will reduce respiratory system damage, and other health impacts from active cigarette smoking.

### Transient Receptor Potential (TRP) Ion Channels and Smoking

Transient Receptor Potential (TRP) ion channels represent a heterogeneous system oriented towards environment perception and participate in sensing visual, gustatory, olfactive, auditive, mechanical, thermal, osmotic, chemical and pruritogenic stimuli. The Transient Receptor Potential family of channels, currently contains more than 50 different channels and 27 of these are found in humans. Transient Receptor Potential channel gating is operated by both the direct action on the channel by a plethora of exogenous and endogenous physicochemical stimuli. A large and significant amount of evidence indicates that the TRPA1 ion channel plays a key role in the detection of pungent or irritant compounds; including compounds contained in different spicy foods, such as allyl isothiocyanate (in mustard oil), horseradish, allicin and diallyl disulfide in garlic, cinnamaldehyde in cinnamon, gingerol (in ginger), eugenol (in cloves), methyl salicylate (in wintergreen), menthol (in peppermint), carvacrol (in oregano), thymol (in thyme and oregano), and the cannabinoid compounds cannabidiol (CBD), cannabichromene (CBC) and cannabinol (CBN) (in marijuana and industrial hemp). In addition, environmental irritants and industry pollutants, such as acetaldehyde, formalin, formaldehyde, hydrogen peroxide, hypochlorite, isocyanates, ozone, carbon dioxide, ultraviolet light, and acrolein (a highly reactive α,β-unsaturated aldehyde present in tear gas, cigarette smoke, smoke from burning vegetation, vaping liquids and vehicle exhaust), have been recognized as TRPA1 activators. A number of TRP channels (TRPA1, TRPV1 and TRPV4) have been linked to sensory perception relevant to a cough response.

Bessac et al. (2008) reported both hypochlorite, the oxidizing mediator of chlorine, and hydrogen peroxide, a reactive oxygen species, activated Ca²⁺ influx and TRPA1 activation in mice cells and that mice cells genetically lacking TRPA1 had no such response. In respiratory tests with TRPA1-deficient mice, they displayed profound deficiencies in hypochlorite- and hydrogen peroxide-induced respiratory depression as well as decreased oxidant-induced pain behavior. These authors concluded that TRPA1 is an oxidant sensor in sensory neurons, initiating neuronal excitation and subsequent physiological responses in vitro and in vivo. Based on their data, they also concluded that TRPA1 activation may also contribute to the effects of chlorine and other TRPA1 agonists on chemosensory nerve endings in the lower airways. Because reactive irritants are efficiently cleared in the upper airways, sensory activation in the lower airways requires higher exposure levels. Extended or high-level exposure to oxidants, such as those experienced in victims of chlorine gas exposures, induce severe pain, cough, mucus secretion, and bronchospasm. These authors also concluded that TRPA1 antagonists or blockers, may be used to suppress sensory neuronal hyper-excitability in airway disease and TRPA1 represents a promising new target for the development of drug candidates with potential antitussive, analgesic, and anti-inflammatory properties. In one embodiment of the present invention are inhaled aerosolized pharmaceutical liquid composition and methods for the treatment for individuals or soldiers exposed to chemical warfare agents that are respiratory irritants, coughing agents, and/or choking agents. Such chemical warfare agents can include tear (lachrymator) agents, vomiting agents, blistering agents (such as nitrogen and sulfur mustard agents and arsenicals (e.g., lewisite)), and choking agents (such as chlorine gas, chloropicrin, diphosgene, phosgene, disulfur decafluoride, perfluoroisobutene, acrolein, and diphenylcyanoarsine).

Kichko et al. (2015) reported that cigarette smoke contains volatile reactive carbonyls such as formaldehyde and acrolein that both activate TRPA1 in vitro and ex vivo in mouse trachea and larynx, as measured by means of calcitonin gene related peptide (CGRP) production, which modulates the production of proinflammatory cytokines. In the trachea, the gas phase of cigarette smoke (gas phase only) and whole cigarette smoke were equally effective in releasing calcitonin gene related peptide, whereas the larynx showed much larger whole cigarette smoke than gas phase responses. They concluded that nicotinic receptors contribute to the sensory effects of cigarette smoke on the trachea, which are dominated by TRPA1, but not TRPV1.

Mukhopadhyay et al. (2016) reported that the TRPA1 ion channel is expressed abundantly on the C fibers that innervate almost entire respiratory tract starting from oral cavity and oropharynx, conducting airways in the trachea, bronchi, terminal bronchioles, respiratory bronchioles and up to alveolar ducts and alveoli, They reported that TRPA1 plays the role of a “chemosensor”; detecting presence of exogenous irritants and endogenous pro-inflammatory mediators that are implicated in airway inflammation and sensory symptoms like chronic cough, asthma, COPD, allergic rhinitis and cystic fibrosis. TRPA1 can remain activated chronically due to elevated levels and continued presence of such endogenous ligands and pro-inflammatory mediators. They also reported that various noxious chemicals and environmental/industrial irritants that activate TRPA1 also are triggers for asthma or reactive airways dysfunction syndrome (RADS) and are known to worsen asthma attacks. They conclude that there is promising evidence to indicate targeting TRPA1 may present a new therapy in treatment of respiratory diseases in near future.

Li et al. (2015) confirmed the important role of lung epithelial TRPA1 in the induction of IL-8 by cigarette smoke extract in primary human bronchial epithelial cells. These in vitro findings, using primary human bronchial epithelial cells, suggest that exposure to cigarette smoke extract initially causes an increase in the extracellular level of reactive oxygen species, which in turn activates lung epithelial TRPA1. TRPA1 activation then transduces this stimulation induced by cigarette smoke into the transcriptional regulation of lung inflammation via an influx of Ca²⁺. They reported that Ca²⁺ influx was prevented by decreasing extracellular reactive oxygen species with the antioxidant radical scavenger, N-acetyl-cysteine. The decrease in Ca²⁺ influx was similar using pretreatment of N-acetyl-cysteine and the experimental synthetic TRPA1 antagonist HC030031.

Yang et al. (2006) demonstrated that exposure of human MonoMac6 cells to cigarette smoke extract at 1% and 2.5%, increased IL-8 and TNF-α production, with significant depletion of glutathione levels associated with increased reactive oxygen species release, in addition to activation of NF-κB. They reported that the inhibition of inhibitor of kappa B (IκB) kinase ablated the cigarette smoke extract-mediated IL-8 release, enabling the authors to propose that this inflammatory process was dependent on the NF-κB pathway. These authors also observed that cigarette smoke extract reduced histone deacetylase (HDAC) activity and HDAC1, HDAC2, and HDAC3 protein levels. When these researchers pretreated cells with glutathione, they reversed cigarette smoke-induced reduction in HDAC levels and significantly inhibited IL-8 release.

Facchinetti et al. (2007) reported that many substances contained in cigarette smoke, including reactive oxygen species, have been proposed to be responsible for the inflammatory process of COPD. These authors reported that acrolein and crotonaldehyde at micromolar concentrations, both α,β-unsaturated aldehydes, contained in aqueous cigarette smoke extract (CSE), evoke the release of the neutrophil chemoattractant IL-8 and of the pleiotropic inflammatory cytokine TNF-α from the human macrophagic cell line U937. They concluded that that α,β-unsaturated aldehydes were major mediators of cigarette smoke-induced macrophage activation, suggesting they contribute to pulmonary inflammation associated with cigarette smoke.

Blocking TRPA1 is emerging as a strategic treatment for a number of respiratory diseases and the role of TRPA1 in airway pathologies has been corroborated by studies using the TRPA1 knock-out (KO) mice and TRPA1 antagonists. In wild-type mice, airway exposure to hypochlorite or hydrogen peroxide evoke respiratory depression as manifested by a reduction in breathing frequency and increase in end expiratory pause, both of which were attenuated in TRPA1 KO mice. Allyl isothiocyanate (AITC), acrolein, crotonaldehyde and cinnamaldehyde are potent TRPA1 agonists and have been shown to induce dose dependent and robust tussive response in guinea pigs which was attenuated by the synthetic TRPA1 antagonist from Hydra Biosciences, HC-030031. Similarly, citric acid induced tussive response in guinea pigs was inhibited by a potent and selective TRPA1 antagonist, GRC 17536. Anti-tussive effects of other TRPA1 antagonists have also been demonstrated in animal cough models.

Takaishi et al. (2012) reported that 1,8-cineole (eucalyptol) activates human TRPM8 (hTRPM8) and is a hTRPA1 antagonist. They also demonstrated that 1,8-cineole did not activate hTRPV1 or hTRPV2. 1,8-cineole is present in Eucalyptus oil from several species in highly varying concentrations (less than 5 percent to greater than 80 percent), in several Rosmarinus officinalis chemotypes (up to −50 percent) and in Salvia lavandulifolia (up to −25 percent). It has been shown that TRPM8 activation decreases inflammation and pain. While TRPM8 activation by menthol was reported by these researchers, it did not decrease human inflammatory response, because it also activated TRPA1, which causes inflammation. Further, application of octanol (a known TRPA1 agonist and skin irritant) on the neck of human subjects followed by 1,8-cineole significantly reduced the irritation of octanol through inhibition of TRPA1 by 1,8-cineole.

As a follow-up to this research, an additional study was published by the same research group (Takaishi, et al., 2014) on the role of several monoterpene analogs of camphor and their ability to inhibit hTRPA1. They reported that 1,8-cineole, camphor, borneol, 2-methylosoborneol, norcamphor and fenchyl alcohol did not activate hTRPA1 and that borneol, 2-methylisoborneol and fenchyl alcohol at 1 mM completely inhibited hTRPA1 activation by menthol and allyl isothiocyanate (AITC from mustard oil) at 1 mM and 10 uM, respectively. It was found that TRPA1 activation by 20 uM AITC was inactivated (IC-50 concentration) in order from lowest to highest concentration by 2-methylosoborneol (0.12 mM), borneol (0.20 mM), fenchyl alcohol 0.32 mM, camphor (1.26 mM) and 1,8-cineole (3.43 mM).

Wang, et al. (2016) reported that cardamonin is a TRAPA1 antagonist (IC50=454 nM), while not affecting TRPV1 and TRPV4. They also reported that cardamonin did not significantly reduce HEK293 cell viability, nor did it impair cardiomyocyte constriction.

In cellular studies, Juergens, et al. (1998) reported that 1,8-cineole, which has been traditionally used to treat symptoms of airway diseases exacerbated by infection, exhibited a 1,8-cineole dose-dependent and highly significant inhibition of production of TNF-α, interleukin-1β (IL-1β), leukotriene B4 (LTB4) and thromboxane B2 (TXB2). In a follow-up clinical study, Juergens et al. (2003) evaluated the anti-inflammatory efficacy of 1,8-cineole by determining its prednisolone equivalent potency in patients with severe asthma. Thirty-two patients with steroid-dependent bronchial asthma were enrolled in a double-blind, placebo-controlled trial. After determining the effective oral steroid dosage during a 2 month run-in phase, subjects were randomly allocated to orally receive either 200 mg of 1,8-cineole 3 times per day or placebo in small gut soluble capsules for 12 weeks. Oral glucocorticosteroids were reduced by 2.5 mg increments every 3 weeks. The primary end point of their investigation was to establish the oral glucocorticosteroid-sparing capacity of 1,8-cineole in patients with severe asthma. They reported reductions in daily prednisolone dosage of 36% with active treatment (range 2.5 to 10 mg, mean: 3.75 mg) were tolerated vs. a decrease of only 7% (2.5 to 5 mg, mean: 0.91 mg) in the placebo group (P=0.006). Twelve of 16 patients in the 1,8-cineole group versus four out of 16 patients in the placebo group achieved a reduction of oral steroids (P=0.012). They concluded that long-term systemic therapy with 1,8-cineole had a significant steroid-saving effect in steroid-depending asthma. They also report that their results provided evidence of the anti-inflammatory activity of 1,8-cineole in asthma and a new rational for its use as mucolytic agent in upper and lower airway diseases. Their research suggested that 1,8-cineole was a strong inhibitor of cytokines and could be a long-term treatment of airway inflammation in bronchial asthma and other steroid-sensitive disorders. The reported a new mechanism of action of 1,8-cineole, which inhibited the production of inflammation mediators in monocytes. They also concluded that their findings explain the effective bronchodilation reported using 1,8-cineole in their clinical studies. Their data revealed similar concentration response curves to a steroid-like mode of action of 1,8-cineole that may be mediated by inhibition of nuclear transcription. Their work suggests the strong anti-inflammatory activity of 1,8-cineole could be a well-tolerated treatment of airway inflammation in obstructive airway disorders, especially in mild bronchial asthma and in more severe forms of asthma, and as a supplementary therapy with the objective of being able to reduce or replace glucocorticosteroids in the long term. In one embodiment of the present invention are inhaled aerosolized pharmaceutical liquid composition and methods treatment for individuals with asthma, COPD and other respiratory diseases to either eliminate or reduce the use of oral or inhaled corticosteriod compounds used in their medical treatment.

Worth et al. (2009) conducted a randomized, placebo-controlled multi-center clinical trial with the concomitant prescription of 1,8-cineole at a dose of 200 mg—3 times per day in capsules orally, on patients with stable chronic obstructive pulmonary disease. The primary hypothesis was that 1,8-cineole would decrease the number, severity and duration of exacerbations. Secondary outcome measures were lung function, severity of dyspnea and quality of life as well as relevant adverse effects. They reported significant improvement of airway resistance after a treatment duration of one week (−23%) and eight weeks (−21%) in placebo-controlled double-blind studies in patients with reversible obstructive ventilatory disorders. They also reported statistically significant reductions the frequency, duration and severity of exacerbations during the study period. Their collective findings underline that 1,8-cineole not only reduced exacerbation rates, but also provides clinical benefits as manifested by improved airflow obstruction, reduced severity of dyspnea and improvement of health status. They also cite a significant decrease of the requirement for systemic glucocorticosteroids in long-term therapy with 1,8-cineole (3×200 mg/day) in a placebo-controlled double-blind study in asthma requiring steroid treatment. Since glucocorticosteroids do not interfere with the release of histamine from mast cells, more research will be needed to determine the effects of 1,8-cineole on histamine release.

In an ex vivo study, Juergens et al. (1998b) investigated the effect of 1,8-cineole capsules (200 mg/day—3 times/day) on arachidonic acid (AA) metabolism in blood monocytes of patients with bronchial asthma. Production of arachidonic acid metabolites, LTB4 and PGE2, from isolated monocytes stimulated with the calcium ionophore A23187 were measured ex vivo; before therapy with 1,8-cineole, after 3 days of treatment (day 4); and 4 days after discontinuation of 1,8-cineole (day 8). The production of LTB4 and PGE2 from monocytes ex vivo was significantly inhibited on day 4 in patients with bronchial asthma (−40.3%, n=10 and −31.3%, p=0.1, n=3 respectively) as well as in healthy volunteers (−57.9%, n=12 and −42.7%, n=8 respectively). These authors concluded that 1,8-cineole was shown to inhibit LTB4 and PGE2, both pathways of arachidonic acid metabolism.

In an additional in vitro study by Juergens et al. (2004) therapeutic concentrations of 1,8-cineole (1.5 μg/mL) significantly inhibited (n=13-19, p=0.0001) cytokine production in lymphocytes of TNFα, IL-4, and IL-5, by 92%, 84%, 70%, and 65%, respectively. Cytokine production in monocytes of TNFα, IL-6, IL-8 was also significantly (n=7-16, p<0.001) inhibited by 99%, 84%, 76%, and 65%, respectively. In the presence of 1,8-cineole (0.15 μg/ml) production of TNFα, IL-1β by monocytes and of IL-1β, TNF-α by lymphocytes was significantly inhibited by 77%, 61% and by 36%, 16%, respectively. These results characterize 1,8-cineole as strong inhibitor of TNFα and IL-1β and suggest smaller effects on chemotactic cytokines. This is increasing evidence for the role of 1,8-cineole to control airway mucus hypersecretion by cytokine inhibition, suggesting long-term treatment to reduce exacerbations in asthma, sinusitis and COPD.

TRPA1 is activated by cigarette smoke and many other environmental pollutants and industrial chemicals. In the respiratory system, TRPA1 is at least in part activated by reactive oxygen species resulting in the production NF-κB and a cascade of neuropeptides; including CGRP and Substance P, leading to the production of proinflammatory cytokines; including, TNFα, IL-4, and IL-5, IL-6 and IL-8. Reactive oxygen species produced in the lungs from cigarette smoke have also been shown to be reduced by the antioxidants, glutathione and N-acetyl cysteine. Further activation of TRPA1 in the respiratory system by reactive oxidant species has clearly been shown to be blocked by TRPA1 antagonists.

In one embodiment of this invention, TRPA1 antagonists are combined with antioxidants in an aerosolizable pharmaceutical liquid composition to decrease respiratory system damage from cigarette smoke, environmental and industrial air pollutants, lung-irritating and/or damaging chemical warfare agents, and respiratory system diseases in a multifunctional manner by combining natural compound antioxidants and natural compound TRPA1 antagonists.

### Transient Receptor Potential Nociceptors and Cancer

Prevarskaya et al., (2007, 2011) and Wu et al., (2010) demonstrated that TRP channels are involved in the regulation of proliferation, differentiation, apoptosis, angiogenesis, migration and invasion during cancer progression, and that the expression and/or activity of these channels is altered in cancers.

Takahashi et al. (2018) reported that TRPA1 is upregulated by nuclear factor erythroid 2—related factor 2 (NRF2) and promotes oxidative-stress tolerance in cancer cells. Cancer cell survival is dependent on oxidative-stress defenses against reactive oxygen species that accumulate during tumorigenesis. Together with the known importance of NRF2 in the induction of reactive oxygen species-neutralizing gene expression, they indicated that cancer cells mobilize a set of adaptive mechanisms, involving TRPA1-mediated non-canonical oxidative-stress defense as well as canonical reactive oxygen species-neutralizing mechanisms, to survive harsh oxidative challenges. In TRPA1-enriched breast and lung cancer spheroids, TRPA1 is critical for survival of inner cells that exhibit reactive oxygen species accumulation. Moreover, TRPA1 promotes resistance to reactive oxygen species-producing chemotherapies, and TRPA1 inhibition suppresses xenograft tumor growth and enhances chemosensitivity. These findings reveal an oxidative-stress defense program involving TRPA1 that could be exploited for targeted cancer therapies.

Wu et al. (2016) reported that in human small cell lung cancer (SCLC), TRPA1 mRNA levels were markedly upregulated in tumor specimens, compared to normal lung tissues and non-small lung cancer samples. In vitro treatment with the TRPA1 agonist, allyl isothiocyanate, a volatile toxic compound, on respiratory system-derived small cell lung cancer cell lines, caused an increment of the concentration of intracellular calcium. In an analysis of expression profile and assessment of TRPA1 expression in a cohort of 124 non-small lung cancer patients, the TRPA1 protein levels could be detected by immunohistochemistry in all cases. In addition to the higher primary tumor, TRPA1 upregulation is independently and negatively predictive disease-specific, distal metastasis-free and local recurrence-free survivals. Additionally, Schaefer et al. (2013) reported that TRPA1 was expressed in a panel of human small cell lung cancer cell lines. They also reported that TRPA1 mRNA was also more highly expressed in tumor samples of small cell lung cancer cell patients as compared to non-small cell lung cancer cell tumor samples or non-malignant lung tissue. Stimulation of small cell lung cancer cells with allyl isothiocyanate resulted in an increase in intracellular calcium concentration. Additionally, these authors reported that the calcium response was inhibited by TRPA1 antagonists. TRPA1 activation in small cell lung cancer cells prevented apoptosis induced by serum starvation and thus promoted cell survival, an effect which could be blocked by inhibition of TRPA1. Conversely, down-regulation of TRPA1 severely impaired anchorage-independent growth of small cell lung cancer cells. Since TRPA1 appears to play a pivotal role for cell survival in small cell lung cancer cells these authors proposed that TRPA1 could represent a promising target for therapeutic interventions. Finally, these authors also concluded that exogenous, inhalable activators of TRPA1 could be able to exert tumor promoting effects in small cell lung cancer cells.

### Cannabinoid Type 2 Receptor Signaling

The CB₂ receptor is the peripheral receptor for cannabinoids. It is mainly expressed in immune tissues, revealing that the endocannabinoid system has an immunomodulatory role. In this respect, the CB₂ receptor has been shown to modulate immune cell functions, both in vitro and in animal models of inflammatory diseases. Numerous studies have reported that mice lacking the CB₂ receptor have an exacerbated inflammatory phenotype. This suggests therapeutic strategies aimed at modulating CB₂ signaling could be promising for the treatment of various inflammatory conditions. CB₂ is mainly expressed in immune cells including neutrophils, eosinophils, monocytes, and natural killer cells Activation of the CB₂ receptors by endocannabinoids or selective synthetic agonists has been shown to protect against tissue damage in various experimental models of ischemic-reperfusion injury, atherosclerosis/cardiovascular inflammation and other disorders by limiting inflammatory cell chemotaxis/infiltration, activation, and related oxidative/nitrosative stress.

It has also been shown that CB₂ was up-regulated in non-small-cell lung cancer tissues and the up-regulation was correlated with tumor size and advanced non-small-cell lung cancer pathological grading (Xu, et al. 2019).

In addition to the CB₂ receptor binding to various phytocannabinoids, including CBD (Ki=2.680 μM), delta-9-THC (Ki=0.035 μM), CBN (Ki=0.096 μM), CB₂ also binds to the endocannabinoids arachidonoyl-ethanolamide (AEA) (Ki=0.371 μM) and 2-arachidonoyl-glycerol (2-AG) (Ki=0.650 Importantly, the CB₂ receptor also binds to β-caryophyllene (BCP) (Ki=0.155 (Turcotte, et al. (2016), which clearly demonstrates it is more effective at lower concentrations than is CBD. β-caryophyllene is found in essential oils of cloves (Syzygium aromaticum), cinnamon (Cinnamomum spp.), black pepper (Piper nigrum L.), and rosemary (Rosmarinus officinalis L) and is available in pure form through distillation from natural sources. β-caryophyllene use in foods has been approved by the U.S. Food and Drug Administration due to its low toxicity. While β-caryophyllene is a powerful CB₂ agonist it is not a cannabinoid compound and is not a CB′ receptor agonist and has no psychoactive properties. The disclosure relates to the use of the β-caryophyllene (BCP), a natural sesquiterpene compound, and its use in the aerosolizable pharmaceutical liquid formulations as a CB₂ agonist.

### Glutathione

Glutathione is an important water soluble antioxidant in plants, animals, fungi, and some bacteria. As such, it is capable of preventing damage to important cellular components caused by reactive oxygen species such as free radicals, peroxides, lipid peroxides, and heavy metals. In lungs, glutathione is important in modulating immune function and participates in the pulmonary epithelial host defense system (Buhl, et al. 1990). Depletion of intracellular glutathione suppresses lymphocyte activation by mitogens, and is important in lymphocyte-mediated cytotoxicity. A number of lung disorders are associated with an increased oxidant burden on the pulmonary epithelial surface and pulmonary epithelial cell damage, including idiopathic pulmonary fibrosis, asbestosis, cigarette smoking, adult respiratory distress syndrome, cystic fibrosis, and acute and chronic bronchitis. Glutathione supplementation is helpful in disorders of other organs associated with an increased oxidant burden, including enhancement of antioxidant protection in epithelial lung fluid.

The intracellular oxidation-reduction (redox) state remains homeostatic in the lungs, and is tightly regulated by intracellular antioxidant systems. Glutathione (γ-L-glutamyl-L-cysteinyl-glycine, glutathione) is the most abundant non-protein thiol amino acid and redox buffer in mammalian cells. Very importantly glutathione provides the first-line defense to reactive oxidant species. Glutathione compounds have multiple biological roles, including cell protection against oxidative stress and several toxic molecules, and are involved in the synthesis and modification of leukotrienes and prostaglandins. As an example, glutathione S-transferases protect cellular DNA against oxidative damage that can lead to an increase of DNA mutations or that induce DNA damage promoting carcinogenesis.

Glutathione S-transferases are able with react and conjugate to a wide range of hydrophobic and electrophilic molecules including many carcinogens, therapeutic drugs, and many products of oxidative metabolism, making them less toxic and predisposed to further modification for discharge from the cell. Glutathione not only directly interacts with reactive oxygen species and acts as a substrate for different enzymes to eliminate endogenous and exogenous compounds, but also it can conjugate with xenobiotics such as chemotherapy agents directly. Because many anticancer chemotherapy drugs are effectively toxic xenobiotic compounds, this can result in high glutathione levels and subsequently, anticancer drug resistance. However, glutathione is also involved in cell protection from free radicals, and in many cellular functions being particularly relevant in regulating carcinogenic mechanisms, including; sensitivity against xenobiotics, ionizing radiation and some cytokines, DNA synthesis and cell proliferation.

In cellular studies, van der Toorn et al, (2007) demonstrated that the gaseous phase of cigarette smoke decreases free sulfhydryl (—SH) groups of glutathione in solution and in airway epithelial cells. They reported that glutathione was irreversibly modified by unsaturated aldehydes that are generated during the combustion of tobacco. In their in vitro experiments it was demonstrated that exposure to cigarette smoke changed almost the entire pool of glutathione to glutathione E-aldehyde components. The enzymatic redox cycle, which is normally activated after oxidative stress and the formation of glutathione disulfide, the oxidized form of glutathione, could not be activated because of the depletion of glutathione into non-reducible glutathione components, with loss of the glutathione pool. This exhaustion of the pool of reduced glutathione may induce a chronic lack of antioxidant protection. Persistent smokers inhale more reactive oxygen species than can be scavenged by residual antioxidants, resulting in increased vulnerability to oxidative stress. This makes the synthesis of glutathione essential for cellular survival and protection of the lung. The development of COPD is associated with increased oxidative stress and reduced antioxidant resources. Cigarette smoking is the most important factor for the development of COPD.

Cellular stress induced by cigarette smoking is critically dependent on the intracellular reduced glutathione concentration. The lung responds to this challenge with adaptive responses that include up regulation of glutathione antioxidant defenses. Gould et al. (2011) demonstrated that the glutathione adaptive response consists of a coordinated response between glutathione synthesis, utilization, recycling, and transport into the lung epithelial lining fluid. The human alveolar surface area has been estimated to vary from 57.22 m² for human alveolar surface (USEPA, 2004) to 102 m² (Chen et al., 2016). Estimates of the thickness of the epithelial lining fluid layer on the alveoli vary significantly in the literature from 0.01μ to 0.3μ (Fröhlich et al., 201). Similarly, estimates of the epithelial lining fluid volume vary from 12 mL to as high as 70 mL, with 25 mL used as a common assumption (Fröhlich et al., 201). Concentrations of individual ingredients in the epithelial lining fluid after inhalation of aerosolized liquids in this present inventions will vary depending on an individual's physiological features (height and weight), the deposition efficiency of the particular nebulizer used, the conditions of the lungs, the unit dose and frequency of dosing.

Elevation in lung epithelial lining fluid glutathione levels is thought to act as a defense mechanism to limit the damaging effects of chronic smoking. Gould et al. (2010) have also shown that age adversely affects the lung glutathione adaptive response to acute cigarette smoking exposure in mice and that this response leads to increases in inflammation in the airways and increased DNA oxidation in the lung. In humans, glutathione levels drop sharply in humans around the age of 45 and this shortly proceeds the age at which COPD develops in chronic smokers.

In human testing, Gould et al. (2015) suggest that steady-state epithelial lining fluid glutathione levels are diminished with age and older smokers have impaired epithelial lining fluid glutathione adaptive responses to cigarette smoking with corresponding increases in inflammation, as evidenced by elevated exhaled nitric oxide (eNO) levels. These authors concluded that it is both glutathione levels and the endogenous ability to increase glutathione levels in response to stimuli that are important factors in the protection of the lung from the damaging effects of cigarette smoking.

Rusnack et al. (2000) used human bronchial epithelial cells (HBEC) from biopsy material obtained from three group of people as follows: those who smoked cigarettes and who had normal pulmonary function, cigarette smokers with normal pulmonary function, and cigarette smokers with COPD. They exposed these HBEC cells for 20 minutes to cigarette smoke or clean air. They also measured intercellular glutathione concentrations in HBECs both before exposure and after exposure to cigarette smoke. Their results indicate when only exposed to air, primary cultures of HBEC derived from smokers with normal pulmonary function and patients with COPD contained significantly more glutathione than did cultures from healthy people who never smoked cigarettes. These results are consistent with subsequent research that indicates cigarette smokers endogenously produce more glutathione in the lungs than non-smokers. When HBEC cells were exposed to cigarette smoke, the concentration of intracellular glutathione in all cultures were significantly lower when compared with those exposure only to air. However, the magnitude of glutathione concentration decrease in HBEC cells exposed to cigarette smoke (mean percent change) was different in the study groups: 72.9% in cells from patients with COPD; 61.4% in cells from healthy never-smokers; and 43.9% in cells from smokers with normal pulmonary function. The decrease of glutathione in cells from patients with COPD was significantly greater than that in cells from healthy never-smokers or smokers with normal pulmonary function. They also reported that increased levels of antioxidant capacity (i.e., higher glutathione concentrations) may protect against oxidant-mediated damage.

Rusnack et al. (2000) also reported HBEC of patients with COPD demonstrated a larger increase in cellular permeability and release of inflammatory cytokine soluble intercellular adhesion molecule-1 (sICAM-1) and IL-1β, compared with a control group of cigarette smokers without COPD. They also observed that the endogenously increased glutathione concentrations in the HBEC of smokers with normal pulmonary function was related to the decrease of epithelial cell permeability and release of inflammatory cytokine IL-1b and sICAM-1.

Buhl, et al. (1990) demonstrated that an aerosol nebulizer application of 4 mL of a 150 mg/mL glutathione solution over a 25 minute period increased glutathione epithelial lung fluid concentrations to a concentration of about 337 which was a 7-fold increase over baseline concentrations (45.7 μM) prior to treatment and remained elevated for a 2-hour period. In contrast, when these authors intravenously administered a 600 mg glutathione solution, they reported no measurable glutathione concentration increases in epithelial lung fluid. Buhl et al. (1990) suggest that aerosol administration of glutathione is a practical way to significantly augment glutathione levels on the epithelial surface of the human lower respiratory tract. They also reported that the aerosol administration of glutathione not only augmented epithelial lung fluid glutathione levels but it did so with no adverse effects. Their results are consistent with Witschi, et al. (1992) who reported that oral administration of glutathione was ineffective at increasing plasma glutathione levels when given to healthy subjects and therefore, it would be doubtful that oral supplementation of glutathione would be helpful at increasing concentrations in the lungs.

Prousky (2008) conducted a literature review to examine the clinical effectiveness of inhaled glutathione as a treatment for various pulmonary diseases and respiratory-related conditions. This author concluded glutathione inhalation is an effective treatment for a variety of pulmonary diseases and respiratory-related conditions. Even very serious and difficult-to-treat diseases, including cystic fibrosis and idiopathic pulmonary fibrosis yielded benefits from inhaled glutathione treatment. This author concluded that glutathione inhalation is very safe and rarely causes major or life-threatening side effects. He stated potential applications of glutathione treatment include Farmer's lung, pre- and post-exercise, multiple chemical sensitivity disorder and cigarette smoking. Prousky (2008) also concluded that glutathione inhalation should not be used as a treatment for primary lung cancer.

Mah et al. (2012) conducted a structural analysis of lead-glutathione complexes and concluded that Pb²⁺ complex formation with glutathione have implications for the rational design of chelating agents for therapeutic treatment of lead poisoning. One problem associated with commonly used chelating agents, including EDTA, is that they are not selective and can also bind essential Fe²⁺, Ca²⁺ and Zn²⁺ metal ions resulting in related toxic effects. These authors concluded that Pb²⁺ prefers to bind a maximum of three glutathione ligands through the cysteine-thiolate group in aqueous solution, suggesting that a specially tailored chelating agent with three sulfur donor atoms available for binding could be very efficient in sequestering Pb²⁺ ions.

### N-Acetyl Cysteine

A water soluble antioxidant widely available for the treatment of patients with chronic obstructive pulmonary disease is N-acetyl cysteine (NAC) and its use is reviewed by Dekhuijzen (2004). Preclinical studies and clinical trials have shown that antioxidant molecules such as small thiol molecules (N-acetyl-L-cysteine and carbocysteine), antioxidant enzymes (glutathione peroxidases), activators of Nrf2-regulted antioxidant defense system (sulforaphane) and vitamins, for example, C, E, and D, can boost the endogenous antioxidant system and reduce oxidative stress. In addition, they may slow the progression of COPD. N-acetyl cysteine exhibits direct and indirect antioxidant properties. The free thiol group in N-acetyl cysteine is capable of interacting with the electrophilic groups of reactive oxygen species. N-acetyl cysteine exerts an indirect antioxidant effect related to its role as a glutathione precursor. Glutathione serves as a central factor in protecting against internal toxic agents (such as cellular aerobic respiration and metabolism of phagocytes) and external agents (such as NO, sulfur oxide and other components of cigarette smoke, and pollution). The sulphydryl group of cysteine neutralizes these agents. Maintaining adequate intracellular levels of glutathione is essential to overcoming the harmful effects of toxic agents. Glutathione synthesis takes place mainly in the liver (which acts as a reservoir) and the lungs. In the case of the depletion of glutathione levels or its increased demand, glutathione levels may be increased by delivering additional cysteine via N-acetyl-L-cysteine. In vivo studies, however, demonstrated when N-acetyl-L-cysteine is administered orally it has very low bioavailability due to rapid metabolism to glutathione among other metabolites. Thus, even though N-acetyl-L-cysteine is very effective in protecting cells of different origins from the toxicity of reactive components in tobacco smoke and reactive oxygen species, a direct scavenging effect by N-acetyl cysteine in vivo, particularly when administered orally, is not likely. As a result, bioavailability of N-acetyl cysteine itself is very low when given through the oral route. A more relevant mechanism in vivo for any protective effect N-acetyl cysteine may exert against toxic species may be due to N-acetyl-L-cysteine acting as a precursor of glutathione and facilitating its biosynthesis. Glutathione will then serve as the protective agent and detoxify reactive species both enzymatically and non-enzymatically.

Antioxidant supplementation has been studied as a method to counter disease-associated oxidative stress. Several antioxidants have been used with varying degrees of success. However, although the commonly used antioxidants, including vitamin C, vitamin K and lipoic acid, can directly neutralize free radicals, they cannot replenish the cysteine required for glutathione synthesis and replenishment. The cysteine prodrug N-acetyl cysteine, which supplies the cysteine necessary for glutathione synthesis, has proven more effective in treating disease-associated oxidative stress. N-acetyl cysteine been clinically used to treat a variety of conditions including drug toxicity (acetaminophen toxicity), human immunodeficiency virus/AIDS, cystic fibrosis, COPD and diabetes.

Schmid et al. (2002) reported the treatment of chronic obstructive pulmonary disease patients with N-acetyl cysteine at a concentration of 1.2 mg/day or 1.8 mg/day for 2 months improved red blood cell shape, reduced H₂O₂ concentrations by 38 to 54% and increased thiol levels by 50 to 68%. Administering N-acetyl-L-cysteine orally (600 mg/day) increased lung lavage glutathione levels (Bridgeman et al. 1991), reduced superoxide production by alveolar macrophages (Linden et al. 1998) and reduced sputum eosinophil cationic protein concentrations and the adhesion of polymorphonuclear leukocytes in COPD patients (DeBacker et al. 1997).

Odewumi et al. (2016) reported that 2.5 mM of N-acetyl cysteine treatment restored the morphology and viability of CdCl₂ treated human lung cells. They concluded that protection against CdCl₂ toxicity was due to the immuno-modulatory effect of N-acetyl cysteine on various cytokines expression in co-treated human lung cells with 2.5 mM N-acetyl cysteine and 75 μM CdCl₂. These authors concluded that N-acetyl cysteine can be used to treat CdCl₂ toxicity in humans after further testing. It is known that N-acetyl cysteine is an effective metal chelator of cadmium with a measured stability constant of 10^{-7.83} M⁻¹ (Romani et al., 2013). Further, Berthon (1995) report stability constants of complexes with cysteine and Pb²⁺ (10^{-12.2}) and Hg²⁺ (10^{-20.5}) are even greater than for Cd²⁺ (10^{-9.89}). These results clearly identify the potential for N-acetyl cysteine to be an effective chelator of cadmium, mercury and lead in epithelial lung fluid and in blood.

In a study of Idiopathic Pulmonary Fibrosis and N-acetyl cysteine therapy, Hargiwara et al. (2000) demonstrated in mice that inhalation of N-acetyl cysteine inhibited lung fibrosis induced by bleomycin, a chemical that reduces molecular oxygen to superoxide and hydroxyl radicals that can then attack DNA and cause strand cleavage. In the lung, inflammation and immune processes are the major pathogenic mechanisms that injure tissue and stimulate fibrosis. These authors concluded that N-acetyl cysteine inhalation is expected to be a potential therapy for interstitial pneumonia because reactive oxygen species are involved in the development of almost all interstitial pneumonia. They also concluded that because N-acetyl cysteine inhibits NF-kB activation, N-acetyl cysteine may repress chemokine production (i.e. IL-8) and intercellular adhesion molecule-1 (ICAM-1) expression through the inactivation of NF-κB, thereby decreasing inflammatory cell accumulation into the lungs.

Rhoden et al. (2004) applied an in vivo model of inhalation exposure to “real world” particles to demonstrate the central role of reactive oxygen species in 0.1μ to 2.5μ size particles to determine particulate air pollution biological effects. These authors demonstrated that N-acetyl cysteine, at a dose sufficient to prevent an increase in reactive oxygen species and accumulation of thiobarbituric reactive substances and to partially reduce protein oxidation, effectively prevented particulate air pollution-induced inflammation. They concluded the preventive effect of N-acetyl cysteine suggests that treatment with low doses of N-acetyl cysteine could be used to ameliorate the toxic effects of particulate air pollution.

### Carbocysteine

Carbocysteine, (S-carboxymethylcysteine) is a thiol containing amino acid compounds and has significant mucolytic, antioxidation and anti-inflammatory properties. Carbocysteine is also effective to preserve alpha-1-antitrypsin activity, which is inactivated by oxidative stress. The inactivation of alpha-1-antitrypsin is associated with extensive tissue damage in patients with chronic emphysema. The antioxidative and anti-inflammatory properties of carbocysteine are reported to play an important role in the long-term treatment of COPD and to reduce exacerbation rates. Carbocysteine has been reported to have efficacy in reducing exhaled interleukin-6 and interleukin-8 concentrations, which improved the ability of clinical variables to predict mortality in patients with COPD.

Lambert et al (2008) reported that in the presence of 2 mM N-acetyl cysteine, the cellular uptake of epigallocatechin-3-gallate (100 μM) increased by 2.5 times. They also reported that this increase in cytosolic levels of epigallocatechin-3-gallate appears to be due to increased stability of epigallocatechin-3-gallate in the presence of N-acetyl cysteine. They suggested that the increase in growth inhibitory activity observed using the combination of epigallocatechin-3-gallate and N-acetyl cysteine may be the result of the activity of an epigallocatechin-3-gallate-2′-N-acetyl cysteine adduct. These authors also reported that the epigallocatechin-3-gallate-2′-N-acetyl cysteine adduct is biologically active and may be more redox active than epigallocatechin-3-gallate alone.

Bucca et al. (1992) reported that chronic treatment with high doses of vitamin C may be expected to improve symptoms of airway irritability, offer protection against airway and lung damage induced by heavy air pollution in industrialized areas, and improve the prognosis of chronic obstructive lung disease.

### Polyphenols and Phytochemicals

Liang et al. (2017) investigated effects of epigallocatechin-3-gallate (50 mg/kg) given orally each day in rats that were randomly divided into either a sham air (SA) or cigarette smoke exposed groups (1 hr/day for 56 days). They measured oxidative stress and inflammatory markers thought analysis of serum and/or bronchoalveolar lavage fluid. (−)-Epigallocatechin-3-gallate treatment ameliorated cigarette smoke-induced oxidative stress and neutrophilic inflammation, as well as airway mucus production and collagen deposition in rats. They concluded (−)-Epigallocatechin-3-gallate has a therapeutic effect on chronic airway inflammation and abnormal airway mucus production via inhibition of the estimated glomerular filtration rate (EGFR) signaling pathway. They also concluded that (−)-Epigallocatechin-3-gallate supplementation may be a promising therapeutic strategy to limit neutrophil recruitment and to treat mucus hypersecretion in the airways of smokers without or with COPD.

Chan et al. (2009) reported that Chinese green tea (Lung Chen) has a protective effect on cigarette smoke-induced airspace enlargement, goblet cell hyperplasia as well as a suppressive effect on systemic and local oxidative stresses in rats. Approximately 80% of the active ingredients in in this green tea was (−)-Epigallocatechin-3-gallate.

Li et al. (2007) reported that pulmonary inflammation is a characteristic of many lung diseases. Increased levels of pro-inflammatory cytokines, such as interleukin-1β (IL-1β) and tumor necrosis factor-α (TNF-α), have been correlated with lung inflammation. These authors demonstrated that various inflammatory agents, including lipopolysaccharide, 12-o-tetradecanoylphorbol-13-acetate, hydrogen peroxide, okadaic acid and ceramide, were able to induce IL-β and TNF-α productions in human lung epithelial cells (A-549), fibroblasts (HFL1), and lymphoma cells (U-937). They reported that berberine, a phytochemical and a protoberberine alkaloid was capable of suppressing inflammatory agents-induced cytokine production in lung cells and that inhibition of cytokine production by berberine was dose-dependent and cell type-independent. The also reported the suppression of cytokine production by berberine resulted from the inhibition of inhibitory NF-κα phosphorylation and degradation. They concluded that berberine has a potential role of in the treatment of pulmonary inflammation.

Xu et al. (2015) studied the effects of berberine, on cigarette smoke-induced airway inflammation and mucus hypersecretion in mice. Mice with exposure to cigarette smoke were intraperitonealy injected with berberine (5 and 10 mg/kg-d). Inflammatory cytokines TNF-α, IL-1β and Monocyte Chemoattractant Protein 1 (MCP-1) levels in bronchoalveolar lavage fluid were analyzed and lung tissue was examined for histopathological lesions and goblet cell hyperplasia. They reported that cigarette smoke exposure significantly increased the release of inflammatory cytokines TNF-α, IL-_1β, MCP-1 and inflammatory cells in bronchoalveolar lavage fluid, and it also induced goblet cell hyperplasia and the expression of mucin-5ac in the airway of mice. When the mice were pretreated with berberine, cigarette smoke-induced airway inflammation and mucus production were inhibited. Cigarette smoke exposure also increased the expression of extracellular signal-regulated kinases (ERK) and P38, while berberine intervention inhibited these changes.

Several additional polyphenolic, phytochemical and natural antioxidant compounds can be incorporated into liquids disclosed in this instant invention that are transferred to gas and aerosol phases for inhalation drug treatment of lung and respiratory tract diseases, including, but not limited to; berberine, catechin, curcumin, epicatechin, epigallocatechin, epigallocatechin-3-gallate, β-carotene, quercetin, kaempferol, luteolin, ellagic acid, resveratrol, silymarin, nicotinamide adenine dinucleotide, thymoquinone, β-caryophyllene and dimethyl sulfoxide.

An embodiment in this present invention is to deliver N-acetyl-L-cysteine, glutathione and plant-based TRPA1 antagonists with polyphenolic, phytochemical and water soluble antioxidants in an aerosolized form inhaled directly to the respiratory tract.

### Taurine

Taurine (2-aminoethanesulfonic acid) is an amino acid compound that is widely distributed in animal tissue and accounts for up to 0.1% of total human body weight. (EFSA Response Letter, EFSA-Q-2007-113, 2009). Taurine, a sulfonic amino acid, is relatively nontoxic and a normal constituent of the human diet. Dietary sources provide most taurine either directly or by synthesis in the liver and brain from methionine or cysteine via cysteic acid or hypotaurine or by cysteamine in the heart and kidney. Taurine stabilizes membranes, modulates calcium transport, and is able to dissipate the toxic effects of hypochlorous acid (HOCl) by the formation of the relatively stable taurochloramine molecule, generated by myeloperoxidases from oxygen radicals. The ability of taurine to conjugate with xenobiotics, retinoic acid, and bile salts and its role as a major free amino acid in regulating the osmolality of cells are also examples of its protective functions. Taurine may protect membranes by detoxification of destructive compounds and/or by directly preventing alterations in membrane permeability. Protective effects of taurine have been extensively studied including its effects against arteriosclerosis, lung injury by oxidant gases, deleterious effects of various drugs such as tauromustine, an antitumor agent, and hepatotoxicity of sulfolithocholate and its promotion of the recovery of leukocytes in irradiated rats. Further, the therapeutic effects of taurine have been used clinically on Alzheimer's disease, macular degeneration, epilepsy, ischemia, obesity, diabetes, hypertension, congestive, heart failure, noxious effects of smoking, toxicity of methotrexate, cystic fibrosis, myocardial infarction, alcoholic craving, and neurodegeneration in elderly. Taurine has also been reported to protect against carbon tetrachloride-induced toxicity. Carbon tetrachloride was widely used as an industrial degreasing compound and as a dry cleaning compound (Birdsdall, 1998).

Patients with cystic fibrosis are deficient in taurine, a condition reflected by a high bile acid glycine/taurine ratio. The cause of this deficiency is thought to be the excessive loss of taurine from the digestive tract. Human neutrophils and lung epithelial cells have particularly high concentrations of taurine at 19 and 14 mM, respectively. Although the concentration of taurine in extracellular fluids is normally low, cystic fibrosis airway secretions are rich in activated neutrophils, neutrophil-derived products, and cell debris, a situation that could conceivably favor high taurine concentrations at the lung epithelial surface. Patients with cystic fibrosis also have very high myeloperoxidase concentrations in their sputum (Cantin, 1994). Multiple studies have shown that hydrogen peroxide is greatly increased in the exhaled breath condensate of COPD subjects compared to healthy controls.

It has been reported that taurine is an important regulator of oxidative stress and decreased taurine content has been shown to trigger a decline in respiratory chain complexes (Li, et al. 2017). Taurine, in conjunction with niacin, has been shown to protect against lung injury induced by various oxidants such as ozone, nitrogen dioxide, amiodarone and paraquat.

Phagocyte lysosomes contain the enzyme myeloperoxidase which catalyzes the oxidant hydrogen peroxide (H₂O₂) found in the lungs of COPD, asthma, cystic fibrosis and other respiratory disease patients, producing highly oxidizing hypochlorous acid (HOCl). Environmental derived reactive oxygen species are common in the lung epithelium. Reactive oxygen species are found in cigarette smoke, combustion of organic matter and air pollutant gases capable of oxidant activity such as ozone and nitrogen dioxide. These reactive oxygen species can deplete oxidant defenses and increase the oxidant burden in the lungs.

Recent evidence demonstrates that taurine chloramine (Tau-Cl) is produced from the myeloperoxidase-catalyzed reaction of taurine and endogenously produced and highly toxic hypochlorous acid. March (1995) concluded that taurine is pivotal in regulating inflammation. In leukocytes, taurine acts to trap chlorinated oxidants (HOCl). Tau-Cl has also been demonstrated to reduce lymphocyte proliferation in another study. Tau-Cl has also been demonstrated to inhibit a great number of cytokines, including; IL-1β, IL-6, IL-8, TNF-α (Marcinkiewicz et al. (2014). Several researchers have also attributed taurine's antioxidant actions to elevations in the activity of antioxidant enzymes and by reducing the amount of damaging neutrophil-generated reactive oxygen species. Taurine indirectly elevates the activity of endogenous antioxidant defenses. Second, taurine serves as an important anti-inflammatory agent through the production of taurine chloramine.

An embodiment in this present invention is to deliver N-acetyl-L-cysteine, glutathione and plant-based TRPA1 antagonists, water soluble antioxidants and taurine in an aerosolized form inhaled directly to the respiratory tract.

### Thiamin

Thiamin (vitamin B1), is a member of the water-soluble family of vitamins and is essential for normal cellular functions. Thiamin deficiency results in oxidative stress and mitochondrial dysfunction. Thiamin also plays a key role in the reduction of cellular oxidative stress and in maintaining mitochondrial health and function. Deficiency of thiamin is detrimental for normal cell physiology and leads to impairment of oxidative energy metabolism (acute energy failure) predisposing the cells to oxidative stress. Nicotine is known to accumulate in the pancreas and has been implicated in the production of free radicals that lead to oxidative stress and consequently pancreatic injury. Thiamine deficiency (less than 75% of the Recommended Daily Allowance (RDA)) was found in over 75% of patients in a clinical study of 163 elderly COPD patient.

### Dexpanthenol

Dexpanthenol is an alcohol derivative of pantothenic acid, a component of the B complex vitamins and an essential component of a normally functioning epithelium. Dexpanthenol is a prodrug to Vitamin B5 and acts as a precursor of coenzyme A, necessary for acetylation reactions and is involved in the synthesis of acetylcholine. Dexpanthenol has a major role in cellular defenses and in repair systems against oxidative stress and inflammation. The use of dexpanthenol as an antioxidant strategy has been reported to be effective for the prevention and treatment of pulmonary fibrosis. Idiopathic pulmonary fibrosis (IPF) is defined as a specific form of chronic progressive lung disease of unknown cause associated with inflammation, oxidative stress, and accumulation of fibroblasts/myofibroblasts, leading to abnormal deposition of extracellular collagen, particularly in the early stage of the disease (Ermis et al. 2013).

In this text, the term “vitamin” encompasses provitamins and related compounds.

### L-Theanine

L-theanine, is a water-soluble amino acid isolated from green tea (Camellia sinensis), has anti-inflammatory activity, antioxidative properties, and hepatoprotective effects. Hwang et al. (2017) reported that treatment with L-theanine dramatically attenuated inflammatory cells in bronchoalveolar lavage fluid (BALF). They also reported that histological studies revealed that L-theanine significantly inhibited mucus production and inflammatory cell infiltration in the respiratory tract and blood vessels. L-theanine administration also significantly decreased the production of IgE, monocyte chemoattractant protein-1 (MCP-1), interleukin (IL)-4, IL-5, IL-13, tumor necrosis factor-alpha (TNF-α), and interferon-gamma (INF-γ) in BALF. L-theanine also markedly attenuated reactive oxygen species and the activation of nuclear factor kappa B (NF-κB) and matrix metalloprotease-9 in BALF. These authors suggested L-theanine alleviates airway inflammation in asthma, which likely occurs via the oxidative stress-responsive NF-κB pathway, highlighting its potential as a useful therapeutic agent for asthma management.

Several studies report that theanine suppresses the growth in hepatoma, prostate cancer, and colon cancer cells (Friedman et al. 2007). The anticancer activity of theanine has been demonstrated against growth of human lung cancer and leukemia cells as well as migration and invasion of human lung cancer cells (Liu et al. 2009). They also reported that theanine significantly suppressed the growth of human lung cancer A549 and leukemia K562 cells in vitro and ex vivo. In addition, they also demonstrated that theanine also significantly inhibited the migration and invasion of A549 cells.

### Resveratrol

Resveratrol has been demonstrated to have anti-inflammatory and anti-asthmatic properties in mouse models of allergic asthma. Although resveratrol is less potent compared to glucocorticoids, it appears to be more effective in suppressing inflammatory activity. The clinical use of glucocorticoids has a high risk of side effects, and the effect of glucocorticoids is controversial, especially in noneosinophilic asthma. Resveratrol has been shown to suppress the development of noneosinophilic asthma. Resveratrol has the potential to be an alternative to corticosteroids for the treatment of non-allergic forms of asthma. Resveratrol hold a great promise as a natural agent, since it has been shown to have beneficial effects in a variety of diseases, including cancer, cardiovascular disease, neurologic disorders as well as obesity.

Anti-inflammatory and antioxidant properties of resveratrol in the lungs have been demonstrated in preclinical models. Resveratrol causes a reduction in lung tissue neutrophilia and proinflammatory cytokines (Birrell et al. 2005). In vitro treatment with resveratrol inhibited the release of inflammatory cytokines from bronchoalveolar lavage fluid macrophages and human bronchial smooth muscle cells isolated from COPD patients. These anti-inflammatory effects of resveratrol were ascribed to the inhibition of NF-kB activation. Resveratrol has also been shown to inhibit autophagy in vitro in human bronchial epithelial cells and in vivo in cigarette smoke-induced COPD mice model (Liu, et al. 2014). These researchers reported cigarette smoke exposure increased the number of pulmonary inflammatory cells, coupled with elevated production of TNF-α and IL-6 in bronchoalveolar lavage fluids. Resveratrol treatment decreased cigarette smoke-induced lung inflammation. Resveratrol restored the activities of superoxide dismutase, GSH peroxidase, and catalase in cigarette smoke-treated mice. The also demonstrated that cigarette smoke significantly enhanced production of NF-κB) and NF-κB DNA binding activity, which was impaired by resveratrol pretreatment. These authors concluded that resveratrol attenuates cigarette smoke-induced lung oxidative injury, which involves decreased NF-κB activity and the elevated Heme Oxygenase 1 (HO-1) expression and activity.

### Nicotinamide Adenine Dinucleotide

Nicotinamide adenine dinucleotide (NAD⁺) is a central metabolic cofactor and coenzyme in eukaryotic cells that plays a key role in regulating cellular metabolism and energy homeostasis. NAD⁺ in its reduced form (i.e. NADH) serves as the primary electron donor in mitochondrial respiratory chain, which involves adenosine triphosphate production by oxidative phosphorylation. The mammalian NAD⁺ biosynthesis occurs via both de novo and salvage pathways, and involves four major precursors, including the essential amino acid 1-tryptophan (Trp), nicotinic acid (NA), nicotinamide (NAM), and nicotinamide riboside (NR). Nicotinamide riboside (NR) is a precursor of NAD+, which is important in regulating oxidative stress. NA, NAM and NR are each a variation of vitamin B3.

Sirtuins are a unique class of NAD⁺-dependent deacetylases that regulate diverse biological functions such as aging, metabolism, and stress resistance. Recently, it has been shown that sirtuins may have anti-inflammatory activities by inhibiting proinflammatory transcription factors such as NF-kB. Serotonin transporter 1 (Sert1) is one of the seven members of the sirtuin family. It has been demonstrated that Sirt1 may also limit the inflammatory process by inhibiting NF-kB and Activator Protein 1 (AP-1), two transcription factors crucially involved in the expression of proinflammatory cytokines such as TNF-α. It is known that lung cells from patients with chronic obstructive pulmonary disease (COPD) and from rats exposed to cigarette smoke displayed reduced expression of Sirt1 associated with increased NF-kB activity and matrix metalloproteinase-9 expression as compared with lung cells from healthy controls.

In one embodiment of this present invention are liquid compositions comprising one or more of NAD⁺, NA, NAM and NR, plant-based TRPA1 antagonists, natural thiol amino acid containing compounds, CB₂ agonists, amino acids, naturally occurring antioxidants, additional vitamins, and bioflavonoid compounds and heavy metal complexing compounds.

Glycerol mononlaurate is a GRAS with demonstrated antimicrobial properties (Schlievert, et al., 1992, Projan et al., 1994) surpressing the growth and virulence of numerous gram positive and gram negative bacteria, fungi, and enveloped viruses (Li et al., 2009). More recently, glycerol monolaurate has been shown to be a potent suppressor of T cell functions and signaling by altering T cell plasma membrane lipid dynamics and is also an immunosuppressant, significantly suppressing the production of IL-2, IFN-γ, TNF-α, and IL-10 in a dose dependent manner (Zhang et al. 2016). In one embodiment, provided for are liquid compositions comprising glycerol mononlaurate as an antimicrobial, antiviral, immunosuppressing and T-Cell inhibiting agent.

### Antioxidants

Oxidants and the imbalance between the cellular redox state and pulmonary defense systems play a role both in the pathogenesis and in the progression of malignant lung diseases. Lung cancer, highly associated with cigarette smoking, is the most common malignancy worldwide, and its incidence is increasing. There is clear evidence that free radicals are linked both to carcinogenesis and tumor behavior. One major hypothesis explaining the importance of oxidants and imbalance of the cellular redox state in lung carcinogenesis is an altered pro-oxidant intracellular environment that facilitates mutations and/or inactivation of tumor suppression genes and activates oncogenes with consequent changes in cell growth, survival and apoptosis (Kinnula et al. 2004).

Wang et al. (2018) reported that concentrations of glutathione is relatively high in many cancer cells such as lung cancer, breast cancer, pancreatic cancer and leukemia. In addition, it has been demonstrated that the anti-apoptosis feature of cancer cells is related to the increase of the intracellular glutathione level. Several reports have shown that decreasing intracellular glutathione content activates various apoptosis related enzymes. Therefore, decreasing concentrations of glutathione is becoming a new strategy for anti-tumor therapy.

Glutathione biochemistry deregulation in tumors has been observed in many different murine and human cancers. In a review by Ortega et al. (2011) it is reported that glutathione has been shown to be important in the protection against tumor microenvironment-related aggression, apoptosis evasion, colonizing ability, and multidrug and radiation resistance. Increased levels of glutathione and resistance to chemotherapeutic agents have been observed (e.g., for platinum containing compounds and alkylating agents, such as cisplatin and melphalan, anthracyclines, doxorubicin, and arsenic). Zu, et al. (2017) states that the depletion of glutathione is thought to be a promising strategy of decreasing chemotherapy resistance and inducing apoptosis through both extrinsic and intrinsic apoptotic pathways.

Xylitol is naturally occurring polyalcohol sugar alcohol present in small amounts in plums, strawberries, cauliflower, and pumpkins. Sugar alcohols are used in the food industry as thickeners and sweeteners, used in place of table sugar. Chukwuma et al. (2017) reported that xylitol exhibited significant in vitro antioxidant free radical nitric oxide and hydroxyl radical scavenging and ferric reducing activities. They also reported in an in vivo study compared to controls, xylitol fed rats were reported to have increased glutathione levels and antioxidant enzyme activities, including increases in superoxide reductase.

Human respiratory syncytial virus (hRSV) is a very common, contagious virus that causes infections of the respiratory tract. While it is the most common cause of bronchiolitis and pneumonia in infants, hRSV is an important pathogen in all age groups. Infection rates are typically higher during the cold winter months, causing bronchiolitis in infants, common colds in adults, and more serious respiratory illnesses such as pneumonia in the elderly and immunocompromised. Xu et al. (2016) reported that in an in vivo study with mice receiving xylitol for 14 days prior to a hRSV virus challenge and for a further 3 day post challenge, significantly greater reductions in lung virus titers were observed in mice receiving xylitol than in the controls receiving phosphate-buffered saline. They also reported fewer CD3⁺ and CD3-CD8⁺ lymphocytes, reflecting a reduced inflammatory status. Go et al. (2020) reported anecdotal evidence in three patients that the combination of xylitol and Grapefruit Seed extract in a commercially available product decreased symptoms in three mild to moderated COVID-19 cases.

Thymoquinone is bioflavonoid volatile oil extracted from seeds of the plant Nigella sativa with antioxidant, anti-inflammatory, neuroprotective, antiallergenic, antiviral, antidiabetic, and anti-carcinogenic properties. In addition, it has been identified to have inhibitory effects on histamine receptors. Thymoquinone has been shown to suppress the production of leukotriene B4, thromboxane B2, and inflammatory mediators via 5-lipoxygenase and cyclooxygenase pathway of arachidonic acid metabolism. Antioxidant and immunomodulatory properties of thymoquinone have also been demonstrated. Thymoquinone has been shown to effectively treat cancer, as well as allergic diseases, including allergic rhinitis, atopic eczema, and asthma. Kalemci, et al. (2013) demonstrated that thymoquinone injection caused a reduction in chronic inflammatory changes in an experimental asthma model created in mice. Azemi et al (2016) reported that mice receiving black seed oil showed a significant decrease in the number of eosinophils, and a potential inhibitory effect on mRNA expression levels of Th2-driven immune response cytokines and mucin, resulting in decreased production of interleukin and mucin in allergic asthma. They concluded that black seed oil has an anti-inflammatory and immunomodulatory effect during the allergic response in the lung, and can be a promising treatment for allergic asthma in humans.

El-Sakkar et al. (2007) induced significant lung inflammation in Guinea pigs as evidenced by the increased levels of IL-8, LTB4, NE, and TNF-α (in bronchoalveolar lavage fluid) and myeloperoxidase (in lung tissue homogenates). Cigarette smoke also resulted in a significant increase in lung tissue glutathione peroxidase activity. Lipid peroxidation was significantly increased in cigarette smoke exposed Guinea pigs as evidenced by an increase in lung tissue malondialdehyde. Pretreatment of cigarette smoke-exposed Guinea pigs with thymoquinone significantly decreased the bronchoalveolar lavage fluid IL-8, but did not significantly change bronchoalveolar lavage fluid Leukotriene B4 (LTB4) levels. The levels of the inflammatory mediators; neutrophil elastase, TNF-α and malondialdehyde were also significantly reduced after thymoquinone pretreatment.

El-Sakkar et al. (2007) also reported that the pretreatment of cigarette smoke-exposed Guinea pigs with epigallocatechin-3-gallate (the major polyphenol in green tea) reduced the inflammatory consequences of exposure to cigarette smoke. This was demonstrated by the significantly reduced levels of IL-8, LTB4, NE, TNF-α (in bronchoalveolar lavage fluid) and myeloperoxidase (in lung tissue homogenate). Epigallocatechin-3-gallate also attenuated cigarette smoke-induced oxidative stress as revealed by the increase of glutathione peroxidase activity, and the significant decreased level of myeloperoxidase in lung tissue homogenates, although superoxide dismutase activity was not significantly affected.

El-Sakkar et al. (2007) concluded that thymoquinone and epigallocatechin-3-gallate have protective effects against cigarette smoke-induced inflammatory and oxidative damage in the guinea pig lungs. They reported that the protective effects on the lungs were likely the result of effects on inflammatory cells, cytokine production, and oxidative stress. They also reported that their results, if extrapolated to humans, would indicate that thymoquinone and epigallocatechin-3-gallate have potential as novel therapeutic agents for chronic obstructive pulmonary disease patients and could be promising in the design and development of new treatment strategies aiming at limiting cellular inflammatory and oxidative damage.

### Electronic Aerosolization Devices

Electronic-cigarettes, also known as vape pens, e-cigars, or vaping devices, are typically used as electronic nicotine delivering systems, which thermally generate an aerosolized mixture containing flavored liquids and nicotine that is inhaled by the user. Electronic thermal aerosolization devices are also used for inhalation of CBD, THC and select vitamins. The extensive diversity of e-cigarettes arises from the various nicotine concentrations present in e-liquids, miscellaneous volumes of e-liquids per product, different carrier compounds, additives, flavors, coil impedances, and battery voltages. Regardless of the exact design, each e-cigarette device has a common functioning system, which is composed of a rechargeable lithium battery, vaporization chamber, and a cartridge. The lithium ion battery is connected to the vaporization chamber that contains an atomizer. In order to deliver nicotine to the lungs, the user inhales through a mouthpiece, and the airflow triggers a sensor that then switches on the atomizer. The atomizer thermally vaporizes liquid nicotine in a small cartridge and delivers it to the lungs.

Ultrasonic vaping devices that do not heat the liquids in an electronic vaporization device as much as typical commercially available e-cigarettes or thermal aerosolization devices are available and can also be used to aerosolize liquids disclosed in this present invention.

Recently, a study was conducted on the nicotine content on 27 e-cigarette liquid formulations acquired in the U.S. It was reported that the nicotine content varied between 6 and 22 mg/L (Peace, 2016). In another study 16 e-cigarettes were selected based on their popularity in the Polish, U.K. and U.S. markets and nicotine vapor generation was evaluated in an automatic smoking machine. Testing conditions were designed to simulate puffing conditions of human electronic cigarette users. The total level of nicotine in vapor generated by 20 series of 15 puffs varied from about 0.5 mg to 15.4 mg. Most of the analyzed electronic cigarette effectively delivered nicotine during the first 150-180 puffs. On an average, 50%-60% of nicotine from a cartridge was vaporized.

The average concentration of nicotine in Juul electronic cigarettes was recently reported to be 60.9 mg/mL, 63.5 mg/mL, and 41.2 mg/mL in un-vaped, vaped, and aerosol samples, respectively. Transfer efficiently for nicotine to the aerosol was between 56%-75% (Omaiye, et al. 2019). Juul reports that each of their flavor pods contain 0.7 mL of liquid.

Because of the formation of toxic compounds inhaled from thermally generated aerosolized liquids containing nicotine, in November 2018, FDA's Center for Tobacco Products (CTP) banned all flavored nicotine e-cigarettes other than tobacco, mint, and menthol flavors. In recent studies, it has been reported that specific flavorant aldehydes compounds, including benzaldehyde, cinnamaldehyde, citral, ethylvanillin, and vanillin, react with other commonly used compounds present in liquids used in vaping, such as, propylene glycol (PG), to form toxic flavor aldehyde PG acetals at room and elevated temperatures. These flavor aldehyde PG acetals were also reported to be detected in commercial e-liquids compounds at ambient temperatures. When these flavor aldehyde PG acetals in e-liquids are subsequently thermally aerosolized and inhaled in vaping devices, they can cause serious health impacts to individuals using these products. Flavor aldehyde PG acetals have also been demonstrated to activate the TRPA1 and aldehyde-insensitive TRPV1 irritant and inflammation-related receptors (Erythropel, et al. 2018). It is clear that activating inflammatory nociceptors TRPA1 and TRPV1 by flavor aldehyde PG acetals in the lungs of individuals using vaping products is extremely unhealthful for these individuals.

In another recent study, the toxic ambient temperature reaction products vanillin PG acetal and vanillin VG acetals were detected in JUUL e-liquids and carried over to e-cigarette generated aerosols at 68.4% and 59%, respectively. Nicotine and benzoic acid were also carried over from JUUL e-liquids to e-cigarette generated aerosols at 98.6% and 82.5%, respectively (Erythropel, et al. 2019).

In one embodiment of this present invention are aerosolizable liquids that contain nicotine that do not contain aldehyde flavorants and do not form toxic flavorant acetals compounds, either at ambient or elevated temperature and are safer to use in e-cigarettes and other thermal liquid aerosolization devices than existing e-liquids available in the market to date. In yet other embodiments of this present inventions are aerosolizable liquids that contain nicotine that provide health benefits to the respiratory system of individuals that are nicotine users. In another embodiment of this present invention are methods of use liquid compositions containing nicotine and plant-based TRPA1 antagonists, natural thiol amino acid containing compounds, CB₂ agonists, amino acids, naturally occurring antioxidants, additional vitamins, bioflavonoid compounds and heavy metal complexing compounds when thermally aerosolized provide a source of nicotine and respiratory health benefits from the non-nicotine components of the composition.

Recently, companies have begun to market thermal aerosolization systems in which vitamins are inhaled to supplement vitamins. Vitamin Vape, Q Sciences, Biovape, and Nutrovape Vita are a sampling of companies that manufacture and sell vaping systems to supplement vitamins. Inhalation is likely an inefficient way to ingest vitamins that may be needed systemically at higher concentrations than can be delivered by vaping. Inhalation is usually reserved as a delivery mechanism for medicines that require very small doses or target the lungs themselves.

### Cigarette Smoking Cessation

The most important way to reduce on-going damage to an active cigarette smoker's general health and specifically their respiratory system is the complete cessation of smoking cigarettes and the withdrawal from exposure and addiction to nicotine. While cessation of cigarette smoking eliminates ongoing respiratory system damage from cigarette smoke, it does not reverse past respiratory system damage from past cigarette smoking, diseases already active in an individual the result of exposure to cigarette smoke and future diseases possible from past smoking activities. Historically, it is well documented that the cumulative exposure to cigarette smoking, generally expressed in pack-years (i.e., the number of packs of cigarettes smoked per day multiplied by the number of years smoked) is a primary factor in the risk of lung cancer and COPD. Recently, it has been shown that smoking duration is more strongly associated with COPD than the composite of pack-years alone (Bhatt et al. 2018). These researchers analyzed cross-sectional data from a large multicenter cohort (10,187 people) of current and former smokers. The primary outcome measure was airflow obstruction, measured by the FEV1/FVC ratio and other parameters including FEV1 alone. They reported a linear relationship between the FEV1/FVC ratio and the number of years of active smoking, revealing that the duration of smoking was more influential than the number of pack-years an individual smoked. Similarly, there was a strong relationship between duration of cigarette smoking and decrease of FEV1 values.

Nicotine replacement therapy (NRT) is an accepted way to quit smoking cigarettes and provides an individual nicotine in the form of gum, patches, sprays, inhalers, or lozenges without the other harmful chemicals in tobacco and their by-products. NRT gums and lozenges are available without a prescription and provide between 2 mg and 4 mg per piece. NRT patches provide a passive time integrated does of nicotine on a daily basis. Nicoderm CQ is a non-prescription patch providing 21 mg per day (Step 1), 14 mg per day (Step 2) and 7 mg per day (Step 3). The Nicotrol patch provides a 3 Step system as well with 15 mg per day (Step 1), 10 mg per day (Step 2) and 5 mg per day (Step 3). NRTs help to relieve some nicotine physical withdrawal symptoms enabling a person to focus more on the psychological aspects of cigarette smoking cessation. Many studies have shown using NRT can nearly double the chances of successful cigarette smoking cessation.

In one embodiment of this present invention, aerosolizable liquid compositions and methods of use of these liquid compositions include a nicotine salt as part of a nicotine replacement therapy cigarette smoking cessation system, while providing simultaneous treatment of the lung and respiratory tract diseases and impact from a person's history of cigarette smoking. In an embodiment of this present invention, is a composition comprising a nicotine salt, a plant-based TRPA1 antagonists, natural thiol amino acid containing compounds, CB₂ agonists, amino acids, naturally occurring antioxidants, vitamins, and flavonoid compounds, and heavy metal complexing compounds.

### Glutathione

The use of glutathione in this present invention and the results reported in Examples 15 and 16 were unexpected as asthma is a condition where the known side effects of inhaled glutathione, including breathlessness, bronchoconstriction, and cough, led researchers and practitioners to not recommend glutathione for asthma (Prousky et al., 2008). The effectiveness of the use of glutathione in this present invention is further unexpected based on research published by Marrades et al. (1997), who reported that inhaled glutathione caused major airway narrowing (changes from baseline: FEV1 of −19% and total pulmonary resistance of +61%) and induced cough (four patients) or breathlessness (three patients). In contrast, control patients treated only with inhaled saline solution had negligible FEV1 changes of −1% and minor total pulmonary resistance change of +17%.

Inhaled glutathione is also known to reduce zinc levels in the blood. Reduced serum zinc levels will reduce immune functioning and potentially increase infection such as bronchitis or pneumonia.

A person of ordinary skill in the art would not recommend inhaled glutathione as it is contraindicated for use with asthma on several medical websites including WebMd (https://www.webmd.com/vitamins/ai/ingredientmono-717/glutathione, “Side Effects & Safety”) in which the side effects for asthma include: “Do not inhale glutathione if you have asthma. It can increase some asthma symptoms.”

A person of ordinary skill in the art would be taught away from the use of combining glutathione with other compounds in our formulations for the treatment of individuals with asthma. Surprising and unexpectedly, the studies leading to the instant invention indicated that the use of glutathione was highly effective at increasing FEV1 levels in patients with documented asthma. One of the asthma patients (Patient 104 in FIG. 19) smoked 2 packs per day of cigarettes for 28 years (56 pack-years) and had unexpected results of 45.1% FEV1 reversibility, and their percent normal FEV1 increased from 67.2% to 97.4% after 53 days of treatment. This is the opposite of what a person of ordinary skill in the art would be taught by Marrades et a. (1997).

### N-Acetyl Cysteine

N-acetylcysteine (NAC) is used as an “antioxidant” in studies examining gene expression, signaling pathways, and outcome in acute and chronic models of lung injury. It is also known that N-acetylcysteine can also undergo auto-oxidation and also behave as an oxidant. Chan et al. (2001) demonstrated that N-acetylcysteine can become an oxidant leading to the activation of nuclear factor kappa B (NF-κB), a key proinflammatory signaling pathway.

According to the online medical website, WebMd (https://www.webmd.com/vitamins/ai/ingredientmono-1018/n-acetyl-cysteine) when N-acetylcysteine is administered by inhalation it can cause inflammation in the mouth, runny nose, drowsiness, clamminess, and chest tightness. Also according to WebMd, there is concern that N-acetylcysteine might cause bronchospasm in people with asthma if inhaled. The National Institutes of Health report that N-acetylcysteine can result in respiratory inflammation, causes running nose, bronchospasm, inflammation of the mouth, and bleeding. A person ordinarily skilled in the art would be taught away for using N-acetylcysteine for the inhalation treatment of individuals with COPD, asthma and, other respiratory diseases because of N-acetylcysteine's known side effects.

It is an unexpected result that the use of N-acetylcysteine in the formulations in this present invention shown in Examples 15 and 16 led to a decrease in respiratory inflammation as evidenced by decreased FEV1 and FVC lung function parameters, given the ability of N-acetylcysteine to function as an oxidant, result in the formation of NF-κB, and cause bronchospasm in people with asthma.

### Vitamin B12

According the health website Healthline (https://www.healthline.com/health/food-nutrition/vitamin-b12-side-effects), side effects of taking vitamin B12, orally or by inhalation, include increased anxiety, pulmonary edema, and congestive heart failure. It has also been reported to increase the risk for tracheal and bronchial swelling. A person ordinarily skilled in the art would be taught against using methylcobalamin (vitamin B12) in a liquid that would be used for inhalation treatment of respiratory diseases, because of methylcobalamin's known side effects. Although methylcobalamin is known to cause increased anxiety in some patients, the individuals who were evaluated in pre-clinical trials as disclosed in Examples 15 and 16 surprisingly and unexpectedly reported significantly lower anxiety levels following treatment.

Interaction of One Component with the Others

Administering the liquid formulations to patients disclosed in Examples 15, by means of thermally induced aerosolization and by means of ultrasonic membrane aerosolization in Example 16 led to surprising and unexpected results, because individual compounds in these formulations have complementary and synergistic effects. For example while the primary use 1,8-cineole in the formulations disclosed in the present invention is a TRPA1 antagonist, it also acts secondarily as a TRPM8 agonist, modulates immune functions, is an antioxidant, is bacteriostatic and fungistatic, and inhibits production of tumor necrosis factor-α (TNF-α), interleukin-1β (IL-1β), interleukin-4 (IL-4), interleukin-5 (IL-5), leukotriene B4 (LTB4), thromboxane B2 (TXB2) and prostaglandin E2 (PGE2). 1,8-cineole has also been demonstrated to reduce anxiety in a human clinical trial for pre-operative patients. Unexpectedly, this anti-anxiety property of 1,8-cineole is very helpful in patients with difficulty breathing, which causes anxiety and in severe cases, panic. Verbal qualitative reports by patients administered formulations in Examples 15 and 16 reported a sense of feeling more relaxed, significantly increased energy levels, greater endurance capabilities under normal activities, as well as under exercising conditions, lower levels of anxiety, and less anxiety compared to taking other medications for their disease treatment. Typical steroid administration by inhalation has side effects including shaking nervousness and burning sensation in the chest area. Unexpectedly, in this present invention, no patients reported any negative side effects associated with the inhalation treatments of the formulations disclosed in Examples 15 and 16.

The primary and secondary roles of 1,8-cineole unexpectedly result in synergy with β-caryophyllene which has a primary role in the formulations disclosed in this present inventions as a CB₂ agonist to reduce inflammation. β-caryophyllene also has secondary roles in this present invention as an antioxidant and also acts as an analgesic, anti-inflammatory, neuroprotective, anti-depressive, anxiolytic, and antioxidant compound, in addition to inhibiting production of pro-inflammatory cytokines, such as TNF-α, IL-1β IL-6. This use of 1,8-cineole and β-caryophyllene together provides different and complementary primary anti-inflammatory functions as a TRPA1 antagonist and a CB₂ agonist, respectively, and 8-cineole and β-caryophyllene unexpectedly complement one another through the synergy of both the primary and secondary properties of each compound. These anti-oxidant properties of 1,8-cineole and β-caryophyllene also unexpectedly act synergistically with glutathione and n-acetyl cysteine that act as the primary antioxidants and thiol containing amino acids in the disclosed formulations.

A person ordinarily skilled in the art would normally have been taught not to use β-caryophyllene formulations disclosed in this present invention as it has been demonstrated to be a TRPA1 agonist (activator) that causes inflammation (Moon et al. 2015). Thus, a person of ordinary skill in the art would have thought that one would not want to include β-caryophyllene in the formulation, because it would agonize the TRPA1 receptor, causing inflammation and coughing.

For compositions set forth herein, components can be, for example, in the following ranges:
1,8-cineole, borneol, camphor, 2-methylisoborneol, fenchyl alcohol, or cardamonin—from about 0.01%, 0.03%, 0.1%, 0.3%, 1%, 3%, or 10% to about 0.03%, 0.1%, 0.3%, 1%, 3%, 10%, or 30%;
glutathione, N-acetyl cysteine, carbocysteine, taurine, or methionine—from about 0.01%, 0.03%, 0.1%, 0.3%, 1%, 3%, or 10% to about 0.03%, 0.1%, 0.3%, 1%, 3%, 10%, 20%, 30%, or 50%;
cobalamin, methylcobalamin, hydroxycobalamin, adenosylcobalamin, cyanocobalamin, cholecalciferol, thiamin, dexpanthenol, biotin, nicotinic acid, nicotinamide, nicotinamide riboside, or ascorbic acid—from about 0.0001%, 0.0003%, 0.001%, 0.003%, 0.01%, 0.03%, 0.1%, 0.3%, 1%, or 3% to about 0.0003%, 0.001%, 0.003%, 0.01%, 0.03%, 0.1%, 0.3%, 1%, 3%, or 10%;
citric acid or ethylenediaminetetraacetic acid (EDTA)—from about 0.0001%, 0.0003%, 0.001%, 0.003%, 0.01%, 0.03%, 0.1%, 0.3%, 1%, or 3% to about 0.0003%, 0.001%, 0.003%, 0.01%, 0.03%, 0.1%, 0.3%, 1%, 3%, or 10%;
berberine, catechin, curcumin, epicatechin, epigallocatechin, epigallocatechin-3-gallate, β—carotene, quercetin, kaempferol, luteolin, ellagic acid, resveratrol, silymarin, nicotinamide adenine dinucleotide, or thymoquinone—from about 0.001%, 0.003%, 0.01%, 0.03%, 0.1%, 0.3%, 1%, or 3% to about 0.003%, 0.01%, 0.03%, 0.1%, 0.3%, 1%, 3%, or 10%;
alanine, leucine, isoleucine, lysine, valine, methionine, L-theanine, or phenylalanine—from about 0.01%, 0.03%, 0.1%, 0.3%, 1%, 3%, or 10% to about 0.03%, 0.1%, 0.3%, 1%, 3%, 10%, 30%, or 50%;
β-caryophyllene, a cannabinoid, cannabidiol, or cannabinol—from about 0.001%, 0.003%, 0.005%, 0.01%, 0.03%, 0.1%, 0.3%, 1%, or 3% to about 0.003%, 0.01%, 0.03%, 0.1%, 0.3%, 1%, 3%, 5%, or 10%;
nicotine—from about 0.001%, 0.003%, 0.01%, 0.03%, 0.1%, 0.3%, 1%, 2.5%, or 3% to about 0.003%, 0.01%, 0.03%, 0.1%, 0.3%, 1%, 2.5%, 3%, or 10%;
a lubricating, emulsifying, or viscosity-increasing compound—from about 0.01%, 0.03%, 0.1%, 0.3%, 1%, 3%, or 10% to about 0.03%, 0.1%, 0.3%, 1%, 3%, 10%, 30%; and
glycerine—from about 1%, 3%, 10%, 30%, or 50% to about 10%, 30%, 50%, 70%, 80%, 90%, 95%, or 98%.

For example, pH values can be from about 5, 5.5, 6, 6.5, 7, 7.2, 7.5, or 8 to about 5.5, 6, 6.5, 7, 7.2, 7.5, 8, or 8.5.

This invention is further described by the figures, the following examples and experiments, which are solely for the purpose of illustrating specific embodiments of this invention, and are not to be construed as limiting the scope of the invention in any way. The compositions of the present invention can comprise, consist essentially of, or consist of the essential as well as the optional ingredients and components described herein. As used herein, “consisting essentially of” means that the composition or component may include additional ingredients, but only if the additional ingredients do not materially alter the basic and novel characteristics of the claimed compositions or methods. All publications cited herein are hereby incorporated by reference in their entirety.

### EXAMPLES

The following examples are offered to illustrate, but not to limit the claimed invention.

### Example 1

A composition and a method of manufacture of a pharmaceutical liquid that is aerosolized, vaporized or both comprising 1,8-cineole, N-acetyl cysteine, glutathione, ascorbic acid, methylcobalamin, an emulsifying agent, vegetable glycerin, water, sodium bicarbonate (as needed) and a preservative (as needed) is disclosed in Example 1. The method of manufacturing consists of mixing an amount of nitrogen purged purified sterile water or isotonic saline solution with ascorbic acid powder or crystals, sodium bicarbonate, and preservative (if needed) and dissolving, then adding amounts of N-acetyl cysteine, glutathione, and methylcobalamin, followed by adding an amount of vegetable glycerin (if needed) and mixing until the liquid composition is homogeneous. Nitrogen gas purging can be used throughout the mixing period to minimize oxygenation of the water and oxidation of the compounds in the mixture. 1,8-cineole is then separately mixed with the emulsifier, and after this mixture is homogeneous, then slowly adding to the mixture and slowly mixing until it is dissolved in the liquid, minimizing the volatilization of the 1,8-cineole. Mixing can be conducted in a zero or low headspace reactor to further minimize volatilization of 1,8-cineole and oxidation of the compounds in the mixture. If an amount of 1,8-cineole is added to the mixture at concentrations greater than the solubility of 1,8-cineole in the mixture, then the 1,8-cineole can be emulsified in the liquid composition with the addition of a suitable emulsifier, for example Tween 20, also known as Polysorbate 20 and polyoxyethylene(20)sorbitan monooleate. Mixing is limited to that required to create a stable single phase homogeneous solution or emulsion and to minimize volatilization 1,8-cineole. Methods of use of the liquid composition in Example 1 include but are not meant to be limited to placing a quantity of the composition in an e-cigarette vaporizing device, an electronic thermal vaporization device, a nebulizer, an ultrasonic nebulizer, an ultrasonic vaping device or an inhaler and inhalation of the aerosolized vapors resulting from creating an aerosolized mixture. The liquid composition that is the TRPA1 antagonist that can be aerosolized or vaporized in Example 1 can optionally be made with borneol or a mixture of 1,8-cineole and borneol in the same or different total concentration range compared to the range when using 1,8-cineole alone. This liquid composition that can be aerosolized is disclosed in Table 1 (in this text, when compositions or mixtures are discussed, the term “percent” (%) usually refers to weight percentage, unless otherwise indicated). The aerosolizable liquid composition can be transferred to containers that can be stored for one or more doses, the containers may or may not have nitrogen gas in the headspace, and the containers may or may not be refrigerated.

TABLE 1
Base Inhalation Liquid

| | Weight | | | |
|---|---|---|---|---|
| Ingredient | Percent (%) | Function | Sources | Secondary Effect |
| 1,8-Cineole | 0.1-10 | TRPA1 | Pure Compound or Essential | TRPM8 Agonist, |
| | | Antagonist | oils of: Eucalyptus polybractea; | modulate immune |
| | | | Eucalyptus globulus; | function, bacteriostatic |
| | | | Eucalyptus radiate; | Fungistatic, inhibition of |
| | | | Eucalyptus camaldulensis; | production of tumor |
| | | | Eucalyptus smithii; | necrosis factor- a (TNF- |
| | | | Eucalyptus globulus; | α), interleukin-1β (IL-1β), |
| | | | Rosmarinus offficinalis | interleukin-4 (IL-4), |
| | | | | interleukin-5 (IL- |
| | | | | 5), leukotriene B4 (LTB4), |
| | | | | thromboxane B2 (TXB2) |
| | | | | and prostaglandin E2 |
| | | | | (PGE2) |
| N-acetyl | 0.1-10 | Antioxidant, | Synthetic | Glutathione precursor, |
| cysteine | | Natural Thiol | | increase epithelial lining |
| | | Amino Acid | | fluid and lung glutathione |
| | | Containing | | concentrations, modulate |
| | | Compound | | immune function, inhibits |
| | | | | NF-kB activation, |
| | | | | modulates immune |
| | | | | function and participates |
| | | | | in the pulmonary |
| | | | | epithelial host defense |
| | | | | system, radionuclide and |
| | | | | heavy metal chelate |
| Glutathione | 0.1-20 | Antioxidant, | Synthetic | Increase epithelial lining |
| | | Natural Thiol | | fluid and lung glutathione |
| | | Amino Acid | | concentrations, modulate |
| | | Containing | | immune function, inhibits |
| | | Compound | | NF-kB activation, |
| | | | | radionuclide and heavy |
| | | | | metal chelate |
| Ascorbic | 0.01-1.0 | Vitamin, Natural | Synthetic | Decrease Vitamin C |
| Acid | | Antioxidant | | deficiency, modulate |
| | | | | immune function, |
| | | | | inhibition of |
| | | | | prostaglandin E2 (PGE2), |
| | | | | decrease in |
| | | | | bronchoconstriction |
| Methyl | 0.001-1.00 | Vitamin, Natural | Synthetic | Decrease Vitamin B12 |
| cobalamin | | Antioxidant | | deficiency the result of |
| | | | | smoking. Reduce cyanide |
| | | | | concentrations in lungs |
| | | | | and serum |
| Vegetable | 0.0-95 | Thickener | Plant-Based Synthetic | Flavor and vapor |
| Glycerin | | | | production, rheology |
| | | | | control, viscosity modifier |
| Emulsifier | 0.1-2.0 | Stable Suspension | Natural or Synthetic | |
| Sterile Water | 5.0-98 | Carrier | Filtered Water | Diluent |
| Sodium | variable | pH Adjustment | Natural Mineral | Natural Buffer in |
| Bicarbonate | | | | Epithelial Cells |
| Preservative | variable | Chemical and | Natural or Synthetic | |
| | | Biological | | |
| | | Stability | | |

### Example 2

A preferred composition and a method of manufacture of a pharmaceutical liquid that is aerosolized, vaporized or both, using a nebulizer comprising 1,8-cineole, N-acetyl cysteine, glutathione, ascorbic acid, methylcobalamin, an emulsifying agent, a sterile saline solution, sodium bicarbonate (as needed) and a preservative (as needed) is disclosed n Example 2. The method of manufacturing consists of mixing 96.09 g of nitrogen purged 0.9% sterile saline solution with 0.01 g of ascorbic acid powder and dissolving the ascorbic acid, then adding 1.35 g of N-acetyl cysteine, 1.35 g of glutathione, 0.003 g of methylcobalamin, and mixing until the liquid composition is homogeneous. This is followed by adding a mixture of 0.80 g of 1,8-cineole and 0.40 g Polysorbate 20 together and slowly mixing until they are dissolved together. Once the 1,8-cineole and Polysorbate 20 are homogeneously mixed, this mixture is added to the liquid mixture and dissolved into the liquid, minimizing the volatilization of the 1,8-cineole. Mixing is limited to that required to create a stable single phase homogeneous solution and to minimize volatilization 1,8-cineole. The pH of the solution is then measured and a quantity of sodium bicarbonate is added to raise the pH to about 7.20. A quantity of a preservative can be added or alternatively the mixture can be refrigerated prior to use. Methods of use of the composition of the liquid composition in Example 2 include, but are not meant to be limited to placing the composition in a an ultrasonic, vibrating mesh or jet nebulizer and inhalation of the vapors resulting from creating an aerosolized mixture. Methods of use of the composition of the liquid in Example 2, include adding about 1 mL to about 5 ml of the mixture to a liquid nebulizer for inhalation by a patient. This liquid composition is disclosed in Table 2.

TABLE 2
Preferred Base Nebulizer Liquid

| | Weight Percent | | |
|---|---|---|---|
| Ingredient | (%) | Function | Primary Effects |
| 1,8-Cineole | 0.80 | Inflammation Blocker, Anti- | TRPA1 Antagonist |
| | | Cancer | |
| N-acetyl cysteine | 1.35 | Increase Epithelial Liquid and | Antioxidant, Natural Thiol Amino |
| | | Lung Tissue Glutathione | Acid Containing Compound |
| | | Concentration | |
| Glutathione | 1.35 | Increase Epithelial Liquid and | Antioxidant, Natural Thiol Amino |
| | | Lung Tissue Glutathione | Acid Containing Compound |
| | | Concentration | |
| Ascorbic Acid | 0.01 | Increase Epithelial Liquid and | Vitamin, Natural Antioxidant |
| | | Lung Tissue Vitamin C | |
| | | Concentration | |
| Methyl cobalamin | 0.00300 | Increase Epithelial Liquid and | Vitamin, Natural Antioxidant |
| | | Lung Tissue Vitamin B12 | |
| | | Concentration | |
| Polysorbate 20 | 0.40 | Stable Suspension | |
| Sterile Saline | 96.09 | Carrier | Isotonic Diluent |
| Water - 0.9% | | | |
| Sodium | variable to | pH Adjustment | Adjust pH to 7.20 |
| Bicarbonate | pH = 7.2 | | |
| Preservative | Variable as | Chemical and Biological | |
| | Needed | Stability | |

### Example 3

A preferred pharmaceutical composition and a method of manufacture of a pharmaceutical liquid that is aerosolized, vaporized or both, in an ultrasonic or thermal vaporization device and includes 1,8-cineole, N-acetyl cysteine, glutathione, ascorbic acid, methylcobalamin, an emulsifying agent, vegetable glycerin, sterile deionized water, sodium bicarbonate (as needed) and a preservative (as needed) is disclosed in Example 3. The method of manufacturing consists of mixing 16.94 g of nitrogen purged sterile deionized water with 0.01 g of ascorbic acid powder and dissolving the ascorbic acid, then adding 1.20 g of N-acetyl cysteine, 1.53 g of glutathione, 0.003 g of methylcobalamin, and then mixing until the liquid composition is homogeneous. This is followed by adding 93.55 g of vegetable glycerin and mixing. This is then followed by adding a mixture of 1.69 g of 1,8-cineole and 1.01 g of Polysorbate 20 together and slowly mixing until they are dissolved together. Once the 1,8-cineole and Polysorbate 20 are homogeneously mixed, this mixture is added to the glycerin-water based mixture and dissolved into the liquid, minimizing the volatilization of the 1,8-cineole. Mixing is limited to that required to create a stable single phase homogeneous solution and to minimize volatilization 1,8-cineole. The pH of the solution is then measured and a quantity of sodium bicarbonate is added to raise the pH to 7.20. A quantity of a preservative can be added or alternatively the mixture can be refrigerated prior to use. The liquid composition in Example 3 may be made with a quantity of vegetable glycerin that is less than 93.55 g and can be decreased by increasing a corresponding mass of nitrogen purged water added. Methods of use of the composition of the liquid composition in Example 3 include but are not meant to be limited to placing the composition in an e-cigarette vaporizing device, an electronic thermal vaporization device, a vaping pen, electronic thermal vaporization device, an ultrasonic vaping device, an electronic vaping mod and inhalation of the vapors resulting from creating an aerosolized mixture. A preferred vaping device is one that has temperature control and the temperature is limited to an upper limit of 200° C. The aerosolizable pharmaceutical liquid composition can be transferred to containers that can be stored for one or more doses, the containers may or may not have nitrogen gas in the headspace, and the containers may or may not be refrigerated. This liquid composition is disclosed in Table 3.

TABLE 3
Preferred Base Vape Liquid

| | Weight | | |
|---|---|---|---|
| Ingredient | Percent (%) | Function | Primary Effects |
| 1,8-Cineole | 1.69 | Inflammation Blocker, Anti- | TRPA1 Antagonist |
| | | Cancer | |
| N-acetyl cysteine | 1.20 | Increase Epithelial Liquid and | Antioxidant, Natural Thiol Amino |
| | | Lung Tissue Glutathione | Acid Containing Compound |
| | | Concentration | |
| Glutathione | 1.53 | Increase Epithelial Liquid and | Antioxidant, Natural Thiol Amino |
| | | Lung Tissue Glutathione | Acid Containing Compound |
| | | Concentration | |
| Ascorbic Acid | 0.01 | Increase Epithelial Liquid and | Vitamin, Natural Antioxidant |
| | | Lung Tissue Vitamin C | |
| | | Concentration | |
| Methyl | 0.003 | Increase Epithelial Liquid and | Vitamin, Natural Antioxidant |
| cobalamin | | Lung Tissue Vitamin B12 | |
| | | Concentration | |
| Polysorbate 20 | 1.01 | Stable Suspension | |
| Vegetable | 93.55 | Thickener | |
| Glycerin | | | |
| Sterile Water | 16.94 | Carrier | Diluent |
| Sodium | variable to | pH Adjustment | Adjust pH to 7.20 |
| Bicarbonate | pH = 7.2 | | |
| Preservative | Variable as | Chemical and Biological | |
| | Needed | Stability | |

### Example 4

A pharmaceutical liquid composition and a method of manufacture of the liquid that is aerosolized, vaporized or both comprising 1,8-cineole, β-caryophyllene, N-acetyl cysteine, glutathione, ascorbic acid, methylcobalamin, an emulsifying agent, vegetable glycerin (as needed), water, sodium bicarbonate (as needed) and a preservative (as needed) is disclosed in Example 4. The method of manufacturing consists of mixing an amount of nitrogen purged purified sterile water or isotonic saline solution with ascorbic acid powder or crystals, sodium bicarbonate (as needed) and preservative (as needed) and dissolving, then adding amounts of N-acetyl cysteine, glutathione, and methylcobalamin followed by adding an amount of vegetable glycerin (as needed) and mixing until the liquid composition is homogeneous. Nitrogen gas purging can be used throughout the mixing period to minimize oxygenation of the water and oxidation of the compounds in the mixture. β-caryophyllene and 1,8-cineole are then separately mixed with the emulsifier, and after this mixture is homogeneous it is slowly added to the mixture and the mixture is slowly mixed until there is dissolution in the liquid, minimizing the volatilization of the 1,8-cineole and the β-caryophyllene. Mixing can be conducted in a zero or low headspace reactor to further minimize volatilization of β-caryophyllene and 1,8-cineole and oxidation of the compounds in the mixture. If an amount of β-caryophyllene and 1,8-cineole is added to the mixture at concentrations greater than the solubility of 1,8-cineole and β-caryophyllene in the mixture, then the β-caryophyllene and 1,8-cineole can be emulsified in the liquid composition with the addition of a suitable emulsifier, for example, Tween 20, also known as Polysorbate 20 and polyoxyethylene(20)sorbitan monooleate. Mixing is limited to that required to create a stable single-phase homogeneous solution or emulsion and to minimize volatilization of β-caryophyllene and 1,8-cineole.

Methods of use of the liquid composition in Example 4 include but are not meant to be limited to placing a quantity of the composition in an e-cigarette vaporizing device, an electronic thermal vaporization device, an ultrasonic vaping device, a nebulizer or an inhaler, and inhaling the aerosolized vapors resulting from creating an aerosolized mixture. The liquid composition component that is the TRPA1 antagonist that can be aerosolized or vaporized in Example 4 can optionally be made with borneol or a mixture of 1,8-cineole and borneol in the same or different total concentration range compared to the concentration range when using 1,8-cineole alone. This liquid composition that can be aerosolized is disclosed in Table 4. The aerosolizable liquid composition can be transferred to containers that can stored for one or more doses, the containers may or may not have nitrogen gas in the headspace, and the containers may or may not be refrigerated.

TABLE 4
Base Inhalation Liquid with β-Caryophyllene

| | Weight | | | |
|---|---|---|---|---|
| Ingredient | Percent (%) | Function | Sources | Secondary Effect |
| 1,8-Cineole | 0.1-10 | TRPA1 | Pure Compound or Essential | TRPM8 Agonist, |
| | | Antagonist | oils of: Eucalyptus polybractea; | modulate immune |
| | | | Eucalyptus globulus; | function, bacteriostatic |
| | | | Eucalyptus radiate; | Fungistatic, inhibition of |
| | | | Eucalyptus camaldulensis; | production of tumor |
| | | | Eucalyptus smithii; | necrosis factor- a (TNF- |
| | | | Eucalyptus globulus; | α), interleukin-1β (IL- |
| | | | Rosmarinus offficinalis | 1β), interleukin-4 (IL-4), |
| | | | | interleukin-5 (IL- |
| | | | | 5), leukotriene B4 |
| | | | | (LTB4), thromboxane B2 |
| | | | | (TXB2) and |
| | | | | prostaglandin E2 (PGE2) |
| β-Caryophyllene | 0.1-10 | CB₂ Agonist | Pure Compound or Essential | Analgesic, anti- |
| | | | oils of: Syzygium aromaticum, | inflammatory, |
| | | | Carum nigrum, Cinnamomum spp., | neuroprotective, anti- |
| | | | Humulus lupulus, Piper nigrum L., | depressive, anxiolytic, |
| | | | Cannabis sativa, Rosmarinus offficinalis, | and anti-nephrotoxicity, |
| | | | Ocimum spp., Origanum vulgare | inhibition of pro- |
| | | | | inflammatory cytokines |
| | | | | productions, such as |
| | | | | TNF-α, IL-1β and IL-6. |
| N-acetyl cysteine | 0.1-10 | Antioxidant, | Synthetic | Glutathione precursor, |
| | | Natural Thiol | | increase epithelial lining |
| | | Amino Acid | | fluid and lung glutathione |
| | | Containing | | concentrations, modulate |
| | | Compound | | immune function, |
| | | | | inhibits NF-kB |
| | | | | activation, modulates |
| | | | | immune function and |
| | | | | participates in the |
| | | | | pulmonary epithelial host |
| | | | | defense system, |
| | | | | radionuclide and heavy |
| | | | | metal chelate |
| Glutathione | 0.1-20 | Antioxidant, | Synthetic | Increase epithelial lining |
| | | Natural Thiol | | fluid and lung |
| | | Amino Acid | | glutathione |
| | | Containing | | concentrations, modulate |
| | | Compound | | immune function, |
| | | | | inhibits NF-kB |
| | | | | activation, radionuclide |
| | | | | and heavy metal chelate |
| Ascorbic Acid | 0.01-10 | Vitamin, | Synthetic | Decrease Vitamin C |
| | | Natural | | deficiency, modulate |
| | | Antioxidant | | immune function, |
| | | | | inhibition of |
| | | | | prostaglandin E2 |
| | | | | (PGE2), decrease in |
| | | | | bronchoconstriction |
| Methylcobalamin | 0.001-1.00 | Vitamin, | Synthetic | Decrease Vitamin B12 |
| | | Natural | | deficiency the result of |
| | | Antioxidant | | smoking. Reduce |
| | | | | cyanide concentrations in |
| | | | | lungs and serum |
| Vegetable | 0.0-95 | Thickener | Plant-Based Synthetic | Flavor and vapor |
| Glycerin | | | | production, rheology |
| | | | | control, viscosity |
| | | | | modifier |
| Emulsifier | 0.1-2.0 | Stable | Natural or Synthetic | |
| | | Suspension | | |
| Water | 5.0-98 | Carrier | Filtered Water | Diluent |
| Sodium | variable | pH Adjustment | Natural Mineral | Natural Buffer in |
| Bicarbonate | | | | Epithelial Cells |
| Preservative | variable | Chemical and | Natural or Synthetic | |
| | | Biological | | |
| | | Stability | | |

### Example 5

A preferred composition and a method of manufacture of a pharmaceutical liquid that is aerosolized, vaporized or both using a nebulizer comprising 1,8-cineole, β-caryophyllene, N-acetyl cysteine, glutathione, ascorbic acid, methylcobalamin, an emulsifying agent, sterile saline solution, sodium bicarbonate (as needed) and a preservative (as needed) is disclosed in Example 5. The method of manufacturing consists of mixing 94.89 g of nitrogen purged 0.9% sterile saline solution with 0.01 g of ascorbic acid powder and dissolving the ascorbic acid, then adding 1.35 g of N-acetyl cysteine, 1.35 g of glutathione, 0.003 g of methylcobalamin and mixing until the liquid composition is homogeneous. This is followed by adding a mixture of 0.80 g of 1,8-cineole, 0.80 g of β-caryophyllene and 0.80 g of Polysorbate 20 to the mixture and slowly mixing until it is dissolved in the liquid, minimizing the volatilization of the 1,8-cineole and β-caryophyllene. Mixing is limited to that required to create a stable single-phase homogeneous solution and to minimize volatilization 1,8-cineole and β-caryophyllene. The pH of the solution is then measured and a quantity of sodium bicarbonate is added to raise the pH to 7.20. A quantity of a preservative can be added or alternatively the mixture can be refrigerated prior to use. Methods of use of the composition of the liquid composition in Example 5 include but are not meant to be limited to placing the composition in a an ultrasonic, vibrating mesh or jet nebulizer and inhalation of the vapors resulting from creating an aerosolized mixture. Methods of use of the composition of the liquid in Example 5, include adding approximately 1 mL to 5 ml of the mixture to a liquid nebulizer for inhalation by a patient. The liquid composition that can be aerosolized or vaporized in Example 5 can optionally be made with borneol or a mixture of 1,8-cineole, β-caryophyllene and borneol in the same total concentration range as 1,8-cineole and β-caryophyllene. This liquid composition is disclosed in Table 5.

TABLE 5
Preferred Base Nebulizer Liquid with β-Caryophyllene

| | Weight | | |
|---|---|---|---|
| Ingredient | Percent (%) | Function | Primary Effects |
| 1,8-Cineole | 0.80 | Inflammation Blocker, Anti- | TRPA1 Antagonist |
| | | Cancer | |
| β-Cary ophyllene | 0.80 | Inflammation Blocker | CB₂ Agonist |
| N-acetyl cysteine | 1.35 | Increase Epithelial Liquid | Antioxidant, Natural Thiol Amino |
| | | and Lung Tissue Glutathione | Acid Containing Compound |
| | | Concentration | |
| Glutathione | 1.35 | Increase Epithelial Liquid | Antioxidant, Natural Thiol Amino |
| | | and Lung Tissue Glutathione | Acid Containing Compound |
| | | Concentration | |
| Ascorbic Acid | 0.01 | Increase Epithelial Liquid | Vitamin, Natural Antioxidant |
| | | and Lung Tissue Vitamin C | |
| | | Concentration | |
| Methylcobalamin | 0.00300 | Increase Epithelial Liquid | Vitamin, Natural Antioxidant |
| | | and Lung Tissue Vitamin | |
| | | B12 Concentration | |
| Polysorbate 20 | 0.80 | Stable Suspension | |
| Sterile Saline Water - | 94.89 | Carrier | Isotonic Diluent |
| 0.9% | | | |
| Sodium Bicarbonate | variable to | pH Adjustment | Adjust pH to 7.20 |
| | pH = 7.2 | | |
| Preservative | Variable as | Chemical and Biological | |
| | Needed | Stability | |

### Example 6

A composition and a method of manufacture of a pharmaceutical liquid that is aerosolized, vaporized or both in an ultrasonic or thermal vaporization device including 1,8-cineole, β-caryophyllene, N-acetyl cysteine, glutathione, ascorbic acid, methylcobalamin, an emulsifying agent, vegetable glycerin, sterile deionized water, sodium bicarbonate (as needed), and a preservative (as needed) is disclosed in Example 6. The method of manufacturing consists of mixing 16.93 g of nitrogen purged sterile deionized water with 0.01 g of ascorbic acid powder and dissolving the ascorbic acid, then adding 1.20 g of N-acetyl cysteine, 1.50 g glutathione, 0.003 g methylcobalamin and mixing until the liquid composition is homogeneous. This is followed by adding 90.72 g of vegetable glycerin and mixing. This is then followed by adding a mixture of 1.69 g of 1,8-cineole, 1.69 g of β-caryophyllene together and slowly mixing until they are dissolved together. Once the 1,8-cineole, β-caryophyllene and Polysorbate 20 are homogeneously mixed, this mixture is added to the glycerin-water based mixture and dissolved into the liquid, minimizing the volatilization of the 1,8-cineole and β-caryophyllene. The pH of the solution is then measured and a quantity of sodium bicarbonate is added to raise the pH to 7.20. A quantity of a preservative can be added or alternatively the mixture can be refrigerated prior to use. The liquid composition in Example 6 may be made with a quantity of vegetable glycerin that is less than 90.72 g and can be decreased by increasing a corresponding mass of nitrogen purged water added.

Methods of use of the composition of the liquid composition in Example 6 include but are not meant to be limited to placing the composition in an e-cigarette vaporizing device, a thermal vaporization device, a vaping pen, an electronic vaping mod, or an ultrasonic vaping device and inhalation of the vapors resulting from creating an aerosolized mixture. A preferred vaping device is one that has temperature control and the temperature is limited to an upper limit of 200° C. The aerosolizable pharmaceutical liquid composition can be transferred to containers that can be stored for one or more doses, the containers may or may not have nitrogen gas in the headspace and the containers may or may not be refrigerated. This liquid composition is disclosed in Table 6.

TABLE 6
Preferred Base Vape Liquid with β-Caryophyllene

| | Weight | | |
|---|---|---|---|
| Ingredient | Percent (%) | Function | Primary Effects |
| 1,8-Cineole | 1.69 | Inflammation Blocker, | TRPA1 Antagonist |
| | | Anti-Cancer | |
| β-Caryophyllene | 1.69 | Inflammation Blocker | CB₂ Agonist |
| N-acetyl cysteine | 1.20 | Increase Epithelial Liquid | Antioxidant, Natural Thiol |
| | | and Lung Tissue | Amino Acid Containing |
| | | Glutathione Concentration | Compound |
| Glutathione | 1.50 | Increase Epithelial Liquid | Antioxidant, Natural Thiol |
| | | and Lung Tissue | Amino Acid Containing |
| | | Glutathione Concentration | Compound |
| Ascorbic Acid | 0.01 | Increase Epithelial Liquid | Vitamin, Natural Antioxidant |
| | | and Lung Tissue Vitamin C | |
| | | Concentration | |
| Methylcobalamin | 0.00300 | Increase Epithelial Liquid | Vitamin, Natural Antioxidant |
| | | and Lung Tissue Vitamin | |
| | | B12 Concentration | |
| Polysorbate 20 | 2.04 | Stable Suspension | |
| Vegetable Glycerin | 90.72 | Thickener | |
| Sterile Water | 16.93 | Carrier | Diluent |
| Sodium Bicarbonate | variable to | pH Adjustment | Adjust pH to 7.20 |
| | pH = 7.2 | | |
| Preservative | Variable as | Chemical and Biological | |
| | Needed | Stability | |

### Example 7

A composition and a method of manufacture of a pharmaceutical liquid that is aerosolized, vaporized or both comprising 1,8-cineole, β-caryophyllene, N-acetyl cysteine, glutathione, ascorbic acid, methylcobalamin, dexapanthenol, L-theanine, taurine, an emulsifying agent, vegetable glycerin (as needed), water, sodium bicarbonate (as needed), and a preservative (as needed) is disclosed in Example 7. The method of manufacturing consists of mixing an amount of nitrogen purged purified sterile water or isotonic saline solution with ascorbic acid powder or crystals, sodium bicarbonate (as needed), and preservative (as needed) and dissolving, then adding amounts of N-acetyl cysteine, glutathione, dexpanthenol, L-theanine, taurine, and methylcobalamin, followed by adding an amount of vegetable glycerin (as needed) and mixing until the liquid composition is homogeneous. Nitrogen gas purging can be used throughout the mixing period to minimize oxygenation of the water and oxidation of the compounds in the mixture. β-caryophyllene and 1,8-cineole are then separately mixed with the emulsifier, and after this mixture is homogeneous, then slowly adding to the mixture and slowly mixing until it is dissolved in the liquid, minimizing the volatilization of the 1,8-cineole and the β-caryophyllene. Mixing can be conducted in a zero or low headspace reactor to further minimize volatilization of β-caryophyllene and 1,8-cineole and oxidation of the compounds in the mixture. If an amount of β-caryophyllene and 1,8-cineole is added to the mixture at concentrations greater than the solubility of 1,8-cineole and β-caryophyllene in the mixture, then the β-caryophyllene and 1,8-cineole can be emulsified in the liquid composition with the addition of a suitable emulsifier, for example, Tween 20, also known as Polysorbate 20 and polyoxyethylene(20)sorbitan monooleate. Mixing is limited to that required to create a stable single phase homogeneous solution or emulsion and to minimize volatilization of β-caryophyllene and 1,8-cineole.

Methods of use of the liquid composition in Example 7 include but are not meant to be limited to placing a quantity of the composition in an e-cigarette vaporizing device, an electronic thermal vaporization device, an ultrasonic vaping device, a nebulizer, or an inhaler and inhalation of the aerosolized vapors resulting from creating an aerosolized mixture. The liquid composition component that is the TRPA1 antagonist that can be aerosolized or vaporized in Example 7 can optionally be made with borneol or a mixture of 1,8-cineole and borneol in the same or different total concentration range compared when using 1,8-cineole alone. This liquid composition that can be aerosolized is disclosed in Table 7. The aerosolizable liquid composition can be transferred to containers that can stored for one or more doses, the containers may or may not have nitrogen gas in the headspace and the containers may or may not be refrigerated.

TABLE 7
Base Liquid with Amino Acids

| | Weight | | | |
|---|---|---|---|---|
| Ingredient | Percent (%) | Function | Sources | Secondary Effect |
| 1,8-Cineole | 0.1-10 | TRPA1 | Pure Compound or Essential | TRPM8 Agonist, modulate |
| | | Antagonist | oils of: Eucalyptus | immune function, bacteriostatic |
| | | | polybractea; Eucalyptus | Fungistatic, inhibition of |
| | | | globulus; Eucalyptus | production of tumor necrosis |
| | | | radiate; Eucalyptus | factor- a (TNF-α), interleukin- |
| | | | camaldulensis; Eucalyptus | 1β (IL-1β), interleukin-4 (IL-4), |
| | | | smithii; Eucalyptus | interleukin-5 (IL-5), leukotriene |
| | | | globulus; Rosmarinus | B4 (LTB4), thromboxane B2 |
| | | | offficinalis | (TXB2) and prostaglandin E2 |
| | | | | (PGE2) |
| β-Caryophyllene | 0.1-10 | CB₂ Agonist | Pure Compound or Essential | Analgesic, anti-inflammatory, |
| | | | oils of: Syzygium | neuroprotective, anti- |
| | | | aromaticum, Carum nigrum, | depressive, anxiolytic, and anti- |
| | | | Cinnamomum spp., | nephrotoxicity, inhibition of |
| | | | Humulus lupulus, Piper | pro-inflammatory cytokines |
| | | | nigrum L., Cannabis sativa, | productions, such as TNF-α, |
| | | | Rosmarinus offficinalis, | IL-1β and IL-6. |
| | | | Ocimum spp., Origanum | |
| | | | vulgare | |
| N-acetyl cysteine | 0.1-10 | Antioxidant, | Synthethic | Glutathione precursor, increase |
| | | Natural Thiol | | epithelial lining fluid and lung |
| | | Amino Acid | | glutathione concentrations, |
| | | Containing | | modulate immune function, |
| | | Compound | | inhibits NF-kB activation, |
| | | | | modulates immune function |
| | | | | and participates in the |
| | | | | pulmonary epithelial host |
| | | | | defense system, radionuclide |
| | | | | and heavy metal chelate |
| Glutathione | 0.1-20 | Antioxidant, | Synthethic | Increase epithelial lining fluid |
| | | Natural Thiol | | and lung glutathione |
| | | Amino Acid | | concentrations, modulate |
| | | Containing | | immune function, inhibits NF- |
| | | Compound | | kB activation, radionuclide and |
| | | | | heavy metal chelate |
| Ascorbic Acid | 0.01-10 | Vitamin, Natural | Vitamin, Natural, | Decrease Vitamin C deficiency, |
| | | Antioxidant | Antioxidant | modulate immune function, |
| | | | | inhibition of prostaglandin E2 |
| | | | | (PGE2), decrease in |
| | | | | bronchoconstriction |
| Methylcobalamin | 0.001-1 | Vitamin, Natural | Vitamin, Natural | Decrease Vitamin B12 |
| | | Antioxidant | Antioxidant | deficiency the result of |
| | | | | smoking. Reduce cyanide |
| | | | | concentrations in lungs and |
| | | | | serum |
| Dexpanthenol | 0.05-10 | Amino Acid, | Synthethic | Anti-inflammatory activity, |
| | | Antioxidant | | synthesis of Acetylcholine. |
| | | | | Inhibit Nitrite and TNF-α, |
| | | | | Inhibit Cell Proliferation of |
| | | | | Lung Cancer |
| L-Theanine | 0.05-10 | Amino Acid, | Synthethic | Anti-inflammatory activity, |
| | | Antioxidant | | antioxidative properties, and |
| | | | | hepatoprotective effects, |
| | | | | decreased the production of |
| | | | | IgE, monocyte chemoattractant |
| | | | | protein-1 (MCP-1), interleukin |
| | | | | (IL)-4, IL-5, IL-13, tumor |
| | | | | necrosis factor-alpha (TNF-α), |
| | | | | and interferon-gamma (INF-γ) |
| Taurine | 0.05-10 | Amino Acid, | Synthethic | Detoxification of destructive |
| | | Dissipate Toxic | | xenobiotic and toxic |
| | | Effects of HOCl | | compounds, preventing |
| | | in Epitheial | | alterations in membrane |
| | | Cenlls | | permeability |
| Vegetable | 0.0-95 | Thickener | Plant-Based Synthetic | Flavor and vapor production, |
| Glycerin | | | | rheology control, viscosity |
| | | | | modifier |
| Emulsifier | 0.1-2.0 | Stable | Natural or Synthetic | |
| | | Suspension | | |
| Sterile Water | 5.0-98 | Carrier | Filtered Water | Diluent |
| Sodium | variable | pH Adjustment | Natural Mineral | Natural Buffer in Epithelial |
| Bicarbonate | | | | Cells |

### Example 8

A preferred composition and a method of manufacture of a pharmaceutical liquid that is aerosolized, vaporized, or both comprising 1,8-cineole, β-caryophyllene, N-acetyl cysteine, glutathione, ascorbic acid, methylcobalamin, dexpanthenol, L-theanine, taurine, an emulsifying agent, sterile saline solution, sodium bicarbonate (as needed), and a preservative (as needed) is disclosed in Example 8. The method of manufacturing consists of mixing 92.69 g of nitrogen purged 0.9% sterile saline solution with 0.01 g of ascorbic acid powder and dissolving the ascorbic acid, then adding 1.35 g of N-acetyl cysteine, 1.35 g glutathione, 0.003 g methylcobalamin, 1.00 g of dexpanthenol, 0.70 g of L-theanine, and 0.50 g of taurine and mixing until the liquid composition is homogeneous. This is followed by adding a mixture of 0.80 g of 1,8-cineole, 0.80 g of β-caryophyllene and 0.80 g of Polysorbate 20 together and slowly mixing until they are dissolved together. This mixture is added to the glycerin-water based mixture and dissolved into the liquid, minimizing the volatilization of the 1,8-cineole and β-caryophyllene. Mixing is limited to that required to create a stable single-phase homogeneous solution and to minimize volatilization of the 1,8-cineole and β-caryophyllene. The pH of the solution is then measured and a quantity of sodium bicarbonate is added to raise the pH to 7.20. A quantity of a preservative can be added or alternatively the mixture can be refrigerated prior to use. Methods of use of the composition of the liquid composition in Example 8 include, but are not meant to be limited to placing the composition in a an ultrasonic, vibrating mesh, or jet nebulizer and inhaling the vapors resulting from creating an aerosolized mixture.

Methods of use of the composition of the liquid in Example 8 include adding approximately 1 mL to 5 ml of the mixture to a liquid nebulizer for inhalation by a patient. The liquid composition that can be aerosolized or vaporized in Example 8 can optionally be made with borneol or a mixture of 1,8-cineole, β-caryophyllene, and borneol in the same total concentration range as 1,8-cineole and β-caryophyllene. This liquid composition is shown in Table 8.

TABLE 8
Preferred Base Nebulizer Liquid with Amino Acids

| | Weight Percent | | |
|---|---|---|---|
| Ingredient | (%) | Function | Primary Effects |
| 1,8-Cineole | 0.80 | Inflammation Blocker, | TRPA1 Antagonist |
| | | Anti-Cancer | |
| β-Caryophyllene | 0.80 | Inflammation Blocker | CB₂ Agonist |
| N-acetyl cysteine | 1.35 | Increase Epithelial Liquid | Antioxidant, Natural Thiol |
| | | and Lung Tissue | Amino Acid Containing |
| | | Glutathione Concentration | Compound |
| Glutathione | 1.35 | Increase Epithelial Liquid | Antioxidant, Natural Thiol |
| | | and Lung Tissue | Amino Acid Containing |
| | | Glutathione Concentration | Compound |
| Ascorbic Acid | 0.01 | Increase Epithelial Liquid | Vitamin, Antioxidant |
| | | and Lung Tissue Vitamin C | |
| | | Concentration | |
| Methyl cobalamin | 0.003 | Increase Epithelial Liquid | Vitamin, Antioxidant |
| | | and Lung Tissue Vitamin | |
| | | B12 Concentration | |
| Dexpanthenol | 1.00 | Synthesis of Acetylcholine. | Provitamin, Cholinergic Agent, |
| | | Inhibit Nitrite and TNF-α, | Natural Antioxidant |
| | | Inhibit Cell Proliferation of | |
| | | Lung Cancer | |
| L-Theanine | 0.70 | Inflammation Blocker | Amino Acid, Natural |
| | | | Antioxidant |
| Taurine | 0.50 | Dissipate toxic effects of | Natural Antioxidant, Natural |
| | | HOCl in Epitheial Cenlls | Thiol Amino Acid Containing |
| | | | Compound |
| Polysorbate 20 | 0.80 | Stable Suspension | |
| Sterile Saline Water - | 92.69 | Carrier | Isotonic Diluent |
| 0.9% | | | |
| Sodium Bicarbonate | variable to | pH Adjustment | Adjust pH to 7.20 |
| | pH = 7.2 | | |
| Preservative | Variable as | Chemical and Biological | |
| | Needed | Stability | |

### Example 9

A composition and a method of manufacture of a pharmaceutical liquid that is aerosolized, vaporized, or both in an ultrasonic or thermal vaporization device comprising 1,8-cineole, β-caryophyllene, N-acetyl cysteine, glutathione, ascorbic acid, methylcobalamin, dexpanthenol, L-theanine, taurine, an emulsifying agent, vegetable glycerin, sterile deionized water, sodium bicarbonate (as needed), and a preservative (as needed) is disclosed in Example 9. The method of manufacturing consists of mixing 16.94 g of nitrogen purged sterile deionized water with 0.01 g of ascorbic acid powder dissolving the ascorbic acid, then adding 1.20 g of N-acetyl cysteine, 1.50 g glutathione, 0.003 g methylcobalamin, 1.00 g of dexapanthenol, 0.70 g of L-theanine, and 0.50 g taurine and mixing until the liquid composition is homogeneous. This is followed by adding 89.99 g of vegetable glycerin and mixing. This is then followed by adding a mixture of 1.70 g of 1,8-cineole, 1.70 g of β-caryophyllene, and 1.70 g Polysorbate 20 and slowly mixing until they are dissolved together. Once the 1,8-cineole, β-caryophyllene, and Polysorbate 20 are homogeneously mixed, this mixture is added to the glycerin-water based mixture and dissolved into the liquid, minimizing the volatilization of the 1,8-cineole and β-caryophyllene. The pH of the solution is then measured and a quantity of sodium bicarbonate is added to raise the pH to 7.20. A quantity of a preservative can be added or alternatively the mixture can be refrigerated prior to use. The liquid composition in Example 9 may be made with a quantity of vegetable glycerin that is less than 89.99 g and can be decreased by increasing a corresponding mass of nitrogen purged water added.

Methods of use of the composition of the liquid composition in Example 9 include but are not meant to be limited to placing the composition in an e-cigarette vaporizing device, a thermal vaporization device, a vaping pen, an electronic vaping mod, or an ultrasonic vaping device and inhalation of the vapors resulting from creating an aerosolized mixture. A preferred vaping device is one that has temperature control and has the temperature limited to an upper limit of 200° C. The aerosolizable pharmaceutical liquid composition can be transferred to containers that can be stored for one or more doses, the containers may or may not have nitrogen gas in the headspace and the containers may or may not be refrigerated. This liquid composition is disclosed in Table 9.

TABLE 9
Preferred Base Vape Liquid with Amino Acids

| | Weight | | |
|---|---|---|---|
| Ingredient | Percent (%) | Function | Primary Effects |
| 1,8-Cineole | 1.70 | Inflammation Blocker, Anti- | TRPA1 Antagonist |
| | | Cancer | |
| β-Caryophyllene | 1.70 | Inflammation Blocker | CB₂ Agonist |
| N-acetyl cysteine | 1.20 | Increase Epithelial Liquid | Antioxidant, Natural Thiol Amino |
| | | and Lung Tissue Glutathione | Acid Containing Compound |
| | | Concentration | |
| Glutathione | 1.50 | Increase Epithelial Liquid | Antioxidant, Natural Thiol Amino |
| | | and Lung Tissue Glutathione | Acid Containing Compound |
| | | Concentration | |
| Ascorbic Acid | 0.01 | Increase Epithelial Liquid | Vitamin, Natural Antioxidant |
| | | and Lung Tissue Vitamin C | |
| | | Concentration | |
| Methylcobalamin | 0.003 | Increase Epithelial Liquid | Vitamin, Natural Antioxidant |
| | | and Lung Tissue Vitamin | |
| | | B12 Concentration | |
| Dexpanthenol | 1.00 | Synthesis of Acetylcholine. | Amino Acid, Antioxidant |
| | | Inhibit Nitrite and TNF-α, | |
| | | Inhibit Cell Proliferation of | |
| | | Lung Cancer | |
| L-Theanine | 0.70 | Inflammation Blocker | Amino Acid, Antioxidant |
| Taurine | 0.50 | Dissipate toxic effects of | Antioxidant |
| | | HOCl in Epitheial Cenlls | |
| Polysorbate 20 | 1.70 | Stable Suspension | |
| Vegetable Glycerin | 89.99 | Thickener | |
| Sterile Water | 16.94 | Carrier | Diluent |
| Sodium Bicarbonate | variable to | pH Adjustment | Adjust pH to 7.20 |
| | pH = 7.2 | | |
| Preservative | Variable as | Chemical and Biological | |
| | Needed | Stability | |

### Example 10

A composition and a method of manufacture of a pharmaceutical liquid that is aerosolized, vaporized or both comprising 1,8-cineole, β-caryophyllene, N-acetyl cysteine, glutathione, ascorbic acid, methyl cobalamin, epigallocatechin, resveratrol, an emulsifying agent, vegetable glycerin (as needed), water, sodium bicarbonate (as needed) and a preservative (as needed) is disclosed in Example 10. The method of manufacturing consists of mixing an amount of nitrogen purged purified sterile water or isotonic saline solution with ascorbic acid powder or crystals, sodium bicarbonate (as needed), and preservative (if needed) and dissolving, then adding amounts of N-acetyl cysteine, glutathione, pre-solubilized epigallocatechin, pre-solubilized resveratrol, and methyl cobalamin, followed by adding an amount of vegetable glycerin (as needed), and mixing until the liquid composition is homogeneous. Nitrogen gas purging can be used throughout the mixing period to minimize oxygenation of the water and oxidation of the compounds in the mixture. β-caryophyllene and 1,8-cineole are then separately mixed with the emulsifier, and after this mixture is homogeneous, then slowly adding to the mixture and slowly mixing until it is dissolved in the liquid, minimizing the volatilization of the 1,8-cineole and the β-caryophyllene. Mixing can be conducted in a zero or low headspace reactor to further minimize volatilization of β-caryophyllene and 1,8-cineole and oxidation of the compounds in the mixture. If an amount of β-caryophyllene and 1,8-cineole is added to the mixture at concentrations greater than the solubility of 1,8-cineole and β-caryophyllene in the mixture, then the β-caryophyllene and 1,8-cineole can be emulsified in the liquid composition with the addition of a suitable emulsifier, for example, Tween 20, also known as Polysorbate 20 and polyoxyethylene(20)sorbitan monooleate. Mixing is limited to that required to create a stable single-phase homogeneous solution or emulsion and to minimize volatilization β-caryophyllene and 1,8-cineole.

Methods of use of the liquid composition in Example 10 include but are not meant to be limited to placing a quantity of the composition in an e-cigarette vaporizing device, an electronic thermal vaporization device, an ultrasonic vaping device, a nebulizer, or an inhaler and inhalation of the aerosolized vapors resulting from creating an aerosolized mixture. The liquid composition component that is the TRPA1 antagonist that can be aerosolized or vaporized in Example 10 can optionally be made with borneol or a mixture of 1,8-cineole and borneol in the same or different total concentration range compared when using 1,8-cineole alone. This liquid composition that can be aerosolized is disclosed in Table 10. The aerosolizable liquid composition can be transferred to containers that can stored for one or more doses, the containers may or may not have nitrogen gas in the headspace, and the containers may or may not be refrigerated.

TABLE 10
Base Liquid with Polyphenols

| | Weight | | | |
|---|---|---|---|---|
| Ingredient | Percent (%) | Function | Sources | Secondary Effect |
| 1,8-Cineole | 0.1-10 | TRPA1 | Pure Compound or Essential | TRPM8 Agonist, modulate immune |
| | | Antagonist | oils of: Eucalyptus | function, bacteriostatic Fungistatic, |
| | | | polybractea; Eucalyptus | inhibition of production of tumor |
| | | | globulus; Eucalyptus radiate; | necrosis factor- a (TNF-α), |
| | | | Eucalyptus camaldulensis; | interleukin-1β (IL-1β), interleukin-4 |
| | | | Eucalyptus smithii; | (IL-4), interleukin-5 (IL-5), leukotriene |
| | | | Eucalyptus globulus; | B4 (LTB4), thromboxane B2 (TXB2) |
| | | | Rosmarinus offficinalis | and prostaglandin E2 (PGE2) |
| β- | 0.1-10 | CB₂ | Pure Compound or Essential | Analgesic, anti-inflammatory, |
| Caryophyllene | | Agonist | oils of: Syzygium aromaticum, | neuroprotective, anti-depressive, |
| | | | Carum nigrum, Cinnamomum | anxiolytic, and anti-nephrotoxicity, |
| | | | spp., Humulus lupulus, Piper | inhibition of pro-inflammatory |
| | | | nigrum L., Cannabis sativa, | cytokines productions, such as TNF-α, |
| | | | Rosmarinus offficinalis, | IL-1β and IL-6. |
| | | | Ocimum spp., Origanum | |
| | | | vulgare | |
| N-acetyl | 0.1-10 | Antioxidant, | Synthethic | Glutathione precursor, increase |
| cysteine | | Natural | | epithelial lining fluid and lung |
| | | Thiol | | glutathione concentrations, modulate |
| | | Amino Acid | | immune function, inhibits NF-kB |
| | | Containing | | activation, modulates immune |
| | | Compound | | function and participates in the |
| | | | | pulmonary epithelial host defense |
| | | | | system, radionuclide and heavy metal |
| | | | | chelate |
| Glutathione | 0.1-20 | Antioxidant, | Synthethic | Increase epithelial lining fluid and |
| | | Natural | | lung glutathione concentrations, |
| | | Thiol | | modulate immune function, inhibits |
| | | Amino Acid | | NF-kB activation, radionuclide and |
| | | Containing | | heavy metal chelate |
| | | Compound | | |
| Ascorbic Acid | 0.01-10 | Vitamin | Vitamin, Natural Antioxidant | Decrease Vitamin C deficiency, |
| | | | | modulate immune function, inhibition |
| | | | | of prostaglandin E2 (PGE2), decrease |
| | | | | in bronchoconstriction |
| Methylcobalamin | 0.001-10 | Vitamin | Vitamin, Natural Antioxidant | Decrease Vitamin B12 deficiency the |
| | | | | result of smoking. Reduce cyanide |
| | | | | concentrations in lungs and serum |
| Epigallocatechin- | 0.05-10 | Polyphenol, | Powder naturally derived | Leads to formation of |
| 3-gallate | | Antioxidant | from leave of | epigallocatechin-3-gallate-2′-N-acetyl |
| | | | Camellia sinensis | cysteine adduct. therapeutic effect on |
| | | | | chronic airway inflammation and |
| | | | | abnormal airway mucus production |
| Resveratrol | 0.1-10 | Polyphenol, | Synthethic | Antibacterial, antifungal, anti-tumor, |
| | | Antioxidant | | anti-inflammatory, activations of |
| | | | | Sirtuin 1 (SIRT1), reduction in lung |
| | | | | tissue neutrophils and |
| | | | | proinflammatory cytokines |
| Vegetable | 0.0-95 | Thickener | Plant-Based Synthetic | Flavor and vapor production, rheology |
| Glycerin | | | | control, viscosity modifier |
| Emulsifier | 0.1-2.0 | Stable | Natural or Synthetic | |
| | | Suspension | | |
| Sterile Water | 5.0-98 | Carrier | Filtered Water | Diluent |
| Sodium | variable | pH | Natural Mineral | Natural Buffer in Epithelial Cells |
| Bicarbonate | | Adjustment | | |
| Preservative | variable | Chemical | Natural or Synthetic | |
| | | and | | |
| | | Biological | | |
| | | Stability | | |

### Example 11

A composition and a method of manufacture of a pharmaceutical liquid that is aerosolized, vaporized, or both comprising 1,8-cineole, β-caryophyllene, cannabidiol, N-acetyl cysteine, glutathione, ascorbic acid, methyl cobalamin, an emulsifying agent, vegetable glycerin, water, sodium bicarbonate (as needed), and a preservative (as needed) is disclosed in Example 11. The method of manufacturing consists of mixing an amount of nitrogen purged purified sterile water or isotonic saline solution with ascorbic acid powder or crystals, sodium bicarbonate, and preservative (if needed) and dissolving, then adding amounts of N-acetyl cysteine, glutathione, and methyl cobalamin, followed by adding an amount of vegetable glycerin (as needed), and mixing until the liquid composition is homogeneous. Nitrogen gas purging can be used throughout the mixing period to minimize oxygenation of the water and oxidation of the compounds in the mixture. Cannabidiol is solubilized in a mixture of β-caryophyllene and 1,8-cineole, with limited mixing to minimize the volatilization loss of β-caryophyllene and 1,8-cineole. Following this step, the cannabidiol, β-caryophyllene, 1,8-cineole mixture is separately mixed with an emulsifier, and after this mixture is homogeneous, then it is slowly added to the mixture and slowly mixed until it is dissolved in the liquid, minimizing the volatilization of the 1,8-cineole and the β-caryophyllene. Mixing can be conducted in a zero or low headspace reactor to further minimize volatilization of β-caryophyllene and 1,8-cineole and oxidation of the compounds in the mixture. Mixing is limited to that required to create a stable single-phase homogeneous solution or emulsion and to minimize volatilization β-caryophyllene and 1,8-cineole.

Methods of use of the liquid composition in Example 11 include, but are not meant to be limited to placing a quantity of the composition in an e-cigarette vaporizing device, an electronic thermal vaporization device, an ultrasonic vaporization device, a nebulizer, or an inhaler and inhalation of the aerosolized vapors resulting from creating an aerosolized mixture. The liquid composition component that is the TRPA1 antagonist that can be aerosolized or vaporized in Example 11 can optionally be made with borneol or a mixture of 1,8-cineole, β-caryophyllene, and/or borneol in the same or a different total concentration range compared to the concentration range when using 1,8-cineole alone. In another embodiment of this liquid composition cannabidiol can be substituted with one or more cannabinoid compounds, including but not limited to 9-Tetrahydrocannabinol (delta-9-THC), 9-THC Propyl Analogue (THC-V), Cannabidiol (CBD), Cannabidiol Propyl Analogue (CBD-V), Cannabinol (CBN), Cannabichromene (CBC), Cannabichromene Propyl Analogue (CBC-V), Cannabigerol (CBG). A liquid composition that can be aerosolized is shown in Table 11. The aerosolizable liquid composition can be transferred to containers that can stored for one or more doses, the containers may or may not have nitrogen gas in the headspace, and the containers may or may not be refrigerated.

TABLE 11
Basic Liquid with CBD

| | Weight | | | |
|---|---|---|---|---|
| Ingredient | Percent (%) | Function | Sources | Secondary Effect |
| 1,8-Cineole | 0.1-10 | TRPA1 | Pure Compound or | TRPM8 Agonist, modulate |
| | | Antagonist | Essential oils of: | immune function, |
| | | | Eucalyptus polybractea; | bacteriostatic Fungistatic, |
| | | | Eucalyptus globulus; | inhibition of production of |
| | | | Eucalyptus radiate; | tumor necrosis factor- a |
| | | | Eucalyptus camaldulensis; | (TNF-α), interleukin-1β |
| | | | Eucalyptus smithii; | (IL-1β), interleukin-4 (IL-4), |
| | | | Eucalyptus globulus; | interleukin-5 (IL-5), |
| | | | Rosmarinus offficinalis | leukotriene B4 (LTB4), |
| | | | | thromboxane B2 (TXB2) and |
| | | | | prostaglandin E2 (PGE2) |
| β-Caryophyllene | 0.1-10 | CB₂ | Pure Compound or | Analgesic, anti- |
| | | Agonist | Essential oils of: | inflammatory, |
| | | | Syzygium aromaticum, | neuroprotective, anti- |
| | | | Carum nigrum, | depressive, anxiolytic, and |
| | | | Cinnamomum spp., | anti-nephrotoxicity, |
| | | | Humulus lupulus, Piper | inhibition of pro- |
| | | | nigrum L., Cannabis | inflammatory cytokines |
| | | | sativa, Rosmarinus | productions, such as TNF-α, |
| | | | offficinalis, Ocimum | IL-1β and IL-6. |
| | | | spp., Origanum vulgare | |
| Cannabidiol | 0.005-5 | Anti- | Natural, Hemp Oil, | Inhibition of production of |
| | | inflammatory | Nanoemulsion, Purified | tumor necrosis factor- a |
| | | | Crystal, Full Spectrum | (TNF-α), interleukin-6 (IL- |
| | | | | 6), macrophage |
| | | | | inflammatory protein (MIP- |
| | | | | 2), Chemokine (C-X-C |
| | | | | motif) ligand 2 (CXCL2). |
| | | | | Inhibition of adenosine |
| | | | | uptake and signaling through |
| | | | | the adenosine A2A receptor. |
| | | | | Anticancer. |
| N-acetyl cysteine | 0.1-10 | Antioxidant, | Synthethic | Glutathione precursor, |
| | | Natural Thiol | | increase epithelial lining fluid |
| | | Amino Acid | | and lung glutathione |
| | | Containing | | concentrations, modulate |
| | | Compound | | immune function, inhibits |
| | | | | NF-kB activation, modulates |
| | | | | immune function and |
| | | | | participates in the pulmonary |
| | | | | epithelial host defense |
| | | | | system, radionuclide and |
| | | | | heavy metal chelate |
| Glutathione | 0.1-20 | Antioxidant, | Synthethic | Increase epithelial lining |
| | | Natural Thiol | | fluid and lung glutathione |
| | | Amino Acid | | concentrations, modulate |
| | | Containing | | immune function, inhibits |
| | | Compound | | NF-kB activation, |
| | | | | radionuclide and heavy |
| | | | | metal chelate |
| Ascorbic Acid | 0.01-10 | Vitamin, | Synthethic | Decrease Vitamin C |
| | | Natural | | deficiency, modulate immune |
| | | Antioxidant | | function, inhibition of |
| | | | | prostaglandin E2 (PGE2), |
| | | | | decrease in |
| | | | | bronchoconstriction |
| Methylcobalamin | 0.001-10 | Vitamin, | Synthethic | Decrease Vitamin B12 |
| | | Natural | | deficiency the result of |
| | | Antioxidant | | smoking. Reduce cyanide |
| | | | | concentrations in lungs and |
| | | | | serum |
| Vegetable | 0.0-95 | Thickener | Plant-Based Synthetic | Flavor and vapor production, |
| Glycerin | | | | rheology control, viscosity |
| | | | | modifier |
| Polysorbate 20 | 0.1-2.0 | Emulsifier | Synthetic | |
| Sterile Water | 5.0-98 | Carrier | Filtered Water | Diluent |
| Sodium | variable | pH | Natural Mineral | Natural Buffer in Epithelial |
| Bicarbonate | | Adjustment | | Cells |
| Preservative | variable | Chemical | Natural or Synthetic | |
| | | and | | |
| | | Biological | | |
| | | Stability | | |

### Example 12

A composition and a method of manufacture of a pharmaceutical liquid that is aerosolized, vaporized, or both comprising 1,8-cineole, β-caryophyllene, nicotine, N-acetyl cysteine, glutathione, ascorbic acid, methyl cobalamin, an emulsifying agent, vegetable glycerin, water, sodium bicarbonate (as needed), and a preservative (as needed) is disclosed in Example 12. The method of manufacturing consists of mixing an amount of nitrogen purged purified sterile water or isotonic saline solution with ascorbic acid powder or crystals, sodium bicarbonate, and a preservative (if needed) and dissolving, then adding amounts of N-acetyl cysteine, glutathione, and methyl cobalamin. Following mixing of this mixture, an amount of a nicotine salt is added to an amount of vegetable glycerin (if used) to solubilize the nicotine salt. The nicotine salt-vegetable glycerin mixture is then added to the water, ascorbic acid n-acetyl cysteine, glutathione mixture and mixed until the liquid composition is homogeneous. Nitrogen gas purging can be used throughout the mixing period to minimize oxygenation of the water and oxidation of the compounds in the mixture. Alternatively, if freebase (unprotonated nicotine) is used in the formulation, the unprotonated nicotine is solubilized in a mixture of β-caryophyllene and 1,8-cineole, with limited mixing to minimize the volatilization loss β-caryophyllene and 1,8-cineole. Following this step, the nicotine, β-caryophyllene, 1,8-cineole mixture is separately mixed with an emulsifier, and after this mixture is homogeneous, then it is slowly adding to the vegetable glycerin-water mixture and slowly mixed until it is dissolved in the liquid, minimizing the volatilization of the 1,8-cineole and the β-caryophyllene. Mixing can be conducted in a zero or low headspace reactor to further minimize volatilization of β-caryophyllene and 1,8-cineole and oxidation of the compounds in the mixture. Mixing is limited to that required to create a stable single-phase homogeneous solution or emulsion and to minimize volatilization β-caryophyllene and 1,8-cineole.

Methods of use of the composition of the liquid composition in Example 12 include, but are not meant to be limited to placing the composition in an e-cigarette vaporizing device, a thermal vaporization device, a vaping pen, an ultrasonic vaping device, or an electronic vaping mod and inhalation of the vapors resulting from creating an aerosolized mixture. A preferred vaping device is one that has temperature control and has the temperature limited to an upper limit of 200° C. The aerosolizable pharmaceutical liquid composition can be transferred to containers that can be stored for one or more doses, the containers may or may not have nitrogen gas in the headspace, and the containers may or may not be refrigerated. This liquid composition is disclosed in Table 12.

TABLE 12
Basic Liquid with Nicotine

| | Weight | | | |
|---|---|---|---|---|
| Ingredient | Percent (%) | Function | Sources | Secondary Effect |
| 1,8-Cineole | 0.1-10 | TRPA1 | Pure Compound or | TRPM8 Agonist, modulate immune |
| | | Antagonist | Essential oils of: | function, bacteriostatic Fungistatic, |
| | | | Eucalyptus polybractea; | inhibition of production of tumor |
| | | | Eucalyptus globulus; | necrosis factor- a (TNF-α), |
| | | | Eucalyptus radiate; | interleukin-1β (IL-1β), interleukin-4 |
| | | | Eucalyptus camaldulensis; | (IL-4), interleukin-5 (IL-5), |
| | | | Eucalyptus smithii; | leukotriene B4 (LTB4), |
| | | | Eucalyptus globulus; | thromboxane B2 (TXB2) and |
| | | | Rosmarinus offficinalis | prostaglandin E2 (PGE2) |
| β-Caryophyllene | 0.1-10 | CB₂ | Pure Compound or | Analgesic, anti-inflammatory, |
| | | Agonist | Essential oils of: | neuroprotective, anti-depressive, |
| | | | Syzygium aromaticum, | anxiolytic, and anti-nephrotoxicity, |
| | | | Carum nigrum, | inhibition of pro-inflammatory |
| | | | Cinnamomum spp., | cytokines productions, such as TNF-α, |
| | | | Humulus lupulus, Piper | IL-1β andIL-6. |
| | | | nigrum L., Cannabis | |
| | | | sativa, Rosmarinus | |
| | | | offficinalis, Ocimum | |
| | | | spp., Origanum vulgare | |
| Nicotine | 0.01-5.0 | Alternative | Natural-Extracted from | |
| | | Nicotine | Nicotiana rustica, | |
| | | Source to | Nicotine Salt, Pure | |
| | | Cigarette | Nicotine or Synthethic, | |
| | | Smoking | Unprotonated nicotine, | |
| | | | protinated nicotine | |
| N-acetyl cysteine | 0.1-10 | Antioxidant, | Synthethic | Glutathione precursor, increase |
| | | Natural Thiol | | epithelial lining fluid and lung |
| | | Amino Acid | | glutathione concentrations, modulate |
| | | Containing | | immune function, inhibits NF-kB |
| | | Compound | | activation, modulates immune |
| | | | | function and participates in the |
| | | | | pulmonary epithelial host defense |
| | | | | system, radionuclide and heavy |
| | | | | metal chelate |
| Glutathione | 0.1-20 | Antioxidant, | Synthethic | Increase epithelial lining fluid and |
| | | Natural Thiol | | lung glutathione concentrations, |
| | | Amino Acid | | modulate immune function, inhibits |
| | | Containing | | NF-kB activation, radionuclide and |
| | | Compound | | heavy metal chelate |
| Ascorbic Acid | 0.01-10 | Vitamin, | Synthethic | Decrease Vitamin C deficiency, |
| | | Natural | | modulate immune function, |
| | | Antioxidant | | inhibition of prostaglandin E2 |
| | | | | (PGE2), decrease in |
| | | | | bronchoconstriction |
| Methylcobalamin | 0.001-10 | Vitamin, | Synthethic | Decrease Vitamin B12 deficiency the |
| | | Natural | | result of smoking. Reduce cyanide |
| | | Antioxidant | | concentrations in lungs and serum |
| Vegetable | 0.0-95 | Thickener | Plant-Based Synthetic | Flavor and vapor production, |
| Glycerin | | | | rheology control, viscosity modifier |
| Polysorbate 20 | 0.1-2.0 | Emulsifier | Synthetic | |
| Sterile Water | 5.0-98 | Carrier | Filtered Water | Diluent |
| Sodium | variable | pH | Natural Mineral | Natural Buffer in Epithelial Cells |
| Bicarbonate | | Adjustment | | |
| Preservative | variable | Chemical | Natural or Synthetic | |
| | | and | | |
| | | Biological | | |
| | | Stability | | |

### Example 13

A composition and a method of manufacture of a pharmaceutical liquid that is aerosolized, vaporized, or both in an ultrasonic vaping device or thermal vaporization device comprising 1,8-cineole, β-caryophyllene, nicotine salt, N-acetyl cysteine, glutathione, ascorbic acid, methyl cobalamin, an emulsifying agent, vegetable glycerin, sterile deionized water, sodium bicarbonate (as needed), and a preservative (as needed) is disclosed in Example 13. The method of manufacturing consists of mixing 16.93 g of nitrogen purged sterile deionized water with 0.01 g of ascorbic acid powder dissolving the ascorbic acid, then adding 1.20 g of N-acetyl cysteine, 1.53 g glutathione, 0.003 g methylcobalamin, and mixing until the liquid composition is homogeneous. 1.75 g of nicotine salt (54% nicotine) is added to 87.93 g vegetable glycerin and mixed until the nicotine salt is dissolved. This is followed by adding a mixture of 1.08 g of 1,8-cineole, 1.08 g β-caryophyllene and 1.18 g Polysorbate 20 together and slowly mixed until they are dissolved together, with limited mixing to minimize the volatilization loss β-caryophyllene and 1,8-cineole. This is followed by adding the vegetable glycerin-nicotine mixture to the β-caryophyllene, 1,8-cineole and Polysorbate 20 mixture and slowly mixed to create a stable single-phase homogeneous solution and to minimize volatilization of 1,8-cineole and β-caryophyllene. The water, glutathione, N-acetyl cysteine, and methylcobalamin are then added and slowly mixed until homogeneous. The pH of the solution is then measured, and a quantity of sodium bicarbonate is added to raise the pH to 7.20. A quantity of a preservative can be added, or alternatively the mixture can be refrigerated prior to use. The liquid composition in Example 13 may be made with a quantity of vegetable glycerin that is less than 87.93 g and can be decreased by increasing a corresponding mass of nitrogen purged water added.

Methods of use of the composition of the liquid composition in Example 13 include but are not meant to be limited to placing the composition in an e-cigarette vaporizing device, a thermal vaporization device, a vaping pen, an ultrasonic vaping device, or an electronic vaping mod and inhalation of the vapors resulting from creating an aerosolized mixture. A preferred vaping device is one that has temperature control and has the temperature is limited to an upper limit of 200° C. The aerosolizable pharmaceutical liquid composition can be transferred to containers that can be stored for one or more doses, the containers may or may not have nitrogen gas in the headspace, and the containers may or may not be refrigerated. This liquid composition is disclosed in Table 13.

TABLE 13
Preferred Base Vape Liquid with Nicotine

| | Weight | | |
|---|---|---|---|
| Ingredient | Percent (%) | Function | Primary Effects |
| 1,8-Cineole | 1.08 | Inflammation Blocker, | TRPA1 Antagonist |
| | | Anti-Cancer | |
| β-Caryophyllene | 1.08 | Inflammation Blocker | CB₂ Agonist |
| Nicotine | 1.75 | Nicotine Salt | Alternative Nicotine Source to |
| | | | Cigarette Smoking |
| N-acetyl cysteine | 1.20 | Increase Epithelial Liquid | Antioxidant, Natural Thiol |
| | | and Lung Tissue | Amino Acid Containing |
| | | Glutathione Concentration | Compound |
| Glutathione | 1.53 | Increase Epithelial Liquid | Antioxidant, Natural Thiol |
| | | and Lung Tissue | Amino Acid Containing |
| | | Glutathione Concentration | Compound |
| Ascorbic Acid | 0.01 | Increase Epithelial Liquid | Vitamin, Natural Antioxidant |
| | | and Lung Tissue Vitamin C | |
| | | Concentration | |
| Methylcobalamin | 0.00300 | Increase Epithelial Liquid | Vitamin, Natural Antioxidant |
| | | and Lung Tissue Vitamin | |
| | | B12 Concentration | |
| Vegetable Glycerin | 87.93 | Thickener | |
| Polysorbate 20 | 1.18 | Emulsifier | |
| Sterile Water | 16.93 | Carrier | Isotonic Diluent |
| Sodium Bicarbonate | variable to | pH Adjustment | Adjust pH to 7.20 |
| | pH = 7.2 | | |
| Preservative | Variable as | Chemical and Biological | |
| | Needed | Stability | |

### Example 14

A preferred composition and a method of manufacture of a pharmaceutical liquid that is aerosolized, vaporized, or both in an ultrasonic vaping device or a thermal vaporization device that is part of a combined smoking cessation and respiratory system health improvement product is disclosed in Example 14. The method for cessation of smoking consists of four separate liquid compositions that are aerosolized and inhaled, each with similar concentrations of N-acetyl cysteine, glutathione, 1,8-cineole, β-caryophyllene, methylcobalamin, an emulsifier, vegetable glycerin, and water.

In this example, cigarette smoking cessation is achieved first by the elimination of the use of combustion cigarettes by the use of ultrasonic vaping device or an electronic thermal liquid aerosolization devices with nicotine replacement therapy. The method of cigarette smoking cessation in this present invention utilizes a nicotine step-down process by which the daily consumption of nicotine is reduced using higher to lower nicotine concentrations over time, leading to the complete elimination of nicotine in the formulation. There are four nicotine reduction steps in this method of cigarette smoking cessation as part of this cigarette smoking and nicotine addiction withdrawal system. The first step to cigarette smoking cessation comprises switching from smoking cigarettes to the use of an electronic thermal liquid aerosolization device to consume nicotine. A unique and distinctive feature of this present invention is that in addition to providing a nicotine replacement therapy leading to the complete withdrawal of an individual from nicotine, this formulation additionally provides health benefits repairing respiratory system damage and disease caused by an individual's history of smoking cigarettes. The health benefits resulting from the inhalation of aerosolized N-acetyl cysteine, glutathione, 1,8-cineole, β-caryophyllene, and methylcobalamin are the result of the multifunctional mechanisms of using a TRPA1 antagonist, a CB₂ agonist, glutathione replacement in the lungs, epithelial lining fluid, and epithelial tissues, antioxidant treatment by the glutathione precursor N-acetyl cysteine, and vitamin B12 replacement therapy.

The method of use of the first of four steps to reduce a person's daily nicotine is the inhalation of approximately 20 mg per day of nicotine by vaporizing the formulation disclosed in Table 14. The formulation of Step 1 is provided in Table 14. Based on an approximated consumption of 1 mL of liquid vaporized using 150 puffs per day from an ultrasonic vaporization device, a thermal liquid aerosolization device; not limited to an electronic vaping device or an e-cigarette, the daily consumption of nicotine is about 20 mg. The daily dose of other non-carrier components of the composition disclosed in Table 14 is as follows: glutathione (19.65 mg); n-acetyl cysteine (13.76 mg); 1,8-cineole (10.87 mg); β-caryophyllene (5.34 mg); and vitamin B12 (9.38 μg). An emulsifier, for example, Polysorbate 20, may be provided at 9.73 mg; sterile deionized water may be provide at 212 mg; and vegetable glycerin may be provided at 1,096 mg. The period of time that a person consumes the composition by aerosolization of the Step 1 formulation disclosed in Table 14 can be variable, depending on a person's smoking history, the nature of their nicotine addiction, their susceptibility to nicotine addiction, their willingness to quit smoking cigarettes, and their psychological support system. The period of time a person would use the Step 1 nicotine replacement composition could vary from as short as two weeks to as long as several months. For example, the period of time at Step 1 may be 40 to 60 days. A person of ordinary skill in the art would recognize that the precise concentrations of each of the components identified in Table 14 could be varied over a range to principally accomplish the same outcomes as using the actual concentrations identified in Table 14. The use of deionized water and vegetable glycerin could also be varied dependent upon the type of liquid aerosolization device used. For example, if a nebulization device or an ultrasonic vaporization device were used to provide an aerosol phase of the liquid composition, the concentration of vegetable glycerin could be greatly reduced or even completely eliminated and made up with a water phase. Similarly, if a nebulization device or an ultrasonic vaporization device were used, deionized water could be replaced with a simple saline solution isotonic with that of epithelial lining fluid of the lungs, approximately 0.9 percent sodium chloride, for example. A person ordinarily skilled in the art would recognize that if an electronic thermal vaporization device, a vaping device, a vape pen, an ultrasonic vaporization device or an e-cigarette were used to deliver the composition in Table 14, then the water phase could predominantly be replaced by vegetable glycerin or another non-aqueous phase carrier. A person ordinarily skilled in the art would also recognize that the concentrations of each component disclosed in Table 14 could be increased or decreased by increasing or decreasing the total liquid volume of the composition to adjust for the specific liquid aerosolization device used and the number of puffs or duration of time required of the device to deliver the approximate dose of 1 mL of the liquid composition identified in Table 14.

An embodiment of the present invention in Step 1 of this smoking cessation system is to provide approximately a similar number of puffs that an individual normally takes when smoking cigarettes prior to using this system. This helps to satisfy the oral fixation associated with smoking cigarettes. A programmable electronic vaporization device can essentially vary the number of puffs used per mL of the liquid composition disclosed in Table 14. A person of ordinary skill in the art would recognize that, if a person wanting to quit smoking cigarettes was unable to progress to the next steps of this system of cigarette smoking cessation, then the health benefits of remaining at Step 1 would be better than if that person returned to smoking cigarettes for a longer term than envisioned in Step 1, including for a period of many years.

TABLE 14
Smoking Cessation Vape Liquid - Step 1

| | Liquid | | Dose at | |
|---|---|---|---|---|
| Compound | Concentration | Units | 150 Puffs | Units |
| Glutathione | 19.65 | mg/mL | 19.650 | mg |
| n-acetyl cysteine | 13.76 | mg/mL | 13.760 | mg |
| 1,8-cineole | 10.87 | mg/mL | 10.870 | mg |
| β-Caryophyllene | 5.34 | mg/mL | 5.340 | mg |
| Nicotine Salt | 20.00 | mg/mL | 20.000 | mg |
| (54% nicotine) | | | | |
| Vitamin B12 | 9.38 | μg/mL | 9.38 | μg |
| Polysorbate 20 | 9.73 | mg/mL | 9.730 | mg |
| Sterile Deionized Water | 212.23 | mg/mL | 212.230 | mg |
| Vegetable Glycerin | 1096.55 | mg/mL | 1096.550 | mg |

As part of the method for cigarette smoking cessation, Step 2 is based on an approximated consumption of 1 mL of liquid vaporized using 125 puffs per day from an ultrasonic vaping device or an electronic thermal liquid aerosolization device. The daily consumption of nicotine is about 14 mg, as disclosed in the composition of Table 15. The period of time a person would use the Step 2 nicotine replacement formulation could vary from as short as 2 weeks to as long as two months, for example, 14 to 30 days. An embodiment of this present invention is for an individual to decrease their oral fixation associated with their cigarette smoking habit and behavior. Therefore, there is a reduction in the number of puffs from 150 puffs per day in Step 1 to 125 puffs per day in Step 2. A person ordinarily skilled in the art would recognize that, if a person wanting to quit smoking cigarettes was unable to progress to the next steps of this system of cigarette smoking cessation, then the health benefits of remaining at Step 2 would be better than if the person returned to smoking cigarettes for a longer term than envisioned in Step 2, including for a period of many years.

TABLE 15
Smoking Cessation Vape Liquid - Step 2

| | Liquid | | Dose at | |
|---|---|---|---|---|
| Compound | Concentration | Units | 125 Puffs | Units |
| Glutathione | 19.69 | mg/mL | 19.69 | mg |
| n-acetyl cysteine | 13.79 | mg/mL | 13.79 | mg |
| 1,8-cineole | 10.90 | mg/mL | 10.90 | mg |
| β-Caryophyllene | 5.35 | mg/mL | 5.35 | mg |
| Nicotine Salt | 14.01 | mg/mL | 14.01 | mg |
| (54% nicotine) | | | | |
| Vitamin B12 | 9.85 | μg/mL | 9.85 | μg |
| Polysorbate 20 | 9.71 | mg/mL | 9.71 | mg |
| Deionized Water | 212.70 | mg/mL | 212.70 | mg |
| Vegetable Glycerin | 1112.88 | mg/mL | 1112.88 | mg |

Dose based on 125 puffs per mL

As part of the method for cigarette smoking cessation, Step 3 is based on an approximated consumption of 1 mL of liquid vaporized using 75 puffs per day from an ultrasonic vaping device or an electronic thermal liquid aerosolization device. The daily consumption of nicotine is about 5 mg, as disclosed in the composition of Table 16. The period of time a person would use the Step 3 nicotine replacement formulation could vary from as short as 2 weeks to as long as 2 months, for example, 14 to 30 days. There is a reduction in the number of puffs from 125 puffs per day in Step 2 to 75 puffs per day in Step 3. A person of ordinary skill in the art would recognize that, if a person wanting to quit smoking cigarettes was unable to progress to the next steps of this system of cigarette smoking cessation, then the health benefits of remaining at Step 3 would be better than if the person returned to smoking cigarettes for a longer term than envisioned in Step 3, including for a period of many years.

TABLE 16
Smoking Cessation Vape Liquid - Step 3

| | Liquid | | Dose at | |
|---|---|---|---|---|
| Compound | Concentration | Units | 75 Puffs | Units |
| Glutathione | 19.76 | mg/mL | 19.76 | mg |
| n-acetyl cysteine | 13.83 | mg/mL | 13.83 | mg |
| 1,8-cineole | 10.93 | mg/mL | 10.93 | mg |
| β-Caryophyllene | 5.36 | mg/mL | 5.36 | mg |
| Nicotine Salt | 5.00 | mg/mL | 5.00 | mg |
| (54% nicotine) | | | | |
| Vitamin B12 | 9.88 | μg/mL | 9.88 | μg |
| Polysorbate 20 | 9.78 | mg/mL | 9.78 | mg |
| Deionized Water | 212.39 | mg/mL | 212.39 | mg |
| Vegetable Glycerin | 1137.42 | mg/mL | 1137.42 | mg |

Dose based on 75 puffs per mL

As part of the method for cigarette smoking cessation, Step 4 is based on an approximated consumption of 1 mL of liquid vaporized using 75 puffs per day from an ultrasonic vaping device or an electronic thermal liquid aerosolization device, with the daily consumption of nicotine totally eliminated, as disclosed in the composition of Table 17. The period of time a person would use the Step 4 nicotine replacement formulation would depend on the respiratory health of the person and the type of respiratory system impairment and lung disease(s) the person has based on the impacts of her or his cigarette smoking history. The period of time a person would use the Step 4 composition could be months, years, or decades.

TABLE 17
Smoking Cessation Vape Liquid - Step 4 - No Nicotine

| | Liquid | | Dose at | |
|---|---|---|---|---|
| Compound | Concentration | Units | 75 Puffs | Units |
| Glutathione | 19.79 | mg/mL | 19.79 | mg |
| n-acetyl cysteine | 13.86 | mg/mL | 13.86 | mg |
| 1,8-cineole | 10.95 | mg/mL | 10.95 | mg |
| β-Caryophyllene | 5.37 | mg/mL | 5.37 | mg |
| Vitamin B12 | 9.90 | μg/mL | 9.90 | μg |
| Polysorbate 20 | 9.80 | mg/mL | 9.80 | mg |
| Deionized Water | 213.77 | mg/mL | 213.77 | mg |
| Vegetable Glycerin | 1151.05 | mg/mL | 1151.05 | mg |

Dose based on 75 puffs per mL

Alternatively, Step 4 can consist of utilizing a nebulizer or an ultrasonic vaping device to provide on-going treatment of respiratory lung diseases associated with an individual's past cigarette consumption history. A nebulizer formulation disclosed in Step 4 could alternatively be a formulation disclosed in Table 2, Table 5, or Table 8, that may be preferred for nebulization following Step 3 in this cigarette smoking cessation system, because they contain β-caryophyllene, which is a CB₂ agonist and helpful with addiction withdrawal.

The method of manufacturing of the four liquid formulations provided in Example 14 includes mixing a quantity of nitrogen purged purified water with a quantity of N-acetyl cysteine, a quantity of glutathione, a quantity of methylcobalamin followed by adding a quantity of vegetable glycerin and mixing until the liquid composition is homogeneous. This is followed by adding a mixture of a quantity of 1,8-cineole, β-caryophyllene and a quantity of Polysorbate 20, previously mixed to the mixture and slowly mixing until it is dissolved in the liquid, minimizing the volatilization of the 1,8-cineole and β-caryophyllene. Mixing is limited to that required to create a stable single-phase homogeneous suspension and to minimize volatilization of 1,8-cineole and β-caryophyllene. The liquid composition that can be aerosolized or vaporized in Example 14 can optionally be made with borneol, β-caryophyllene or a mixture of 1,8-cineole and one or more of borneol and β-caryophyllene in the same total concentration range as 1,8-cineole alone presented in Example 14. The pH of each liquid composition should be measured and the pH should be adjusted to 7.20 with sodium bicarbonate. If the liquid composition is not manufactured under sterile conditions, then a preservative can be added to improve the physical, chemical, and biological stability of the formulations. The liquid composition in Example 14 may be made with a quantity of vegetable glycerin that is less than the amounts disclosed in Tables 14, 15, 16, and 17 and can be decreased by increasing a corresponding mass of nitrogen purged water added.

### Example 15

A pre-clinical trial was conducted on five patients that were either current or ex-cigarette smokers historically diagnosed with either asthma or COPD. A preferred liquid pharmaceutical composition was vaporized using commercially available electronic thermal vaping pens with a 3.0 mL refillable tank, a 1300 mAH rechargeable lithium ion battery, and a 0.5 Ohm coil operating at 3.7 volts (Kanger Tech® SUBVOD-Kit™). Patients inhaled at least puffs per day for up to a 73-day period. Spirometry tests including Forced Expiratory Volume after 1 second (FEV1) and Forced Vital Capacity (FVC) measurements were made before treatment, during treatment, and at the end of treatment. Spirometry is the most frequently performed pulmonary function test and plays an important role in diagnosing the presence and type of lung abnormality, classifying its severity and evaluating treatment outcomes. Patients were also interviewed with respect to their breathing capabilities, energy levels, and general well-being and health.

The procedure followed by each patient consisted of a preferred liquid composition, disclosed in Table 18, being placed into a vape pen tank with a dropper and then the on button being depressed on the side of the vape pen to actuate heating of the coil while the patients inhaled the aerosolized liquids through an attached mouthpiece.

TABLE 18
Pre-clinical Trial Liquid Composition

| | Liquid | | Dose at | |
|---|---|---|---|---|
| Compound | Concentration | Units | 40 Puffs | Units |
| Glutathione | 19.09 | mg/mL | 10.18 | mg |
| n-acetyl cysteine | 14.16 | mg/mL | 7.55 | mg |
| 1,8-cineole | 19.94 | mg/mL | 10.63 | mg |
| Vitamin B12 | 39.33 | μg/mL | 20.98 | μg |
| Polysorbate 20 | 11.89 | mg/mL | 6.34 | mg |
| Deionized Water | 199.84 | mg/mL | 106.58 | mg |
| Vegetable Glycerin | 1103.89 | mg/mL | 400.89 | mg |

Dose based on 75 puffs per mL and 40 puffs per day used by patients

Patients inhaled at least 75 puffs from the vape pen on a daily basis. Prior to commencing treatments, each patient's past and current history of cigarette smoking, age, height, weight, gender, and race was recorded as part of the testing to allow the calculation of normal FEV1 and FVC values. All individuals had a history of cigarette smoking and only 1 patient currently smoked cigarettes as indicated in Table 19. The patients were diagnosed with either COPD or asthma as indicated in Table 19. Prior to the liquid aerosolization treatment, each patient underwent spirometry testing to measure FEV1 and FVC to provide baseline conditions. These results were compared to calculated normal values using the method of Hankinson et al. (1999) from the National Institute for Occupational Safety and Health, Centers for Disease Control and Prevention. Patient histories and spirometry test results are summarized in Table 19. Using the normal FEV1 values calculated for each individual based on age, height, sex, and race and their baseline FEV1 measurements prior to treatment, percent normal FEV1 values for each patient were calculated to provide baseline conditions to compare treatment results.

TABLE 19
CuraBreath Pre-Clinical Test Data

| | Patient Identification | | | | | |
|---|---|---|---|---|---|---|
| Parameters | ID | 102 | 103 | 104 | 105 | 106 |
| Sex | M/F | F | M | F | F | M |
| Height | cm | 161 | 185 | 152 | 156 | 176 |
| Age | year | 61 | 45 | 67 | 39 | 38 |
| Weight | lb. | 140 | 210 | 107 | 117 | 181 |
| Historical Patient Diagnosis | | COPD | COPD | Asthma | COPD | COPD |
| Active Cigarette Smoker | | Yes | No | No | No | No |
| Years Active Smoker | year | 20 | 15 | 28 | 10 | 6 |
| Calculated Normal FEV1 | L | 2.47 | 4.44 | 1.98 | 2.80 | 4.18 |
| Calculated Normal FVC | L | 3.20 | 5.67 | 2.60 | 3.40 | 5.22 |
| Calculated Normal FEV1/FVC ratio | % | 77.19 | 78.31 | 76.15 | 82.35 | 80.08 |
| Baseline Patient Measurement, | day | 0.00 | 0.00 | 0.00 | 0.00 | 0.00 |
| Time (days) | | | | | | |
| Measured FEV1 | L | 1.70 | 2.84 | 1.33 | 1.90 | 2.82 |
| Measured FVC | L | 2.16 | 3.55 | 1.63 | 2.23 | 3.55 |
| Measured Percent FEV1/FVC | % | 78.70 | 80.00 | 81.60 | 85.20 | 79.44 |
| Ratio | | | | | | |
| Percent Normal FEV1 | % | 68.83 | 63.96 | 67.17 | 67.86 | 67.46 |
| Percent Normal FVC | % | 67.50 | 62.61 | 62.69 | 65.59 | 68.01 |
| Interim Patient Measurement, | day | 21 | 21 | 21 | 21 | 21 |
| Time (days) | | | | | | |
| Measured FEV1 | L | 2.01 | 3.35 | 1.6 | 2.28 | 3.3 |
| Measured FVC | L | 2.45 | 4.1 | 1.98 | 2.61 | 4.12 |
| Measured Percent FEV1/FVC Ratio | % | 82.04 | 81.71 | 80.81 | 87.36 | 80.10 |
| Percent Normal FEV1 | % | 81.38 | 75.45 | 80.81 1 | 81.43 | 78.95 |
| Percent Normal FVC | % | 76.56 | 72.31 | 76.15 | 76.76 | 78.93 |
| Final Patient Measurement, | day | 42 | 73 | 53 | 53 | 53 |
| Time (days) | | | | | | |
| Measured FEV1 | L | 2.25 | 4.16 | 1.93 | 2.55 | 3.92 |
| Measured FVC | L | 2.6 | 4.98 | 2.23 | 2.95 | 4.7 |
| Measured Percent FEV1/FVC | % | 86.54 | 83.53 | 86.55 | 86.44 | 83.40 |
| Ratio | | | | | | |
| Percent Normal FEV1 | % | 91.09 | 93.69 | 97.47 | 91.07 | 93.78 |
| Percent Normal FVC | % | 81.25 | 87.83 | 85.77 | 86.76 | 90.04 |
| Overall Percent FEV1 Reversibility | % | 32.35 | 46.48 | 45.11 | 34.21 | 39.01 |
| Overall Percent FVC Reversibility | % | 20.37 | 40.28 | 36.81 | 32.29 | 32.39 |

Females generally have a smaller lung capacities than males, and it can be seen in Table 19 that the 3 female patients had lower baseline FEV1 capacities (baseline values before treatment for FEV1 of 1.33 L to 1.70 L) than the 2 male patients (baseline values before treatment for FEV1 of 2.82 L to 2.84 L). The normal FEV1 values for the female patients were calculated to be 1.98 L to 2.80 L. The normal FEV1 values for the male patients were 4.18 L and 4.44 L. Each patient had substantially lower baseline FEV1 values than what would be normal for a healthy individual. For the 5 patients, the percent normal FEV1 values prior to treatment varied from 63.96% to 68.83%. For example, individuals with COPD that have percent normal FEV1 values less than 80 percent are classified with GOLD 2 moderate COPD. Based on these values, it was evident that each patient exhibited significant airway restriction. FVC baseline capacities for all patients were also significantly lower than what would be normal values for healthy individuals, varying from 62.61% to 68.01%.

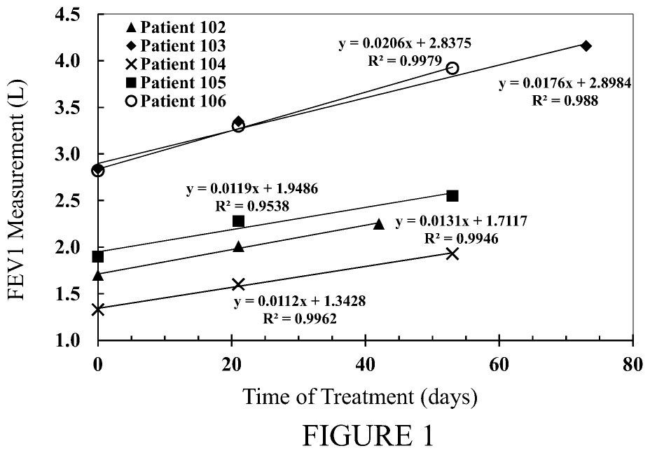

Spirometry tests following the inhalation respiratory treatment were repeated after 21 days of treatment and at the end of treatment, which varied from 42 days to 73 days, for each individual. Results of FEV1 spirometry testing were graphed for each patient with results displayed in FIG. 1. It is clear that the rate of increase in FEV1 value improvement overtime was linear and significant. Female patients had FEV1 reversibility values following the entire treatment period of 32.35%, 34.21%, and 45.11%. Female patients also had an increase in their Forced Vital Capacity (FVC) following the entire treatment period of 20.37%, 32.29%, and 36.81%. Patient 102, who was a 61 year old female diagnosed with COPD, had smoked cigarettes for at least 28 years, and still was an active smoker at the time when these tests were conducted, had increases in FEV1 and FVC of 32.45% and 20.37%, respectively. Patient 104 was a female diagnosed with asthma and was the oldest person in the pre-clinical study at 67 years and had smoked 2 packs of cigarettes for 28 years. Patient 104 had the highest FEV1 reversibility at 45.11%.

Males generally have larger lung capacities and this is evident from review of results presented in Table 19 and FIG. 1. From review of FIG. 1, it can be seen that there was also a linear rate of FEV1 improvement over time with a substantial improvement in spirometry results. Male patients had FEV1 reversibility values following the entire treatment period of 46.48% and 39.01%, for patients 103 and 106, respectively. Male patients also had an increase in their Forced Vital Capacity (FVC) following the entire treatment period of 40.28% and 32.39%, respectively. Patient 103 was male, 45 years old, had smoked cigarettes for 15 years and was not an active smoker at the time when these tests were conducted.

Various organizations are associated with the assessment of improvement of patients with COPD. FEV1 results reported in our pre-clinical tests indicate a significant improvement when compared to FEV1 improvement assessment criteria established by these organizations as follows: America College of Chest Physicians—FEV1>15%; American Thoracic Society—FEV1 or FVC>12%; and >0.200 L; GOLD—>12% and >0.200 L. The pre-clinical test results presented in FIG. 19 indicate FEV1 reversibility varying from 32.35% to 46.48%; FVC reversibility varying from 20.37% to 40.28%; and improvement in FEV1 values varying from 0.55 L to 1.32 L.

### Example 16

A pre-clinical trial was conducted on a single patient using a preferred aerosolizable liquid that was nebulized using a commercially available portable ultrasonic mesh-type nebulizer with a 5.0 mL refillable liquid reservoir and a rechargeable lithium ion battery (Flyp nebulizer, Convexity Scientific, Inc.). The patient was a 49 year old male, 174.86 cm in height, with a history of diagnosed mild to moderate asthma. The patient was prone to about 10 to 15 asthma attacks per year requiring medication caused by seasonal allergies, induced by cold air and induced by exercise. The patient typically used albuterol, a bronchodilator, as a rescue-type inhaler during these events and periodically also used fluticasone furoate, an inhalable corticosteroid powder. The patient also required the use of prednisone, an oral corticosteroid, about 1 to 2 times per year for the most serious asthma attacks.

Prior to first nebulizing a preferred liquid composition, the patient reported moderate asthma symptoms consisting of a sensation of constriction of the chest and difficulty breathing and taking a full breath. The patient had been inhaling albuterol and fluticasone furoate on a daily basis for one week prior to using the nebulizer fluid, without substantive relief of symptoms. Based on his prior experience with asthma and his symptoms, he reported that he thought he would need to use prednisone, if the symptoms continued. Using the portable ultrasonic mesh nebulizer, the patient nebulized 1 mL of a liquid comprising the following glutathione 1.10% (w/w), N-acetyl cysteine 1.10% (w/w), 1,8-cineole 0.80% (w/w), β-caryophyllene 0.80% (w/w), methylcobalamin 0.003% (w/w), Polysorbate 20 0.3% (w/w), and sterile saline water solution (0.9% saline) 95.3% (w/w). Within 30 minutes following nebulization the patient reported that his chest felt significantly more relaxed and less constricted, he was able to breathe more fully, and he felt more energetic. He was completely able to stop taking albuterol and fluticasone furoate following the nebulization treatment. After this single nebulization event, the patient reported his symptoms remained improved over the next week, although there was a lessening in the extent of improvement after about 4 to 5 days. Three days following nebulization of the pharmaceutical liquid, the patent underwent spirometry testing. The normal spirometry values for the patient were calculated to be FEV1=3.81 L and FVC=4.89 (Hankinson, 1999). Measured spirometry values three days after the single nebulization treatment were FEV1=2.99 L and FVC=3.65 L, with percent normal values for FEV1=78.4% and FVC=74.6%.

The patient then began a 7-day period of daily nebulization treatment one week after the single nebulization treatment. Prior to starting the 8-day treatment period the patient underwent baseline spirometry testing with the following results: FEV1=3.09 L and FVC=3.57 L with percent normal values for FEV1=81.0% and FVC=73.0%. The patient nebulized increasing amounts of a nebulizer liquid for 8 days comprising the following: glutathione—0.70% (w/w); N-acetyl cysteine 0.70% (w/w); methylcobalamin—0.003% (w/w); and sterile saline water solution (0.9% saline)—98.4% (w/w). On days 1 through 3, 1.5 mL was nebulized and on days 4 through 8, 3.0 mL was nebulized. Following nebulizing the liquid composition on day 7, spirometry tests were conducted on the patient. Spirometry results following nebulizing 3.0 mL of the liquid were FEV1=3.39 L and FVC=3.84 L, with percent normal values for FEV1=86.8% and FVC=78.5.0%. Compared to the first baseline spirometry values the percent FEV1 reversibility was calculated to be 12% and the percent FVC reversibility was 5.2%. The improvement of the FEV1/FVC % ratio increased from 81.9% to 88.3% compared to the first patient spirometry results. It is apparent that this patient was using a greater percentage of their lung capacity in the second of the spirometry tests.

The patient reported that even given only one nebulization treatment during the first week of treatment followed by 11 days of only moderate nebulization treatment he did not experience any asthma attacks and did not have to take his prescription bronchodilator or any corticosteroid of any time during the test period. The patient reported that he had more energy and could breathe easier and more fully.

### Compositions and Methods for Treating Viral and Bacterial Infection Through Inhalation Therapy

The 1918 H1N1 Spanish flu, infected approximately 5% of the world's population and killed 2%. The case fatality rates for the 1957 H2N2 Asian influenza, the 1968 H3N2 Hong Kong influenza, and the 2009 H1N1 pandemic influenza were reported to be lower, with an estimated rate of 0.2% or less. Between 1997 and 2014, several unprecedented epizootic avian influenza viruses (e.g., H5N1, H7N9, and H10N8) crossed the species barrier to cause human death. They pose a threat of human-to-human transmission. These infections in humans can be accompanied by an aggressive pro-inflammatory response and insufficient control of an anti-inflammatory response, a combination of events called ‘cytokine storm’ (Liu et al. 2016).

The cytokine storm can be a major factor in the development Acute Respiratory Distress Syndrome secondary to the COVID-19 disease. The progression of COVID-19 into the lungs is also a leading causal factor requiring the use of mechanical ventilators that are in short supply at a national level. Acute Respiratory Distress is also a key factor in COVID-19 patient mortality. Acute Respiratory Distress Syndrome is characterized by extreme fluid accumulation in the lungs resulting in severely limited mass transfer of oxygen through the thick mucolytic liquid layer in comparison to the very thin epithelial lining fluid of healthy individuals. According to a leading professor, Dr. Liu Liang, from Tongji Medical College who conducted 12 autopsies on patients who have died from COVID-19, “they found a large amount of mucous in the lungs. . . . The secretion is very sticky. It attaches to the lung like a paste.”

In 2019 the SARS-CoV-2 virus and the associated COVID-19 disease became a global pandemic infecting millions of people with a substantial mortality rate. In a study of COVID-19 patients from China, a large cohort of >44,000 persons showed that illness severity can range from mild to critical (Wu eta al. (2020):
Mild to moderate (mild symptoms up to mild pneumonia): 81%
Severe (dyspnea, hypoxia, or >50% lung involvement on imaging): 14%
Critical (respiratory failure, shock, or multi-organ system dysfunction): 5%

Epidemiologic studies have documented SARS-CoV-2 transmission during the pre-symptomatic incubation period and asymptomatic transmission has been suggested. Virological studies have also detected SARS-CoV-2 with RT-PCR low cycle thresholds, indicating larger quantities of viral RNA, and cultured viable virus among persons with asymptomatic and pre-symptomatic SARS-CoV-2 infection. The exact degree of SARS-CoV-2 viral RNA shedding that results in risk of transmission is uncertain. Risk of transmission is thought to be greatest when patients are symptomatic since viral shedding is greatest at the time of symptom onset and declines over the course of several days to weeks.

According to the CDC (2020), for patients who developed severe COVID-19 disease, the medium time to dyspnea varied from 5 to 8 days, the median time to acute respiratory distress syndrome (ARDS) varied from 8 to 12 days, and the median time to ICU admission ranged from 10 to 12 days. Some patients may rapidly deteriorate one week after illness onset. Among all hospitalized patients, a range of 26% to 32% of patients were admitted to the ICU. Among all patients, a range of 3% to 17% developed ARDS compared to a range of 20% to 42% for hospitalized patients and 67% to 85% for patients admitted to the ICU. Mortality among patients admitted to the ICU ranges from 39% to 72% depending on the study. The median length of hospitalization among survivors was 10 to 13 days.

Severe and critically ill COVID-19 patients frequently are diagnosed with ARDS, multi-organ damage involving cardiac injury, coagulopathy, thrombosis, neurological impairment, gastrointestinal tract and kidney dysfunction, and have high mortality rates (Huang et al., 2020). High mortality rates of COVID-19 patients is frequently associated with SARS-CoV-2 infection-induced hyperinflammation in the respiratory tract the result of excessive immune system response causing the cytokine release syndrome (CRS), commonly referred to as the cytokine storm. Moore and June (2020) reported up to 20% of COVID-19 patients progress to ARDS, similar to CRS-induced ARDS and secondary hemophagocytic lymphohistiocytosis (sHLH) observed in patients with SARS-CoV and MERS-CoV (Moore et al., 2020). CRS was found to be the major cause of morbidity in patients infected with SARS-CoV and MERS-CoV (Channappanavar et al., 2017). Elevated serum concentrations of the cytokines IL-1β, IL-6 and IL-8 and other inflammatory cytokines are hallmarks of severe MERS-CoV infections (Fehr et al., 2017). The Cytokine Release Syndrome is also reported to be common in patients with COVID-19, and elevated serum IL-6 correlates with respiratory failure, ARDS, and adverse clinical outcomes (Chen et al., 2020, Ruan et al., 2020). Elevated serum C-reactive protein (CRP), an acute phase protein that increases following IL-6 secretion by macrophages and T-cells, is a biomarker of severe betacoronavirus infection and now specifically with COVID-19 (Chen et al., 2020). Given this experience, therapeutics based on suppressing CRS are critically needed to decrease the incidence of CRS-related ARDS and consequential mortality and chronic illnesses the result of COVID-19 (Moore et al., 2020).

Huang et al. (2020) recently reported that 100% of 41 hospital admitted COVID-19 patients had significantly higher initial plasma levels of cytokines and pro-inflammatory factors, including; IL-1β, ILl-Ra, IL-7, IL-8, IL-9, IL-10, basic FGF, GCSF, GMCSF, IFN-γ, IP10, MCP1, MIP1A, MIP1B, PDGF, TNF-α, and VEGF in both ICU patients and non-ICU patients in Wuhan, China than in healthy adults. This is characteristic of the cytokine storm. The mechanism of the cytokine storm with the influenza virus was reported by Liu et al. (2016) and is summarized here. Respiratory epithelial cells, the primary targets for influenza virus, are also the choreographers of cytokine amplification during infection. Following primary exposure, progeny viruses that proliferate within these cells can infect other cells, including alveolar macrophages. Inflammatory responses are triggered when infected cells die by apoptosis or necrosis. The initial response of the organism to harmful stimuli is acute inflammation and is characterized by increasing blood flow (sic, likely dilatation), which enables plasma and leukocytes to reach extra-vascular sites of injury, elevating local temperatures, and causing pain. Liu et al. (2016) also reported that the acute inflammatory response is additionally marked by the activation of pro-inflammatory cytokines or chemokines. These pro-inflammatory cytokines or chemokines can lead to the recruitment of inflammatory cells. Then, an increasing expression of inflammatory, antiviral, and apoptotic genes occurs, accompanied by abundant immune cell infiltration and tissue damage. These mechanism are summarized by Liu et al. (2016). The cytokine storm in the lung following severe influenza infection has been summarized by Liu et al. (2016). (1) Viruses infect lung epithelial cells and alveolar macrophages to produce progeny viruses and release cytokines/chemokines (mainly contains interferons). (2) Cytokine/chemokine-activated macrophages and virally infected dendritic cells lead to a more extensive immune response and the initiation of cytokine storm. (3) Released chemokines attract more inflammatory cells to migrate from blood vessels into the site of inflammation, and these cells release additional chemokines/cytokines to amplify the cytokine storm.

Hoffman et al. (2020) and Zhou et al. (2020) both independently reported that SARS-CoV-2 uses the host SARS-CoV receptor angiotensin-converting enzyme 2 (ACE2) for entry and the host cell type 2 transmembrane serine protease serine protease TMPRSS2 for S protein priming. TMPRSS2 present in host cells, promotes viral uptake by cleaving ACE2 and activating the SARS-CoV-2 S protein controlling viral entry (Sungnack et al, 2020). In the lungs, 83% of ACE2-expressing cells are alveolar epithelial type II cells (AECII), suggesting these cells can serve as a reservoir for viral invasion and facilitate viral replication in the lung (Zhou et al. 2020). Hamming et al. (2004) reported ACE2 is abundantly present in humans in the epithelia of the lung and small intestine. They also found ACE2 was present in many other human organs, including; oral and nasal mucosa, nasopharynx, lung, stomach, small intestine, colon, skin, lymph nodes, thymus, bone marrow, spleen, liver, kidney, and brain and present in arterial and venous endothelial cells and arterial smooth muscle cells in all of the organs studied. Sungnak et al. (2020) reported that the high expression of ACE2 and viral entry-associated protease in nasal goblet and ciliated cells implicates them as a significant loci of initial infection and possible reservoirs for dissemination within and between individuals. They also reported that the ACE2-TMPRSS2 co-expression in additional barrier surface tissues, such as the esophagus, ileum and colon could explain viral fecal shedding with the potential fecal-oral transmission.

A study of 3,762 individuals from 56 countries with COVID-19 symptoms lasting longer than 28 days was recently reported in December 2020 (Davis et al., 2020). Of the patients in the study, only 8.4% were ever hospitalized, 34.9% visited and ER or urgent care facility and 56.7% were not hospitalized. While about 75% of the individuals reported dyspnea at any point in their infection (typically in week 2 following symptom onset), 40% still reported shortness of breath 6 months after their initial symptoms. In a study of led by the CDC, 294 non-hospitalized COVID-19 outpatients in the U.S., 33% reported dyspnea upon COVID-19 testing and 30% still reported dyspnea 14 to 21 days following their initial COVID-19 test (Tenforde, et al., 2020). Patients with longer-term symptoms of the COVID-19 disease are commonly referred to and COVID-19 long haulers or long-COVID-19. A person ordinarily skilled in the art would recognize that these terms describe patients with symptoms lasting longer than 28 days, longer than 3 months, longer than 6 months, longer than 1 years, longer than many years. One embodiment of this present invention include pharmaceutical compositions and methods of treatment for therapies to treat patients with long-COVID-19 and longer-term symptoms of patients following other viral respiratory infections, bacterial respiratory infections, or combinations of them.

Discussed herein is a method to disrupt, neutralize, treat or antagonize cytokine proliferation in individuals who potentially have been exposed to, have been exposed to, have symptoms of, or are recovering from one or more respiratory viral or bacterial diseases, or a combination of both. One of the compounds in this present invention is 1,8-cineole. 1,8-cineole, at a concentration of (1.5 μg/mL) inhibits (n=13-19, p=0.0001) cytokine production in lymphocytes of TNF-α, IL-4, and IL-5, by 92%, 84%, 70%, and 65%, respectively. Cytokine production in monocytes of TNF-α, IL-1β, IL-6, IL-8 is (n=7-16, p<0.001) was inhibited by 99%, 84%, 76%, and 65%, respectively. A single dose in one formulation in this present invention at 2 mL contains 15 mg of 1,8-cineole. The target concentration of this compound, delivered by aerosolization, in the epithelial lining fluid of the lungs results in an estimated concentration of 614 μg/mL (assuming 100% delivery). Nebulization of aerosolized liquids can result in a 70 to 85% deposition efficiency in the lower respiratory tract.

Another compound in this present invention is β-caryophyllene. β-caryophyllene has been reported to heal lung epithelial tissue associated with acute lung injury. β-caryophyllene at 102 μg/mL inhibits lipopolysaccharide (LPS)-stimulated IL-1β and TNF-α expression in human whole blood. IL-1β and TNF-α inhibition is reversed when a specific receptor selective antagonist is used, validating the mechanism of action of this compound, including being a CB₂ agonist. The target concentration of this compound, delivered by aerosolization, in the epithelial lining fluid of the lungs results in an estimated concentration of 603 μg/mL (assuming 100% delivery). A single dose in one formulation in this present invention at 2 mL contains 15 mg and the target concentration of this compound delivered by aerosolization in the epithelial lining fluid of the lungs results in a concentration of 603 μg/mL.

One mechanism of action of 1,8-cineole and β-caryophyllene used in embodiments of the invention is to directly antagonize the formation of cytokines in patients with one or more respiratory diseases caused by viral and/or bacterial pathogenic agents. The pathogenic agents can be one or more of, but not limited to respiratory viruses and the diseases associated with these viruses, including but limited to adenovirus (Adeno) and rhinovirus, which cause illness year-round. Respiratory viruses include, but are not limited to the following: adenovirus, influenza A (H1N1, H1N2 and H3N2), influenza B (FluB), influenza C (FluC), parainfluenza virus (HPIV1, HPIV2, HPIV3, HPIV4), respiratory syncytial virus (RSV), human coronavirus (HCoV-229E, HCoV-NL63, HCoV-HKUJ, HCoVOC4), human metapneumovirus (hMPV) and the severe acute respiratory syndrome-associated CoVs, SARS-CoV-1 and 2019 SARS-CoV-2.

The pathogenic agents can be one or more of, but not limited to bacteria and the respiratory diseases associated with these bacteria, including; Bordetella pertussis, Chlamydophila pneumoniae, Mycoplasma pneumoniae, Streptococcus pneumoniae, Klebsiella pneumoniae, Staphylococcus aureus (MSSA and MRSA), Pseudomonas aeruginosa, Escherichia coli, Haemophilus influenza, Legionella pneumophila and Acinetobacter and Enterobacter species.

Another mechanism of action used in embodiments of the invention to treat respiratory diseases is to rebalance the antioxidant/oxidant ratio in epithelial cells and epithelial lining fluid of patients with viral and/or bacterial respiratory disease. The endogenous production of reactive oxygen species (ROS) and reactive nitrogen species (RNS) produced in viral and bacterial respiratory diseases, including influenza A virus and SARS-CoV3CL infections can cause a rapid influx of inflammatory cells, resulting in further increased reactive oxygen species production, cytokine expression, and acute lung injury. Proinflammatory stimuli are known to induce intracellular reactive oxygen species by activating NADPH oxidase activity. Reactive oxygen species (ROS) can play a central role in inflammatory responses and viral replication. Antioxidants that exert antiviral and anti-inflammatory effects may be effective for the treatment of the cytokine storm induced by severe influenza. Two natural endogenous antioxidant compounds are presented herein, including glutathione and n-acetyl cysteine (NAC, direct antioxidant and glutathione precursor), e.g., for aerosolization and inhalation in patients. A single dose in one formulation of the invention at 2 mL contains 22 mg each of glutathione and n-acetyl cysteine and the target concentrations of these compounds delivered by aerosolization in the epithelial lining fluid of the lungs results in concentrations of 889 μg/mL. One mechanism of these compounds in formulations of the invention is to increase the natural concentrations of glutathione in the epithelial lining fluid (ELF) to 889 μg/mL which is about 7 times that present in younger smokers that have a stimulated endogenous production of this same compound to counterbalance ROS associated with cigarette smoke in the lungs. Glutathione inhalation increases glutathione levels in ELF. Glutathione scavenges ROS and RNS in lungs. Glutathione can be endogenously increased in human bronchial epithelial cells of cigarette smokers with normal pulmonary function and can be related to decreases in epithelial cell permeability and release of inflammatory cytokine IL-1β and sICAM-1.

N-acetyl-cysteine has direct antioxidant properties and has an indirect role as a precursor in glutathioine synthesis. Lung epithelial TRPA1 can have a role in the induction of IL-8 by cigarette smoke extract in primary human bronchial epithelial cells. Cigarette smoke extract can cause increased ROS, which then can activate lung epithelial TRPA1. Ca²⁺ influx can be prevented by decreasing ROS with n-acetyl-cysteine. The Ca²⁺ influx decrease with n-acetyl-cysteine can be similar to that with synthetic TRPA1 antagonist HC030031.

1,8-cineole, a TRPM8 agonist, can be a natural antagonist of human TRPA1 nociceptors and does not activate hTRPV1 or hTRPV2. TRPA1, activated by 20 uM AITC, can be inactivated by 1,8-cineole with an IC50 concentration of 3.43 mM (528 mg/L). In humans and in guinea pigs activation of the TRPA1 ion channel can evoke a tussive (i.e., coughing) response. This can pertain to the pathogenesis of respiratory diseases and to the treatment of cough, because of the central role of and TRPA1 activation by a wide range of irritant and chemical substances, for example, by exogenous agents, endogenously produced mediators during inflammation, and/or oxidant stress. TRPA1 channels can be a target for antitussive drugs. Antagonizing TRPA1 is one mechanism of action that 1,8-cineole provides in an embodiment of the invention. Antagonizing TRPA1 can reduce the frequency of coughing and decrease the proliferation of cytokines, chemokines, and other pro-inflammatory factors in the respiratory tract of individuals with viral and/or bacterial respiratory diseases.

1,8-cineole can scavenge free radicals at low concentrations: 10⁻⁵ M [1.54 mg/L] strongly inhibits superoxide (O₂⁻) (−53%, p<0.001), partially inhibits super oxide dismutase (SOD) (−28%, p=0.0039), and inhibits hydrogen peroxide (H₂O₂) at 10⁻¹⁰ M [15.4 ng/L] (−48%, p=0.0274). Total cellular antioxidant activity of LPS-stimulated 8-isoprostanes increases dose-dependently from 10⁻⁶ M (−42%, p=0.0288) to 10⁻⁵ M (−84%, p<0.0001), comparable to TNF-α.

β-caryophyllene can scavenge radicals (e.g., as determined by DPPH (1,1-diphenyl-2-picrylhydrazine) and FRAP (ferric reducing/antioxidant power) antioxidant assays); β-caryophyllene can have stronger antioxidant properties than ascorbic acid, at 1.25 μM compared to 1.50 μM, respectively. Radical scavenging by β-caryophyllene is also observed in a FRAP radical assay with 3.23 μM for β-caryophyllene compared to 3.80 μM for ascorbic acid. A mechanism of action used in embodiments of this invention provided by 1,8-cineole and other compounds is to scavenge ROS associated free radical species, including but not limited to superoxide, hydroxyl radical, perhydroxyl radical, and singlet (e.g., singlet oxygen), as well as the oxidant hydrogen peroxide, and any of their organic reaction radicals. Another mechanism of action provided by 1,8-cineole and other compounds in an embodiment of the invention is to scavenge RNS-associated free radical species, including, but not limited to nitric oxide, peroxynitrite, nitrogen dioxide, dinitrogen trioxide, and any of their organic reaction radicals.

The antioxidant properties of vitamin B12 or cobalamin (e.g., cyanocobalamin, hydroxocobalamin, adenosylcobalamin, and/or methylcobalamin), a component in embodiments of the invention, can result from a combination of direct and indirect effects: stimulation of methionine synthase activity, direct reaction with ROS and RNS, a glutathione sparing effect, and a modification of signaling molecules. Vitamin B12 and the thiolatocobalamins exhibit a marked antioxidant activity at pharmacological concentrations and can afford cellular protection against oxidative stress.

Comparison of inhaled (four 1 mg doses once weekly for 28 days) and oral (1 mg/day for 28 days) vitamin B12 supplementation in 40 people (none of whom are vitamin B12 deficient), divided into exercise and non-exercise groups, indicates the following. Patients who inhale B12 by nebulization over 4 weeks increase their serum B12 levels by an average of 28.5% for those who exercise and 62% for those who do not exercise. Patients who ingest B12 orally increase B12 levels by 20.5% (exercise) and 23.8% (non-exercise). Nebulizing vitamin B12 has a clinical effect and nebulization of vitamin B12 can increase a patient's serum B12 level.

Additionally, three of the compounds in and embodiment of the invention are highly mucolytic (glutathione, N-acetyl cysteine, and 1,8-cineole) and inhalation directly to the lungs through aerosolization with a nebulizer is an effective method to deliver these compounds to the epithelial lining fluid in the lungs.

The inclusion of two endogenous antioxidants, glutathione and N-acetyl cysteine, in an embodiment of the invention boosts the concentrations of these natural compounds in the lungs, increasing their natural ability to fight (reduce) the production of ROS and RNS associated with COPD, asthma, and COVID-19. Three additional antioxidant compounds, 1,8-cineole, β-caryophyllene, and methylcobalamin, in an embodiment of the invention have free radical scavenging properties and help to clear mucous from the lungs.

Five of the compounds, N-acetyl cysteine, glutathione, 1,8-cineole, β-caryophyllene, and methylcobalamin, in compositions that are embodiments of the invention have anti-viral properties and one, β-caryophyllene, has strong anti-bacterial properties. Methylcobalamin may be an inhibitor of the RNA-dependent-RNA polymerase activity of the SCV2-nsp12 enzyme in the SARS-CoV-2 coronavirus. Without being bound by theory, vitamin B12 (methylcobalamin) may bind to the active site of the nsp12 protein and prevent association with RNA (ribonucleic acid) and NTP (nucleoside triphosphate) and thus inhibit the RdRP (RNA-dependent RNA polymerase) activity of nsp12 and be an effective inhibitor of the nsp12 protein. The nsp12 enzyme is critical for the replication of the viral enzyme; the inhibition of this enzyme can result in lower viral titers and reduce the severity of the COVID-19 disease. Vitamin B12 supplementation can improve rates of sustained viral response in patients chronically infected with hepatitis C virus receiving additional treatment. In an embodiment of the invention vitamin B12, for example, in the form of methylcobalamin and/or cobalamin, is in a liquid that is aerosolized to treat viral and/or bacterial infection, for example, through vitamin B12's direct antiviral and/or antibacterial properties and/or secondarily through another mechanism, including, but not limited to antioxidant scavenging of ROS and RNS, counteracting vitamin B12 deficiency, treating anemia, and/or treating shortness of breath.

N-acetylcysteine (NAC) can inhibit replication of human influenza A viruses. The H5N1 influenza A virus is associated with viral pneumonia, lymphopenia, high viral loads in the respiratory tract, and hyper-induction of cytokines and chemokines (cytokine storm). N-acetylcysteine at 5 mM (816 mg/L) to 15 mM (2,448 mg/L) reduces H5N1-induced cytopathogenic effects, virus-induced apoptosis, and viral yields, 24 hrs post-infection. N-acetylcysteine also decreases the production of proinflammatory molecules (CXCL8, CXCL10, CCL5, interleukin-6 (IL-6)) in H5N1-infected A549 cells and reduces monocyte migration towards supernatants of H5N1-infected A549 cells. Antiviral and anti-inflammatory mechanisms of N-acetylcysteine can include inhibition of activation of oxidant sensitive pathways including transcription factor NF-κB and mitogen activated protein kinase p38. The synthetic pharmacological inhibitor (i.e., BAY 11-7085) of NF-κB exerts similar effects to those of NAC in H5N1-infected cells. In an embodiment of the invention N-acetylcysteine is in liquids that are aerosolized to treat viral and/or bacterial infections, for example, through direct antiviral, antibacterial, and/or antibiofilm properties and/or secondarily through another mechanisms including, but not limited to antioxidant scavenging of ROS and RNS, cytokine antagonism, and/or mucolysis.

Cytotoxic T lymphocyte lines show improved (increased) proliferation after glutathione ELF levels are augmented pre-nebulization to post-nebulization by HIV patients inhaling nebulized glutathione. Glutathione deficiency can contribute to increased HIV (human immunodeficiency virus) replication and increasing dysfunction of the immune system. Glutathione (GSH) can inhibit replication of parainfluenza-1, herpes simplex-1, and HIV-1 viruses by a direct effect on the envelope glycoproteins. In an embodiment of the invention glutathione is in liquids that are aerosolized to treat viral and/or bacterial infections, for example, through direct antiviral, antibacterial, and/or antibiofilm properties and/or secondarily through other mechanisms including, but not limited to antioxidant scavenging of ROS and RNS and cytokine antagonism.

1,8-cineole can activate anti-viral transcription factor Interferon Regulatory Factor 3 (IRF3) and reduce pro-inflammatory NF-κB in cells in a human ex vivo model of rhinosinusitis. 1,8-cineole can protect against influenza A virus infection in mice and also can decrease the levels of IL-4, IL-5, IL-10, and MCP-1 in nasal lavage fluids and the levels of IL-1β, IL-6, TNF-α, and IFN-γ in lung tissues of mice infected with influenza virus (IL=interleukin, MCP=monocyte chemoattractant protein, TNF=tumor necrosis factor, IFN=interferon). 1,8-cineole can reduce the expression of NF-κB p65, intercellular adhesion molecule (sICAM-1), and vascular cell adhesion molecule (VCAM)-1 in lung tissues (NF-κB=nuclear factor kappa-light-chain-enhancer of activated B cells). 1,8-cineole can inhibit the avian coronavirus (IBV) with an IC₅₀ of 0.61 mM. In silico simulations indicate the binding site is located at the N terminus of phosphorylated nucleocapsid (N) protein. In an embodiment of the invention 1,8-cineole is in liquids that are aerosolized to treat viral and/or bacterial infections, for example, through direct antiviral, antibacterial, and/or antibiofilm properties and/or secondarily through other mechanisms including, but not limited to antioxidant scavenging of ROS and RNS, cytokine antagonism, and/or mucolysis. β-caryophyllene, an anti-inflammatory compound in an embodiment of the invention, exhibits a strong antibacterial effect against E. coli (MTCC 732), with a Minimum Inhibitory Concentration (MIC) value of 9.0±2.2 μM. The MIC antibiotic properties of this compound compared to the antibiotic Kanamycin (KYN) against several bacterial strains are as follow: S. aureus —3 μM (β-caryophyllene) versus 8 μM (KYN); K. pneumoniae (14 μM of β-caryophyllene) versus 2 μM (KYN); P. aeruginosa —7 μM (β-caryophyllene) versus 9 μM (KYN). β-caryophyllene has antiviral properties and can be effective against Herpes simplex virus type 1 (HSV-1) with an IC₅₀=0.25 μM and DENV-2 (Dengue Virus 2) with an IC₅₀=22.5 μM (4.6 mg/L) and was non-cytostatic with a selectivity index value of 71.1. β-caryophyllene can act very early in the steps of the viral replication cycle. In 2 mL of a formulation disclosed in an embodiment of the invention, a single dose of 13-caryophyllene is 15 mg, and the target concentration of this compound delivered by aerosolization can result in a concentration of 603 μg/mL in the epithelial lining fluid of the lungs. In an embodiment of the invention β-caryophyllene is in liquids that are aerosolized to treat viral and/or bacterial infections, for example, through direct antiviral, antibacterial, and/or antibiofilm properties and/or secondarily through other mechanisms including but not limited to antioxidant scavenging of ROS and RNS, cytokine antagonism, and/or mucolysis. Other naturally occurring plant extract compounds having antibacterial properties, including thymol, geraniol, and alkylamides, can be incorporated in the pharmaceutical compositions in this present invention.

β-caryophyllene shows cytotoxic potential in the human lung carcinoma (A549) cell line, a model of non-small cell lung cancer (NSCLC). About 80 to 85% of lung cancer cases are associated with NSCLC. When the cancer grade is higher than stage I, chemotherapeutic treatment is recommended for NSCLC. Despite substantial progress in the oncology field as a whole, outcomes following treatment for lung cancer are still poor. The cytotoxic IC50 value for β-caryophyllene in the A549 cell line is 28.18+/−1.96 μg/mL and in NCI-H358 cells is 31.19+/−2.01 μg/mL. A549 and H358 cells are cell models for NSCLC. β-caryophyllene induces A549 and NCI-H358 lung cancer cells' death via apoptosis, rather than by non-specific necrosis, and β-caryophyllene induces cell cycle arrest at the G1 phase in human lung cancer cell lines. β-caryophyllene may serve as a cancer chemoprevention agent for NSCLC, and β-caryophyllene has the potential to reduce or delay the occurrence of malignancy.

The two antioxidant compounds glutathione and N-acetyl cysteine, in embodiments of the invention, have anti-viral properties. The in vitro extracellular addition of glutathione to HIV cell cultures slows viral replication in both lymphocyte and monocyte cell lines and prevents activation of viral replication by TNF-α and IL-6. Glutathione can inhibit replication of parainfluenza-1, herpes simplex-1, and HIV-1 viruses by a direct effect on the envelope glycoproteins in certain cell populations.

The mechanisms of action of compounds discussed herein can be measured in a clinical setting, e.g., in a hospital, that may be associated with multi-center testing on patients infected by viruses (e.g., SARS-CoV-2) and bacteria, suffering from associated diseases (e.g., COVID-19), and with varying symptom levels.

### EXAMPLES

The following examples are provided as illustrations of embodiments of the invention, and not as limitations of the invention as claimed.

### Example A1

An embodiment of the invention is a liquid pharmaceutical composition that includes 1,8-cineole, β-caryophyllene, N-acetyl cysteine, glutathione, methylcobalamin, an emulsifying agent, and 0.9% saline sterile water and of which an example is set forth in Table A1, below. For example, sodium bicarbonate, sodium hydroxide, or a mixture of the two can be used to adjust the pH of the liquid pharmaceutical composition, e.g., to a pH of 7.2. Optionally, a preservative can be included in the composition. A liquid pharmaceutical composition that can be aerosolized is set forth in Table A1 (in this text, when compositions or mixtures are discussed, the term “percent” (%) refers to weight percentage, unless otherwise indicated). The liquid pharmaceutical composition comprising a TRPA1 antagonist and a CB₂ agonist, with mucolytic, antiviral, antibiotic, and anti-inflammatory properties that can be aerosolized, nebulized, or vaporized can optionally include borneol or a mixture of 1,8-cineole, β-caryophyllene, and/or borneol in the same or a different total concentration range as the concentration range of 1,8-cineole alone.

A method of manufacturing the liquid pharmaceutical composition includes preparing an aqueous phase mixture by taking 0.9% saline purified sterile water or nitrogen gas purged 0.9% saline purified sterile water and adding to it amounts of N-acetyl cysteine, glutathione, and methylcobalamin, followed by mixing until the liquid composition is homogeneous and all ingredients are dissolved. Nitrogen gas purging can be used throughout the mixing period to minimize oxygenation of the water and oxidation of the compounds in the mixture. This aqueous mixture can then be filtered through a filter with a 0.22 μm or less pore size to ensure sterilization. To further ensure sterilization and stability of the compounds in the formulation a quantity of a preservative can be added and/or the mixture can be refrigerated prior to use.

Amounts of 1,8-cineole and β-caryophyllene can be separately mixed with an emulsifier, and after this mixture is homogeneous, this mixture can be filtered to ensure sterilization. The oil phase mixture formed can then be slowly added to the aqueous phase mixture and slowly mixed until the liquids are dissolved in each other, minimizing the volatilization of the 1,8-cineole and β-caryophyllene. Mixing can be conducted in a zero or low headspace reactor or a nitrogen purged vessel to further minimize volatilization of 1,8-cineole and β-caryophyllene and oxidation of the compounds in the mixture. If an amount of 1,8-cineole or β-caryophyllene is added to the mixture at concentrations greater than the solubility of 1,8-cineole or β-caryophyllene in the mixture, then the 1,8-cineole and β-caryophyllene can be emulsified in the liquid composition with the addition of a suitable emulsifier, for example Tween 20, also known as Polysorbate 20 and polyoxyethylene(20)sorbitan monooleate. Mixing can be limited to that required to create a stable single phase homogeneous solution or emulsion and to minimize volatilization of the 1,8-cineole and/or β-caryophyllene. The aerosolizable liquid composition can be transferred to containers that can be stored for one or more doses; the containers may or may not have nitrogen gas in the headspace; and the containers may or may not be refrigerated.

In a method of use the liquid pharmaceutical composition can be aerosolized, nebulized, and/or vaporized. Methods of use of the liquid composition include, but are not limited to placing a quantity of the composition in a liquid aerosolization device, for example, a nebulizer, an ultrasonic nebulizer, an ultrasonic mesh nebulizer, or an inhaler, creating an aerosolized or nebulized mixture, and a patient inhaling the resultant vapors with the aerosolized or nebulized liquid pharmaceutical composition.

Methods of use of this liquid pharmaceutical composition include nebulization treatment of patients with lower respiratory diseases that are viral or bacterial, or a combination of the two, in origin. The lower respiratory disease being treated can be exacerbated by additional respiratory risk factors including cigarette smoking, marijuana smoking, and/or exposure to indoor or outdoor air pollution, and administration of this liquid pharmaceutical composition can treat the effects of such additional risk factors, as well as the viral and/or bacterial infection or disease.

TABLE A1
Table 1 - Base Viral Nebulizer Liquid

| | Weight | | | |
|---|---|---|---|---|
| | Percent | | | |
| Ingredient | (%) | Function | Sources | Secondary Effect |
| 1,8-Cineole | 0.1-5 | TRPA1 Antagonist, Anti- | Pure Compound or Essential oils of: | Mucolytic, TRPM8 Agonist, |
| | | Inflammatory, Antiviral | Eucalyptus polybractea; Eucalyptus | modulates immune function, |
| | | | globulus; Eucalyptus radiate; | bacteriostatic Fungistatic, |
| | | | Eucalyptus camaldulensis; | inhibition of production of |
| | | | Eucalyptus smithii; Eucalyptus | tumor necrosis factor- a |
| | | | globulus; Rosmarinus offficinalis | (TNF-α), interleukin-1β |
| | | | | (IL-1β), interleukin-4 (IL-4), |
| | | | | interleukin-5 (IL-5), |
| | | | | leukotriene B4 (LTB4), |
| | | | | thromboxane B2 (TXB2) and |
| | | | | prostaglandin E2 (PGE2) |
| β-Caryophyllene | 0.1-5 | CB₂ Agonist, | Pure Compound or Essential oils of: | Analgesic, neuroprotective, |
| | | Anti-Inflammatory, | Syzygium aromaticum, Carum | anti-depressive, anxiolytic, |
| | | Antibacterial, Antiviral | nigrum, Cinnamomum spp., Humulus | and anti-nephrotoxicity, |
| | | | lupulus, Piper nigrum L., Cannabis | inhibition of pro-inflammatory |
| | | | sativa, Rosmarinus offficinalis, | cytokines productions, such as |
| | | | Ocimum spp., Origanum vulgare | TNF-α, IL-1β and IL-6 |
| N-acetyl cysteine | 0.1-20 | Antiviral, Reactive Oxygen | Synthethic | Glutathione precursor, increase |
| | | Species Antioxidant, | | epithelial lining fluid and |
| | | Mucolytic, Natural Thiol | | lung glutathione concentrations, |
| | | Amino Acid Containing | | modulate immune function, |
| | | Compound | | inhibits NF-KB activation, |
| | | | | modulates immune function and |
| | | | | participates in the pulmonary |
| | | | | epithelial host defense system, |
| | | | | radionuclide and heavy metal chelate |
| Glutathione | 0.1-20 | Increase Epithelial Liquid | Synthethic | Modulate immune function, |
| | | Glutathione Concentration | | inhibits NF-KB activation, |
| | | Reactive Oxygen Species | | radionuclide and heavy metal |
| | | Antioxidant, Natural Thiol | | chelate |
| | | Amino Acid Containing | | |
| | | Compound | | |
| Methylcobalamin | 0.00001-1.00 | Antioxidant, Antiviral, | Synthethic | Decrease Vitamin B12 deficiency |
| | | Increase Epithelial Liquid | | the result of age and smoking. |
| | | and Lung Tissue Vitamin | | Reduce cyanide concentrations |
| | | B12 Concentration | | in lungs and serum |
| Emulsifier | 0.1-2.0 | Emulsifier | Natural or Synthetic | Creates Stable Suspension |
| Sterile Saline | 50.0-99 | Carrier | Filtered Water | Isotonic Diluent |
| Water - 0.9% | | | | |
| Sodium Bicarbonate | variable | pH Adjustment | Natural Mineral | Natural Buffer in Epithelial Cells |

### Example A2

An embodiment of the invention is a liquid pharmaceutical composition that includes 1,8-cineole, β-caryophyllene, N-acetyl cysteine, glutathione, methylcobalamin, and an emulsifying agent and of which an example is set forth in Table A2, below. A sterile saline solution can be included in the composition. For example, sodium bicarbonate, sodium hydroxide, or a mixture of the two can be used to adjust the pH of the liquid pharmaceutical composition, e.g., to a pH of 7.2. The liquid pharmaceutical composition that can be aerosolized, nebulized, or vaporized can optionally include borneol or a mixture of 1,8-cineole, β-caryophyllene, and/or borneol in the same or a different total concentration range as 1,8-cineole and β-caryophyllene.

A method of manufacturing the liquid pharmaceutical composition includes preparing an aqueous phase mixture by mixing 95.59 g of nitrogen purged 0.9% sterile saline solution with 1.11 g of N-acetyl cysteine, 1.11 g of glutathione, and 0.00067 g of methylcobalamin and mixing until the (aqueous) mixture is homogeneous. A separate oil phase mixture can be prepared by mixing 0.77 g of 1,8-cineole, 0.75 g of β-caryophyllene, and 0.67 g of Polysorbate 20 together and slowly mixing until the (oil) mixture is homogenous. The oil phase mixture formed can then be slowly added to the aqueous phase mixture and slowly mixed until the liquids are dissolved in each other, minimizing the volatilization of the 1,8-cineole and β-caryophyllene. Mixing can be limited to that required to create a stable single-phase homogeneous solution and to minimize volatilization of 1,8-cineole and β-caryophyllene. The pH of the solution can then be measured and a quantity of sodium bicarbonate, sodium hydroxide, or a combination of the two can be added to raise the pH to 7.20. A quantity of a preservative can be added and/or the liquid pharmaceutical composition can be refrigerated prior to use.

In a method of use the liquid pharmaceutical composition can be aerosolized, nebulized, and/or vaporized. Methods of use of the liquid composition include, but are not limited to placing a quantity of the composition in an ultrasonic, vibrating mesh, or jet nebulizer and a patient inhaling the aerosolized, nebulized, or vaporized vapors resulting from the aerosolized mixture created. In a method of use approximately 1 mL to 5 mL of the liquid composition can be placed into a liquid nebulizer for inhalation by a patient. An optimal aerosol particle size range created by a nebulizer can be between 2 μm (microns) and 5 μm to ensure maximum deposition of the aerosolized particles in the lower respiratory tract to reach the epithelial lining fluid and epithelial cells of the alveoli. Alternatively, if the aerosolized liquid is desired to be retained in the upper respiratory tract, a larger particle size range and distribution may be desired, and the nebulizer can produce particles of a size in the range of from 5 to 10 μm. Methods of use of the liquid composition include nebulization treatment of patients with lower respiratory diseases that are viral or bacterial, or a combination of the two, in origin. These lower respiratory diseases can be exacerbated by additional respiratory risk factors including cigarette smoking, marijuana smoking, or exposure to indoor or outdoor air pollution.

Table A2 shows the individual ingredient mass (in mg) present in a single 2 mL dose of the liquid pharmaceutical composition, as well as the maximum concentration achievable in the epithelial lining fluid based on an average total epithelial lining fluid volume of 25 mL. The concentration of each compound in the epithelial lining fluid from a nebulized 2 mL dose of the liquid composition disclosed in Table A2 is based on an assumption of 100 percent of the nebulized liquid reaching and being retained in the epithelial lining fluid. For example, a nebulizer device and use with the liquid composition disclosed in Table A2 can result in at least 80 percent of the composition reaching and being retained in the epithelial lining fluid and can result in at least 90 percent of the composition reaching and being retained in the epithelial lining fluid.

TABLE A2
Table 2 - Preferred Base Viral Nebulizer Liquid

| | Weight | | | Single | | Concentration | |
|---|---|---|---|---|---|---|---|
| | Percent | | | Maximum | | in Epithelial | |
| Ingredient | (%) | Mechanism of Action | Primary Effects | Dose at 2 mL | Units | Lining Fluid | Units |
| Glutathione | 1.11 | TRPA1 Antagonist, Antiviral, | Anti-Inflammatory, | 22.2 | mg | 0.89 | mg/ml |
| | | Reactive Oxygen Species | Antiviral | | | | |
| | | Antioxidant, Mucolytic | | | | | |
| N-acetyl cysteine | 1.11 | CB2 Antagonist, Antiviral, | Anti-Inflammatory, | 22.2 | mg | 0.89 | mg/ml |
| | | Antibacterial, Reactive Oxygen | Antiviral | | | | |
| | | Species Antioxidant | | | | | |
| 1,8-Cineole | 0.77 | Glutathione Precusor, Antiviral, | Antioxidant, | 15.4 | mg | 0.61 | mg/ml |
| | | Reactive Oxygen Species | Mucolytic, Antiviral | | | | |
| | | Antioxidant, Mucolytic | | | | | |
| β-Caryophyllene | 0.75 | Increase Epithelial Liquid and | Antioxidant, Natural | 15.1 | mg | 0.60 | mg/ml |
| | | Lung Tissue Glutathione | Thiol Amino Acid | | | | |
| | | Concentration | Containing Compound | | | | |
| Methylcobalamin | 0.00067 | Antioxidant, Antiviral, Increase | Vitamin Supplement, | 0.013 | mg | 0.0005 | mg/ml |
| | | Epithelial Liquid and Lung Tissue | Antiviral | | | | |
| | | Vitamin B12 Concentration | | | | | |
| Polysorbate 20 | 0.67 | Emulsifier | Creates Stable Suspension | 13.3 | mg | 0.53 | mg/ml |
| Sterile Saline | 95.59 | Carrier | Isotonic Diluent | 1911.8 | mg | 76.47 | mg/ml |
| Water - 0.9% | | | | | | | |
| Sodium | Variable to | pH Adjustment | Adjust pH to 7.20 | variable | | variable | |
| Bicarbonate | pH = 7.2 | | | | | | |

### Example A3

In an embodiment of the invention the liquid pharmaceutical composition set forth in Table A2 was made without sodium bicarbonate, sodium hydroxide, or a preservative and was manufactured without using any nitrogen purging. The pH of the composition was 7.0. 30 mL of this composition was made and placed in an amber glass dropper bottle with a calibrated glass dropper as the cap. The solution was refrigerated at 2° C.

In March 2020, a patient (a 47-year old healthy female) had symptoms of continual dry coughing, headaches, body aches, loss of sense of smell, congestion, and lethargy for 5 days, and on the 5^{th} day with these symptoms she began to have shortness of breath. These were all symptoms of the SARS-CoV-2 virus and the COVID-19 disease. The patient was concerned that the shortness of breath symptoms could become acute respiratory distress associated with viral pneumonia. She began nebulizing (1 mL) of the liquid pharmaceutical composition set forth in Example A2 and Table A2 on Mar. 27, 2020 at 2:00 μm. The nebulizer used was a Facelake FL800 Intelligent Mesh Nebulizer with a specification of 80% of nebulizer particles being less than 5 μm (an optimal range for deposition in the lower respiratory tract). After the initial nebulization dose the patient reported cessation of coughing and reported feeling much better. At 4:30 μm. she nebulized a second 1 mL dose of the liquid composition. She reported coughing a little before nebulization but not after and reported that she felt better and could take “breaths a little deeper than I was to beforehand” She nebulized a third dose (1.5 mL) of the liquid composition at 8:30 μm. Her fourth nebulized dose of the liquid composition was taken on Mar. 28, 2020 at 12:45 μm. (2.0 mL dose) and she reported that she, “stopped twice for minor coughs, but overall it went well. I did it before taking a shower and I think it was helpful. I also slept better last night and did not have any trouble breathing, plus it was also the first night in at least 5 nights that I did not wear a Breathe Right strip on my nose, and I was able to breathe fine. I am definitely feeling better.” Her fifth nebulized dose (2.0 mL) was taken on Mar. 28, 2020 at 5:25 μm. and she reported that, “I stopped once for minor cough and then was more careful about breathing more slowly and didn't have to stop again. It definitely makes me feel like it's working because it makes me very mindful of the breaths I'm taking and I feel like I'm taking deep breaths with it, which also makes me feel better psychologically. I still have minor coughing here and there throughout the day . . . my symptoms have materially improved” After 7 days the patient was symptom free and reported that she was feeling great.

On Apr. 3, 2020, 8 days following the beginning of this patient's nebulization of the liquid pharmaceutical composition set forth in Example A2 and Table A2, she had a COVID-19 test (Cobas® 8800 SARS-CoV-2 test manufactured by Roche) using a nasal swab collection. The test results were reported as non-detected indicating that the patient's “negative test result for this test means that SARSCoV-2 RNA was not present in the specimen above the limit of detection” (Cobas® SARS-CoV-2—Molecular Systems, Inc., Fact Sheet For Healthcare Providers, Mar. 31, 2020). This indicates that the SARS-CoV-2 virus associated RNA was no longer detectable in the patient.

On Apr. 17, 2020, following the patient's full recovery, she had a blood test for SARS-CoV-2 IgG and SARS-COVID-2 IgM antibodies to confirm whether or not she had past exposure to the SARS-CoV-2 virus. The Coronavirus SARS-CoV-2 COVID-19 IgG antibody lab test is for the detection of IgG antibodies against SARS-CoV-2 from human clinical specimens. The Coronavirus SARS-CoV-2 COVID-19 IgM antibody lab test is for the detection of IgM antibodies against SARS-CoV-2 from human clinical specimens. IgG and IgM blood-based serology testing helps to identify people who have been exposed to COVID-19 SARS-CoV-2 and may have developed some level of immunity, but potentially have mild to no symptoms. On Apr. 19, 2020 results of COVID-19, IgG serum test results were reported as reactive indicating positive results that the patient was exposed to SARS-CoV-2. On Apr. 19, 2020 results of COVID-19, IgM serum test results were reported as reactive indicating positive results that the patient was exposed to SARS-CoV-2.

In summary, these results confirm that the patient who was treated as disclosed above by nebulization of the liquid pharmaceutical composition in Example A2 and Table A2, had had the COVID-19 disease (caused by the SARS-CoV-2 virus), which was consistent with her symptoms. That is, twenty-three days following the patient's initial use of the liquid pharmaceutical composition disclosed in Example A2 and Table A2, the patient was tested for SARS-CoV-2 antibodies. It was confirmed that the patient tested positive for SARS-CoV-2 antibodies indicating the she had been exposed to the SARS-CoV-2 virus and her symptoms confirmed that she had had the COVID-19 disease. However, eight days following the beginning of nebulizing the liquid pharmaceutical composition of Example A2 and Table A2, COVID-19 testing indicated that the COVID-19 disease was no longer detectable in this patient; that is, the patient had recovered from COVID-19.

### Example A4

An embodiment of the invention is a liquid pharmaceutical composition that includes 1,8-cineole, β-caryophyllene, N-acetyl cysteine, glutathione, methylcobalamin, an emulsifying agent, and a sterile saline solution and of which an example is set forth in Table A3, below. For example, sodium bicarbonate, sodium hydroxide, or a mixture of the two can be used to adjust the pH of the liquid pharmaceutical composition, e.g., to a pH of 7.2. The liquid pharmaceutical composition that can be aerosolized, nebulized, or vaporized can optionally include borneol or a mixture of 1,8-cineole, β-caryophyllene, and/or borneol in the same or a different total concentration range as 1,8-cineole and β-caryophyllene.

A method of manufacturing the liquid pharmaceutical composition includes preparing an aqueous phase mixture by mixing 90.68 g of nitrogen purged 0.9% sterile saline solution with 2.22 g of N-acetyl cysteine, 3.33 g of glutathione, and 0.00133 g of methylcobalamin and mixing until the (aqueous) mixture is homogeneous. A separate oil phase mixture can be prepared by mixing 1.23 g of 1,8-cineole, 1.21 g of β-caryophyllene, and 1.33 g of Polysorbate 20 together and slowly mixing until the (oil) mixture is homogenous. The oil phase mixture formed can then be slowly added to the aqueous phase mixture and slowly mixed until the liquids are dissolved in each other, minimizing the volatilization of the 1,8-cineole and β-caryophyllene. Mixing can be limited to that required to create a stable single-phase homogeneous solution and to minimize volatilization of 1,8-cineole and β-caryophyllene. The pH of the solution can then be measured and a quantity of sodium bicarbonate, sodium hydroxide, or a combination of the two can be added to raise the pH to 7.20. A quantity of a preservative can be added and/or the mixture can be refrigerated prior to use.

In a method of use the liquid composition can be placed into an ultrasonic, vibrating mesh, or jet nebulizer and a patient can inhale the aerosolized, nebulized, or vaporized vapors resulting from creating an aerosolized mixture. In a method of use approximately 1 mL to 5 mL of the liquid composition can be place into a liquid nebulizer for inhalation by a patient. The optimal aerosol particle size range created by a nebulizer can be between 2 μm (microns) and 5 μm to ensure maximum deposition of the aerosolized particles in the lower respiratory tract to reach the epithelial lining fluid and epithelial cells of the alveoli. Alternatively, if the aerosolized liquid is desired to be retained in the upper respiratory tract, a larger particle size range and distribution may be desired, and the nebulizer can produce particles of a size in the range of from 5 to 10 μm. Methods of use of the liquid composition include nebulization treatment of patients with lower respiratory diseases that are viral in origin and in an advanced stage, for example, requiring mechanical ventilation of or other assistance in breathing for the patient. These lower respiratory diseases can also be additionally exacerbated by additional respiratory risk factors including respiratory bacterial infections, cigarette smoking, marijuana smoking, or exposure to indoor or outdoor air pollution.

Table A3 shows the individual ingredient mass (in mg) present in a single 2 mL dose of the liquid composition, as well as the maximum concentration achievable in the epithelial lining fluid based on an average total epithelial lining fluid volume of 25 mL. The concentration of each compound in the epithelial lining fluid from a nebulized 2 mL dose of the liquid composition disclosed in Table A3 is based on an assumption of 100 percent of the nebulized liquid reaching and being retained in the epithelial lining fluid. For example, a nebulizer device and use with the liquid composition disclosed in Table A3 can result in at least 80 percent of the composition reaching and being retained in the epithelial lining fluid and can result in at least 90 percent of the composition reaching and being retained in the epithelial lining fluid.

TABLE A3
Table 3- Preferred Viral Respiratory Challenged Nebulizer Liquid

| | Weight | | | Single | | Concentration | |
|---|---|---|---|---|---|---|---|
| | Percent | | | Maximum | | in Epithelial | |
| Ingredient | (%) | Mechanism of Action | Primary Effects | Dose at 2 mL | Units | Lining Fluid | Units |
| Glutathione | 2.22 | TRPA1 Antagonist, Antiviral, | Anti-Inflammatory, | 44.4 | mg | 1.78 | mg/ml |
| | | Reactive Oxygen Species | Antiviral | | | | |
| | | Antioxidant, Mucolytic | | | | | |
| N-acetyl cysteine | 3.33 | CB2 Antagonist, Antiviral, | Anti-Inflammatory, | 66.6 | mg | 2.66 | mg/ml |
| | | Antibacterial, Reactive Oxygen | Antiviral | | | | |
| | | Species Antioxidant | | | | | |
| 1,8-Cineole | 1.23 | Glutathione Precusor, TRPA1 | Antioxidant, | 24.6 | mg | 0.98 | mg/ml |
| | | Antagonist, Antiviral, Reactive | Mucolytic, | | | | |
| | | Oxygen Species Antioxidant, | Antiviral | | | | |
| | | Mucolytic | | | | | |
| β-Caryophyllene | 1.21 | Increase Epithelial Liquid and | Antioxidant, Natural | 24.1 | mg | 0.97 | mg/ml |
| | | Lung Tissue Glutathione | Thiol Amino Acid | | | | |
| | | Concentration | Containing Compound | | | | |
| Methylcobalamin | 0.00133 | Antioxidant, Antiviral, Increase | Vitamin | 0.027 | mg | 0.0011 | mg/ml |
| | | Epithelial Liquid and Lung Tissue | Supplement, | | | | |
| | | Vitamin B12 Concentration | Antiviral | | | | |
| Polysorbate 20 | 1.33 | Emulsifier | Creates Stable | 26.7 | mg | 1.07 | mg/ml |
| | | | Suspension | | | | |
| Sterile Saline | 90.68 | Carrier | Isotonic Diluent | 1813.6 | mg | 72.54 | mg/ml |
| Water - 0.9% | | | | | | | |
| Sodium | Variable to | pH Adjustment | Adjust pH to 7.20 | variable | | variable | |
| Bicarbonate | pH = 7.2 | | | | | | |

### Example A5

An embodiment of the invention is a liquid pharmaceutical composition that includes 1,8-cineole, β-caryophyllene, N-acetyl cysteine, glutathione, methylcobalamin, and an emulsifying agent and of which an example is set forth in Table A4, below. A sterile saline solution can be included in the composition. For example, sodium bicarbonate, sodium hydroxide, or a mixture of the two can be used to adjust the pH of the liquid pharmaceutical composition, e.g., to a pH of 7.2. The liquid pharmaceutical composition that can be aerosolized, nebulized, or vaporized can optionally include borneol or a mixture of 1,8-cineole, β-caryophyllene, and/or borneol in the same or a different total concentration range as 1,8-cineole and β-caryophyllene.

A method of manufacturing the liquid pharmaceutical composition includes preparing an aqueous phase mixture by mixing 90.85 g of nitrogen purged 0.9% sterile saline solution with 1.11 g of N-acetyl cysteine, 3.33 g of glutathione, and 0.00133 g of methylcobalamin and mixing until the (aqueous) mixture is homogeneous. A separate oil phase mixture can be prepared by mixing 1.23 g of 1,8-cineole, 1.81 g of β-caryophyllene and 1.67 g of Polysorbate 20 together and slowly mixing until the (oil) mixture is homogenous. The oil phase mixture can then be slowly added to the aqueous phase mixture and slowly mixed until the liquids are dissolved in each other, minimizing the volatilization of the 1,8-cineole and β-caryophyllene. Mixing can be limited to that required to create a stable single-phase homogeneous solution and to minimize volatilization of 1,8-cineole and β-caryophyllene. The pH of the solution can then be measured and a quantity of sodium bicarbonate, sodium hydroxide or a combination of the two can be added to raise the pH to 7.20. A quantity of a preservative can be added and/or alternatively the liquid pharmaceutical composition can be refrigerated prior to use.

In a method of use the liquid pharmaceutical composition can be aerosolized, nebulized, and/or vaporized. Methods of use of the liquid composition include, but are not limited to placing a quantity of the composition in an ultrasonic, vibrating mesh or jet nebulizer and a patient inhaling the aerosolized, nebulized, or vaporized vapors resulting from the aerosolized mixture created. In a method of use of approximately 1 mL to 5 ml of the liquid composition can be placed into a liquid nebulizer for inhalation by a patient. An optimal aerosol particle size range created by a nebulizer can be between 2 μm (microns) and 5 μm to ensure maximum deposition of the aerosolized particles in the lower respiratory tract to reach the epithelial lining fluid and epithelial cells of the alveoli. Alternatively, if the aerosolized liquid is desired to be retained in the upper respiratory tract, a larger particle size range and distribution may be desired, and the nebulizer can produce particles of a size in the range of from 5 to 10 μm. Methods of use of the liquid composition include nebulization treatment of patients with lower respiratory diseases that are bacterial in origin and in an advanced stage, for example, requiring mechanical ventilation of or other assistance in breathing for the patient. These lower respiratory diseases can also be additionally exacerbated by additional respiratory risk factors including respiratory viral infections, cigarette smoking, marijuana smoking, or exposure to indoor or outdoor air pollution. For example, bacterial bronchitis may follow a viral upper respiratory infection. Mycoplasma pneumoniae, Chlamydia pneumoniae, and Bordetella pertussis infection (which causes whooping cough) are among the bacteria that cause acute bronchitis. Bacterial causes of acute bronchitis are more likely when many people are affected (an outbreak or pandemic).

Table A4 shows the individual ingredient mass (in mg) present in a single 2 mL dose of the liquid composition, as well as the maximum concentration achievable in the epithelial lining fluid based on an average total epithelial lining fluid volume of 25 mL. The concentration of each compound in the epithelial lining fluid from a nebulized 2 mL dose of the liquid composition disclosed in Table A4 is based on an assumption of 100 percent of the nebulized liquid reaching and being retained in the epithelial lining fluid. For example, a nebulizer device and use with the liquid composition disclosed in Table A3 can result in at least 80 percent of the composition reaching and being retained in the epithelial lining fluid and can result in at least 90 percent of the composition reaching and being retained in the epithelial lining fluid.

TABLE A4
Table 4 - Preferred Bacterial Pneumonia Nebulizer Liquid

| | Weight | | | Single | | Concentration | |
|---|---|---|---|---|---|---|---|
| | Percent | | | Maximum | | in Epithelial | |
| Ingredient | (%) | Mechanism of Action | Primary Effects | Dose at 2 mL | Units | Lining Fluid | Units |
| Glutathione | 1.11 | TRPA1 Antagonist, Antiviral, | Anti-Inflammatory, | 22.3 | mg | 0.89 | mg/ml |
| | | Reactive Oxygen Species | Antiviral | | | | |
| | | Antioxidant, Mucolytic | | | | | |
| N-acetyl cysteine | 3.33 | CB2 Antagonist, Antiviral, | Anti-Inflammatory, | 66.6 | mg | 2.66 | mg/ml |
| | | Antibacterial, Reactive Oxygen | Antiviral | | | | |
| | | Species Antioxidant | | | | | |
| 1,8-Cineole | 1.23 | Glutathione Precusor, TRPA1 | Antioxidant, | 24.6 | mg | 0.98 | mg/ml |
| | | Antagonist, Antiviral, Reactive | Mucolytic, | | | | |
| | | Oxygen Species Antioxidant, | Antiviral | | | | |
| | | Mucolytic | | | | | |
| β-Caryophyllene | 1.81 | Increase Epithelial Liquid and | Antioxidant, Natural | 36.2 | mg | 1.45 | mg/ml |
| | | Lung Tissue Glutathione | Thiol Amino Acid | | | | |
| | | Concentration | Containing Compound | | | | |
| Methylcobalamin | 0.00133 | Antioxidant, Antiviral, Increase | Vitamin | 0.027 | mg | 0.0011 | mg/ml |
| | | Epithelial Liquid and Lung Tissue | Supplement, Antiviral | | | | |
| | | Vitamin B12 Concentration | | | | | |
| Polysorbate 20 | 1.67 | Emulsifier | Creates Stable Suspension | 33.3 | mg | 1.33 | mg/ml |
| Sterile Saline | 90.85 | Carrier | Isotonic Diluent | 1817.0 | mg | 72.68 | mg/ml |
| Water - 0.9% | | | | | | | |
| Sodium | Variable to | pH Adjustment | Adjust pH to 7.20 | variable | | variable | |
| Bicarbonate | pH = 7.2 | | | | | | |

### Example A6

An embodiment of the invention is a liquid pharmaceutical composition that includes 1,8-cineole, β-caryophyllene, N-acetyl cysteine, glutathione, methylcobalamin, L-theanine, taurine, an emulsifying agent, and 0.9% saline sterile water and of which an example is set forth in Table A5, below. For example, sodium bicarbonate, sodium hydroxide, or a mixture of the two can be used to adjust the pH of the liquid pharmaceutical composition, preferably to a pH of 7.2. Optionally, a preservative can be included in the composition. A liquid composition that can be aerosolized is set forth in Table A5. The liquid pharmaceutical composition comprising a TRPA1 antagonist and a CB₂ agonist, with mucolytic, antiviral, antibiotic, and anti-inflammatory properties, with amino acids that may be deficient in patients with respiratory diseases, and that can be aerosolized, nebulized, or vaporized can optionally include borneol or a mixture of 1,8-cineole, β-caryophyllene, and/or borneol in the same or a different total concentration range as the concentration range of 1,8-cineole alone.

A method of manufacturing the liquid composition includes preparing an aqueous phase mixture by taking 0.9% saline purified sterile water or nitrogen gas purged 0.9% saline purified sterile water and adding to it amounts of N-acetyl cysteine, glutathione, methylcobalamin, L-theanine, and taurine, followed by mixing until the liquid composition is homogeneous and all ingredients are dissolved. Nitrogen gas purging can be used throughout the mixing period to minimize oxygenation of the water and oxidation of the compounds in the mixture. This aqueous mixture can then be filtered through a filter with a 0.22 μm or less pore size ensure sterilization. To further ensure sterilization and stability of the compounds in the formulation a quantity of a preservative can be added and/or the mixture can be refrigerated prior to use.

Amounts of 1,8-cineole and β-caryophyllene can be separately mixed with an emulsifier, and after this mixture is homogeneous, this mixture can be filtered to insure sterilization. The oil phase mixture formed can then be slowly added to the aqueous phase mixture and slowly mixed until the liquids are dissolved in each other, minimizing the volatilization of the 1,8-cineole and β-caryophyllene. Mixing can be conducted in a zero or low headspace reactor or a nitrogen purged vessel to further minimize volatilization of 1,8-cineole and β-caryophyllene and oxidation of the compounds in the mixture. If an amount of 1,8-cineole or β-caryophyllene is added to the mixture at concentrations greater than the solubility of 1,8-cineole or β-caryophyllene in the mixture, then the 1,8-cineole and β-caryophyllene can be emulsified in the liquid composition with the addition of a suitable emulsifier, for example Tween 20, also known as Polysorbate 20 and polyoxyethylene(20)sorbitan monooleate. Mixing can be limited to that required to create a stable single phase homogeneous solution or emulsion and to minimize volatilization of the 1,8-cineole and/or β-caryophyllene. The aerosolizable liquid composition can be transferred to containers that can be stored for one or more doses; the containers may or may not have nitrogen gas in the headspace; and the containers may or may not be refrigerated.

In a method of use the liquid pharmaceutical composition can be aerosolized, nebulized, and/or vaporized. Methods of use of the liquid composition include, but are not limited to placing a quantity of the composition in a liquid aerosolization device, for example, a nebulizer, an ultrasonic nebulizer, an ultrasonic mesh nebulizer, or an inhaler, creating an aerosolized or nebulized mixture, and a patient inhaling the resultant vapors with the aerosolized or nebulized liquid pharmaceutical composition.

Methods of use of this liquid composition include nebulization treatment of patients with lower respiratory diseases that are viral or bacterial, or a combination of the two, in origin. The lower respiratory disease being treated can be exacerbated by additional respiratory risk factors including cigarette smoking, marijuana smoking, and/or exposure to indoor or outdoor air pollution, and administration of this liquid pharmaceutical composition can treat the effects of such additional risk factors, as well as the viral and/or bacterial infection or disease.

TABLE A5
Table 5 - Base Viral Amino Acid Nebulizer Liquid

| | Weight | | | |
|---|---|---|---|---|
| | Percent | | | |
| Ingredient | (%) | Function | Sources | Secondary Effect |
| 1,8-Cineole | 0.1-5 | TRPA1 Antagonist, Anti- | Pure Compound or Essential oils of: | Mucolytic, TRPM8 Agonist, |
| | | Inflammatory, Antiviral | Eucalyptus polybractea; Eucalyptus | modulates immune function, |
| | | | globulus; Eucalyptus radiate; | bacteriostatic Fungistatic, |
| | | | Eucalyptus camaldulensis; | inhibition of production |
| | | | Eucalyptus smithii; Eucalyptus | of tumor necrosis factor- a |
| | | | globulus; Rosmarinus offficinalis | (TNF-α), interleukin-1β |
| | | | | (IL-1β), interleukin-4 |
| | | | | (IL-4), interleukin-5 (IL-5), |
| | | | | leukotriene B4 (LTB4), |
| | | | | thromboxane B2 (TXB2) and |
| | | | | prostaglandin E2 (PGE2) |
| β-Caryophyllene | 0.1-5 | CB₂ Agonist, | Pure Compound or Essential oils of: | Analgesic, neuroprotective, |
| | | Anti-Inflammatory, | Syzygium aromaticum, Carum | anti-depressive, anxiolytic, |
| | | Antibacterial, Antiviral | nigrum, Cinnamomum spp., Humulus | and anti-nephrotoxicity, |
| | | | lupulus, Piper nigrum L., Cannabis | inhibition of pro-inflammatory |
| | | | sativa, Rosmarinus offficinalis, | cytokines productions, such |
| | | | Ocimum spp., Origanum vulgare | as TNF-α, IL-1β and IL-6 |
| N-acetyl cysteine | 0.1-20 | Antiviral, Reactive Oxygen | Synthethic | Glutathione precursor, increase |
| | | Species Antioxidant, | | epithelial lining fluid and |
| | | Mucolytic, Natural Thiol | | lung glutathione concentrations, |
| | | Amino Acid Containing | | modulate immune function, |
| | | Compound | | inhibits NF-KB activation, |
| | | | | modulates immune function and |
| | | | | participates in the pulmonary |
| | | | | epithelial host defense system, |
| | | | | radionuclide and heavy metal chelate |
| Glutathione | 0.1-20 | Increase Epithelial Liquid | Synthethic | Modulate immune function, |
| | | Glutathione Concentration | | inhibits NF-KB activation, |
| | | Reactive Oxygen Species | | radionuclide and heavy metal chelate |
| | | Antioxidant, Natural Thiol | | |
| | | Amino Acid Containing | | |
| | | Compound | | |
| Methylcobalamin | 0.00001-1.00 | Antioxidant, Antiviral, | Synthethic | Decrease Vitamin B12 deficiency |
| | | Increase Epithelial Liquid | | the result of age and smoking. |
| | | and Lung Tissue Vitamin | | Reduce cyanide concentrations |
| | | B12 Concentration | | in lungs and serum |
| L-Theanne | 0.1-10 | Amino Acid, Antioxidant | Synthethic | Anti-inflammatory, antioxidative, |
| | | | | hepatoprotective effects, |
| | | | | decreaseds IgE, monocyte |
| | | | | chemoattractant protein-1 (MCP-1), |
| | | | | interleukin (IL)-4, IL-5, IL-13, |
| | | | | tumor necrosis factor-alpha |
| | | | | (TNF-α), and interferon-gamma |
| | | | | (INF-γ) |
| Taurine | 0.1-10 | Amino Acid, Dissipate Toxic | Synthethic | Natural Antioxidant, Natural |
| | | Effects of HOCl in Epitheial | | Thiol Amino Acid Containing |
| | | Cenlls | | Compound |
| Emulsifier | 0.1-2.0 | Emulsifier | Natural or Synthetic | Creates Stable Suspension |
| Sterile Saline | 50.0-99 | Carrier | Filtered Water | Iso to nic Diluent |
| Water - 0.9% | | | | |
| Sodium Bicarbonate | variable | pH Adjustment | Natural Mineral | Natural Buffer in Epithelial Cells |

### Example A7

An embodiment of the invention is a liquid pharmaceutical composition that includes 1,8-cineole, β-caryophyllene, N-acetyl cysteine, glutathione, methylcobalamin, L-theanine, taurine, and an emulsifying agent and of which an example is set forth in Table A6, below. A sterile saline solution can be included in the composition. For example, sodium bicarbonate, sodium hydroxide, or a mixture of the two can be used to adjust the pH of the liquid pharmaceutical composition, e.g., to a pH of 7.2. The liquid pharmaceutical composition that can be aerosolized, nebulized, or vaporized can optionally include borneol or a mixture of 1,8-cineole, β-caryophyllene, and/or borneol in the same or a different total concentration range as 1,8-cineole and β-caryophyllene.

A method of manufacturing the liquid pharmaceutical composition includes preparing an aqueous phase mixture by mixing 84.18 g of nitrogen purged 0.9% sterile saline solution with 1.11 g of N-acetyl cysteine, 3.33 g of glutathione, 0.00133 g of methylcobalamin, 3.33 g of L-theanine, and 3.33 g of taurine and mixing until the (aqueous) mixture is homogeneous. A separate oil phase mixture can be prepared by mixing 1.23 g of 1,8-cineole, 1.81 g of β-caryophyllene, and 1.67 g of Polysorbate 20 together and slowly mixing until the (oil) mixture is homogenous. The oil phase mixture formed can then be slowly added to the aqueous phase mixture and slowly mixed until the liquids are dissolved in each other, minimizing the volatilization of the 1,8-cineole and β-caryophyllene. Mixing can be limited to that required to create a stable single-phase homogeneous solution and to minimize volatilization of 1,8-cineole and β-caryophyllene. The pH of the solution can then be measured and a quantity of sodium bicarbonate, sodium hydroxide, or a combination of the two can be added to raise the pH to 7.20. A quantity of a preservative can be added and/or the mixture can be refrigerated prior to use.

In a method of use the liquid pharmaceutical composition can be aerosolized, nebulized, and/or vaporized. Methods of use of the liquid composition include, but are not limited to placing a quantity of the composition in an ultrasonic, vibrating mesh, or jet nebulizer and a patient inhaling the aerosolized, nebulized, or vaporized vapors resulting from the aerosolized mixture created. In a method of use approximately 1 mL to 5 ml of the liquid composition can be placed into a liquid nebulizer for inhalation by a patient. An optimal aerosol particle size range created by a nebulizer can be between 2 μm (microns) and 5 μm to ensure maximum deposition of the aerosolized particles in the lower respiratory tract to reach the epithelial lining fluid and epithelial cells of the alveoli. Alternatively, if the aerosolized liquid is desired to be retained in the upper respiratory tract, a larger particle size range and distribution may be desired, and the nebulizer can produce particles of a size in the range of from 5 to 10 μm. Methods of use of the liquid composition include nebulization treatment of patients with lower respiratory diseases that are viral or bacterial, or a combination of the two, in origin. These lower respiratory diseases can be exacerbated by additional respiratory risk factors, including cigarette smoking, marijuana smoking, or exposure to indoor or outdoor air pollution. Metabolomic spillover can result in systemic amino acid deficiencies or lower than normal concentrations of amino acids in epithelial lining fluid, epithelial cells, and other respiratory structures and cells in the lower respiratory tract, and administration of the liquid pharmaceutical composition to a patient (through inhalation of the aerosolized, nebulized, and/or vaporized composition) can ameliorate such systemic amino acid deficiencies (for example, by reducing or eliminating a deficiency).

Table A6 shows the individual ingredient mass (in mg) present in a single 2 mL dose of the liquid pharmaceutical composition, as well as the maximum concentration achievable in the epithelial lining fluid based on an average total epithelial lining fluid volume of 25 mL. The concentration of each compound in the epithelial lining fluid from a nebulized 2 mL dose of the liquid composition disclosed in Table A6 is based on an assumption of 100 percent of the nebulized liquid reaching and being retained in the epithelial lining fluid. For example, a nebulizer device and use with the liquid composition disclosed in Table A6 can result in at least 80 percent of the composition reaching and being retained in the epithelial lining fluid and can result in at least 90 percent of the composition reaching and being retained in the epithelial lining fluid.

TABLE A6
Table 6. Preferred Base Nebulizer Liquid with Amino Acids

| | | | | | | Concen- | |
|---|---|---|---|---|---|---|---|
| | | | | | | tration in | |
| | Weight | | | Single | | Epithelial | |
| | Percent | | | Maximum | | Lining | |
| Ingredient | (%) | Function | Primary Effects | Dose at 2 mL | Units | Fluid | Units |
| Glutathione | 1.11 | Mechanism of Action | Primary Effects | 22.3 | mg | 0.89 | mg/ml |
| N-acetyl cysteine | 3.33 | TRPA1 Antagonist, Antiviral, | Anti-Inflammatory, | 66.6 | mg | 2.66 | mg/ml |
| | | Reactive Oxygen Species | Antiviral | | | | |
| | | Antioxidant, Mucolytic | | | | | |
| 1,8-Cineole | 1.23 | CB2 Antagonist, Antiviral, | Anti-Inflammatory, | 24.6 | mg | 0.98 | mg/ml |
| | | Antibacterial, Reactive Oxygen | Antiviral | | | | |
| | | Species Antioxidant | | | | | |
| β-Caryophyllene | 1.81 | Glutathione Precusor, TRPA1 | Antioxidant, | 36.2 | mg | 1.45 | mg/ml |
| | | Antagonist, Antiviral, Reactive | Mucolytic, Antiviral | | | | |
| | | Oxygen Species Antioxidant, | | | | | |
| | | Mucolytic | | | | | |
| Methylcobalamin | 0.0013 | Increase Epithelial Liquid and | Antioxidant, Natural | 0.0267 | mg | 0.0011 | mg/ml |
| | | Lung Tissue Glutathione | Thiol Amino Acid | | | | |
| | | Concentration | Containing Compound | | | | |
| L-Theanine | 3.33 | Decreases Mucous Production, | Amino Acid, | 66.7 | mg | 2.67 | mg/ml |
| | | Anti-Inflammatory | Natural Antioxidant | | | | |
| Taurine | 3.33 | Dissipate Toxic Effects of | Natural Antioxidant, | 66.7 | mg | 2.67 | mg/ml |
| | | HOCl in Epitheial Cells | Natural Thiol | | | | |
| | | | Amino Acid | | | | |
| | | | Containing Compound | | | | |
| Polysorbate 20 | 1.67 | Emulsifier | Creates Stable Suspension | 33.3 | mg | 1.33 | mg/ml |
| Sterile Saline | 84.18 | Carrier | Isotonic Diluent | 1683.7 | mg | 67.35 | mg/ml |
| Water - 0.9% | | | | | | | |
| Sodium | Variable to | pH Adjustment | Adjust pH to 7.20 | variable | | variable | |
| Bicarbonate | pH = 7.2 | | | | | | |

### Example A8

An embodiment of the invention is a liquid pharmaceutical composition that includes 1,8-cineole, β-caryophyllene, glutathione, methylcobalamin, and an emulsifying agent and of which an example is set forth in Table A7, below. A sterile saline solution can be included in the composition. For example, sodium bicarbonate, sodium hydroxide, or a mixture of the two can be used to adjust the pH of the liquid pharmaceutical composition, e.g., to a pH of 7.2. The liquid pharmaceutical composition that can be aerosolized, nebulized, or vaporized can optionally include borneol or a mixture of 1,8-cineole, β-caryophyllene, and/or borneol in the same or a different total concentration range as 1,8-cineole and β-caryophyllene.

A method of manufacturing the liquid pharmaceutical composition includes preparing an aqueous phase mixture by mixing 91.17 g of nitrogen purged 0.9% sterile saline solution with 2.22 g of glutathione and 0.00133 g of methylcobalamin and mixing until the (aqueous) mixture is homogeneous. A separate oil phase mixture can be prepared by mixing 3.07 g of 1,8-cineole, 1.21 g of β-caryophyllene, and 2.33 g of Polysorbate 20 together and slowly mixing until the (oil) mixture is homogenous. The oil phase mixture formed can then be slowly added to the aqueous phase mixture and slowly mixed until the liquids are dissolved in each other, minimizing the volatilization of the 1,8-cineole and β-caryophyllene. Mixing is limited to that required to create a stable single-phase homogeneous solution and to minimize volatilization of 1,8-cineole and β-caryophyllene. Mixing can be limited to that required to create a stable single-phase homogeneous solution and to minimize volatilization of 1,8-cineole and β-caryophyllene. The pH of the solution can then be measured and a quantity of sodium bicarbonate, sodium hydroxide, or a combination of the two can be added to raise the pH to 7.20. A quantity of a preservative can be added and/or the mixture can be refrigerated prior to use.

In a method of use the liquid pharmaceutical composition can be aerosolized, nebulized, and/or vaporized. Methods of use of the liquid composition include, but are not limited to placing a quantity of the composition in an ultrasonic, vibrating mesh or jet nebulizer and a patient inhaling the aerosolized, nebulized, or vaporized vapors resulting from the aerosolized mixture created. In a method of use approximately 1 mL to 5 mL of the liquid composition can be placed into a liquid nebulizer for inhalation by a patient. The optimal aerosol particle size range created by a nebulizer is between 2 μm (microns) and 5 μm, to ensure maximum deposition of the aerosolized particles in the lower respiratory tract to reach the epithelial lining fluid and epithelial cells of the alveoli. Alternatively, if the aerosolized liquid is desired to be retained in the upper respiratory tract, a larger particle size range and distribution may be desired, and the nebulizer can produce particles of a size in the range of from 5 to 10 μm. Methods of use of the liquid composition include nebulization treatment of patients with lower respiratory diseases that are viral or bacterial, or a combination of the two, in origin. These lower respiratory diseases can be exacerbated by additional respiratory risk factors including cigarette smoking, marijuana smoking, or exposure to indoor or outdoor air pollution. Metabolomic spillover can result in systemic amino acid deficiencies or lower than normal concentrations of amino acids in epithelial lining fluid, epithelial cells, and other respiratory structures and cells in the lower respiratory tract, and administration of the liquid pharmaceutical composition to a patient (through inhalation of the aerosolized, nebulized, and/or vaporized composition) can ameliorate such systemic amino acid deficiencies (for example, by reducing or eliminating a deficiency).

Table A7 shows the individual ingredient mass (in mg) present in a single 2 mL dose of the liquid pharmaceutical composition, as well as the maximum concentration achievable in the epithelial lining fluid based on an average total epithelial lining fluid volume of 25 mL. The concentration of each compound in the epithelial lining fluid from a nebulized 2 mL dose of the liquid composition disclosed in Table A7 is based on an assumption of 100 percent of the nebulized liquid reaching and being retained in the epithelial lining fluid. For example, a nebulizer device and use with the liquid composition disclosed in Table A7 can result in at least 80 percent of the composition reaching and being retained in the epithelial lining fluid and can result in at least 90 percent of the composition reaching and being retained in the epithelial lining fluid.

TABLE A7
Table 7 - Preferred Base Viral Nebulizer Liquid

| | Weight | | | Single | | Concentration | |
|---|---|---|---|---|---|---|---|
| | Percent | | | Maximum | | in Epithelial | |
| Ingredient | (%) | Mechanism of Action | Primary Effects | Dose at 2 mL | Units | Lining Fluid | Units |
| Glutathione | 2.22 | TRPA1 Antagonist, Antiviral, | Anti-Inflammatory, | 44.4 | mg | 1.78 | mg/ml |
| | | Reactive Oxygen Species | Antiviral | | | | |
| | | Antioxidant, Mucolytic | | | | | |
| 1,8-Cineole | 3.07 | CB2 Antagonist, Antiviral, | Anti-Inflammatory, | 61.5 | mg | 2.46 | mg/ml |
| | | Antibacterial, Reactive Oxygen | Antiviral | | | | |
| | | Species Antioxidant | | | | | |
| β-Caryophyllene | 1.21 | Increase Epithelial Liquid and | Antioxidant, Natural | 24.1 | mg | 0.97 | mg/ml |
| | | Lung Tissue Glutathione | Thiol Amino Acid | | | | |
| | | Concentration | Containing Compound | | | | |
| Methylcobalamin | 0.00133 | Antioxidant, Antiviral, Increase | Vitamin | 0.027 | mg | 0.0011 | mg/ml |
| | | Epithelial Liquid and Lung Tissue | Supplement, Antiviral | | | | |
| | | Vitamin B12 Concentration | | | | | |
| Polysorbate 20 | 2.33 | Emulsifier | Creates Stable Suspension | 46.7 | mg | 1.87 | mg/ml |
| Sterile Saline | 91.17 | Carrier | Isotonic Diluent | 1823.3 | mg | 72.93 | mg/ml |
| Water - 0.9% | | | | | | | |
| Sodium | Variable to | pH Adjustment | Adjust pH to 7.20 | variable | | variable | |
| Bicarbonate | pH = 7.2 | | | | | | |

### Example A9

FDA regulatory status, presence in food and key toxicity values of selected ingredients in the liquid pharmaceutical compositions disclosed in Tables A1 through A7 are shown in Table A8, below. All of the compounds identified in Table A8 are found in existing Over-The-Counter (OTC) no-prescription drugs taken orally. All of the compounds identified in Table A8 are Generally Recognized as Safe and Effective (GRAS), with the exception of N-acetyl cysteine, which is present in OTC dietary supplements. Similarly, all of the compounds identified in Table A8 are present in foods either naturally or are approved FDA food and flavor additives, with the exception of N-acetyl cysteine. Daily dietary intake values of glutathione, N-acetyl cysteine, and Polysorbate 20 through oral means in foods are greater than of these compounds in a 2 mL/dose taken 4 times per day of the liquid pharmaceutical composition set forth in Table A2. The daily intake values in foods of three other compounds are lower in foods than they are in a 2 mL/dose taken 4 times per day of the liquid pharmaceutical composition set forth in Table A2. These three other compounds include the following: 1,8-cineole, which would be about 25 times greater in the administered liquid pharmaceutical composition than in an average daily dietary intake, β-caryophyllene which would be about 100 times greater in the administered liquid pharmaceutical composition than in an average daily dietary intake; and methylcobalamin which would be about 10 times greater in the administered liquid pharmaceutical composition than in an average daily dietary intake.

From a toxicity standpoint the No Observed Adverse Exposure Levels (NOAEL) for all of the ingredients that have established NOAELs are many orders of magnitude higher than those that would be inhaled at doses in a 2 mL/dose taken 4 times per day of the liquid pharmaceutical composition. For example, the NOAEL value for 1,8-cineole taken orally in mice over a 28-day period is 562.5 mg/kg (body weight). For a 62 kg adult human the NOAEL would be 34,875 mg per day. This is a factor 697.5 times higher than the 50 mg/day dose taken by inhalation of 1,8-cineole from a 2 mL/dose taken 4 times per day of the nebulized liquid pharmaceutical composition set forth in Table A2.

The NOAEL value for β-caryophyllene taken orally in mice over a 90-day period is greater than 700 mg/kg (body weight), the highest dose tested. For a 62 kg adult human the NOAEL would be 43,400 mg per day. This is a factor 868 times higher than the 50 mg/day dose taken by inhalation of β-caryophyllene from a 2 mL/dose taken 4 times per day of the nebulized liquid pharmaceutical composition set forth in Table A2.

The NOAEL value for methylcobalamin taken orally in mice is greater than 500 mg/kg (body weight), the highest dose tested. For a 62 kg adult human the NOAEL would be 31,000 mg per day. This is a factor 596,254 times higher than the 52 μg/day dose taken by inhalation of methylcobalamin from a 2 mL/dose taken 4 times per day of the nebulized liquid pharmaceutical composition set forth in Table A2.

The NOAEL value for Polysorbate 20 taken orally in rats is greater than 2,500 mg/kg (body weight), the highest dose tested. For a 62 kg adult human the NOAEL would be 155,000 mg per day. This is a factor 2,980 times higher than the 52 mg/day dose taken by inhalation of Polysorbate 20 from a 2 mL/dose taken 4 times per day of the nebulized liquid pharmaceutical composition disclosed in Table A2.

There is no NOAEL value for glutathione as it is an endogenously produced compound in humans and is present in all cells. There is no NOAEL value for N-acetyl cysteine as it is a synthetic source of cysteine and an endogenously produced compound in humans and is present in all cells.

The LD₅₀ values of the compounds disclosed in Table A2 and reported in Table A8 are all large values, as follows: glutathione—5000 mg/kg; N-acetyl cysteine—5050 mg/kg; 1,8-cineole—2,480 mg/kg; β-caryophyllene>5,000 mg/kg; methylcobalamin—none established; Polysorbate 20-3,850 mg/kg. The United States Environmental Protection Agency (USEPA) identifies categories of safety for compounds with various LD₅₀ values. Compounds with LD₅₀ values greater than 2,000 mg/kg (body weight) are considered by the USEPA to be practically non-toxic.

A person of skill in the art would be taught from consideration of the published toxicity values, use in foods, and daily dietary intake values of the compounds set forth in Table A8, that these compounds are safe and practically non-toxic. Therefore, it is surprisingly and unexpected that compounds in Table A8 are toxic to pathogenic viruses and bacteria, while at the same time being safe for humans.

TABLE A8
Table 8. Safety and Regulatory Status of Nebulized Liquid Ingredients

| | | | | | | Inspiritol | | |
|---|---|---|---|---|---|---|---|---|
| | | | | | Daily | Single | | |
| | | | | | Dietary | 2 mL Dose | | |
| | | | OTC | Present | Intake in | (4 × Doses/ | | |
| Ingredient | CAS No. | FDA Status | Availability | in Food | Food (US)* | day) | LD 50 | NOAEL |
| Glutathione | 70-18-8 | GRAS (inhalation, | Yes/Oral | Yes/Natural | 8.4 mg/ | 22 mg | 5,000 mg/kg | N/E |
| (GSH) | | intravenous | (500 mg) | | kg body | (88 mg/day) | (oral, mouse ) | |
| | | prescribed) | | | wt/day | | | |
| | | Inhalation | | | (521 mg) | | | |
| | | Dose 600 mg | | | | | | |
| | | -prescribed | | | | | | |
| | | in medicine | | | | | | |
| n-acetyl- | 616-91-1 | FDA Approved | Yes/Oral | No/Flavor & | 4.2 mg/kg | 22 mg | 5,050 mg/kg | N/E |
| cysteine | | Drug | (600 mg) | Supplement | body | (88 mg/day) | (oral, rat) | |
| (NAC) | | (inhalation, | | | wt/day | | | |
| | | intravenous | | | (260 mg)† | | | |
| | | prescribed) | | | | | | |
| | | Inhalation Dose | | | | | | |
| | | 600-1000 mg | | | | | | |
| | | (Mucomyst) | | | | | | |
| 1,8-cineole | 470-82-6 | GRAS | Yes/Mouth Wash | Yes/Natural | 33 μg/kg body | 15 mg | 2,480 mg/kg | 562.5 mg/kg |
| (EUC) | | | (92 mg/mL)/ | & Flavor | wt/day (2 mg) | (50 mg/day) | (oral, rat) | (oral, mice) |
| | | | Cough (varies) | | | | | 28-d |
| B-Cary- | 87-44-5 | GRAS | Sold Non-OTC | Yes/Natural | 8 μg/kg body | 15 mg | >5,000 mg/kg | >700 mg/kg |
| ophyllene | | | (30 mg patch) | & Flavor | wt/day | (50 mg/day) | (oral, rat) | (oral, rat) |
| (BCP) | | | | | (0.5 mg) | | | 90 d |
| Methyl- | 13422-55-4 | GRAS (inhalation, | Yes/Oral | Yes/ | 3 μg/day | 13 μg | None | 500 mg/kg |
| cobalamin | | intravenous | (500 μg) | Natural & | (women); | (52 μg/day) | Established | (oral) |
| (B12) | | prescribed) | | Supplement | 5 μg/day | | | |
| | | Inhalation | | | (men) | | | |
| | | Dose 1000 μg | | | | | | |
| | | prescribed in | | | | | | |
| | | medicine | | | | | | |
| Polysorbate | 9005-64-5 | GRAS inactive | Yes/Many | Yes/ | Adults - | 13 mg | 3.850 mg/kg | 2,500 mg/kg |
| 20 (PS20) | | ingredient | Products | Emulsifier | 0.6 mg/kg | (52 mg/day) | (oral, mouse ) | (oral, rat) |
| | | (inhalation, | | Additive | body wt/day | | | |
| | | intravenous | | | (37 mg); | | | |
| | | prescribed) | | | Child - | | | |
| | | | | | 18.1 mg/kg | | | |
| | | | | | body wt/day | | | |
| | | | | | (1,122 mg) | | | |
| Sterile | 7647-14-5 | GRAS | Yes/Many | N/A | N/A | 1,913 mg | N/A | N/A |
| Saline | | | Products | | | (7.652 | | |
| Water | | | | | | mg/day) | | |
| (0.9%) | | | | | | | | |

Notes:
*daily intake for 62 kg person;
†based on cystine in food;
N/A—Not Applicable;
N/E—None Established;
LD50—Lethal Dose where 50% mortaility of tested animals;
NOAEL—No Observed Adverse Exposure Level

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

### Example A10

In an embodiment of this present invention the liquid pharmaceutical composition set forth in Table A2 was made without sodium bicarbonate, sodium hydroxide, or a preservative and was manufactured without using any nitrogen purging. The pH of the composition was 7.0. The formulation was transferred into 10 mL sterile glass septum sealed vials. The solution was refrigerated at 2° C. In addition to the COVID-19 patient treated in Example A3, three additional COVID-19 patients with various respiratory and other symptoms were treated and described below.

Patient 1: 55 year old female non-hospitalized patient with two confirmed COVID-19 tests taken Nov. 4 and 7, 2020.

Symptoms: Severe chest pain, fever, dry coughing, extremely fatigued and anxiety. Symptoms began on Nov. 2, 2020 with chest pain and increased through Nov. 10, 2020. Patient lost sense of taste on Nov. 11, 2020. The patient reported literally having to crawl on the floor to go to the bathroom.

Initial Dosing: The patient began nebulizing the liquid pharmaceutical composition disclosed in Table A2 on Nov. 7, 2020 (1 mL, 2 times per day) and increased dosing to 1.5 mL, every 4 hours on Nov. 8, 2020, and again increased dosing to 2 mL, every 4 hours on Nov. 9, 2020 and finally 3 mL, 4 times per day on Nov. 10, 2020 through Nov. 12, 2020. Treatment continued through Nov. 16, 2020 with 3 mL/dose but with less frequent dosing.

Results: The patient began oxygen saturation measurements on Nov. 9, 2020 with 95% oxygen. Oxygen saturation measurements increased, as follows: 11/10/2020-96%; 11/11/2020-95% to 96%; 11/13/2020-97% to 98%. She reported that her weakness began to lessen on Nov. 10, 2020, continued to lessen on Nov. 11, 2020 and continued to decrease each following day. She reported that her chest pain began to decrease on Nov. 12, 2020 and continued to improve each day. The patient reported the loss of sense of taste on Nov. 11, 2020, but it lasted only one day. This patient reported no adverse reactions or symptoms associated with nebulization of this pharmaceutical formulation.

COVID-19 Testing: On Nov. 16, 2020, the patient received a negative COVID-19 test. This patient has reported fully recovering from COVID-19, with no on-going symptoms.

Patient 2: 45 year old female Registered Nurse non-hospitalized patient with confirmed COVID-19 test.

Symptoms: Symptoms began with body aches and a low-grade temperature on the first day symptoms began in December 2020. Symptoms progressed to a headache on the second day and the patient was COVID-19 tested at the hospital in which she worked, which was positive, then she isolated at home. On day three and four she had severe sinus congestion and on day five lost her sense of taste and smell and started to experience coughing and at about that same time her sinus congestion cleared up.

Initial Dosing: On day 7 from the commencement of symptoms the patient began nebulization of the liquid pharmaceutical composition disclosed in Table A2 made without sodium bicarbonate, sodium hydroxide, or a preservative and was manufactured without using any nitrogen purging, with treatments (2 mL, twice per day) which continued for 6 days through day 12.

Results: The patient would cough more after nebulization treatments, but overall reported breathing easier. After discussions with Employee Health (at the hospital she works) she was assured she was no longer contagious and came out of isolation on day 12. Her coughing and fatigue persisted for about an additional 2 weeks. Other than brief coughing after inhalation, this patient reported no adverse reactions or symptoms associated with nebulization of this pharmaceutical formulation.

COVID-19 Testing: This patient did not receive a negative COVID-19 test after Inspiritol treatment, but doctors at her hospital assured her that she was no longer contagious after day 12 following her initial symptoms.

Patient 3: 83 year old female non-hospitalized patient with confirmed COVID-19 test.

Symptoms: Symptoms began on Mar. 16, 2021, four days following receiving her first COVID-19 vaccination. Initial symptoms were fever (102.9° F.), vomiting, coughing, extreme fatigue and a severe sore throat. She was brought to a hospital by ambulance, given intravenous electrolyte fluids, an anti-nausea medication, tested positive for COVID-19 and was sent home. The fever decreased, but was not absent, after taking acetaminophen. After about 10 days her symptoms changed to include shortness of breath, with body aches and a low-grade temperature. The patient reported that her oxygen saturation level (taken with an at-home pulse oximeter) was 91% to 92% prior to nebulization therapy.

Initial Dosing: On Apr. 6, 2021 this patient began nebulization of the liquid pharmaceutical composition disclosed in Table A2 made without sodium bicarbonate, sodium hydroxide, or a preservative and was manufactured without using any nitrogen purging, with treatments consisting of 3 mL, twice per day, which continued through Apr. 17, 2021.

Results: The patient would cough for one to two minutes after most nebulization treatments, but overall reported breathing easier. Other than brief coughing after inhalation, this patient reported no adverse reactions or symptoms associated with nebulization of this pharmaceutical formulation. Following the first nebulization treatment on Apr. 6, 2021, this patient reported that her oxygen saturation increased to 96% and by Apr. 9, 2021 she reported her levels increased to 97% to 98%. As of Apr. 14, 2021, the patient reported her breathing had improved and no longer had shortness of breath unless she was exerting herself. She also reported that she felt less fatigued and no longer had any other symptoms. Her oxygen saturation levels were reported to stabilized around 97% to 98%. This patient reported that her symptoms were “pretty much resolved.”

COVID-19 Testing: This patient received no follow-up COVID-19 testing following nebulization inhalation treatment.

### Example A11

Selected blood tests were conducted on nine patients before and after nebulization inhalation of the pharmaceutical composition disclosed in Table A2 made without sodium bicarbonate, sodium hydroxide, or a preservative and was manufactured without using any nitrogen purging. At the time of testing Patient 211 was a 67 year old female in good health, with a frequent cough, the result of exposure and allergies to horse dander and hay dust associated with her profession. The patient nebulized the liquid pharmaceutical composition at an average dosing of 3.2 mL/day for 6 days over an 8 day period. At the time of testing Patient 212 was a 67 year old male in good health, and although not symptomatic has a family history of autoimmune diseases, including Sjogren's syndrome. The patient nebulized the liquid composition at an average dosing of 4.6 mL/day each day over a 9 day period. At the time of testing Patient 213 was a 48 year old female in good health. The patient nebulized the liquid pharmaceutical composition at an average dosing of 3.0 mL/day for 11 days over a 23 day period. At the time of testing Patient 214 was a 51 year old male with chronic moderate asthma. The patient nebulized the liquid pharmaceutical composition at an average dosing of 3.0 mL/day for 21 days over a 23 day period. At the time of testing Patient 215 was a 43 year old male with chronic moderate to severe allergenic asthma and other health conditions. The patient nebulized the liquid pharmaceutical composition at an average dosing of 3.0 mL/day for 31 days over a 37 day period. At the time of testing Patient 216 was a 64 year old male with chronic severe allergenic asthma. The patient nebulized the liquid pharmaceutical composition at an average of 6.0 mL/day for a 20 day period. At the time of testing Patient 217 was a 16 year old female with exercise induced asthma and in otherwise excellent health. The patient nebulized the liquid pharmaceutical composition at an average of 6.0 mL/day for a 9 day period. At the time of testing Patient 218 was a 49 year old female with period melanoma and in otherwise excellent health. The patient nebulized the liquid pharmaceutical composition at an average dosing of 6.0 mL/day for a 9 day period. At the time of testing Patient 219 was a 51 year old male in good health. The patient nebulized the liquid pharmaceutical composition at an average dosing of 6.0 mL/day for a 9 day period. With the exception of patient 216, all patients used vibrating mesh nebulizers for aerosolization of the pharmaceutical composition disclosed in Table A2. Patient 216 used a jet nebulizer.

Before nebulization inhalation of the pharmaceutical composition patients went to various hospitals, clinics or medical offices to have various blood tests conducted. Following nebulization inhalation, the patient's blood test samples were conducted generally one day following the inhalation period. None of the patients reported any adverse reactions to inhalation of the nebulized liquid composition, other than one individual coughed a few times for about 1 minute or less following inhalation at the very end of the nebulization period.

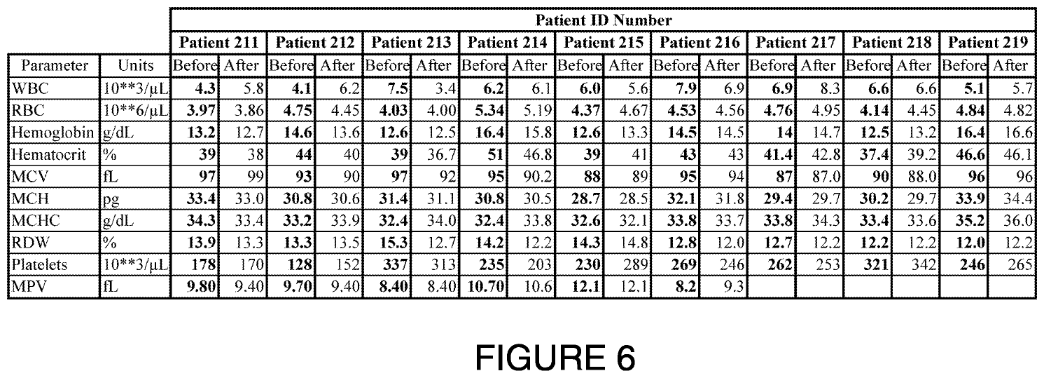

Complete blood count (CBC) parameters were analyzed for all patients before and after the inhalation period. With the exception of patients 217, 218 and 219 who did not have mean platelet volume reported, all CBC parameters were measured before and after the inhalation period as reported in FIG. 6. Comparisons of the test results were made to the US FDA Toxicity Grading Scale table for laboratory abnormalities and no clinical abnormalities were reported for any of the patients either before or after nebulization inhalation (USFDA, 2007). Of note patient 212 had an increase in platelets from below normal at 128 (10³/μL) prior to the inhalation period to 152 (10³/μL) one day following the inhalation period, which was in the normal range. All other patients had platelet concentrations within normal ranges with 5 other exhibiting slight decreases and 3 others exhibiting slight decreases. Patient 212 also had in had an increase in white blood cell counts from below normal prior to the inhalation period at 4.3 (10³/μL) to 5.8 (10³/μL) following the inhalation period, which was in the normal range. All other patients had white blood cell concentrations within normal ranges with 5 other exhibiting slight increases, 2 with no change and 2 others exhibiting decreases following nebulization. All other parameters for all patients had variabilities within ranges typical of temporal changes. Some parameters were slightly below or above normal before nebulization inhalation and similarly some parameters were slightly below or above normal ranges following nebulization inhalation.

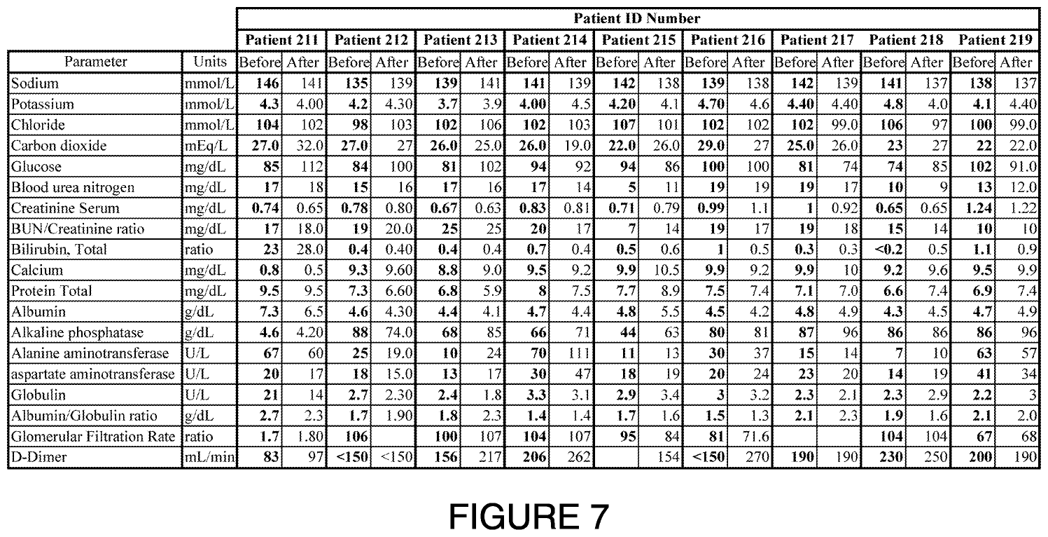

Comprehensive metabolic panel (CMP) parameters were analyzed for all patients before and after the inhalation period are reported in FIG. 7. With respect to liver function, alkaline phosphatase (ALP), alanine aminotransferase (ALT), aspartate aminotransferase (AST) and bilirubin are parameters in the CMP used for assessment. Patients 211, 212, 213, 215, 216, 217, and 218 had these parameters reported in normal ranges both before and after the nebulization inhalation period. Patient 214 had an above normal ALT result prior to the nebulization inhalation period and after as well, with an increase from 70 g/dL to 114 g/dL measured one day following the inhalation period. Patient 214 also had a normal AST value before the nebulization inhalation period of 30 U/L to an above normal value of 47 U/L following treatment. The normal ranges of ALT values is 7-52 U/L. The normal ranges of AST values is 11-39 U/L. According to USFDA (2007), patient 214 ALT values before and after nebulization inhalation were Mild Grade 1 and their AST value was Mild Grade 1 following treatment. Patient 214 reportedly consumes about 1,500 mg/day on NSAIDs which could also affect liver function values and the patient had been periodically fasting during the period when the initial testing occurred. There were no other clinical abnormalities for patient 214 with respect to liver functioning. Patient 219 had an above normal ALT result prior to the nebulization inhalation period and after as well, with a decrease reported from 63 g/dL to 47 g/dL. Patient 219 also had an above normal AST value before the nebulization inhalation period of 41 U/L to a below normal value of 34 U/L measured one day following the inhalation period. The normal ranges of ALT values is 0-44 U/L. The normal ranges of AST values is 0-40 U/L. According to USFDA (2007), patient 219's ALT value before nebulization inhalation was Mild Grade 1 and normal following inhalation treatment and their AST value was normal both before and after inhalation therapy. With respect to kidney functioning, blood urea nitrogen (BUN) and creatinine are representative CMP parameters, all patients had reported BUN and creatinine levels in the normal range before and after nebulizer inhalation, with the exception of patient 215 with a reported below normal BUN level of 5 mg/dL (normal range is 6 to 24 mg/dL) measured before the inhalation period and a normal value of 11 mg/dL following treatment. Patient 217 had a reported above normal BUN level of 19 mg/dL (normal range is 5 to 18 mg/dL) measured before the inhalation period and a normal value of 17 mg/dL following treatment.

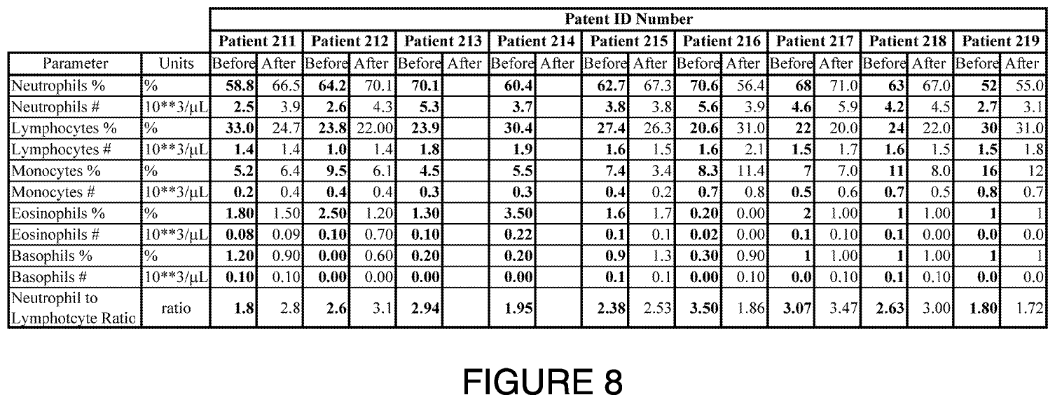

Automated differential analyses of white blood cells types were conducted before and after the inhalation period on 7 of the 9 patients as reported in FIG. 8 of the composition disclosed in Table A2 made without sodium bicarbonate, sodium hydroxide, or a preservative and was manufactured without using any nitrogen purging. Because of laboratory errors, automated differential analyses were not accurate following the inhalation period for blood tests taken from patients 213 and 214. None of the 7 patients had any reported values of automated differential parameters outside of accepted ranges of values. A comparison was made to the reported number of lymphocytes, neutrophils and eosinophils for each of the 7 patients with USFDA (2007) laboratory abnormality values of these parameters. None of the number of lymphocytes, neutrophils and eosinophils reported for the 7 patients reflect any laboratory abnormalities of these parameters compared to the USFDA (2007) laboratory abnormality values. In all cases the number of neutrophils and lymphocytes either remained the same or slightly increased or decreased following the inhalation period, with the exception of neutrophils which decreased in patient 216 from 5.6 10³/μL before the inhalation period to 3.9 10³/μL following the inhalation period and for the number of lymphocytes in patient 218 which decreased from 1.6 10³/μL before the inhalation period to 1.5 10³/μL following the inhalation period. Of the 7 patients who had automated differential tests conducted before and after nebulization inhalation period, 5 patients had steady or increased lymphocyte counts, with an average increase of 0.28 10³/μL and 2 patients had an average decrease of 0.2 10³/μL.

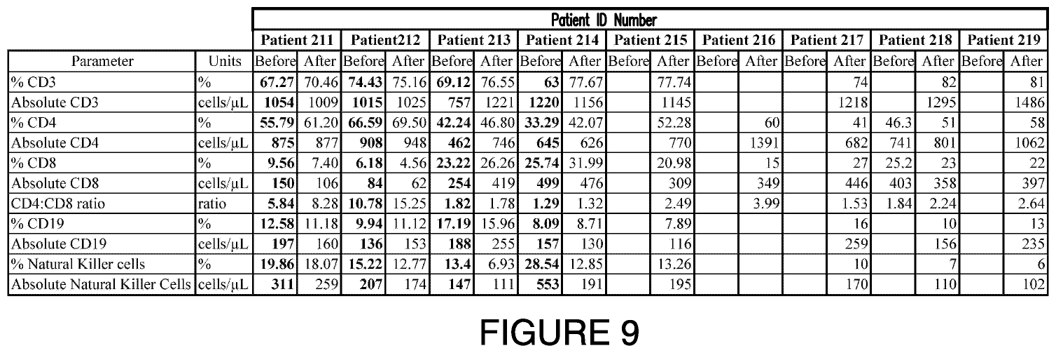

Analysis of lymphocyte subsets is an important indicator of the detection of cell immunity as well as humoral immune status and it reflects the immune function, and its homeostatic level on the whole. Lymphocyte subset analyses was conducted before and after the nebulization inhalation period on 5 of the 9 patients and for 4 of the 9 patients, only following the nebulization inhalation period. Lymphocyte subset blood test results are reported in FIG. 9. Interpretation of lymphocyte subset analyses is complex as differences in immune system responses in humans is variable given the variability of individual innate and adaptive immune systems responses to diseases and other stimuli. CD4⁺ T-cells are considered “helper” cells because they do not neutralize infections but rather trigger the body's response to infections. Absolute CD4⁺ T-cell counts increased following inhalation therapy in 4 of the 5 patient tested with an average increase of 34 cells/μL and decreased in 1 patient by 19 cells/μL. All CD4⁺ T-cell counts in the 5 patients test before and after the nebulization period were in the normal range and 3 of the additional patients tested only following the nebulization period were also in the normal range. The % CD4 T-cells increased in all 5 of the patients tested following the nebulization inhalation period by an average of 5.3% compared to results prior to the inhalation period. Absolute CD8⁺ cells counts decreased in 4 of 5 patients by an average of 33 cells/μL and increased in 1 patient by 165 cells/μL. CD8⁺ cell counts were below normal before and after the inhalation period in patients 211 and 212 and in the normal range for the other 5 patients following the inhalation period. The percent CD8⁺ decreased by an average of 2.0% in 3 of 5 patients and increased by an average of 4.7%. The CD4/CD8 ratio increased in 4 of 5 patients by an average of 1.83 and decreased in 1 patient by 0.04. Absolute killer cells were tested in 4 of the 9 patients an all decreased by an average of 121 cells/4, and percent absolute killer cells decreased by 12.2%. Glucocorticoid compounds are well known to have an inhibitory effect on natural killer cell functions (Nair et al. 1984). Additionally the production of IFN-γ, a key natural killer cell cytokine, is inhibited by glucocorticoids.

### INCORPORATION BY REFERENCE

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the compositions and methods of the present invention, where the term comprises is used with respect to the compositions or recited steps of the methods, it is also contemplated that the compositions and methods consist essentially of, or consist of, the recited compositions or steps or components. Furthermore, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

Furthermore, it should be recognized that in certain instances a composition can be described as being composed of the components prior to mixing, or prior to a further processing step such as drying, binder removal, heating, sintering, etc. It is recognized that certain components can further react or be transformed into new materials.

All percentages and ratios used herein are on a volume (volume/volume) or weight (weight/weight) basis as shown, or otherwise indicated.

Many compounds and/or classes of compounds of the present disclosure may have one or more than one physiological, pharmacological, chemical, or biological role. It can be appreciated that in some instances, a compound and/or class of compounds may be referred to by a specific role while inherently having characteristics of one or more other roles. In other instances, a compound and/or class of compounds may be included in a composition comprising one or more other compounds and/or class of compounds, each having separate roles, each overlapping but distinct roles, or each having similar or identical roles. It can be appreciated that a given compound and/or class of compounds may have a predominantly different role in different compositions depending upon several factors including the chemical environment, additives, other components of the composition, dilution, environmental factors, and the like. Referring to a compound or class of compounds may not necessarily limit the role of that compound and it can be appreciated that a person skilled in the art would be able to ascertain an appropriate understanding of the role of a given compound or class of compound in a given composition. Some non-limiting roles of compounds and classes of compounds may include plant extract antibacterial, antibacterial, antiviral, plant extract antioxidant, antioxidant, TRPA1 antagonist, mucolytic, chelating or chelating agent, cannabinoid type 2 (CB2) receptor agonist, anti-inflammatory, amino acid, thiol amino acid, vitamin, carrier, lubricating, emulsifying, pH adjusting, preservative, viscosity-increasing, and any other physiological, pharmacological, chemical, and/or biological roles.

### REFERENCES

Agency for Toxic Substances & Disease Registry (ATSDR) (2012). Toxicological Profile for Cadmium. Atlanta, GA, U.S. Department of Health and Human Services, Public Health Service.
Allocati, N., Masulli, M., Di Ilio C., and Federici, L. (2018). Glutathione transferases: substrates, inhibitors and pro-drugs in cancer and neurodegenerative diseases. Oncogenesis, 7:8 DOI 10.1038/s41389-017 0025-3.
Alderman S. L., Song C., Moldoveanu S. C., and Cole, C. K. (2014). Particle size distribution of e-cigarette aerosols and the relationship to Cambridge filter pad collection efficiency. Beiträge Zur Tabakforsch Intl/Contrib Tob Res 26:183-90.
American Association for Respiratory Care. (2017). A Guide to Aerosol Delivery Devices for Respiratory Therapists, 4th Edition.
American Thoracic Society. Standardization of spirometry, 1994 update. (1995). Am J Respir Crit Care Med. 152: 1107-1136.
Ames, B. N.; Gold, L. S.; Willett, W. C. (1995). The causes and prevention of cancer. Proc. Natl. Acad. Sci. USA. 92, 5258-5265.
Astani, A, Reichling J, Schnitzler P. (2009). Screening for antiviral activities of isolated compounds from essential oils. Evidence Based Complementary Alternative Medicine, Vol. 2011, Article ID 253643.
Atkuri, K. R., Mantovani, J. J., Herzenberg, L. A., and Herzenberg, L. A. (2007). N-Acetylcysteine—a safe antidote for cysteine/glutathione deficiency. Curr Opin Pharmacol. 7(4):355-9.
Barnes, P. J. (2001). Th2 cytokines and asthma: an introduction. Respir Res. 2001; 2(2): 64-65.
Barnes, P. J. (2004). Mediator of Chronic Obstructive Pulmonary Disease. Pharmacol Rev. 56:515-48.
Barnes, P. J. (2008). The cytokine network in asthma and chronic obstructive pulmonary disease. J Clin Invest. 2008; 118(11):3546-3556.
Beigel J H, Farrar J, Han A M, et al. Avian influenza A (H5N1) infection in humans [published correction appears in N Engl J Med. 2006 Feb. 23; 354(8):884]. N Engl J Med. 2005. 353(13):1374-1385.
Benowitz, N. L., Jacob, P. (1984). Daily intake of nicotine during cigarette smoking. Clinical Pharmacology and Therapeutics. 35: 499-504.
Berthon, G. (1995). Critical evaluation of the stability constants of metal complexes of amino acids with polar side chains (Technical Report). Pure Appl. Chem. 67 (7): 1117-1240.
Bessac, B. F., Sivula, M., von Hehn, C. A., Escalera, J., Cohn, L., and Jordt S. E. (2008). TRPA1 is a major oxidant sensor in murine airway sensory neurons. J. Clin. Invest. 118 1899-1910.
Bhatt, S. P., Kim, Y., Harrington, K. F., et al. (2018). Smoking duration alone provides stronger risk estimates of chronic obstructive pulmonary disease than pack-years. Thorax. 73:414-421.
Birdsall, T. C (1998). Therapeutic applications of taurine. Altern Med Rev. 3:128-136.
Birrell, M. A., McCluskie, K., Wong, S, et al. (2005). Resveratrol, an extract of red wine, inhibits lipopolysaccharide induced airway neutrophilia and inflammatory mediators through an Nf-kappaB-independent mechanism. FASEB. 19:840-841.
Birrell, MA, Belvisi, MG, Grace, M, et al. TRPA1 agonists evoke coughing in guinea pig and human volunteers. Am J Respir Crit Care Med. 2009; 180:1042e7.
Bouayed J, Bohn T. (2010). Exogenous antioxidants-Double-edged words in cellular redox state. Oxid Med Cell Longev 3:228-37.
Bridgeman M. M., Marsden, M., MacNee, W., Flenley, D. C., and Ryle, A. P. (1991). Cysteine and glutathione concentrations in plasma and bronchoalveolar lavage fluid after treatment with N-acetylcysteine. Thorax. 46: 39-42.
Bucca, C., G. Rolla, W. Arossa, E. Carta, F. Nebiolo & S. Baldi. (1989). Effect of ascorbic acid on increased bronchial responsiveness during upper airway infection. Respiration 55: 214-219.
Bucca, C., Rolla, G., Oliva, A., and Farina, J. C. (1990). Effect of vitamin C on histamine bronchial responsiveness of patients with allergic rhinitis. Ann. Allergy 65: 311-314.
Bucca, C., G. Rolla, A. Oliva, J. C. Farina. (1992). Effect of Vitamin C on Transient Increase of Bronchial Responsiveness in Conditions Affecting the Airways. in: Beyond Deficiency: New Views on the Function and Health Effects of Vitamins, Annals of the New York Academy of Sciences. Volume 669, Issue 1.
Bucca, C., G. Rolla, A. Oliva, M. Bugiani, W. Arossa, J. C. Farina & E. Concina. (1989). Effect of vitamin C on lung function changes induced by urban smog. XVI World Congress on Diseases of the Chest, 1989. Abstracts Book, p. 270S.
Bucca, C., G. Rolla, E. Carta, W. Arossa & M. Bugiani. (1989). Effects of vitamin C on airway responsiveness to inhaled histamine in heavy smokers. Eur. Respir. J. 2: 229-233.
Buhl, R., Vogelmeier, C., Crittenden, M., Hubbard, R. C., Hoyt, R. F., Wilson, E. M., Cantin, A. M. and Crystal, R. G. (1990). Augmentation of glutathione in the fluid lining the epithelium of the lower respiratory tract by directly administering glutathione aerosol. Proc Natl Acad Sci USA 1990; 87:4603-4607.
Bump, E. A., and Brown, J. M. (1990). Role of glutathione in the radiation response of mammalian cells in vitro and in vivo. Pharmacol. Ther. 47, 117-136.
Cantin, A. M., North, S. L., Hubbard, R. C. (1987). Crystal R G. Normal alveolar epithelial lining fluid contains high levels of glutathione. J Appl Physiol. 63:152-7.
Cantin, A. M. (1994). Taurine modulation of hypochlorous acid-induced lung epithelial cell injury in vitro. Role of anion transport. J Clin Investig., 93: 606-614.
Carr, T. F., Zeki, A. A., and Kraft, M. (2018). Eosinophilic and non-eosinophilic asthma. Am J Respir Crit Care Med. 197:22-37.
Centers for Disease Control and Prevention. Current Cigarette Smoking Among Adults—United States, 2005-2016. (2018). Morbidity and Mortality Weekly Report. 67(2):53-9.
Centers for Disease Control and Prevention. (2020). Coronavirus 2019 (COVID-19). https://www.cdc.gov/coronavirus/2019-ncov/hcp/clinical-guidance-management-patients.html (accessed Apr. 13, 2020).
Chan, E. D., Riches, D. W., and White, C. W. (2001). Redox paradox: effect of N-acetylcysteine and serum on oxidation reduction-sensitive mitogen-activated protein kinase signaling pathways, Am. J. Respir. Cell. Mol. Biol. vol. 24, pp. 627-632.
Chan, K. H., Ho, S. P., Yeung, S. C., So, W. H., Cho, C. H., and Koo, M. W. (2009). Chinese green tea ameliorates lung injury in cigarette smoke-exposed rats. Respir Med. 103:1746-1754.
Chan, K. H., Chan, S. C., Yeung, S. C., Man, R. Y., Ip, M. S., and Mak, J. C. (2012) Inhibitory effect of Chinese green tea on cigarette smoke-induced up-regulation of airway neutrophil elastase and matrix metalloproteinase-12 via antioxidant activity. Free Radic Res. 46:1123-1129.
Channappanavar R, Perlman S. Pathogenic human coronavirus infections: causes and consequences of cytokine storm and immunopathology. Semin Immunopathol. 2017. 39(5):529-539.
Chen L, Liu H G, Liu W, Liu J, Liu K, Shang J, Deng Y, Wei S. [Analysis of clinical features of 29 patients with 2019 novel coronavirus pneumonia]. Zhonghua Jie He He Hu Xi Za Zhi. 2020 Feb. 6; 43(0):E005.
Cheung C Y, Poon L L, Ng I H, et al. Cytokine responses in severe acute respiratory syndrome coronavirus-infected macrophages in vitro: possible relevance to pathogenesis. J Virol. 2005. 79(12):7819-7826.
Chow C W, Herrera Abreu M T, Suzuki T, Downey G P. Oxidative stress and acute lung injury. Am J Respir Cell Mol Biol. 2003. 29 (4):427-431.
Chukwuma C I, Islam S. Xylitol Improves Anti-Oxidative Defense System In Serum, Liver, Heart, Kidney And Pancreas Of Normal And Type 2 Diabetes Model Of Rats. Acta Pol Pharm. 2017 May; 74(3):817-826. PMID: 29513951.
Chung, Kyung-Sook; Hong, Joo Young; Lee, Jeong-Hun; Lee, Hae-Jun; Park, Ji Yeon; Choi, Jung-Hye; Park, Hee-Juhn; Hong, Jongki; Lee and Kyung-Tae. (2019). β-Caryophyllene in the Essential Oil from Chrysanthemum boreale Induces G1 Phase Cell Cycle Arrest in Human Lung Cancer Cells. Molecules, 24, 3754
Conway, J. G., Neptun, D. A., Garvey, L. K., and Popp, J. A. (1987). Carcinogen treatment increases glutathione hydrolysis by gamma-glutamyl transpeptidase. Carcinogenesis. 8, 999-1004.
Crook, T. R., Souhami, R. L., Whyman, G. D., and McLean, A. E. (1986). Glutathione depletion as a determinant of sensitivity of human leukemia cells to cyclophosphamide. Cancer Res. 46, 5035-5038.
Dahham, S S, Tabana, Y M, Iqbal, M A, Ahamed, M B, Ezzat, M O, Majid, A S, Majid, A M. (2015). The anticancer, antioxidant and antimicrobial properties of the sesquiterpene β-caryophyllene from the essential oil of Aquilaria crassna. Molecules, 20, 11808-11829.
De Backer, W., van Overveld, F., and Vandekerckhove, K. (1997). Sputum ECP levels in COPD patients decrease after treatment with N-acetylcysteine (NAC). Eur Respir J. 12:225s.
DeGraff, W. G., Russo, A., and Mitchell, J. B. (1985). Glutathione depletion greatly reduces neocarzinostatin cytotoxicity in Chinese hamster v79 cells. J. Biol. Chem. 260, 8312-8315.
Dekhuijzen, P. N. (2004). Antioxidant properties of N-acetylcysteine: their relevance in relation to chronic obstructive pulmonary disease. Eur Respir J. 23(4):629-636.
EFSA Response Letter, EFSA-Q-2007-113, 2009
EI-Sakkar, M. G., and Barghash, A. A. (2006). Study of the Effects of Thymoquinone and Epigallocatechin Gallate on Cigarette Smoke-Induced Oxidative Stress and Inflammatory Responses in Guinea Pigs. JMRI 27(2): 128-35.
Estrela, J. M., Obrador, E., Navarro, J., Lasso De la Vega, M. C. and Pellicer, J. A. (1995). Elimination of ehrlich tumours by atp-induced growth inhibition, glutathione depletion and x-rays. Nat. Med., 1, 84-88.
Ermis, H., Parlakpinar, H., Gulbas, G., Vardi, N., Polatm A., Cetin, A., et al. (2013). Protective effect of dexpanthenol on bleomycin-induced pulmonary fibrosis in rats. Naunyn Schmiedebergs Arch Pharmacol. 386:1103-10.
Erythropel, H. C., Jabba, S. V., De Winter, T. M., et al. (2018). Formation of flavorant-propylene glycol adducts with novel toxicological properties in chemically unstable e-cigarette liquids. Nicotine Tob Res. 1-11.
Erythropel, H. C., Davis, L. M, and De Winter, T. M., et al. (2019). Flavorant-Solvent Reaction Products and Menthol in JUUL E-Cigarettes and Aerosol. Am J Prev Med 2019; 57(3):425-427
Facchinetti, F., Amadei, F., Geppetti, P., Tarantini, F., Di Serio, C., Dragotto, A., Gigli, P. M., Catinella, S., Civelli, M., and Patacchini, R. (2007). Alpha, beta-unsaturated aldehydes in cigarette smoke release inflammatory mediators from human macrophages. Am J Respir Cell Mol Biol. 37:617-23.
Friedman, M., Mackey, B. E., Kim, H. J., Lee, I. S., Lee, K. R., Lee, S. U., Kozukue, E., and Kozukue, N. (2007). Structure-activity relationships of tea compounds against human cancer cells. J Agric Food Chem 55:243-253.
GBD 2016 Causes of Death Collaborators (2017). Global, regional, and national age-sex specific mortality for 264 causes of death, 1980-2016: a systematic analysis for the global burden of disease study 2016. Lancet. 390, 1151-1210.
Geiler, J, Michaelis, M, Naczk, P, Leutz, A, Langer, K, et al. (2010) N-acetyl-Lcysteine (NAC) inhibits virus replication and expression of pro-inflammatory molecules in A549 cells infected with highly pathogenic H5N1 influenza A virus. Biochem Pharmacol 79: 413-420.
Gilmour, M. I., Jaakkola, M. S., London, S. J., Nel, A. E., Rogers, C. A. (20090. How exposure to environmental tobacco smoke, outdoor air pollutants, and increased pollen burdens influences the incidence of asthma. Environ Health Perspect. 114:627-633.
Go C C, Pandav K, Sanchez-Gonzalez M A, Ferrer G. Potential Role of Xylitol Plus Grapefruit Seed Extract Nasal Spray Solution in COVID-19: Case Series. Cureus. 2020 Nov. 3; 12(11):e11315.
Gould, N. S., Min, E., Gauthier, S., Chu, H. W., Martin, R., and Day, B. J. (2010). Aging adversely affects the cigarette smoke induced glutathione adaptive response in the lung. Am. J. Respir. Crit. Care Med. 182, 1114-1122.
Gould, N. S., Min, E., Gauthier, S., Martin, R. J., and Day, B. J. (2011b). Lung glutathione adaptive responses to cigarette smoke exposure. Respir. Res. 12, 133.
Gould, N. S., Min, E., Huang, J., Chu, H., Good, J., Martin, R., and Day, B. J. (2015). Glutathione Depletion Accelerates Cigarette Smoke-Induced Inflammation and Airspace Enlargement. Toxicological Sciences, 147(2), 466-474.
Graham, A. L., Allen, J. E. & Read, A. F. Evolutionary causes and consequences of immunopathology. Annu. Rev. Ecol. Evol. Sci. 36, 373-397 (2005).
Grandjean, E. M., Berthet, P., Ruffmann, R., and Leuenberger, P. (2000). Efficacy of oral long-term N-acetylcysteine in chronic bronchopulmonary disease: a meta-analysis of published double-blind, placebo controlled clinical trials. Clin Ther. 2000 Feb. 22(2):209-21.
Global Initiative for Chronic Obstructive Lung Disease. (2019). Global strategy for the diagnosis, management, and prevention of chronic obstructive pulmonary disease 2019 report
Hagiwara, S-I., Ishii. Y., and Kitamura, S. (2000) Aerosolized administration of N-acetylcysteine attenuates lung fibrosis induced by bleomycin in mice. Am J Respir Crit Care Med. 162:225-231.
Hamming I, Timens W, Bulthuis M L C, Lely A T, Navis G J, van Goor H (2004) Tissue distribution of ACE2 protein, the functional receptor for SARS coronavirus: a first step in understanding SARS pathogenesis. J Pathol. 203:631-663.
Hankinson, J. L., Odencrantz, J. R. and Fedan, K. B. (1999). Spirometric reference values from a sample of the general US population. Am J Respir Crit Care Med, 159.
Hassan, F., Xu. X., Nuovo, G., et al. (2014). Accumulation of metals in GOLD4 COPD lungs is associated with decreased CFTR levels. Respir Res 2014; 15: 69.
Hoffmann M, Kleine-Weber H, Schroeder S, et al. SARS-CoV-2 cell entry depends on ACE2 and TMPRSS2 and is blocked by a clinically proven protease inhibitor. Cell. 2020; 181(2):271-280.
Holroyd, K J, Buhl, R, Borok, Z, Roum, J H, Bokser, A D, Grimes, G J, Czerski, D, Cantin, A M, Crystal, R G. Correction of glutathione deficiency in the lower respiratory tract of HIV seropositive individuals by glutathione aerosol treatment. Thorax. 48: 985-989, 1993.
Hu X, Ivashkiv L B. Cross-regulation of signaling pathways by interferon-gamma: implications for immune responses and autoimmune diseases. Immunity. 2009. 31(4):539-550.
Huang, C., Wang, Y., Li, X., Ren, L., Zhao, J., Hu, Y., et al. (2020). Clinical features of patients infected with 2019 novel coronavirus in Wuhan, China. Lancet. 395, 497-506.
Hwang, Y. P., Jin, S. W., Choi, J. H., Choi, C. Y., Kim, H. G., Kim, S. J., Kim, Y., Lee, K. J., Chung, Y. C., and Jeong, H. G. (2017). Inhibitory effects of L-theanine on airway inflammation in ovalbumin-induced allergic asthma. Food Chem. Toxicol. 99, 162-169.
Imai Y, Kuba K, Neely G G, et al. Identification of oxidative stress and Toll-like receptor 4 signaling as a key pathway of acute lung injury. Cell. 2008. 133(2):235-249.
Iwasaki A, Medzhitov R. Regulation of adaptive immunity by the innate immune system. Science. 2010. 327(5963):291-295.
Jong, C. J.; Azuma, J.; Schaffer, S. Mechanism underlying the antioxidant activity of taurine: Prevention of mitochondrial oxidant production. Amino Acids 2012, 42, 2223-2232.
C. J. Jong, T. Ito, H. Prentice, J. Y. Wu, S. W. Schaffer, Role of mitochondria and endoplasmic reticulum in taurine-deficiency-mediated apoptosis, Nutrients 9 (8) (2017).
Juergens, U. R., Stober, M., and Vetter H. (1998). Inhibition of cytokine production and arachidonic acid metabolism by eucalyptol (1.8-cineol) in human blood monocytes in vitro. Eur J Med Res. 3: 508-510.
Juergens, U. R., Stober, M., and Vetter H. (1998). Steroid-like inhibition of monocyte arachidonic acid metabolism and IL-1b production by eucalyptol (1.8-cineol) (in German). Atemw-Lungenkrkh. 24: 3-11.
Juergens, U. R., Schmidt-Schilling, L., Kleuver, T., and Vetter, H. (1998). Antiinflammatory effects of eucalyptol (1.8-cineol) in bronchial asthma: inhibition of arachidonic acid metabolism in human blood monocytes ex vivo. Eur J Med Res. 3: 407-412.
Juergens, U. R.; Engelen, T.; Racke, K.; Stoeber, M.; Gillissen, A.; Vetter, H. (2004). Inhibitory activity of 1,8-cineol (eucalyptol) on cytokine production in cultured human lymphocytes and monocytes. Pulm. Pharmacol. Ther. 17, 281-287.
Juergens, U. R.; Engelen, T.; Racke, K.; Stoeber, M.; Gillissen, A.; Vetter, H. (2004). Inhibitory monocytes. Pulm. Pharmacol. Ther. 17, 281-287.
Kalra, J., Chaudhary, A. K., Prasad, K. (1991). Increased production of oxygen free radicals in cigarette smokers. Int J Exp Pathol. 72:1-7.
Kalemci, S., Micili, S. C., Acar, T., et al. (2013). Effectiveness of thymoquinone in the treatment of experimental asthma. Clin Ter. 164(3):155-158.
Kichko, T. I., Kobal, G., and Reeh, P. W. (2015). Cigarette smoke has sensory effects through nicotinic and TRPA1 but not TRPV1 receptors on the isolated mouse Trachea and Larynx. Am. J. Physiol. Lung Cell. Mol. Physiol. 309, 812-820.
Khomich, O., S Kochetkov, B Bartosch, A Ivanov. Redox Biology of Respiratory Viral Infections. Viruses. 2018, 10, 392.
Kim E S, Choe P G, Park W B, Oh H S, Kim E J, Nam E Y, Na S H, Kim M, Song K H, Bang J H, Park S W, Kim H B, Kim N J, Oh M D. Clinical Progression and Cytokine Profiles of Middle East Respiratory Syndrome Coronavirus Infection. J Korean Med Sci. 2016 Nov. 31 (11):1717-1725.
Kinnula, V. L., Paakko, P. and Soini, Y. (2004.) Antioxidant enzymes and redox regulating thiol proteins in malignancies of human lung. FEBS Lett 569, 1-6.
Knudson, R. J., Slatin, R. C., Lebowitz, M. D., et al. (1976). The maximal expiratory flow-volume curve. Normal standards, variability, and effects of age. Am Rev Respir Dis. 113:587-600.
Kozlowski L T, Mehta N Y, Sweeney C T, et al. (1998). Filter ventilation and nicotine content of tobacco in cigarettes from Canada, the United Kingdom, and the United States. Tob Control. 7(4):369-375.
Lacy P, Stow J L. Cytokine release from innate immune cells: association with diverse membrane trafficking pathways. Blood. 2011. 118 (1):9-18.
Lambert, J. D., Sang, S., Yang, C. S. (2008). N-Acetylcysteine enhances the lung cancer inhibitory effect of epigallocatechin-3-gallate and forms a new adduct. Free Radic Biol Med. 44:1069-1074.
Lau S K P, Lau C C Y, Chan K H, et al. Delayed induction of proinflammatory cytokines and suppression of innate antiviral response by the novel Middle East respiratory syndrome coronavirus: implications for pathogenesis and treatment. J Gen Virol. 2013. 94 (Pt 12):2679-2690.
Law H K, Cheung C Y, Ng H Y, et al. Chemokine up-regulation in SARS-coronavirus-infected, monocyte-derived human dendritic cells. Blood. 2005.106 (7):2366-2374.
Lee, C. H., Chen, J. C., Hsiang, C. Y., Wu, S. L., Wu, H. C. and Ho, T. Y. (2007). Berberine suppresses inflammatory agents-induced interleukin-1beta and tumor necrosis factor-alpha productions via the inhibition of IkappaB degradation in human lung cells. Pharmacol. Res. 56, 193-201.
Leonard, S. S., Wang, S., Shi, X., Jordan, B. S., Castranovaa, V., and Dubick, M. A. (2000). Wood smoke particles generate free radicals and cause lipid peroxidation, DNA damage, NFkB activation and TNF-α release in macrophages. Toxicology 150 147-157.
Lew T W, Kwek T K, Tai D, et al. Acute respiratory distress syndrome in critically ill patients with severe acute respiratory syndrome. JAMA. 2003. 290 (3):374-380.
Li, N., Sioutas, C., Cho, A., Schmitz, D., Misra, C., et al. (2009) Ultrafine particulate pollutants induce oxidative stress and mitochondrial damage. Environ Health Perspect. 111:455-460.
Li Q, Estes J D, Schlievert P M, Duan L, Brosnahan A J, Southern P J, Reilly C S, Peterson M L, Schultz-Darken N, Brunner K G, Nephew K R, Pambuccian S, Lifson J D, Carlis J V, Haase A T. Glycerol monolaurate prevents mucosal SIV transmission. Nature. 2009 Apr. 23; 458(7241):1034-8.
Li, Y.; Lai, Y.; Wang, Y.; Liu, N.; Zhang, F.; Xu, P. 1, 8-Cineol Protect Against Influenza-Virus-Induced Pneumonia in Mice. Inflammation. 2016, 39, 1582-1593.
Li D, Zhang L, Zhou J, Chen H. Cigarette smoke extract exposure induces EGFR-TKI resistance in EGFR-mutated NSCLC via mediating Src activation and EMT. Lung Cancer. 2016; 93:35-42.
Li, X., Hongbao Yang, H., Sun, H. et al. (2017). Taurine ameliorates PM-induced emphysema. Proceedings of the National Academy of Sciences November 2017, 114 (45) E9655-E9664.
Liang, Y., Liu, K., Yeung, S., Li, X., Ip, M., and Mak J. (2017). (−)-Epigallocatechin-3-gallate Reduces Cigarette Smoke-Induced Airway Neutrophilic Inflammation and Mucin Hypersecretion in Rats. Front Pharmacol. September 6; 8:618.
Lin, A., Liu, M., Ko, H., Perng, H., Lee, T., and Kou, Y. (2015) Lung Epithelial TRPA1 Transduces the Extracellular ROS into Transcriptional Regulation of Lung Inflammation Induced by Cigarette Smoke: The Role of Influxed Ca²⁺. Mediators of Inflammation, vol. 2015, Article ID 148367.
Linden, M., Wieslander, E., Eklund, A., Larsson, K., Brattsand, R. (1998). Effects of oral N-acetylcysteine on cell content and macrophage function in bronchoalveolar lavage from healthy smokers. Eur Respir J 1: 645-650.
Liu, H., Ren, J., Chen, H., et al. (2014). Resveratrol protects against cigarette smoke induced oxidative damage and pulmonary inflammation. J Biochem Mol Toxicol. 28:465-471.
Liu, L., Poon, R., Chen, L., Frescura, A. M., Montuschi, P., et al. (2009). Acute effects of air pollution on pulmonary function, airway inflammation, and oxidative stress in asthmatic children. Environ. Health Perspect. 117: 668-674.
Liu, Q. Duan, H., Luan, J., Yagasaki, K. and Zhang, G. (2009). Effects of theanine on growth of human lung cancer and leukemia cells as well as migration and invasion of human lung cancer cells. Cytotechnology. 59(3), 211.
Liu, Q.; Zhou, Y. H.; Yang, Z. Q. The cytokine storm of severe influenza and development of immunomodulatory therapy. Cell Mol. Immunol. 2016 January, 13(1), 3-10; doi: 10.1038/cmi.2015.74.
Lloyd, C. M., and Hessel, E. M. (2010). Functions of T Cells in Asthma: More than Just T(H)2 Cells. Nat. Rev. Immunol. 10:838-848.
Marcinkiewicz, J., and Kontny, E. (2014). Taurine and inflammatory diseases. Amino Acids. 46, 7-20.
Marrades, R. M., Roca, J., Barbera, J. A., de Jover, L., MacNee, W., and Rodriguez-Roisin, R. (1997). Nebulized glutathione induces bronchoconstriction in patients with mild asthma. Am J Respir Crit Care. 156:425-30.
Mena, S., Benlloch, M., Ortega, A., Carretero, J., Obrador, E., Asensi, M., Petschen, I., Brown, B. D., and Estrela, J. M. (2007). Bcl-2 and glutathione depletion sensitizes b16 melanoma to combination therapy and eliminates metastatic disease. Clin. Cancer Res. 13, 2658-2666.
Meng, Q, Son, Y, Kipen, H, Laskin, D, Schwander, S., et al. (2017). American Journal of Respiratory and Critical Care Medicine; New York. Vol. 195, 1.
Migliore E, Berti G, Galassi C, Pearce N, Forastiere F, et al. (2009). SIDRIA-2 Collaborative Group. Respiratory symptoms in children living near busy roads and their relationship to vehicular traffic: results of an Italian multicenter study (SIDRIA 2). Environ Health. 8:27.
Min C K, Cheon S, Ha N Y, Sohn K M, Kim Y, Aigerim A, et al. Comparative and kinetic analysis of viral shedding and immunological responses in MERS patients representing a broad spectrum of disease severity. Sci Rep. 2016. 6:25359.
Mitchell, J. B.; Russo, A. (1987). The role of glutathione in radiation and drug induced cytotoxicity. Br. J. Cancer Suppl. 8, 96-104. Cancers. 3, 1300.
Moldeus, P., Berggren, M., and Graffstrom, R. (1985). N-Acetylcysteine protection against the toxicity of cigarette smoke and cigarette smoke condensates in various tissues and cells in vitro. Eur J Respir Dis. 66: Suppl. 139, 123-129.
Moon, H., Kim, M. J., Son, H. J., Kweon, H-J., Kim, J. T., Kim, Y., et al. (2015) Five hTRPA1 Agonists Found in Indigenous Korean Mint, Agastache rugosa. PLoS ONE 10(5): e0127060.
Moore, J. B., and June, C. H. (2020). Cytokine release syndrome in severe COVID-19. Science. 368, 473-474.
Müller, J.; Greiner, J. F.; Zeuner, M.; Brotzmann, V.; Schafermann, J.; Wieters, F.; Widera, D.; Sudho, H.; (2006). 1,8-cineol potentiates IRF3mediated antiviral response in human stem cells and an ex vivo model of rhinosinusitis. Clinical Science.
Mukhopadhyay, I., Gomes, P., Aranake, S., Shetty, M., Karnik, P., Damle, M., Kuruganti, S., Thorat, S., Khairatkar-Joshi, N. (2011) Expression of functional TRPA1 receptor on human lung fibroblast and epithelial cells. J Recept Signal Transduct Res. 31:350-358.
Nair, M. P. & Schwartz, S. A. Immunomodulatory effects of corticosteroids on natural killer and antibody-dependent cellular cytotoxic activities of human lymphocytes. J. Immunol. 132, 2876-2882 (1984).
Narayanan, N.; Nair, D. T. Vitamin B12 May Inhibit RNA-Dependent-RNA Polymerase Activity of nsp12 from the SARS-CoV-2 Virus. Preprints 2020, 2020030347.
Nencioni, L et al. (2003) Influenza A virus replication is dependent on an antioxidant pathway that involves GSH and Bcl-2. FASEB J. 17, 758-760 10.1096/fj 0.02-0508fj e
Nie, Y., Huang, C., Zhong, S., Wortley, M. A., Luo, Y., Luo, W., Xie, Y., Lai, K., Zhong, N., and Nie, Y. (2016). Cigarette smoke extract (CSE) induces transient receptor potential ankyrin 1(TRPA1) expression via activation of HIF1α in A549 cells. Free Radic Biol Med. 99, 498-507.
Ng D L, Al Hosani F, Keating M K, et al. Clinicopathologic, Immunohistochemical, and Ultrastructural Findings of a Fatal Case of Middle East Respiratory Syndrome Coronavirus Infection in the United Arab Emirates, April 2014. Am J Pathol. 2016. 186 (3):652-658.
Obrador, E., Carretero, J., Esteve, J. M., Pellicer, J. A., Pascual, A., Petschen, I., and Estrela, J. M. (2001). Glutamine potentiates tnf-alpha-induced tumor cytotoxicity. Free Radic. Biol. Med. 31, 642-650.
Obrador, E., Carretero, J., Ortega, A., Medina, I., Rodilla, V., Pellicer, J. A., and Estrela, J. M. (2002). Gamma-glutamyl transpeptidase overexpression increases metastatic growth of b16 melanoma cells in the mouse liver. Hepatology. 35, 74-81.
Odewumi, C. O., L. M. Latinwo, M. L. Ruden, V. L. D. Badisa, S. Fils-Aime and R. B. Badisa, (2015). Modulation of cytokines and chemokines expression by NAC in cadmium chloride treated human lung cells. Environ. Toxicol. 31(11): 1612-1616.
Odewumi, C. O., Fils-Aime, S., Badisa, V. L., Latinwo, L. M., Ruden, M. L., Ikediobi, C., and Badisa R. B. (2015). Chemoprotective effect of monoisoamyl 2, 3-dimercaptosuccinate (MiADMS) on cytokines expression in cadmium chloride treated human lung cells. Environ Toxicol. 30(6):704-11.
Omaiye, E. E., McWhirter, K., Luo, W., Pankow, J., and Talbot, P. (2019). High-Nicotine Electronic Cigarette Products: Toxicity of JUUL Fluids and Aerosols Correlates Strongly with Nicotine and Some Flavor Chemical Concentrations. Chemical Research in Toxicology. 32 (6), 1058-1069.
Ortega, A., Carretero, J., Obrador, E., and Estrela, J. M. (2008). Tumoricidal activity of endothelium-derived no and the survival of metastatic cells with high glutathione and bcl-2 levels. Nitric. Oxide. 19, 107-114.
Ortega, A. L., Mena, S., Estrela, J. M. (2011). Glutathione in cancer cell death. Cancers. 3:1285.
Osterholm, M. T. Preparing for the Next Pandemic. N Engl J Med 2005; 352:1839-1842.
Projan, S. J., Brown-Skrobot, S., Schlievert, P. M., Vandenesch, F. & Novick, R. P. Glycerol monolaurate inhibits the production of beta-lactamase, toxic shock toxin-I, and other staphylococcal exoproteins by interfering with signal transduction. Journal of bacteriology 176, 4204-4209 (1994).
Prousky, J. (2007). The treatment of pulmonary diseases and respiratory-related conditions with inhaled (nebulized or aerosolized) glutathione. Evidence Based Complementary and Alternative Medicine. 5(1) 27-35.
Peace, M. R. et al. Concentration of nicotine and glycols in 27 electronic cigarette formulations. J Anal Toxicol. 40, 403-7 (2016).
Prousky, J. (2007). The treatment of pulmonary diseases and respiratory-related conditions with inhaled (nebulized or aerosolized) glutathione, Evidence Based Complementary and Alternative Medicine. 5(1) 27-35.
Qin C, Zhou L, Hu Z, et al. Dysregulation of Immune Response in Patients With Coronavirus 2019 (COVID-19) in Wuhan, China. Clin Infect Dis. 2020.71 (15):762-768.
Rahman, I., and MacNee, W. (1999). Lung glutathione and oxidative stress: implications in cigarette smoke-induced airway disease. Am J Physiol 277 (6 Pt 1):L1067-88.
Ramani, S., Raspor, B. and Arbneshi, T. (2013). Electrochemical Study of Cadmium (II) Complexation with Cysteine. American Journal of Analytical Chemistry. Vol. 4 No. 10.
Rhoden, C. R., Lawrence, J., Godleski, J. J., and Gonzalez-Flecha. B. (2004). N-acetylcysteine prevents lung inflammation after short-term inhalation exposure to concentrated ambient particles. Toxicol Sci 0.79: 296-303.
Ribeiro, A., Ferraz-de-Paula, V., Pinheiro, M. L., Vitoretti, L. B., Mariano-Souza, D. P., Quinteiro-Filho, W. M., et al. (2012). Cannabidiol, a non-psychotropic plant-derived cannabinoid, decreases inflammation in a murine model of acute lung injury: role for the adenosine A(2A) receptor. Eur J Pharmacol. 678:78-85.
Richter, P. A., Bishop, E. E., Wang, J., and Swahn, M. H. (2009). Tobacco smoke exposure and levels of urinary metals in the U.S. youth and adult population: the National Health and Nutrition Examination Survey (NHANES) 1999-2004. Int J Environ Res Public Health. 6: 1930-46.
Rusznak, C., Mills, P. R., Devalia, J. L., Sapsford, R. J., Davies, R. J., Lozewicz, S. (2000)/Effect of cigarette smoke on the permeability and IL-1beta and sICAM-1 release from cultured human bronchial epithelial cells of never-smokers, smokers, and patients with chronic obstructive pulmonary disease. Am J Respir Cell Mol Biol. 23:530-536.
Sahiner, U. M., Birben, E., Erzurum, S. Sackesen, C. and Kalayci, O. (2011), Oxidative stress in asthma. World Allergy Organization Journal, 4:151-8.
Sahu, S. K., Tiwari, M., Bhangare, R. C., and Pandit, G. G. (2013). Particle Size Distribution of Mainstream and Exhaled Cigarette Smoke and Predictive Deposition in Human Respiratory Tract. Aerosol Air Qual Res. 13:324-32.
Santini, M. T., Straface, E., Cipri, A., Peverini, M., Santulli, M., and Malorni, W. (1997). Structural alterations in erythrocytes from patients with chronic obstructive pulmonary disease. Haemostasis. 27: 201-210.
Schaefer, E. A., Stohr, S., Meister, M., Aigner, A., Gudermann, T. and Buech, T. R. (2013). Stimulation of the chemosensory TRPA1 cation channel by volatile toxic substances promotes cell survival of small cell lung cancer cells. Biochemical pharmacology. 85(3):426-438.
Schlievert, P. M., Deringer, J. R., Kim, M. H., Projan, S. J. & Novick, R. P. Effect of glycerol monolaurate on bacterial growth and toxin production. Antimicrobial agents and chemotherapy 36, 626-631 (1992).
Schmid, G., Li, B. E., Straface, E., et al. (2002). N-acetylcysteine (NAC) counteracts erythrocyte damage and is useful in the management of COPD. Am J Respir Crit Care Med. 165: A227.
Selin L K, Varga S M, Wong I C, Welsh R M. Protective heterologous antiviral immunity and enhanced immunopathogenesis mediated by memory T cell populations. J Exp Med. 1998; 188(9):1705-1715.
Shinya K, Gao Y, Cilloniz C, Suzuki Y, Fujie M, Deng G et al. Integrated clinical, pathologic, virologic, and transcriptomic analysis of H5N1 influenza virus-induced viral pneumonia in the rhesus macaque. J Virol. 2012; 86: 6055-6066.
Siddiqi, H. K., & Mehra, M. R. (2020). COVID-19 illness in native and immunosuppressed states: A clinical-therapeutic staging proposal. The Journal of heart and lung transplantation: the official publication of the International Society for Heart Transplantation, 39(5), 405-407.
Straface, E., Matarrese, P., Gambardella, L. et al. (2000). N-acetylcysteine counteracts erythrocyte alterations occurring in chronic obstructive pulmonary disease,” Biochemical and Biophysical Research Communications, vol. 279, no. 2, pp. 552-556.
Sundblad, B. M., Ji, J., Levänen, B., et al. (2016). Extracellular cadmium in the bronchoalveolar space of long-term tobacco smokers with and without COPD and its association with inflammation. Int J Chron Obstruct Pulmon Dis. 11: 1005-1013.
Sungnak W, Huang N, Becavin C, et al; HCA Lung Biological Network. SARS-CoV-2 entry factors are highly expressed in nasal epithelial cells together with innate immune genes. Nat Med. 2020; 26(5) 681-687.
Takaishi, M, Fujita, F, Uchida, K, Yamamoto, S, Sawada, Shimizu M, Hatai, Uotsu C, Shimizu, M, Tominaga, M (2012). 1,8-cineole, a TRPM8 agonist, is a novel natural antagonist of human TRPA1. Mol Pain 8:86.
Takaishi, M, Uchida, K, Fujita, F, Tominaga, M (2014). Inhibitory effects of monoterpenes on human TRPA1 and the structural basis of their activity. J Physiol Sci 64:47-57.
Tisoncik J R, Korth M J, Simmons C P, Farrar J, Martin T R, Katze M G. Into the eye of the cytokine storm. Microbiol Mol Biol Rev. 2012; 76: 16-32.
Tumpey T M, Basler C F, Aguilar P V, et al. Characterization of the reconstructed 1918 Spanish influenza pandemic virus. Science. 2005.310 (5745):77-80.
Turcotte, C., Blanchet, M. R., Laviolette, M., Flamand, N. (2016). The CB₂ receptor and its role as a regulator of inflammation. Cell Mol Life Sci. 73(23):4449-4470.
U.S. Food and Drug Administration. Guidance for Industry: toxicity grading scale for healthy adult and adolescent volunteers enrolled in preventative vaccine clinical trials; 2007
U.S. Department of Health and Human Services. (2006). The health consequences of involuntary exposure to tobacco smoke: a report of the surgeon general; U.S. Department of Health and Human Services, Centers for Disease Control and Prevention, National Center for Chronic Disease Prevention and Health Promotion, Office on Smoking and Health: Atlanta, GA, USA.
U.S. Department of Health and Human Services. (2014). The Health Consequences of Smoking-50 Years of Progress: A Report of the Surgeon General. Atlanta: U.S. Department of Health and Human Services, Centers for Disease Control and Prevention, National Center for Chronic Disease Prevention and Health Promotion, Office on Smoking and Health. 165: A227.
Wang, L., Qu, G., Gao, Y., Su, L., Ye, Q., Jiang, F., Zhao,′B., and Miao, J. B. (2018). A small molecule targeting glutathione activates Nrf2 and inhibits cancer cell growth through promoting Keap-1 S-glutathionylation and inducing apoptosis. RSC Adv. 8, 792.
Wang, S., Zhai, C., Zhang, Y., Yu, Y., Zhang, Y., Ma, L., Li, S. and Qiao, Y. et al. (2016). Cardamonin, a Novel Antagonist of hTRPA1 Cation Channel, Reveals Therapeutic Mechanism of Pathological Pain. Molecules. August 29; 21 (9).
Welsh R M, Che J W, Brehm M A, Selin L K. Heterologous immunity between viruses. Immunol Rev. 2010. 235 (1):244-266.
Welsh R M, Selin L K. No one is naive: the significance of heterologous T-cell immunity. Nat Rev Immunol. (2002) 2:417-26.
Wiersinga W J, Rhodes A, Cheng A C, Peacock S J, Prescott H C. Pathophysiology, Transmission, Diagnosis, and Treatment of Coronavirus Disease 2019 (COVID-19): A Review. JAMA. Published online Jul. 10, 2020.
Witschi, A., Reddy, S., Stofer, B., and Lauterburg, B. H. (1992). The systemic availability of oral glutathione. Eur J Clin Pharmacol. 43: 667-9.
Wittmann, M., Petro, W., Kaspar, P., Repges, R., Dethlefsen, U. (1998). Therapy with expectorants: a double-blind randomised study comparing ambroxol and cineol (in German). Atemw-Lungenkrkh. 24: 67-74.
Wong C K, Lam C W, Wu A K, et al. Plasma inflammatory cytokines and chemokines in severe acute respiratory syndrome. Clin Exp Immunol. 2004. 136 (1):95-103.
World Health Organization. (2011). WHO Report on the Global Tobacco Epidemic, 2011. Geneva: World Health Organization.
World Health Organization. (2018) https://www.who.int/respiratory/asthma/en/30
Wu, Y. T., Yen, S. L., Li, C. F. (2016). Overexpression of transient receptor protein cation channel subfamily a member 1, confers an independent prognostic indicator in nasopharyngeal carcinoma. J Cancer. 7(10):1181-1188.
Xu, D., Wan, C., Wang, T., Tian, P., Li, D., Wu, Y., Fan, S., Chen, L., Shen, Y., and Wen, F. (2015). Berberine attenuates cigarette smoke-induced airway inflammation and mucus hypersecretion in mice. Int J Clin Exp Med. 8, 8641-8647.
Xu M L, Wi G R, Kim H J, Kim H J. Ameliorating Effect of Dietary Xylitol on Human Respiratory Syncytial Virus (hRSV) Infection. Biol Pharm Bull. 2016; 39 (4):540-6.
Xu, S., Ma, H., Bo, Y., and Shao, M. (2019). The oncogenic role of CB₂ in the progression of non-small-cell lung cancer. Biomedicine & Pharmacotherapy 117, 109080.
Xu, X., Bishop, E. E., Kennedy, S. M., Simpson, S. A., and Pechacek, T. F. (2014). Annual Healthcare Spending Attributable to Cigarette Smoking: An Update. American Journal of Preventive Medicine. 48(3):326-333.
Wu Z, McGoogan J M. Characteristics of and Important Lessons From the Coronavirus Disease 2019 (COVID-19) Outbreak in China: Summary of a Report of 72314 Cases From the Chinese Center for Disease Control and Prevention. Jama. 2020.
Yadav, N. and Chandra, H. (2017). Suppression of inflammatory and infection responses in lung macrophages by eucalyptus oil and its constituent 1,8-cineole: Role of pattern recognition receptors TREM-1 and NLRP3, the MAP kinase regulator MKP-1, and NFκB. PLoS One. 12:e0188232.
Yang X, Yu Y, Xu J, et al. Clinical course and outcomes of critically ill patients with SARS-CoV-2 pneumonia in Wuhan, China: a single-centered, retrospective, observational study [published correction appears in Lancet Respir Med. 2020.
Yang, Z. et al. Anti-infectious bronchitis virus (IBV) activity of 1,8-cineole: effect on nucleocapsid (N) protein. J Biomol Struct Dyn. 28, 323-330 (2010).
Yao, K. S., Godwin, A. K., Johnson, S., Ozols, R. F., O'Dwyer, P. J., and Hamilton, T. C. (1995). Evidence for altered regulation of gamma-glutamylcysteine synthetase gene expression among cisplatin-sensitive and cisplatin-resistant human ovarian cancer cell lines. Cancer Res. 55, 4367-4374.
Ye Q, Wang B, Mao J. The pathogenesis and treatment of the ‘Cytokine Storm’ in COVID-19. J Infect. 2020. 80 (6):607-613.
Yu Z., Huang, S., Shen, H., Ma, M., Zhu, B., and Zhang, D. (2017). Detection of Glutathione in Oral Squamous Cell Carcinoma Cells With a Fluorescent Probe During the Course of Oxidative Stress and Apoptosis. J Oral Maxillofac Surg 75:223.e1-223.e10.
Zhang M S, Sandouk A, Houtman J C. Glycerol Monolaurate (GML) inhibits human T cell signaling and function by disrupting lipid dynamics. Sci Rep. 2016 Jul. 26; 6:30225.
Zhang, W, Wan, J, Qian, K, Liu, X, Xiao, Z, Sun, J et al. Clinical characteristics of human infection with a novel avian-origin influenza A(H10N8) virus. Chin Med J 2014; 127: 3238-3242.
Zheng, J. P., Kang, J., Huang, S. G., et al. (2008). Effect of carbocisteine on acute exacerbation of chronic obstructive pulmonary disease (PEACE Study): a randomised placebo-controlled study. Lancet, 371:2013.
Zhou, P., Yang, X., Wang, X. et al. A pneumonia outbreak associated with a new coronavirus of probable bat origin. Nature. 579, 270-27 (2020).
U.S. Pat. No. 10,022,303. Inhalable pharmaceutical compositions, Bosch, et al. Jul. 17, 2018.
U.S. Pat. No. 9,968,586. Mast cell stabilizers treatment for systemic disorders, Gerhart, et al. May 15, 2018.
U.S. Pat. No. 9,744,314. Aerosols for sinunasal drug delivery, Keller, et al. Aug. 29, 2017.
U.S. Pat. No. 7,887,859. Method of treating epiphora, Friedlaender, et al. Feb. 15, 2011.
U.S. Pat. No. 8,148,356. Acetylcysteine composition and uses thereof, Pavliv. Apr. 3, 2013.
U.S. Pat. No. 9,216,162. N-acetylcysteine amide (NAC amide) in the treatment of diseases and conditions associated with oxidative stress, Goldstein. Dec. 12, 2015.
U.S. Pat. No. 9,265,749. Methods for the treatment of systemic disorders treatable with mast cell stabilizers, including mast cell related disorders. Gerhart et al. Feb. 23, 2016.
U.S. Pat. No. 9,364,512. Aloe vera based vaping compositions, Drummond, III. Jun. 14, 2016.
U.S. Patent Application 2014/0206765. N-acetylcysteine compositions and methods for the treatment and prevention of cysteine/glutathione deficiency in diseases and conditions, Andrus, et al. Mar. 31, 2005.
U.S. Pat. No. 6,566,401. N-acetylcysteine compositions and methods for the treatment and prevention of drug toxicity, Herzenberg et al. May 20, 2003.
U.S. Pat. No. 9,744,200. System for producing a terpene-enhanced cannabinoid concentrate, Tucker, et al. Aug. 29, 2017.
U.S. Patent Application 2016/0250270. Compositions comprising combinations of purified cannabinoids, with at least one flavonoid, terpene, or mineral, Wendschuh et al. Sep. 1, 2016.
U.S. Patent Application 2017/002797. Cannabinoid composition and method for treating pain, Mukunda et al. Feb. 2, 2017.
U.S. Patent Application 2017/007185. Inhalable nicotine formulations and methods of making and using the same. Stenzler et al. Mar. 16, 2016.

1. A method of treating an infectious viral or bacterial respiratory infection or disease, comprising:
administering to a patient's respiratory tract a therapeutically effective amount of a liquid pharmaceutical composition in an aerosolized or nebulized form, wherein the liquid pharmaceutical composition comprises:
a plant extract comprising one or more Transient Receptor Potential Cation Channel, Subfamily A, member 1 (TRPA1) antagonists;
one or more plant extract antibacterial compounds;
one or more plant extract antiviral compounds; and
one or more plant extract antioxidants.

2. The method of claim 1, wherein the one or more TRPA1 antagonists are each a compound selected from the group consisting of 1,8-cineole, borneol, camphor, 2-methylisoborneol, fenchyl alcohol, cardamonin, and combinations thereof.

3. The method of claim 2, wherein the TRPA1 antagonist is 1,8-cineole.

4. The method of claim 1, wherein the one or more plant extract antibacterial compounds are each selected from the group consisting of β-caryophyllene, geraniol, thymol, glycerol monolaurate, xylitol, an alkylamide, and combinations thereof.

5. The method of claim 1, wherein the one or more plant extract antiviral compounds are each selected from the group consisting of β-caryophyllene, 1,8-cineole, glutathione, glycerol monolaurate, N-acetyl cysteine, thymoquinone, xylitol and combinations thereof.

6. The method of claim 1 wherein the one or more plant extract antioxidants are each selected from the group consisting of berberine, catechin, curcumin, epicatechin, epigallocatechin, epigallocatechin-3-gallate, β-carotene, quercetin, kaempferol, luteolin, ellagic acid, resveratrol, silymarin, nicotinamide adenine dinucleotide, thymoquinone, glutathione, n-acetyl cysteine, xylitol, and combinations thereof.

7. The method of claim 1, wherein the liquid pharmaceutical composition further comprises a mucolytic compound.

8. The method of claim 7, wherein the mucolytic compound comprises one or more compounds each selected from the group consisting of 1,8-cineole, N-acetyl cysteine and combinations thereof.

9. The method of claim 1, wherein the liquid pharmaceutical composition further comprises a chelating agent.

10. The method of claim 9, wherein the chelating agent is glutathione.

11. The method of claim 1, wherein the liquid pharmaceutical composition further comprises a plant extract cannabinoid type 2 (CB2) receptor agonist.

12. The method of claim 11, wherein the CB2 agonist is selected from the group consisting of β-caryophyllene, cannabidiol, and alkylamide compounds, and combinations thereof.

13. The method of claim 1, wherein the liquid pharmaceutical composition further comprises an anti-inflammatory compound.

14. The method of claim 13, wherein the anti-inflammatory compound is selected from the group consisting of 1,8-cineole, cannabidiol, glycerol monolaurate, β-caryophyllene, resveratrol, thymoquinone, curcumin, quercetin, and combinations thereof.

15. The method of claim 1, wherein the liquid pharmaceutical composition further comprises a chelating agent selected from the group consisting of citric acid, ascorbic acid, ethylenediaminetetraacetic acid (EDTA), and combinations thereof.

16. The method of claim 1, wherein the liquid pharmaceutical composition further comprises an amino acid selected from the group consisting of leucine, isoleucine, valine, glutamine, glutamic acid, glycine, arginine, L-theanine, phenylalanine, tryptophan, and combinations thereof.

17-45. (canceled)

46. A liquid pharmaceutical composition, comprising:
0.1 to 5 wt % 1,8-cineole;
0.1 to 2.5 wt % β-caryophyllene;
1.0 to 15 wt. % xylitol;
1.0 to 15 wt. % Polysorbate 20;
0.001 to 0.1 wt. % citric acid;
0.75 to 0.9 wt % sodium chloride; and
38 to 97.05 wt % sterile purified water.

47. The liquid pharmaceutical composition of claim 46, further comprising
0.1 to 5 wt % N-acetyl cysteine; and
0.1 to 5 wt % glutathione.

48. The liquid pharmaceutical composition of claim 46, further comprising:
0.001 to 1 wt % sodium bicarbonate;
0.00001 to 1 wt % L-theanine;
0.00001 to 1 wt % taurine; and
0.05 to 5.0 wt. % glycerol monolaurate.

49. A liquid pharmaceutical composition, comprising:
1.0 wt % 1,8-cineole;
0.5 wt % β-caryophyllene;
5.0 wt. % xylitol;
1.0 wt. % Polysorbate 20;
0.07 wt. % citric acid;
8.23 wt % sodium chloride;
91.5 wt % sterile purified water.

50-52. (canceled)
